(19) United States
(12) Patent Application Publication
(10) Pub. No.: US 2024/0189289 A1
(43) Pub. Date: Jun. 13, 2024

(54) INHIBITORS OF EPHB3 SIGNALING

(71) Applicant: The Brigham and Women`s Hospital, Inc., Boston, MA (US)

(72) Inventors: Francisco J. Quintana, Jamaica Plain, MA (US); Kevin Julian Hodgetts, Framingham, MA (US); Veit Johannes Rothhammer, Schweinfurt (DE); Cristina Gutierrez Vazquez, Madrid (ES)

(21) Appl. No.: 18/278,078

(22) PCT Filed: Mar. 4, 2022
(86) PCT No.: PCT/US2022/018894

§ 371 Date: Aug. 21, 2023

Related U.S. Application Data
(60) Provisional application No. 63/156,700, filed on Mar. 4, 2021.

(51) Int. Cl. A61K 31/437; A61K 31/444; A61K 31/4545; A61K 31/501; A61K 31/506; A61P 25/00

(52) U.S. Cl. CPC A61K 31/437; A61K 31/444; A61K 31/4545; A61K 31/501; A61K 31/506; A61P 25/00

(57) ABSTRACT

The present application provides EphB3 kinase inhibitors, useful intreating neurodegenerative or demyelinating diseases or conditions such as autoimmune encephalomyelitis and multiple sclerosis.

### CLAIM OF PRIORITY

This application claims priority U.S. Patent Application Ser. No. 63/156,700, filed on Mar. 4, 2021, the entire contents of which are hereby incorporated by reference.

### TECHNICAL FIELD

This invention relates to compounds useful in inhibiting Ephrin receptor B3 (“EphB3”) kinase activity, and in particular to heterocyclic compounds useful in treating neurodegenerative diseases such as autoimmune encephalomyelitis (“AE”), multiple sclerosis (“MS”), amyotrophic lateral sclerosis (“ALS”), Alzheimer's disease (“AD”), demyelinating disorders, and other diseases driven by glial activation.

### BACKGROUND

There are numerous deadly diseases affecting current human population. For example, neurodegenerative diseases affect a significant segment of population, especially the elderly. Multiple sclerosis is one of the most common neurodegenerative disorder that affects approximately 2.3 million people world-wide with an estimated socioeconomic burden of more than $1 billion.

### SUMMARY

The cross-talk between astrocytes and microglia controls nervous system (“CNS”) inflammation and neurodegeneration, including central nervous system and (“CNS”) and peripheral nervous system (“PNS”). The present disclosure shows that Eph receptor signaling participates in bi-directional microglia-astrocyte communication to promote CNS pathology in various neuronal diseases, including AE and MS. More specifically, EphB3 receptor signaling in astrocytes activates mTOR, driving pro-inflammatory activities in AE. Moreover, reverse signaling via the EphB3 ligand Ephrin-B3 boosts NF-κB activation and pro-inflammatory activities in microglia. The compounds described herein advantageously penetrate CNS and inhibit EphB3 kinase activity, thereby suppressing astrocyte and microglia cell-contact dependent pro-inflammatory responses and ameliorating acute and chronic AE and MS.

In one general aspect, the present disclosure provides a method of treating a neurodegenerative or a demyelinating disease or condition, the method comprising administering to a subject in need thereof a therapeutically effective amount of a compound of Formula (I):

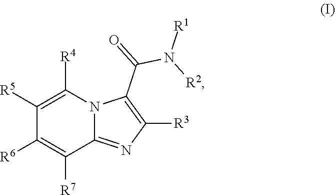

or a pharmaceutically acceptable salt thereof.

In another general aspect, the present disclosure provides a compound of Formula (Ia):

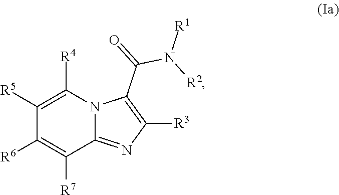

or a pharmaceutically acceptable salt thereof.

In yet another general aspect, the present disclosure provides a compound of Formula (Ib):

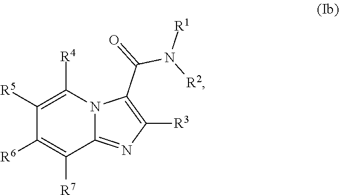

or a pharmaceutically acceptable salt thereof.

In yet another general aspect, the present disclosure provides a compound of Formula (Id):

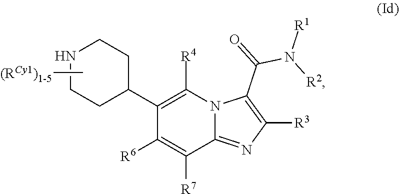

or a pharmaceutically acceptable salt thereof.

In yet another general aspect, the present disclosure provides compound of Formula (Ie):

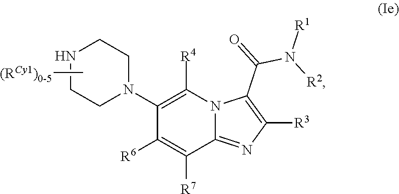

or a pharmaceutically acceptable salt thereof.

In yet another general aspect, the present disclosure provides a compound of Formula (If):

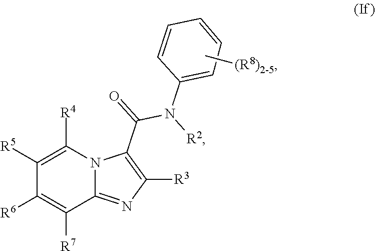

or a pharmaceutically acceptable salt thereof.

In yet another general aspect, the present disclosure provides compound of Formula (Ig):

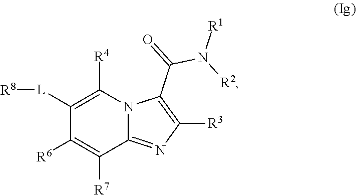

or a pharmaceutically acceptable salt thereof.

In yet another general aspect, the present disclosure provides a compound of Formula (Ih):

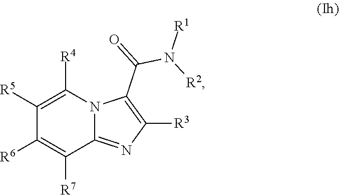

or a pharmaceutically acceptable salt thereof.

In yet another general aspect, the present disclosure provides a compound of Formula (Ii):

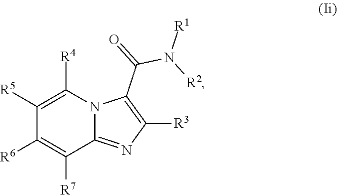

or a pharmaceutically acceptable salt thereof.

In yet another general aspect, the present disclosure provides a method of treating a neurodegenerative disease or condition, the method comprising administering to a subject in need thereof a therapeutically effective amount of a compound of Formula (II):

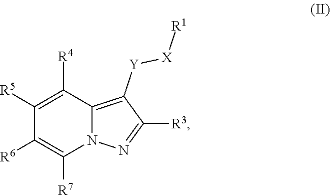

or a pharmaceutically acceptable salt thereof.

In yet another general aspect, the present disclosure provides a compound of Formula (IIa):

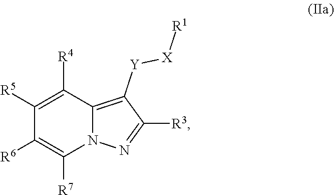

or a pharmaceutically acceptable salt thereof.

In yet another general aspect, the present disclosure provides a method of treating a neurodegenerative or a demyelinating disease or condition, the method comprising administering to a subject in need thereof a therapeutically effective amount of a compound of Formula (III):

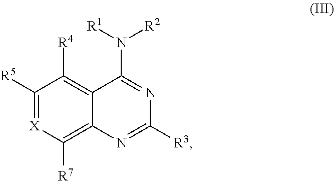

or a pharmaceutically acceptable salt thereof.

In yet another general aspect, the present disclosure provides a compound of Formula (IIIa):

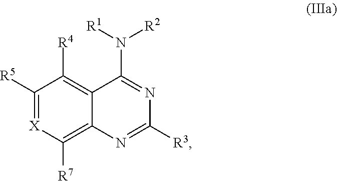

or a pharmaceutically acceptable salt thereof.

In yet another general aspect, the present disclosure provides a pharmaceutical composition comprising a compound as disclosed herein, or a pharmaceutically acceptable salt thereof, and a pharmaceutically acceptable carrier.

In yet another general aspect, the present disclosure provides a method of treating a neuronal system injury characterized by EphB3 kinase activity, the method comprising administering to a subject in need thereof a therapeutically effective amount of a compound of as disclosed herein, or a pharmaceutically acceptable salt thereof, or a pharmaceutical composition comprising same.

In yet another general aspect, the present disclosure provides a method of treating a cancer, the method comprising administering to a subject in need thereof a therapeutically effective amount of a compound of as disclosed herein, or a pharmaceutically acceptable salt thereof, or a pharmaceutical composition comprising same.

In yet another general aspect, the present disclosure provides a method selected from:
inhibiting EphB3 receptor signaling in an astrocyte cell of a subject; and/or
inhibiting EphB3 tyrosine kinase in an astrocyte cell of a subject; and/or
inhibiting pro-inflammatory response in an astrocyte cell of a subject; and/or
inhibiting production of TNF-α in an astrocyte cell of a subject; and/or
reducing production of IL6 in an astrocyte cell of a subject; and/or
reducing production of CCl2 in an astrocyte cell of a subject; and/or
inhibiting phosphorylation of AKT in astrocyte cell of a subject; and/or
inhibiting activation of mTOR pathway in an astrocyte cell of a subject; and/or
inhibiting production of mitochondrial reactive oxygen species (ROS) in an astrocyte cell of a subject; and/or
inhibiting pro-inflammatory response in a microglial cell of a subject; and/or
   restoring a homeostatic state or a normal activation state of glial cell or a neuron cell of a subject; and/or
inhibiting NFkB activation in a microglial cell of a subject; and/or
inhibiting production of TNF-α in a microglial cell of a subject; and/or
inhibiting demyelination of a neuron cell in a subject; and/or
reducing pro-inflammatory T cells in a central nervous system of a subject; and/or
reducing pro-inflammatory macrophages in a central nervous system of a subject; and/or
   reducing pro-inflammatory macrophages in a peripheral nervous system of a subject; and/or
inhibiting CNS inflammation in a subject; and/or
   inhibiting PNS inflammation in a subject; and/or
inhibiting neurodegeneration in a subject,
the method comprising administering to the subject a therapeutically effective amount of a compound as disclosed herein, or a pharmaceutically acceptable salt thereof, or a pharmaceutical composition comprising same.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the present application will be apparent from the following detailed description and figures, and from the claims.

### DESCRIPTION OF DRAWINGS

FIG. 1A shows chemical structure and physicochemical characteristics of compound A38.

FIG. 1B is a bar graph showing lactate dehydrogenase (LDH) release in the culture medium in the presence and absence of compound A38. n=3.

FIG. 1C is a bar graph showing inhibition of EphB3 kinase activity by A38 determined using a cell-free radiometric kinase activity assay n=2. ***P<0.001, **P<0.01, as determined by one-way ANOVA followed by Dunnett's post-hoc test.

FIG. 1D contains line plots and graphs of EphB3 protein expression determined by flow cytometry in neonatal astrocyte-microglia co-cultures where astrocytes are depicted in red and microglia in blue.

FIG. 1E contains images of immunostaining analysis of the co-localization of EphB3 and GFAP⁺ astrocytes.

FIG. 1F contains images of immunostaining analysis of the co-localization of Ephrin-B3 and TMEM119⁺ microglia.

FIG. 1G contains bar graphs of quantification in healthy control (HC) or MS brain tissue in Lesions or Normal Appearing White Matter (NAWM) close to lesion (20-50 m from lesion) and NAWM far from lesion (>500 m). ****P<0.0001, **P<0.01, *P<0.05, ns=not significant as determined by One-way ANOVA followed by Dunnett's or Tukey's post-hoc tests.

FIG. 2A is a bar graph showing 116 mRNA expression determined by qPCR in neonatal murine astrocytes stimulated with TNFα and IL-1β in the presence of the indicated concentrations of A38 or C9 (n=2).

FIG. 2B contains bar graphs showing IL-6, CCL2 and TNFα concentration measured by ELISA in supernatants of neonatal murine astrocytes stimulated as in FIG. 2A with the indicated concentrations of A38 (n=2).

FIG. 2C contains bar graphs of Nos2 and TNFα mRNA expression determined by qPCR in neonatal murine microglia stimulated with LPS in the presence of A38 (n=3). Data are representative of 3 independent experiments.

FIG. 2D shows bar graphs of Csf2, Il6, Ccl2, TNFα and Nos2 mRNA expression determined by qPCR in neonatal murine astrocytes stimulated with TNFα and IL-1β in plates coated with Ephrin-B3-Fc chimera (Fc-EphrinB3).

FIG. 2E shows bar graphs of TL-6, CCL2 and TNFα concentration in supernatants of neonatal murine astrocytes stimulated with TNFα and IL-1β in plates coated FcEphrin-B3. (n=3) Data are representative of two independent experiments.

FIG. 2F is a bar graph showing decrease in neurotoxicity in primary mouse astrocytes that were activated with TNFα and IL-1β and treated with A38 or C9. 24 h later, media was replaced, cells were extensively washed and new medium was added; 48 h later astrocyte-conditioned media (ACM) was collected. ACM was added to the mouse neuron cell line N2A pre-activated with IFNγ and cytotoxicity was determined by quantifying lactate dehydrogenase (LDH) release after 24 h (n=3).

FIG. 2G is a bar graph showing migration assay of splenic CD11b⁺ monocytes performed using ACM (n=2). Data are representative of two independent experiments. ****P<0.0001, ***P<0.001, **P<0.01, *P<0.05 as determined by one-way ANOVA followed by Dunnett's post-hoc test.

FIG. 3A is a diagram of the pseudotyped RV-based approach used. Glycoprotein G (G)-deficient RV, expressing a mCherry was pseudotyped with EnvA to restrict primary infection to astrocytes transgenically expressing the EnvA receptor TVA. Astrocytes co-express G-protein to allow viral replication. Hence, EnvARabΔ-mCherry RV injected into Gfap^{RABVgp4/TVA} mice first infect GFAP⁺ astrocytes (central red cell), where they replicate to then infect cells in contact with the astrocytes infected initially (surrounding cells also depicted in red).

FIG. 3B contains graphs and line plots of mCherry expression in astrocytes determined by flow cytometry.

FIG. 3C show bar graphs of quantification of mCherry colocalization with GFAP⁺ (left panel) and Iba1⁺ cells (right panel) as determined by immunostaining as show in FIG. 3D. ****P<0.0001 as determined by unpaired t test.

FIG. 3D contains images of GFAP, Iba and mCherry immunostaining of naïve (top) or experimental autoimmune encephalomyelitis (“EAE”) (bottom) mice.

FIG. 3E shows a heatmap of differentially expressed genes.

FIG. 3F contains line plots of relevant pathways selected from gene set enrichment analysis (GSEA) of same RNAseq dataset as in FIG. 3E.

FIG. 3G is a bar graph showing relevant pathways selected from ingenuity pathways analysis of the genes differentially expressed genes. Positive z score indicates pathways upregulated in mCherry⁺ compared to mCherry⁻. Data are representative of two independent experiments.

FIG. 3H contains images of electron micrographs of naïve (left) and EAE (right) spinal cords. Microglia (MG) cells exhibit elongated and dark nuclei with clumped chromatin and dark cytoplasm, and astrocytic cells (AS) are characterized by pale nuclei that are usually regular in shape and usually with a thin rim of heterochromatin beneath the nuclear membrane. In naïve spinal cord, microglia are evenly distributed while astrocytes are physically more apart from microglia and surrounded by myelinated fibers and other parts of the neuropil. In EAE spinal cord, the density of both microglia and astrocytes is increased and both cell types establish physical contacts. Green arrow: intact myelin; red arrow: myelin destruction; blue arrow: remyelination.

FIG. 4A contains line plots showing EAE in C57Bl/6J mice injected with lentiviral constructs targeting EphB3 in astrocytes (red), EfnB3 in microglia (blue) or non-targeting control (black). Data are representative of 3 independent experiments. ****P<0.0001, as determined by two-way ANOVA analysis of variance. Ctrl (n=24), pGFAP shEphB3 (n=17), pCD11b shEfnB3 (n=22).

FIG. 4B is a bar graph showing pro-inflammatory monocytes in the CNS of C57Bl/6J mice injected with lentiviral vectors targeting EphB3 in astrocytes (red), EfnB3 in microglia (blue) or non-targeting control (black) (n=5). ****P<0.0001, ***P<0.001, as determined by one-way ANOVA followed by Dunnett's post-hoc test.

FIG. 4C is a bar graph showing relevant pathways selected from ingenuity pathways analysis of genes differentially expressed in astrocytes following EfnB3 knockdown in microglia.

FIG. 4D shows Heatmap of differentially expressed genes in astrocytes following the knockdown of EphB3 in astrocytes.

FIG. 4E shows Heatmap of differentially expressed genes in astrocytes following the knockdown of EfnB3 in microglia.

FIG. 4F shows Heatmap of differentially expressed genes in the microglia following the knockdown of EphB3 in astrocytes.

FIG. 4G shows Heatmap of differentially expressed genes in the microglia following the knockdown of EfnB3 in microglia.

FIG. 4H contains bar graphs of relevant pathways selected from ingenuity pathway analysis of the genes differentially expressed in microglia following EphB3 knockdown in astrocytes (left) and EfnB3 knockdown in microglia (right).

FIG. 4I contains images of acetylated p65 (ac-p65) and Iba1 immunostaining of EAE CNS following administration of control knockdown construct, or constructs targeting EfnB3 in microglia or EphB3 astrocytes.

FIG. 4J is a bar graph showing quantification of Iba1⁺ cells positive for ac-p65 in the three conditions as in FIG. 4F. (n=5) ****P<0.0001, *P<0.05 as determined by one-way ANOVA followed by Tukey's post-hoc test.

FIG. 4K is a bar graph showing astrocytes treated with control or EphB3-targeting siRNA and pre-treated with TNFα and IL-10, were co-cultured ON with microglia, and microglial Nos2 expression was determined by qPCR. (n=2).

FIG. 4L contains bar graphs of Il1b, Il6 and Nos2 mRNA expression determined by qPCR.

FIG. 4M contains bar graphs of IL-6 and CCL2 quantified by ELISA in supernatants (n=4). Data are representative or two independent experiments.****P<0.0001, **P<0.01, *P<0.05 as determined by T test.

FIG. 5A contains line plots showing EAE in C57Bl/6J mice treated twice a day with vehicle or A38 20 mg/kg injected intraperitoneally starting at the peak of the disease (n=5). Data are representative of 2 independent experiments. *P<0.05, as determined by two-way ANOVA analysis of variance.

FIG. 5B is a bar graph showing monocytes in the CNS of C57Bl/6J mice treated as in FIG. 5A. (n=5) **P<0.01, as determined by one-way ANOVA followed by Tukey's post-hoc test.

FIG. 5C shows Heatmap of differentially expressed genes in astrocytes.

FIG. 5D contains line plots of Gene set enrichment analysis (GSEA) of same RNAseq data set.

FIG. 5E contains bar graphs of Ingenuity pathways analysis of genes differentially expressed in astrocytes from A38 treated mice compared to vehicle: Z score.

FIG. 5F contains heatmaps of relevant selected pathways.

FIG. 5G shows a heatmap of differentially expressed genes in the microglia.

FIG. 5H contains line plots of pathways selected from gene set enrichment analysis (GSEA).

FIG. 5I contains bar graphs of Ingenuity pathways analysis of genes differentially expressed in microglia from A38 treated mice compared to vehicle: the Z score.

FIG. 5J contains heatmaps of relevant selected pathways.

FIG. 6A contains line plots of network analysis of the effects of A38 on astrocytes.

FIG. 6B contains images of Western blot analysis of phosphorylated and total protein p85a and p55a PIK3R1 gene products, AKT, S6 and p65 as well as GAPDH (loading control) in murine neonatal astrocytes primary cultures activated for 30 minutes with TNFα and IL-1β in the presence of the indicated compounds A38, Class I PI3K isoforms inhibitor ZSTK74 (Z74) and rapamycin (Rapa).

FIG. 6C contains line plots and bar graphs of analysis of S6 phosphorylation determined by intracellular staining and flow cytometry on astrocytes stimulated as in FIG. 6B.

FIG. 6D contains bar graphs of 16, Ccl2, Tnfa, Csf2 and Nos2 mRNA expression determined by qPCR in neonatal murine astrocytes stimulated or not with TNFα and IL-1β in the presence of A38 or rapamycin (n=4) Data are representative of 3 independent experiments.

FIG. 6E contains line plots of Seahorse Mitochondrial stress test performed on astrocytes pre-treated ON with A38 or rapamycin (Rapa). Data representative of 3 independent experiments.

FIG. 6F shows bar graphs of quantification of basal mitochondrial respiration, maximal mitochondrial respiration and ATP-linked respiration calculated from Mito stress assay from FIG. 6E.

FIG. 6G is a bar graph showing mitochondrial ROS measured by Mitsox staining after ON treatment with A38 or Rapa and TNFα and IL-1β (n=6). Data representative of 3 independent experiments. ****P<0.0001, ***P<0.001, **P<0.01, *P<0.05 as determined by one-way ANOVA followed by Tukey's post-hoc test.

FIG. 7A is a bar graph showing evaluation of apoptosis in cells after treatment with A38 by the analysis of caspase-3/7 activation.

FIG. 7B contains bar graphs of Efnb1, Efnb2 and Efnb3 mRNA expression determined by qPCR in microglia from naïve or EAE mice (n=4) Data are representative of 2 independent experiments. *P<0.05 as determined by unpaired t test.

FIG. 7C is a bar graph of 116 mRNA expression determined by qPCR in neonatal murine astrocytes stimulated with TNFα and IL-1β in the presence of 10 M of C9 (n=2).

FIG. 7D contains bar graphs of IL-6, CCL2 and TNFα concentration measured by ELISA in supernatants of neonatal murine astrocytes stimulated as in FIG. 7B with the indicated concentrations of C9 (n=2).

FIG. 7E contains bar graphs of CSF2, IL6, CCL2, IL1b, NOS2 and TNF mRNA determined by qPCR (n=4).

FIG. 7F contains bar graphs of IL-6, CCL2 and TNFα concentration measured by ELISA in astrocytes supernatants (n=3).

FIG. 7G contains bar graphs showing fetal human astrocytes stimulated with IL-1β and plate-bound human Ephrin-B3-FcChimera (n=4), and CSF2, IL6, CCL2, IL1b, NOS2 and TNF mRNA determined by qPCR. Data are representative or two independent experiments. ****P<0.0001, ***P<0.001, **P<0.01, *P<0.05 as determined by One-way ANOVA followed by Tukey's or Dunnett's post-hoc test.

FIG. 8 contains images of mCherry expression in GFAP⁺ cells (Astrocytes) was analyzed 3 days post injection and is highlighted with white arrowheads. GfapRABVgp4 TVA mice were injected with glycoprotein G-deficient RV expressing the fluorescent protein mCherry and pseudotyped with EnvA.

FIG. 9A contains bar graphs of EphB3 expression in astrocytes (left) and Ephrin-B3 expression in microglia (right) determined by immunofluorescence following treatment with pGFAP-shEphB3, pCD11b-shEfnB3 or control lentiviral vectors. *P<0.05 as determined by Unpaired t test.

FIG. 9B is a bar graph showing Ephb3 mRNA expression in astrocytes following treatment with pGFAP-shEphB3, pCD11b-shEfnB3 or control lentiviral vectors. *P<0.05 as determined by One-way ANOVA followed by Dunnett's post-hoc test.

FIG. 9C contains images of immunostaining of GFAP and EGFP on spinal cord of mice transduced with Gfap-driven EGFP lentivirus. Representative images from n=5 mice analyzed.

FIG. 9D contains images of representative spinal cord sections in EAE C57Bl/6J mice treated with A38 and pGFAP-shEphB3 or control (shCtrl) lentiviral vectors (hematoxylin and eosin staining).

FIG. 9E contains images of representative spinal cord sections in EAE C57Bl/6J mice treated with A38 and pGFAP-shEphB3 or control (shCtrl) lentiviral vectors (luxol Fast blue (LFB) staining for demyelination).

FIG. 9F contains images of representative spinal cord sections in EAE C57Bl/6J mice treated with A38 and pGFAP-shEphB3 or control (shCtrl) lentiviral vectors (silver staining for axonal loss).

FIG. 9G contains bar graphs of quantification of demyelination and axonal loss. ****P<0.0001, ***P<0.001, as determined by one-way ANOVA followed by Dunnett's post-hoc test.

FIG. 9H contains bar graphs showing CD4⁺ T cells in EAE C57Bl/6J mice treated with lentiviral knockdown constructs targeting EphB3 in astrocytes (red), EfnB3 in microglia (blue) or control (black) as in FIG. 4 (n=5). Data are representative of 3 independent experiments (T cells in CNS).

FIG. 9I contains bar graphs showing CD4⁺ T cells in EAE C57Bl/6J mice treated with lentiviral knockdown constructs targeting EphB3 in astrocytes (red), EfnB3 in microglia (blue) or control (black) as in FIG. 4 (n=5). Data are representative of 3 independent experiments (T cells in spleen, as analyzed by flow cytometry).

FIG. 9J is a bar graph showing splenocytes proliferation assay 28 days after EAE induction.

FIG. 9K contains bar graphs of IFNγ and IL-17 production by splenocytes as determined by ELISA.

FIG. 9L contains line plots showing EAE development in C57Bl/6J mice treated with lentiviral knockdown constructs targeting EphB3 in astrocytes, EfnB3 in microglia or both at the same time (n=5). Data are representative of 2 independent experiments.

FIG. 9M contains line plots showing EAE development in C57Bl/6J mice treated with lentiviral knockdown constructs targeting EfnB3 in astrocytes (n=5) Data are representative of 2 independent experiments.

FIG. 9N contains bar graphs of analysis of CNS infiltrating T cells by flow cytometry showing CD4 T cells in EAE C57Bl/6J mice treated with A38 as shown in FIG. 6.

FIG. 9O contains line plots showing splenocyte proliferation assay 28 days after EAE induction.

FIG. 9P contains bar graphs of IFNγ, IL-17 and IL-1β production by splenocytes as determined by ELISA.

FIG. 9Q contains line plots showing EAE development in C57Bl/6J mice treated with A38 and lentiviral knockdown constructs targeting EphB3 in astrocytes (n=5) Data are representative of 2 independent experiments.

FIG. 10A contains line plots and bar graphs showing EAE development in NOD mice treated with A38 and lentiviral knockdown constructs targeting EphB3 in astrocytes during the progressive phase (left); linear regression of progressive phase shown (right) (n=6). Data are representative of 2 independent experiments. ****P<0.0001, **P<0.01, ns=not significant as determined by regression slope t test.

FIG. 10B contains bar graph of monocytes in the CNS of NOD mice treated with A38 and lentiviral vectors targeting EphB3 as performed in FIG. 10E (n=6). ***P<0.001, **P<0.01 as determined by one-way ANOVA followed by Tukey's post-hoc test.

FIG. 10C contains bar graphs of CNS infiltrating T cells in NOD EAE mice treated with A38 and lentiviral knockdown constructs targeting EphB3 in astrocytes as shown in (FIG. 10A) (n=6). Data are representative of 2 independent experiments.

FIG. 10D shows Heatmap of differentially expressed genes in astrocytes from control, A38 treated and EphB3 knockdown mice.

FIG. 10E shows Heatmap of differentially expressed genes in microglia from control, A38 treated and EphB3 knockdown mice.

FIG. 10F contains bar graphs of Ingenuity pathway analysis of differentially expressed genes in glial cells from mice treated with A38 or EphB3 knockdown lentivirus showing comparison Z score of selected pathways in astrocytes.

FIG. 10G contains bar graphs of ingenuity pathway analysis of differentially expressed genes in glial cells from mice treated with A38 or EphB3 knockdown lentivirus showing comparison of Z score of selected pathways in microglia.

FIG. 11A contains images of Western blot analysis of NF-κB subunit p65 and loading controls GAPDH and histone 3 in subcellular fractions corresponding to cytoplasm and nucleus, respectively, of neonatal murine astrocytes activated for 30 min with TNFα and IL-1β in the presence of A38, Class I PI3K isoforms inhibitor ZSTK74 (Z74) and C9. Images of Western blot analysis (Unstimulated) is shown on the left, and images of Western blot analysis (TNFα and IL-1β activated) is shown on the right.

FIG. 11B is a bar graph showing mitochondrial ROS measured by Mitosox staining after ON treatment in astrocytes with TNFα and IL-1β and Rotenone and Antimycin A (Rot, AA) when stated (n=3).

FIG. 11C contains bar graphs of 16, Csf2 and Cc/2mRNA expression determined by qPCR in cells treated as in FIG. 11B (n=3). Data representative of 3 independent experiments. **P<0.01, *P<0.05 as determined by unpaired Student T test.

FIG. 12A contains line plots showing EAE development on C57Bl/6 mice treated with A38 or Control starting from day 3 (n=5 mice per group). Data are mean±s.e.m. and representative of 2 independent experiments. **P<0.01 by two-way analysis of variance (ANOVA).

FIG. 12B contains line plots showing EAE development on C57Bl/6 mice treated with A38 or Control starting from day 21 (n=5 mice per group). Data are mean±s.e.m. and representative of 2 independent experiments. **P<0.01 by two-way analysis of variance (ANOVA).

FIG. 13A contains bar graphs showing CNS infiltrating mononuclear cells isolated and analyzed by FACS following treatment as performed in FIG. 12A and FIG. 12B. Data are mean±s.e.m. and representative of 2 independent experiments.

FIG. 13B contains bar graphs showing CNS splenic mononuclear cells isolated and analyzed by FACS following treatment as performed in FIG. 12A and FIG. 12B. Data are mean±s.e.m. and representative of 2 independent experiments.

FIG. 14 is a bar graph showing CNS infiltrating pro-inflammatory monocytes were quantified by FACS following treatment as performed in FIG. 12A and FIG. 12B.

FIG. 15A is a bar graph showing TNFα quantification in culture supernatants in the presence and absence of compound A38.

FIG. 15B contains bar graphs showing expression of nos2, csf2 and ccl2 quantified by qPCR in culture supernatants in the presence and absence of compound A38.

### DETAILED DESCRIPTION

Astrocytes are abundant glial cells in the central nervous system (CNS). These cells play a role in development and homeostasis associated with the control of synaptic activity, metabolism, and the blood-brain barrier. Astrocytes also contribute to the pathology of neurologic disorders through their intrinsic neurotoxic activity, the recruitment of pro-inflammatory monocytes, the induction of microglial neurotoxic activity and the decreased support of neuron metabolism.

Astrocytes and microglia mutually regulate their activities through secreted factors. For example, IL-1α, TNFα and C1q produced by microglia induce a neurotoxic phenotype in astrocytes. Moreover, microglial VEGF-B and TGFα promote and suppress, respectively, astrocyte pro-inflammatory activities. Conversely, astrocyte-produced IL-33 promotes synapse engulfment by microglia.

The experimental results provided in the present disclosure show that microglia and astrocytes establish cell contacts during CNS inflammation.

Specifically, bi-directional signaling involving membrane-bound Ephrin receptor B3 (EphB3) in astrocytes and its membrane-bound ligand Ephrin-B3 in microglia promotes pathology in AE and in MS. Hence, described herein are compounds that may inhibit EphB3 signaling and therefore are capable of ameliorating acute and chronic progressive AE and MS. Pharmaceutical compositions, dosage forms, and methods of using these compounds are also described.

Therapeutic Compounds

In some embodiments, the present disclosure advantageously provides compounds capable of penetrating the blood-brain barrier. In some embodiments, the compound has molecular weight of less than about 400. In some embodiments, the compound has cLogP from about 0 to about 5, and polar surface area (PSA) from about 40 to about 80. In some embodiments, the compound contains no more than 2H-bond donor atoms in its structure, and no more than 8H-bond acceptors. In some embodiments, the compound is water-soluble and orally bioavailable.

Compounds of Formula (I)

In some embodiments, the present disclosure provides a compound of Formula (I):

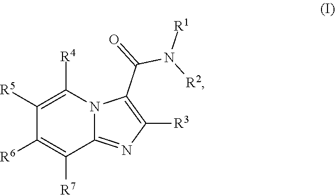

or a pharmaceutically acceptable salt thereof, wherein:
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is optionally substituted with 1, 2, or 3 substituents independently selected from R^{Cy2}; each R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}, wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₃ alkyl, and C₁₋₃ haloalkyl, wherein said C₁₋₃ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

In some embodiments, R¹ is selected from C₆₋₁₀ aryl and 5-14 membered heteroaryl, each of which is optionally substituted with 1 or 2 substituents independently selected from halo and OR^{a1}.

In some embodiments, R² is H.

In some embodiments, R³ is selected from H and C₁₋₃ alkyl.

In some embodiments, R⁵ is selected from NR^{c1}R^{d1} OR^{a1}, C₁₋₆ alkyl, and Cy¹, wherein said C₁₋₆ alkyl is optionally substituted with NR^{c1}R^{d1}.

In some embodiments, Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

In some embodiments, R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

In some embodiments, R⁴ is selected from H, halo, CN, OR^{a1}, and C₁₋₃ haloalkyl.

In some embodiments, R⁴ is H.

In some embodiments, R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

In some embodiments, R⁷ is H.

In some embodiments, R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁶ is H.

In some embodiments:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is selected from NR^{c1}R^{d1}, OR^{a1}, C₁₋₆ alkyl, and Cy¹, wherein said C₁₋₆ alkyl is optionally substituted with NR^{c1}R^{d1};
Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁴, R⁶, and R⁷ are each H.

In some embodiments, the compound of Formula (I) is selected from any one of the compounds listed in Table A:

TABLE A

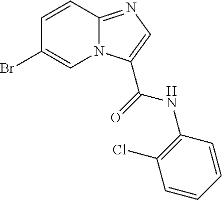 [SMILES: O=C(Nc1ccccc1Cl)c1cnc2ccc(Br)cn12]

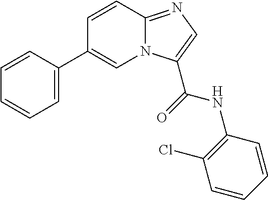 [SMILES: O=C(Nc1ccccc1Cl)c1cnc2ccc(-c3ccccc3)cn12]

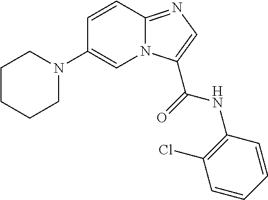 [SMILES: O=C(Nc1ccccc1Cl)c1cnc2ccc(N3CCCCC3)cn12]

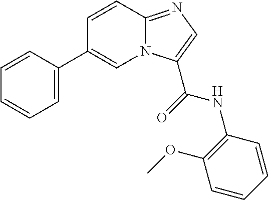 [SMILES: COc1ccccc1NC(=O)c1cnc2ccc(-c3ccccc3)cn12]

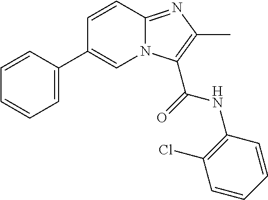 [SMILES: Cc1nc2ccc(-c3ccccc3)cn2c1C(=O)Nc1ccccc1Cl]

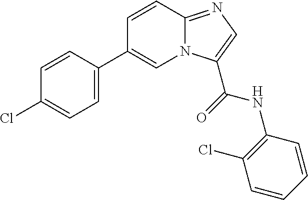 [SMILES: O=C(Nc1ccccc1Cl)c1cnc2ccc(-c3ccc(Cl)cc3)cn12]

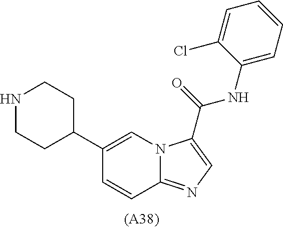

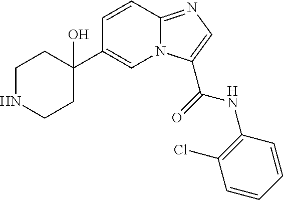 [SMILES: O=C(Nc1ccccc1Cl)c1cnc2ccc(C3(O)CCNCC3)cn12]

or a pharmaceutically acceptable salt thereof.

In some embodiments, the compound of Formula (I) is selected from any one of the compounds 101-119 disclosed herein, or a pharmaceutically acceptable salt thereof.

Compounds of Formula (Ia)

In some embodiments, the present disclosure provides a compound of Formula

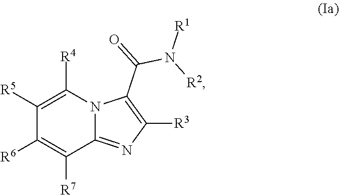

or a pharmaceutically acceptable salt thereof, wherein:
R⁵ is 5-14 membered heteroaryl, optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, C₁₋₆ alkyl, and C₁₋₆ haloalkyl, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, and C₁₋₃ haloalkyl, wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, and a reactive electrophilic warhead group, wherein said C₁₋₆ alkyl is optionally substituted with 1, 2, or 3 substituents independently selected from halo, CN, OR^{a1}, and NR^{c1}R^{d1}; R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

In some embodiments, R¹ is selected from C₆₋₁₀ aryl and 5-14 membered heteroaryl, each of which is optionally substituted with 1 or 2 substituents independently selected from halo and OR^{a1}.

In some embodiments, R² is H.

In some embodiments, R³ is selected from H and C₁₋₃ alkyl.

In some embodiments, R⁵ is 5-6 membered heteroaryl, optionally substituted with 1, 2, or 3 substituents independently selected from OR^{a1}, C(O)R^{b1}, C(O)NR^{c1}R^{d1}, and C₁₋₆ alkyl, wherein said C₁₋₆ alkyl is optionally substituted with OR^{a1} or NR^{c1}R^{d1}.

In some embodiments, R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁶ is H.

In some embodiments, R⁴ is selected from H, halo, CN, OR^{a1}, and C₁₋₃ haloalkyl.

In some embodiments, R⁴ is H.

In some embodiments, R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, and C₁₋₃ haloalkyl, wherein said C₁₋₃ alkyl is optionally substituted with 1 or 2 independently selected R⁹.

In some embodiments, R⁷ is H.

In some embodiments:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is 5-6 membered heteroaryl, optionally substituted with 1, 2, or 3 substituents independently selected from OR^{a1}, C(O)R^{b1}, C(O)NR^{c1}R^{d1}, and C₁₋₆ alkyl, wherein said C₁₋₆ alkyl is optionally substituted with OR^{a1} or NR^{c1}R^{d1};
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1};
R⁴ is selected from H, halo, CN, OR^{a1}, and C₁₋₃ haloalkyl; and
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, and C₁₋₃ haloalkyl, wherein said C₁₋₃ alkyl is optionally substituted with 1 or 2 independently selected R⁹.

In some embodiments, R⁴, R⁶, and R⁷ are each H.

In some embodiments, the compound of Formula (Ia) is selected from any one of the following compounds:

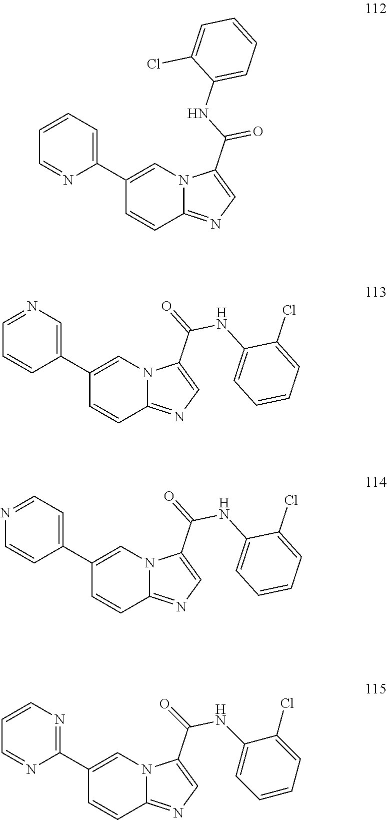 [SMILES: O=C(Nc1ccccc1Cl)c1cnc2ccc(-c3ccccn3)cn12 ; O=C(Nc1ccccc1Cl)c1cnc2ccc(-c3cccnc3)cn12 ; O=C(Nc1ccccc1Cl)c1cnc2ccc(-c3ccncc3)cn12 ; O=C(Nc1ccccc1Cl)c1cnc2ccc(-c3ncccn3)cn12]

or a pharmaceutically acceptable salt thereof.

In some embodiments, the compound of Formula (Ia) is selected from any one of the following compounds:

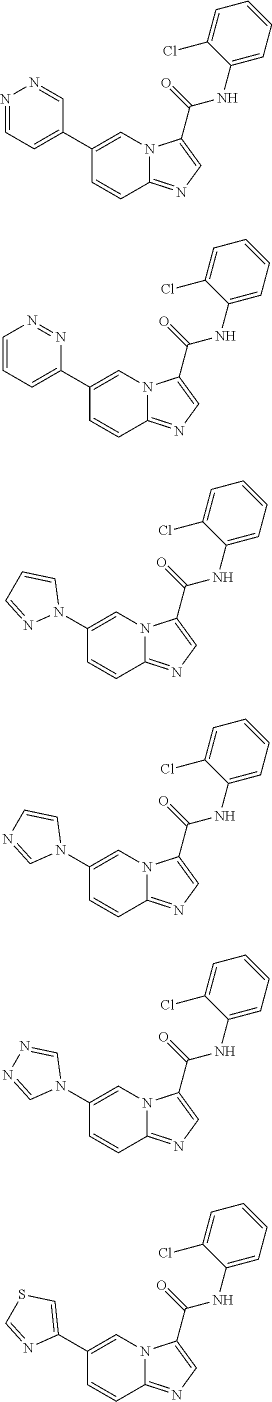 [SMILES: O=C(Nc1ccccc1Cl)c1cnc2ccc(-c3ccnnc3)cn12 ; O=C(Nc1ccccc1Cl)c1cnc2ccc(-c3cccnn3)cn12 ; O=C(Nc1ccccc1Cl)c1cnc2ccc(-n3cccn3)cn12 ; O=C(Nc1ccccc1Cl)c1cnc2ccc(-n3ccnc3)cn12 ; O=C(Nc1ccccc1Cl)c1cnc2ccc(-n3cnnc3)cn12 ; O=C(Nc1ccccc1Cl)c1cnc2ccc(-c3cscn3)cn12]

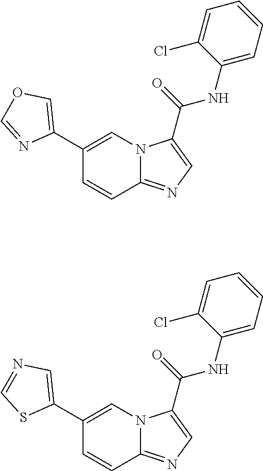 [SMILES: O=C(Nc1ccccc1Cl)c1cnc2ccc(-c3cocn3)cn12 ; O=C(Nc1ccccc1Cl)c1cnc2ccc(-c3cncs3)cn12]

or a pharmaceutically acceptable salt thereof.

Compound of Formula (Ib)

In some embodiments, the present disclosure provides a compound of Formula

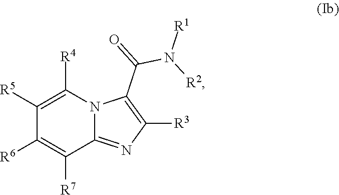

or a pharmaceutically acceptable salt thereof, wherein:
R¹ is selected from 5-14 membered heteroaryl and 4-10 membered heterocycloalkyl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

In some embodiments, R¹ is 5-6 membered heteroaryl, optionally substituted with 1, 2, or 3 substituents independently selected from halo, CN, C₁₋₃ alkoxy, C₁₋₃ alkyl, and C₁₋₃ haloalkyl.

In some embodiments, R² is H.

In some embodiments, R³ is selected from H and C₁₋₃ alkyl.

In some embodiments, R⁵ is selected from NR^{c1}R^{d1}, OR^{a1}, C₁₋₆ alkyl, and Cy¹, wherein said C₁₋₆ alkyl is optionally substituted with NR^{c1}R^{d1}.

In some embodiments, Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

In some embodiments, R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

In some embodiments, R⁴ is selected from H, halo, CN, OR^{a1}, and C₁₋₃ haloalkyl.

In some embodiments, R⁴ is H.

In some embodiments, R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

In some embodiments, R⁷ is H.

In some embodiments, R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁶ is H.

In some embodiments:
R¹ is 5-6 membered heteroaryl, optionally substituted with 1, 2, or 3 substituents independently selected from halo, CN, C₁₋₃ alkoxy, C₁₋₃ alkyl, and C₁₋₃ haloalkyl;
R² is H;
R³ is selected from H and C₁₋₃ alkyl;
R⁵ is selected from NR^{c1}R^{d1}, OR^{a1}, C₁₋₆ alkyl, and Cy¹, wherein said C₁₋₆ alkyl is optionally substituted with NR^{c1}R^{d1};
Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{d1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁴, R⁶, and R⁷ are each H.

In some embodiments, the compound of Formula (Ib) is selected from any one of the following compounds

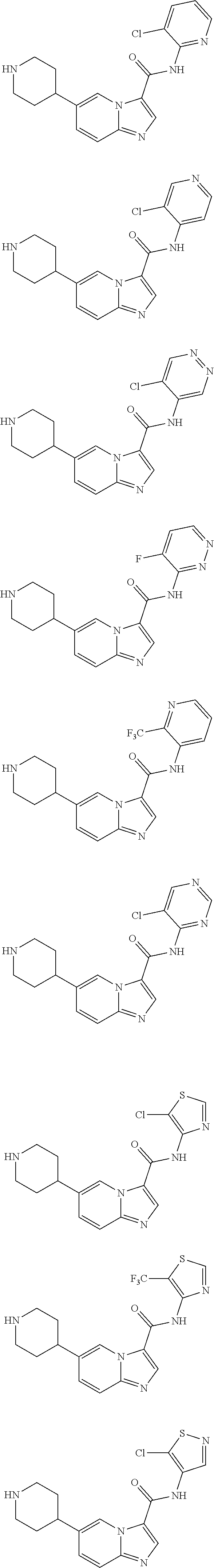

or a pharmaceutically acceptable salt thereof.

Compound of Formula (Ic)

In some embodiments, the present disclosure provides a compound of Formula

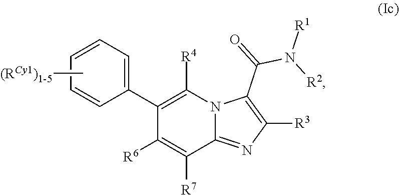

or a pharmaceutically acceptable salt thereof, wherein:
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

In some embodiments, the compound is not:

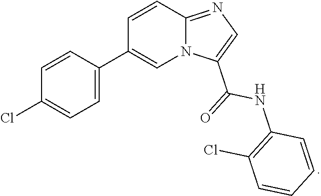 [SMILES: O=C(Nc1ccccc1Cl)c1cnc2ccc(-c3ccc(Cl)cc3)cn12]

In some embodiments, R¹ is selected from C₆₋₁₀ aryl and 5-14 membered heteroaryl, each of which is optionally substituted with 1 or 2 substituents independently selected from halo and OR^{a1}.

In some embodiments, R² is H.

In some embodiments, R³ is selected from H and C₁₋₃ alkyl.

In some embodiments, R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

In some embodiments, R⁴ is selected from H, halo, CN, OR^{a1}, and C₁₋₃ haloalkyl.

In some embodiments, R⁴ is H.

In some embodiments, R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

In some embodiments, R⁷ is H.

In some embodiments, R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁶ is H.

In some embodiments:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁴, R⁶, and R⁷ are each H.

In some embodiments, the compound is selected from any one of the following compounds:

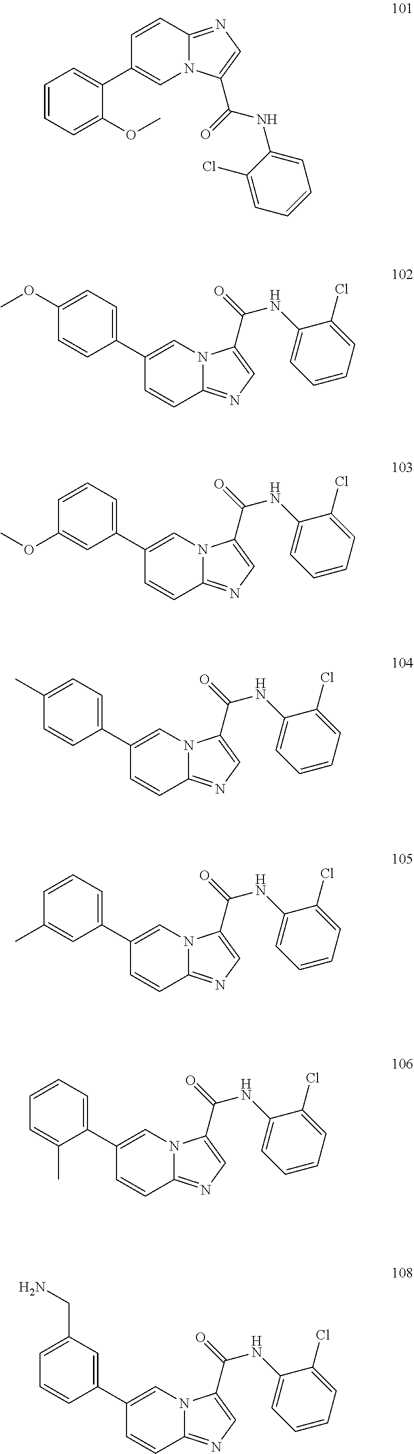

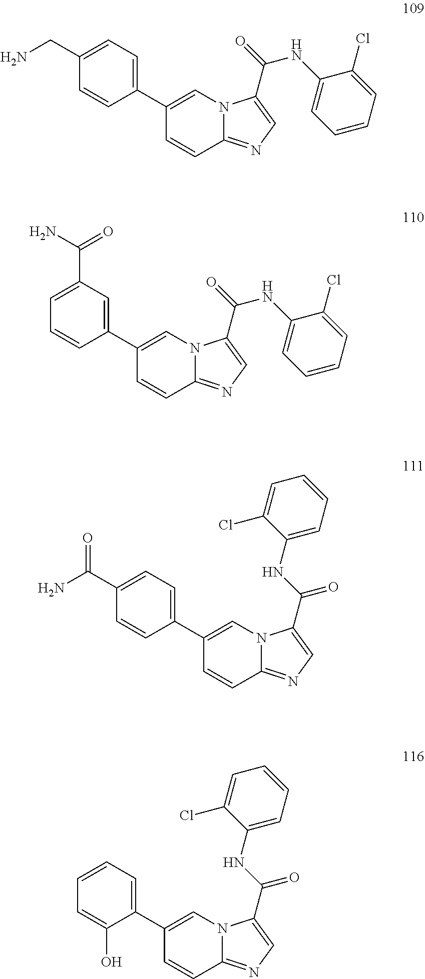 [SMILES: NCc1ccc(-c2ccc3ncc(C(=O)Nc4ccccc4Cl)n3c2)cc1 ; NC(=O)c1cccc(-c2ccc3ncc(C(=O)Nc4ccccc4Cl)n3c2)c1 ; NC(=O)c1ccc(-c2ccc3ncc(C(=O)Nc4ccccc4Cl)n3c2)cc1 ; O=C(Nc1ccccc1Cl)c1cnc2ccc(-c3ccccc3O)cn12]

or a pharmaceutically acceptable salt thereof.

In some embodiments, the compound is selected from any one of the following compounds:

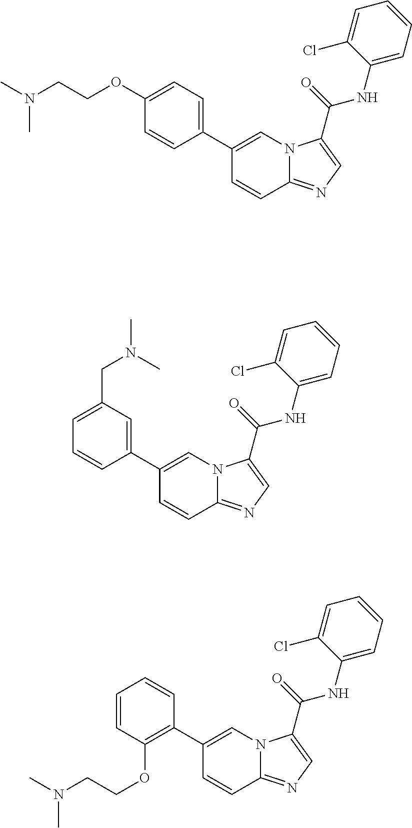 [SMILES: CN(C)CCOc1ccc(-c2ccc3ncc(C(=O)Nc4ccccc4Cl)n3c2)cc1 ; CN(C)Cc1cccc(-c2ccc3ncc(C(=O)Nc4ccccc4Cl)n3c2)c1 ; CN(C)CCOc1ccccc1-c1ccc2ncc(C(=O)Nc3ccccc3Cl)n2c1]

or a pharmaceutically acceptable salt thereof.

Compound of Formula (Id)

In some embodiments, the present disclosure provides a compound of Formula (Id):

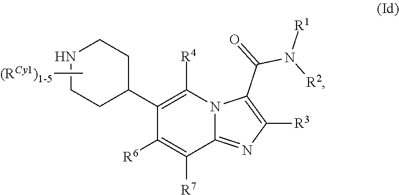

or a pharmaceutically acceptable salt thereof, wherein:
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

In some embodiments, the compound is not:

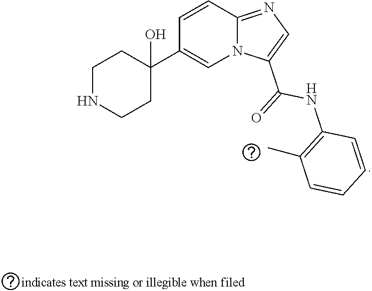 [SMILES: Cc1ccccc1NC(=O)c1cnc2ccc(C3(O)CCNCC3)cn12]

In some embodiments, R¹ is selected from C₆₋₁₀ aryl and 5-14 membered heteroaryl, each of which is optionally substituted with 1 or 2 substituents independently selected from halo and OR^{a1}.

In some embodiments, R² is H.

In some embodiments, R³ is selected from H and C₁₋₃ alkyl.

In some embodiments, R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

In some embodiments, R⁴ is selected from H, halo, CN, OR^{a1}, and C₁₋₃ haloalkyl.

In some embodiments, R⁴ is H.

In some embodiments, R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

In some embodiments, R⁷ is H.

In some embodiments, R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁶ is H.

In some embodiments:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁴, R⁶, and R⁷ are each H.

In some embodiments, the compound of Formula (Id) is:

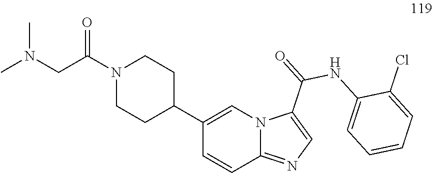 [SMILES: CN(C)CC(=O)N1CCC(c2ccc3ncc(C(=O)Nc4ccccc4Cl)n3c2)CC1]

or a pharmaceutically acceptable salt thereof.

In some embodiments, the compound of Formula (Id) is selected from any one of the following compounds:

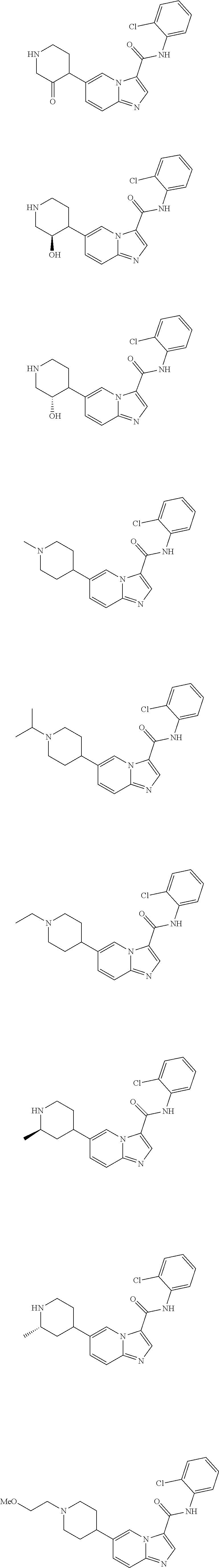

or a pharmaceutically acceptable salt thereof.

Compound of Formula (Ie)

In some embodiments, the present disclosure provides a compound of Formula

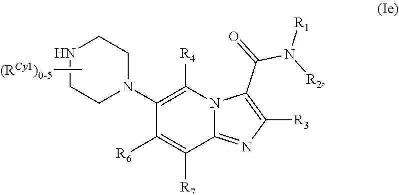

or a pharmaceutically acceptable salt thereof, wherein:
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

In some embodiments, R¹ is selected from C₆₋₁₀ aryl and 5-14 membered heteroaryl, each of which is optionally substituted with 1 or 2 substituents independently selected from halo and OR^{a1}.

In some embodiments, R² is H.

In some embodiments, R³ is selected from H and C₁₋₃ alkyl.

In some embodiments, R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

In some embodiments, R⁴ is selected from H, halo, CN, OR^{a1}, and C₁₋₃ haloalkyl.

In some embodiments, R⁴ is H.

In some embodiments, R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

In some embodiments, R⁷ is H.

In some embodiments, R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁶ is H.

In some embodiments:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁴, R⁶, and R⁷ are each H.

In some embodiments, the compound of Formula (Ie) is selected from any one of the following compounds:

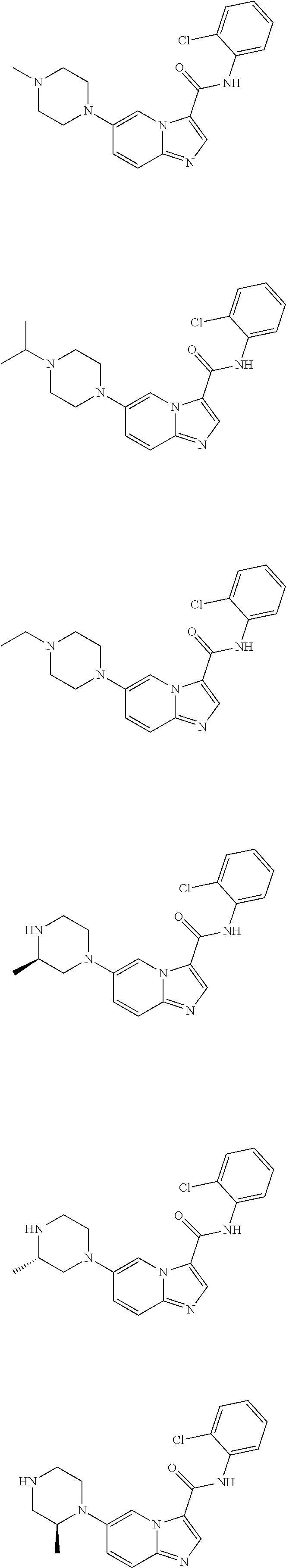 [SMILES: CN1CCN(c2ccc3ncc(C(=O)Nc4ccccc4Cl)n3c2)CC1 ; CC(C)N1CCN(c2ccc3ncc(C(=O)Nc4ccccc4Cl)n3c2)CC1 ; CCN1CCN(c2ccc3ncc(C(=O)Nc4ccccc4Cl)n3c2)CC1 ; C[C@@H]1CN(c2ccc3ncc(C(=O)Nc4ccccc4Cl)n3c2)CCN1 ; C[C@H]1CN(c2ccc3ncc(C(=O)Nc4ccccc4Cl)n3c2)CCN1 ; C[C@H]1CNCCN1c1ccc2ncc(C(=O)Nc3ccccc3Cl)n2c1]

or a pharmaceutically acceptable salt thereof.

Compound of Formula (If)

In some embodiments, the present disclosure provides a compound of Formula

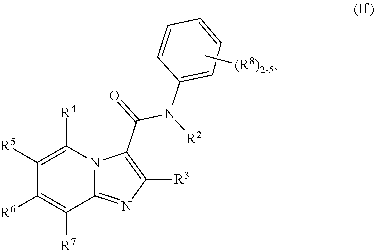

or a pharmaceutically acceptable salt thereof, wherein:
each R⁸ is independently selected from halo, CN, OR^{a1}, C₁₋₃ alkyl, and C₁₋₃ haloalkyl, wherein said C₁₋₃ alkyl is optionally substituted with 1 or 2 independently selected R^{g};
or any two adjacent R⁸ groups together with the carbon atoms to which they are attached from a ring selected from 5-6 membered heteroaryl and 4-6 membered heterocycloalkyl, each of which is optionally substituted with 1, 2, or 3 substituents independently selected from R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1}; each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

In some embodiments, each R⁸ is independently selected from halo, CN, C₁₋₃ alkoxy, C₁₋₃ alkyl, and C₁₋₃ haloalkyl, wherein said C₁₋₃ alkyl is optionally substituted with R^{g}.

In some embodiments, any two adjacent R⁸ groups together with the carbon atoms to which they are attached form a 5-6 membered heteroaryl ring, optionally substituted with 1 or 2 substituents independently selected from R^{g}.

In some embodiments, any two adjacent R⁸ groups together with the carbon atoms to which they are attached form a 4-6 membered heterocycloalkyl, optionally substituted with 1 or 2 substituents independently selected from R^{g}.

In some embodiments, R² is H.

In some embodiments, R³ is selected from H and C₁₋₃ alkyl.

In some embodiments, R⁵ is selected from NR^{c1}R^{d1} OR^{a1}, C₁₋₆ alkyl, and Cy¹, wherein said C₁₋₆ alkyl is optionally substituted with NR^{c1}R^{d1}.

In some embodiments, Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

In some embodiments, R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

In some embodiments, R⁴ is selected from H, halo, CN, OR^{a1}, and C₁₋₃ haloalkyl.

In some embodiments, R⁴ is H.

In some embodiments, R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

In some embodiments, R⁷ is H.

In some embodiments, R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁶ is H.

In some embodiments:
R² is H;
R³ is selected from H and C₁₋₃ alkyl;
R⁵ is selected from NR^{c1}R^{d1}, OR^{a1}, C₁₋₆ alkyl, and Cy¹, wherein said C₁₋₆ alkyl is optionally substituted with NR^{c1}R^{d1};
Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁴, R⁶, and R⁷ are each H.

In some embodiments, the compound of Formula (If) is selected from any one of the following compounds:

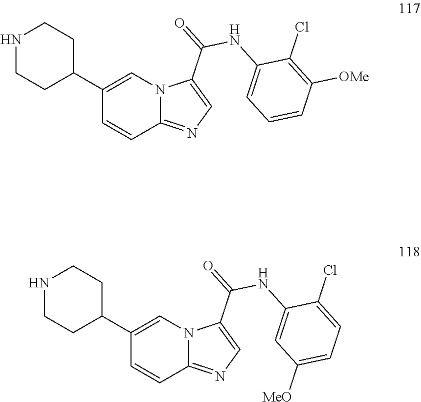 [SMILES: COc1cccc(NC(=O)c2cnc3ccc(C4CCNCC4)cn23)c1Cl ; COc1ccc(Cl)c(NC(=O)c2cnc3ccc(C4CCNCC4)cn23)c1]

or a pharmaceutically acceptable salt thereof.

In some embodiments, the compound of Formula (If) is selected from any one of the following compounds:

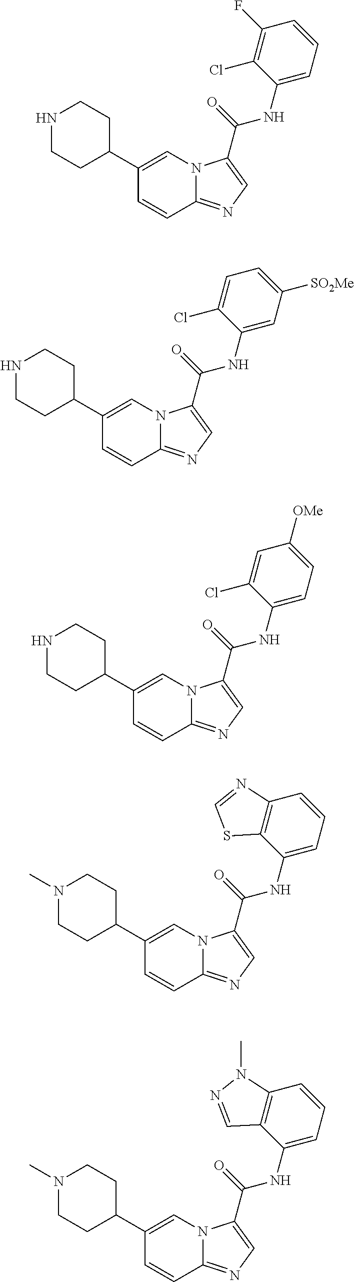 [SMILES: O=C(Nc1cccc(F)c1Cl)c1cnc2ccc(C3CCNCC3)cn12 ; CS(=O)(=O)c1ccc(Cl)c(NC(=O)c2cnc3ccc(C4CCNCC4)cn23)c1 ; COc1ccc(NC(=O)c2cnc3ccc(C4CCNCC4)cn23)c(Cl)c1 ; CN1CCC(c2ccc3ncc(C(=O)Nc4cccc5ncsc45)n3c2)CC1 ; CN1CCC(c2ccc3ncc(C(=O)Nc4cccc5c4cnn5C)n3c2)CC1]

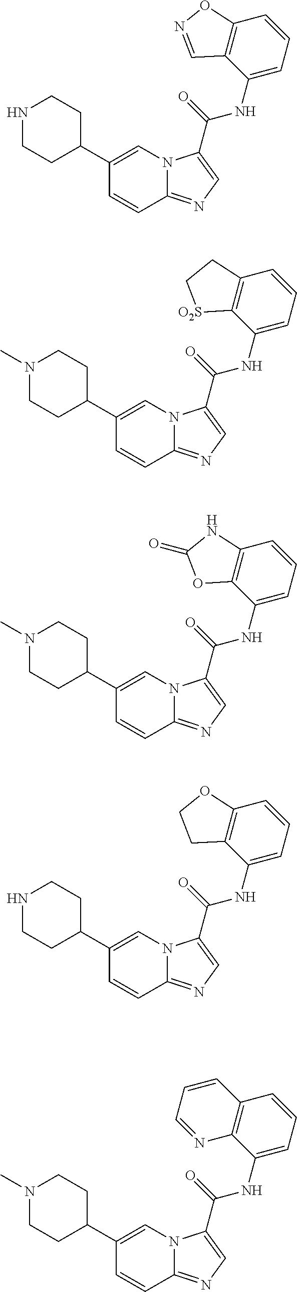 [SMILES: O=C(Nc1cccc2oncc12)c1cnc2ccc(C3CCNCC3)cn12 ; CN1CCC(c2ccc3ncc(C(=O)Nc4cccc5c4S(=O)(=O)CC5)n3c2)CC1 ; CN1CCC(c2ccc3ncc(C(=O)Nc4cccc5[nH]c(=O)oc45)n3c2)CC1 ; O=C(Nc1cccc2c1CCO2)c1cnc2ccc(C3CCNCC3)cn12 ; CN1CCC(c2ccc3ncc(C(=O)Nc4cccc5cccnc45)n3c2)CC1]

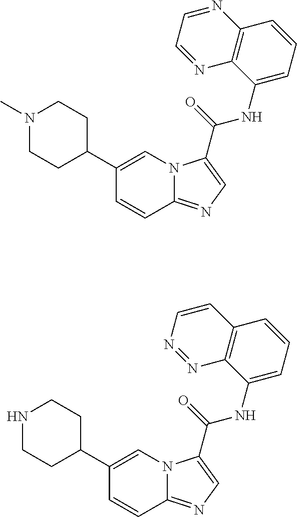 [SMILES: CN1CCC(c2ccc3ncc(C(=O)Nc4cccc5nccnc45)n3c2)CC1 ; O=C(Nc1cccc2ccnnc12)c1cnc2ccc(C3CCNCC3)cn12]

or a pharmaceutically acceptable salt thereof.

Compound of Formula (Ig)

In some embodiments, the present disclosure provides a compound of Formula (Ig):

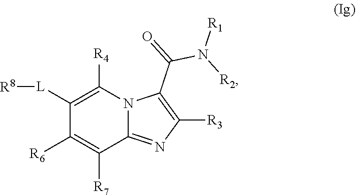

or a pharmaceutically acceptable salt thereof, wherein:
L is selected from C₁₋₆ alkylene, O—C₁₋₆ alkylene, and NH—C₁₋₆ alkylene, each of which is optionally substituted with 1 or 2 independently selected R⁹;
R⁸ is selected from NR^{c1}R^{d1}, C(O)NR^{c1}R^{d1}, and OC(O)NR^{c1}R^{d1};
each R⁹ is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

In some embodiments, L is selected from C₁₋₆ alkylene, O—C₁₋₆ alkylene, and NH—C₁₋₆ alkylene.

In some embodiments, R⁸ is NR^{c1}R^{d1}.

In some embodiments, R¹ is selected from C₆₋₁₀ aryl and 5-14 membered heteroaryl, each of which is optionally substituted with 1 or 2 substituents independently selected from halo and OR^{a1}.

In some embodiments, R² is H.

In some embodiments, R³ is selected from H and C₁₋₃ alkyl.

In some embodiments, R⁴ is selected from H, halo, CN, OR^{a1}, and C₁₋₃ haloalkyl.

In some embodiments, R⁴ is H.

In some embodiments, R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

In some embodiments, R⁷ is H.

In some embodiments, R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁶ is H.

In some embodiments:
L is selected from C₁₋₆ alkylene, O—C₁₋₆ alkylene, and NH—C₁₋₆ alkylene;
R⁸ is NR^{c1}R^{d1};
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

In some embodiments, R⁴, R⁶, and R⁷ are each H.

In some embodiments, the compound of Formula (Ig) is selected from any one of the following compounds:

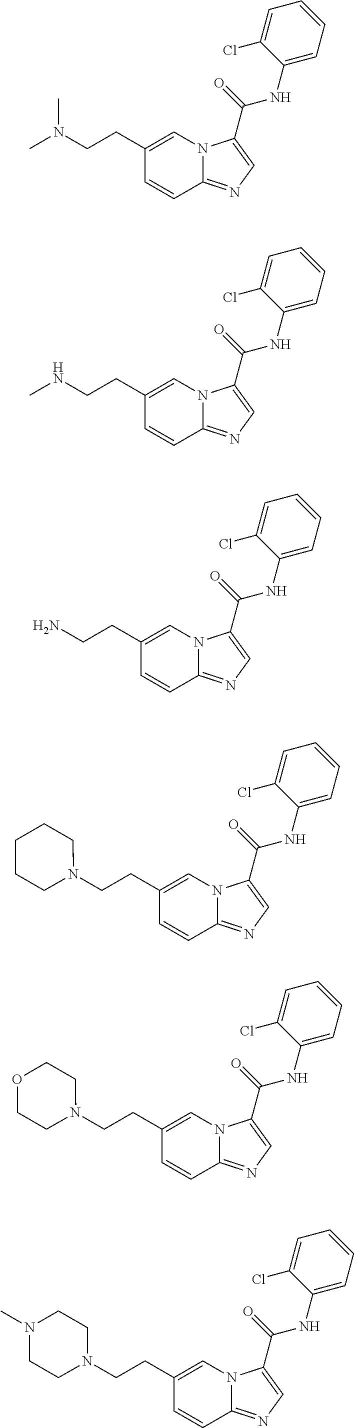 [SMILES: CN(C)CCc1ccc2ncc(C(=O)Nc3ccccc3Cl)n2c1 ; CNCCc1ccc2ncc(C(=O)Nc3ccccc3Cl)n2c1 ; NCCc1ccc2ncc(C(=O)Nc3ccccc3Cl)n2c1 ; O=C(Nc1ccccc1Cl)c1cnc2ccc(CCN3CCCCC3)cn12 ; O=C(Nc1ccccc1Cl)c1cnc2ccc(CCN3CCOCC3)cn12 ; CN1CCN(CCc2ccc3ncc(C(=O)Nc4ccccc4Cl)n3c2)CC1]

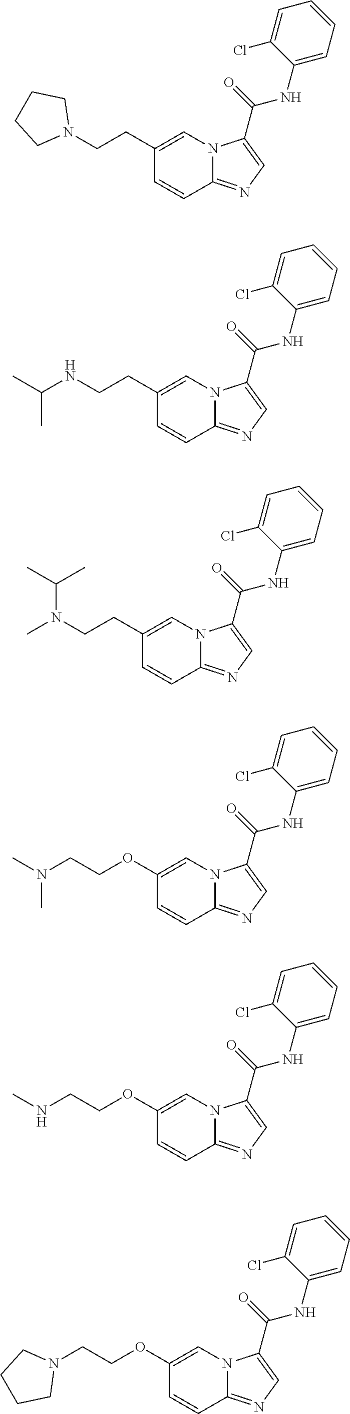 [SMILES: O=C(Nc1ccccc1Cl)c1cnc2ccc(CCN3CCCC3)cn12 ; CC(C)NCCc1ccc2ncc(C(=O)Nc3ccccc3Cl)n2c1 ; CC(C)N(C)CCc1ccc2ncc(C(=O)Nc3ccccc3Cl)n2c1 ; CN(C)CCOc1ccc2ncc(C(=O)Nc3ccccc3Cl)n2c1 ; CNCCOc1ccc2ncc(C(=O)Nc3ccccc3Cl)n2c1 ; O=C(Nc1ccccc1Cl)c1cnc2ccc(OCCN3CCCC3)cn12]

or a pharmaceutically acceptable salt thereof.

Compound of Formula (Ih)

In some embodiments, the present disclosure provides a compound of Formula

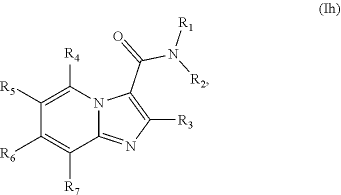

or a pharmaceutically acceptable salt thereof, wherein:
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴, R⁶, and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
provided that at least one of R⁴, R⁶, and R⁷ is selected from halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

In some embodiments, R¹ is selected from C₆₋₁₀ aryl and 5-14 membered heteroaryl, each of which is optionally substituted with 1 or 2 substituents independently selected from halo and OR^{a1}.

In some embodiments, R² is H.

In some embodiments, R³ is selected from H and C₁₋₃ alkyl.

In some embodiments, R⁵ is selected from NR^{c1}R^{d1} OR^{a1}, C₁₋₆ alkyl, and Cy¹, wherein said C₁₋₆ alkyl is optionally substituted with NR^{c1}R^{d1}.

In some embodiments, Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

In some embodiments, R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

In some embodiments, R⁴, R⁶, and R⁷ are each independently selected from H, halo, CN, C₁₋₃ alkoxy, S(O)₂R^{b1}, S(O)R^{b1}, C(O)NR^{c1}R^{d1}, C(O)OR^{a1}, C₁₋₃ alkyl, C₁₋₃ haloalkyl, and 5-6 membered heteroaryl, wherein said C₁₋₃ alkyl is optionally substituted with R^{g}.

In some embodiments:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is selected from NR^{c1}R^{d1}, OR^{a1}, C₁₋₆ alkyl, and Cy¹, wherein said C₁₋₆ alkyl is optionally substituted with NR^{c1}R^{d1};
Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴, R⁶, and R⁷ are each independently selected from H, halo, CN, OH, C₁₋₃ alkoxy, S(O)₂R^{b1}, S(O)R^{b1}, C(O)NR^{c1}R^{d1}, C(O)OR^{a1}, C₁₋₃ alkyl, C₁₋₃ haloalkyl, and 5-6 membered heteroaryl, wherein said C₁₋₃ alkyl is optionally substituted with R^{g}.

In some embodiments, the compound of Formula (Ih) is selected from any one of the following compounds:

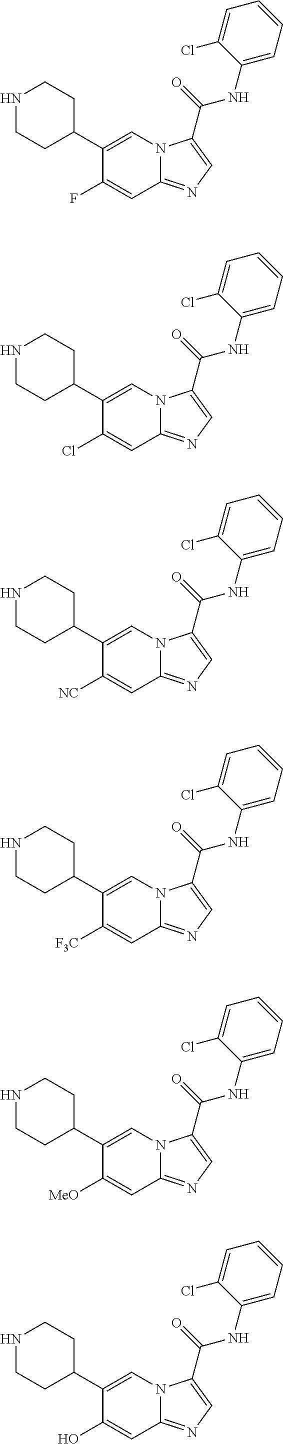 [SMILES: O=C(Nc1ccccc1Cl)c1cnc2cc(F)c(C3CCNCC3)cn12 ; O=C(Nc1ccccc1Cl)c1cnc2cc(Cl)c(C3CCNCC3)cn12 ; N#Cc1cc2ncc(C(=O)Nc3ccccc3Cl)n2cc1C1CCNCC1 ; O=C(Nc1ccccc1Cl)c1cnc2cc(C(F)(F)F)c(C3CCNCC3)cn12 ; COc1cc2ncc(C(=O)Nc3ccccc3Cl)n2cc1C1CCNCC1 ; O=C(Nc1ccccc1Cl)c1cnc2cc(O)c(C3CCNCC3)cn12]

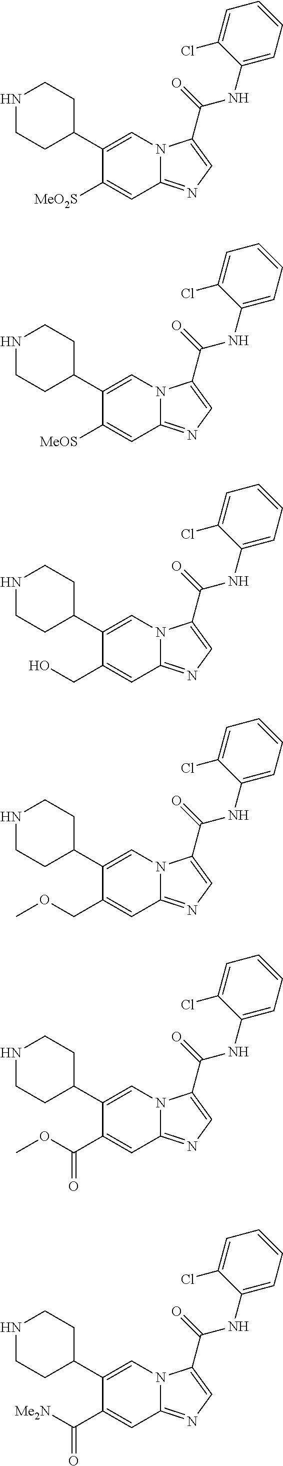

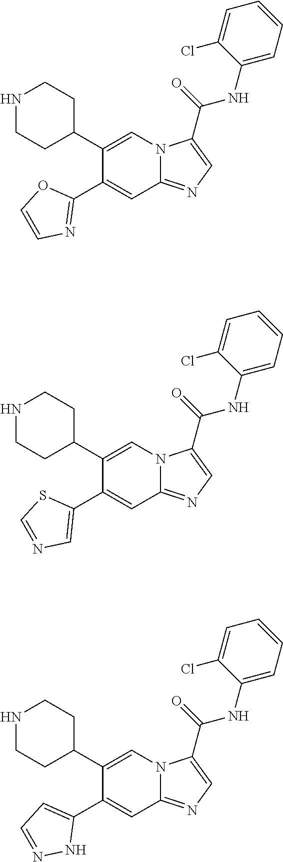 [SMILES: O=C(Nc1ccccc1Cl)c1cnc2cc(-c3ncco3)c(C3CCNCC3)cn12 ; O=C(Nc1ccccc1Cl)c1cnc2cc(-c3cncs3)c(C3CCNCC3)cn12 ; O=C(Nc1ccccc1Cl)c1cnc2cc(-c3ccn[nH]3)c(C3CCNCC3)cn12]

or a pharmaceutically acceptable salt thereof.

Compound of Formula (Ii)

In some embodiments, the present disclosure provides a compound of Formula

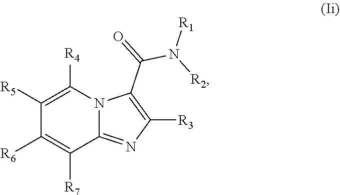

or a pharmaceutically acceptable salt thereof, wherein:
R⁶ is a reactive electrophilic warhead group;
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1}; each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g};
or any two R^{c1} and R^{d1}, together with the N atom to which they are attached, for a 4-7 membered heterocycloalkyl ring, which is optionally substituted with 1, 2, or 3 substituents independently selected from R^{g};
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

In some embodiments, warhead groups described herein for Formula (Ii) can be used in a compound of any one of the Formulae disclosed herein.

In some embodiments, R⁶ is a warhead group of formula:

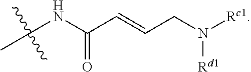

In some embodiments, R⁶ is a warhead group selected from:

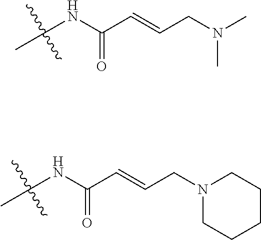

In some embodiments, R is a warhead group of formula:

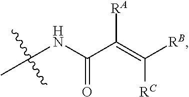

wherein:
R^{A}, R^{B}, and R^{C} are each independently selected from H, halo, CN, and C₁₋₃ alkyl.

In some embodiments, R⁶ is a warhead selected from:

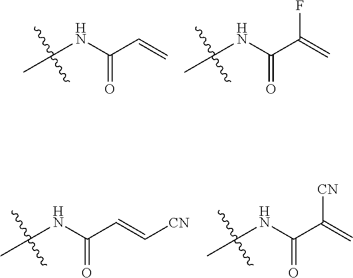

In some embodiments, ein R⁶ is a warhead group of formula:

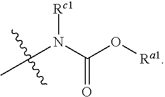

In some embodiments, R⁶ is a warhead selected from:

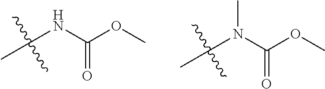

In some embodiments, R⁶ is a warhead group of formula:

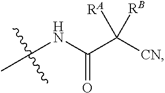

wherein:
R^{A} and R^{B} are each independently selected from H and C₁₋₃ alkyl, or
R^{A} and RB, together with the carbon atom to which they are attached, for a C₃₋₅ cycloalkyl ring.

In some embodiments, R⁶ is a warhead selected from:

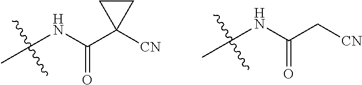 [SMILES: *NC(=O)C1(C#N)CC1 ; *NC(=O)CC#N]

In some embodiments, R⁶ is a warhead group of formula:

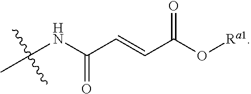

In some embodiments, R⁶ is a warhead group of formula:

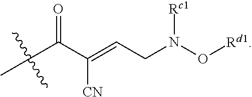

In some embodiments, R⁶ is a warhead group of formula:

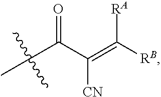

wherein:
R^{A} and R^{B} are each independently selected from H, C(O)NR^{c1}R^{d1}, and C(O)OR^{a1}.

In some embodiments, R⁶ is a warhead selected from:

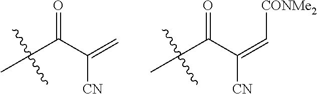

In some embodiments, R⁶ is a warhead of formula:

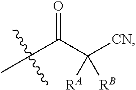

wherein:
R^{A} and R^{B} are each independently selected from H and C₁₋₃ alkyl, or
R^{A} and R^{B}, together with the carbon atom to which they are attached, for a C₃₋₅ cycloalkyl ring.

In some embodiments, R⁶ is a warhead selected from:

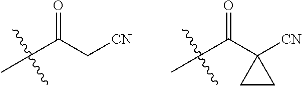 [SMILES: *C(=O)CC#N ; *C(=O)C1(C#N)CC1]

In some embodiments, R⁶ is a warhead of formula:

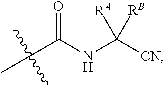

wherein:
R^{A} and R^{B} are each independently selected from H and C₁₋₃ alkyl, or
R^{A} and RB, together with the carbon atom to which they are attached, for a C₃₋₅ cycloalkyl ring.

In some embodiments, R⁶ is a warhead selected from:

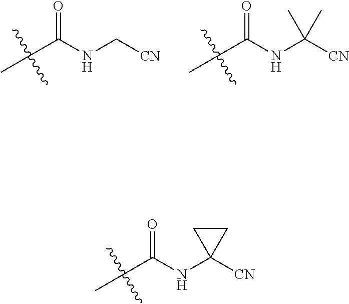

In some embodiments, R is a warhead group of formula:

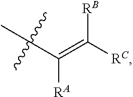

wherein:
R^{A}, R^{B}, and R^{C} are each independently selected from H, CN, C(O)NR^{c1}R^{d1}, and C(O)OR^{a1}, provided that at least one of R^{A}, R^{B}, and R^{C} is selected from CN, C(O)NR^{c1}R^{d1}, and C(O)OR^{a1}.

In some embodiments, R⁶ is a warhead selected from:

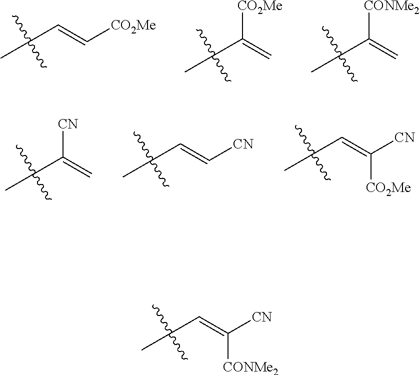

In some embodiments, R⁶ is a warhead selected from a moiety of any one of the following formulae:

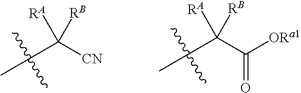

wherein R^{A} and R^{B} are each independently selected from H and C₁₋₃ alkyl.

In some embodiments, R⁶ is a warhead selected from:

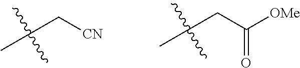

In some embodiments, R is a warhead of formula:

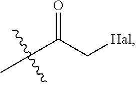

wherein Hal is a halo.

In some embodiments, R⁶ is a warhead selected from:

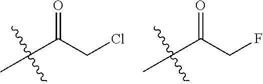 [SMILES: *C(=O)CCl ; *C(=O)CF]

In some embodiments, R is a warhead of formula:

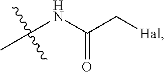

wherein Hal is a halo.

In some embodiments, R⁶ is a warhead selected from:

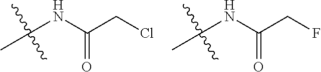 [SMILES: *NC(=O)CCl ; *NC(=O)CF]

In some embodiments, R⁶ is a warhead of formula:

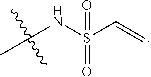 [SMILES: *NS(=O)(=O)C=C]

In some embodiments, R⁶ is a warhead which is a 5-6 membered heteroaryl, which is substituted with a reactive group selected from halo, CN, ethynyl, and vinyl.

In some embodiments, R⁶ is a warhead selected from:

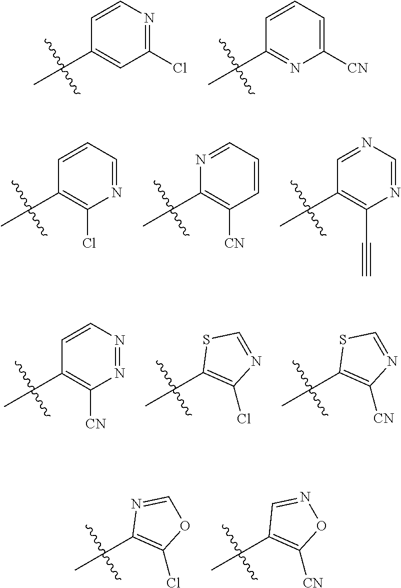

In some embodiments, R is selected from C₆₋₁₀ aryl and 5-14 membered heteroaryl, each of which is optionally substituted with 1 or 2 substituents independently selected from halo and OR^{a1}.

In some embodiments, R² is H.

In some embodiments, R³ is selected from H and C₁₋₃ alkyl.

In some embodiments, R⁵ is selected from NR^{c1}R^{d1} OR^{a1}, C₁₋₆ alkyl, and Cy¹, wherein said C₁₋₆ alkyl is optionally substituted with NR^{c1}R^{d1}.

In some embodiments, Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

In some embodiments, R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

In some embodiments, R⁴ is selected from H, halo, CN, OR^{a1}, and C₁₋₃ haloalkyl.

In some embodiments, R⁴ is H.

In some embodiments, R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

In some embodiments, R⁷ is H.

In some embodiments:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is selected from NR^{c1}R^{d1}, OR^{a1}, C₁₋₆ alkyl, and Cy¹, wherein said C₁₋₆ alkyl is optionally substituted with NR^{c1}R^{d1};
Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl; and
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

In some embodiments, the compound of Formula (Ii) is selected from any one of the following compounds:

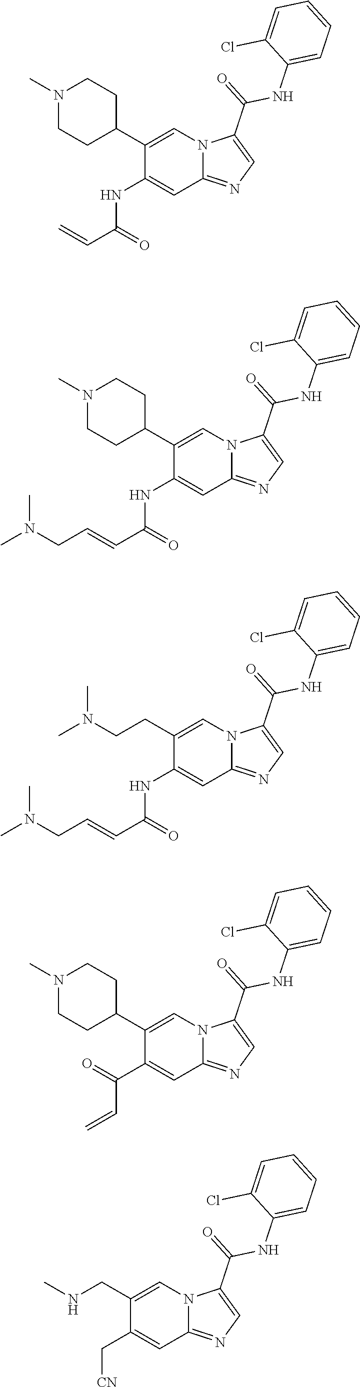 [SMILES: C=CC(=O)Nc1cc2ncc(C(=O)Nc3ccccc3Cl)n2cc1C1CCN(C)CC1 ; CN(C)C/C=C/C(=O)Nc1cc2ncc(C(=O)Nc3ccccc3Cl)n2cc1C1CCN(C)CC1 ; CN(C)C/C=C/C(=O)Nc1cc2ncc(C(=O)Nc3ccccc3Cl)n2cc1CCN(C)C ; C=CC(=O)c1cc2ncc(C(=O)Nc3ccccc3Cl)n2cc1C1CCN(C)CC1 ; CNCc1cn2c(C(=O)Nc3ccccc3Cl)cnc2cc1CC#N]

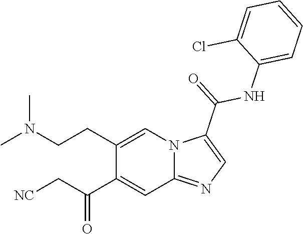 [SMILES: CN(C)CCc1cn2c(C(=O)Nc3ccccc3Cl)cnc2cc1C(=O)CC#N]

or a pharmaceutically acceptable salt thereof.

Compound of Formula (II)

In some embodiments, the present disclosure provides a method of treating a neurodegenerative disease or condition, the method comprising administering to a subject in need thereof a therapeutically effective amount of a compound of Formula (II):

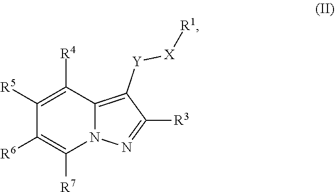

or a pharmaceutically acceptable salt thereof, wherein:
X and Y are each independently selected from C(═O) and NR²;
R¹ is selected from C₆₋₁₀ aryl, C₆₋₁₀ aryl-C₁₋₃ alkyl, 4-10 membered heterocycloalkyl, and 5-14 membered heteroaryl, each of which is optionally substituted with 1, 2, or 3 substituents independently selected from halo, CN, OR^{a1}, S(O)₂R^{b1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, phenyl, carboxy, C₁₋₆ alkoxycarbonyl, amino, C₁₋₆ alkylamino, and di(C₁₋₆ alkyl)amino.

In some embodiments, X is C(═O) and Y is NH.

In some embodiments, X is NH and Y is C(═O).

In some embodiments, R¹ is selected from C₆₋₁₀ aryl and C₆₋₁₀ aryl-C₁₋₃ alkyl, each of which is optionally substituted with 1 or 2 substituents independently selected from halo, CN, OR^{a1}, S(O)₂R^{b1}, C₁₋₃ alkyl, and C₁₋₃ haloalkyl.

In some embodiments, R³ is selected from H and C₁₋₃ alkyl.

In some embodiments, R⁵ is selected from halo, NR^{c1}R^{d1} OR^{a1}, C₁₋₆ alkyl, and Cy¹, wherein said C₁₋₆ alkyl is optionally substituted with NR^{c1}R^{d1}.

In some embodiments, Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

In some embodiments, R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

In some embodiments, R⁴ is selected from H, halo, C₁₋₃ alkyl, CN, OR^{a1}, and C₁₋₃ haloalkyl.

In some embodiments, R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

In some embodiments, R⁶ is selected from H, halo, CN, OR^{a1}, and C₁₋₆ alkyl.

In some embodiments:
R¹ is selected from C₆₋₁₀ aryl and C₆₋₁₀ aryl-C₁₋₃ alkyl, each of which is optionally substituted with 1 or 2 substituents independently selected from halo, CN, OR^{a1}, S(O)₂R^{b1}, C₁₋₃ alkyl, and C₁₋₃ haloalkyl;
R³ is selected from H and C₁₋₃ alkyl;
R⁵ is selected from halo, NR^{c1}R^{d1}, OR^{a1}, C₁₋₆ alkyl, and Cy¹, wherein said C₁₋₆ alkyl is optionally substituted with NR^{c1}R^{d1};
Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, C₁₋₃ alkyl, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, and C₁₋₆ alkyl.

In some embodiments, the compound of Formula (II) is selected from any one of the compounds listed in Table C:

TABLE C

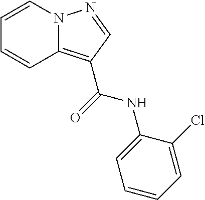 [SMILES: O=C(Nc1ccccc1Cl)c1cnn2ccccc12]

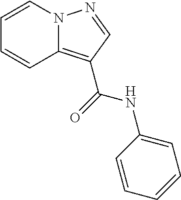 [SMILES: O=C(Nc1ccccc1)c1cnn2ccccc12]

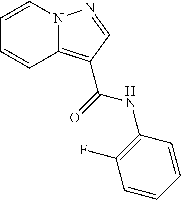 [SMILES: O=C(Nc1ccccc1F)c1cnn2ccccc12]

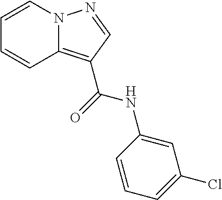 [SMILES: O=C(Nc1cccc(Cl)c1)c1cnn2ccccc12]

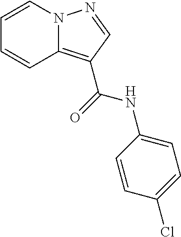 [SMILES: O=C(Nc1ccc(Cl)cc1)c1cnn2ccccc12]

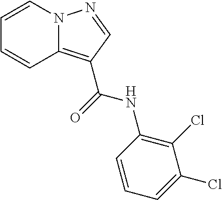 [SMILES: O=C(Nc1cccc(Cl)c1Cl)c1cnn2ccccc12]

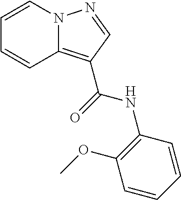 [SMILES: COc1ccccc1NC(=O)c1cnn2ccccc12]

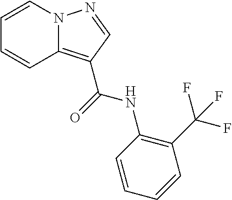 [SMILES: O=C(Nc1ccccc1C(F)(F)F)c1cnn2ccccc12]

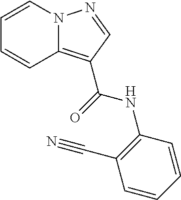 [SMILES: N#Cc1ccccc1NC(=O)c1cnn2ccccc12]

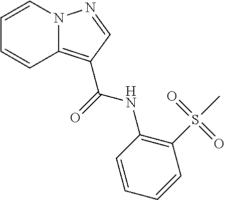 [SMILES: CS(=O)(=O)c1ccccc1NC(=O)c1cnn2ccccc12]

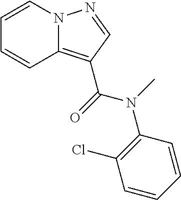 [SMILES: CN(C(=O)c1cnn2ccccc12)c1ccccc1Cl]

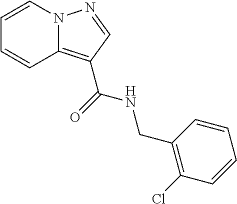 [SMILES: O=C(NCc1ccccc1Cl)c1cnn2ccccc12]

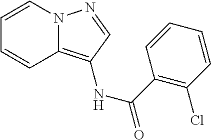 [SMILES: O=C(Nc1cnn2ccccc12)c1ccccc1Cl]

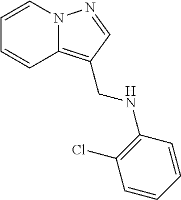 [SMILES: Clc1ccccc1NCc1cnn2ccccc12]

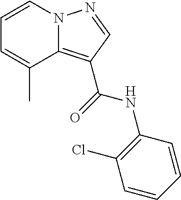 [SMILES: Cc1cccn2ncc(C(=O)Nc3ccccc3Cl)c12]

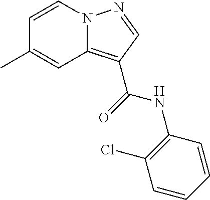 [SMILES: Cc1ccn2ncc(C(=O)Nc3ccccc3Cl)c2c1]

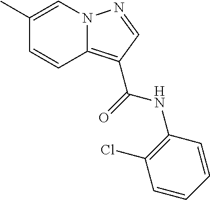 [SMILES: Cc1ccc2c(C(=O)Nc3ccccc3Cl)cnn2c1]

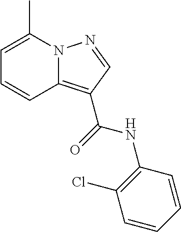 [SMILES: Cc1cccc2c(C(=O)Nc3ccccc3Cl)cnn12]

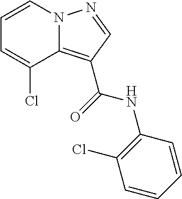 [SMILES: O=C(Nc1ccccc1Cl)c1cnn2cccc(Cl)c12]

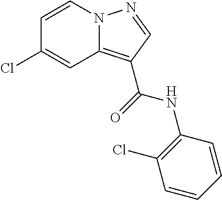 [SMILES: O=C(Nc1ccccc1Cl)c1cnn2ccc(Cl)cc12]

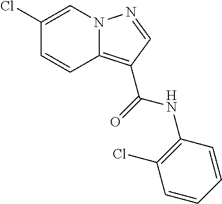 [SMILES: O=C(Nc1ccccc1Cl)c1cnn2cc(Cl)ccc12]

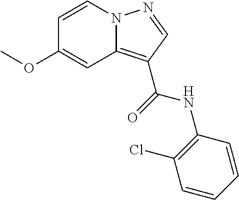 [SMILES: COc1ccn2ncc(C(=O)Nc3ccccc3Cl)c2c1]

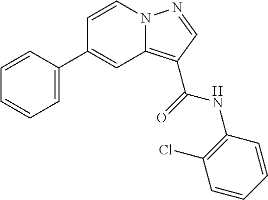 [SMILES: O=C(Nc1ccccc1Cl)c1cnn2ccc(-c3ccccc3)cc12]

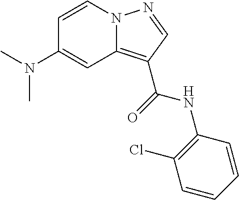 [SMILES: CN(C)c1ccn2ncc(C(=O)Nc3ccccc3Cl)c2c1]

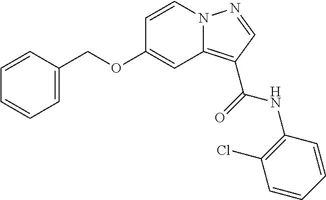 [SMILES: O=C(Nc1ccccc1Cl)c1cnn2ccc(OCc3ccccc3)cc12]

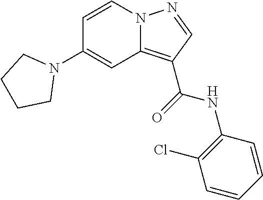 [SMILES: O=C(Nc1ccccc1Cl)c1cnn2ccc(N3CCCC3)cc12]

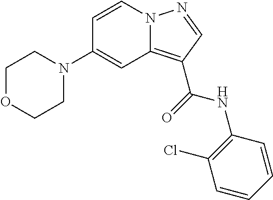 [SMILES: O=C(Nc1ccccc1Cl)c1cnn2ccc(N3CCOCC3)cc12]

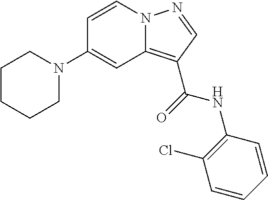 [SMILES: O=C(Nc1ccccc1Cl)c1cnn2ccc(N3CCCCC3)cc12]

or a pharmaceutically acceptable salt thereof.

In some embodiments, the present disclosure provides a compound selected from any one of the compounds listed in Table B:

TABLE B

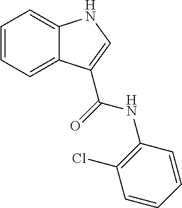 [SMILES: O=C(Nc1ccccc1Cl)c1c[nH]c2ccccc12]

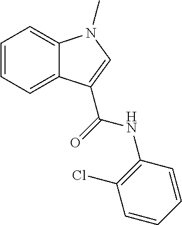 [SMILES: Cn1cc(C(=O)Nc2ccccc2Cl)c2ccccc21]

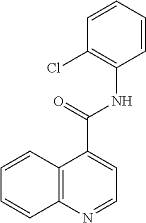 [SMILES: O=C(Nc1ccccc1Cl)c1ccnc2ccccc12]

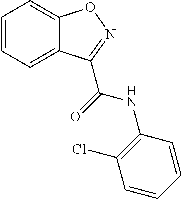 [SMILES: O=C(Nc1ccccc1Cl)c1noc2ccccc12]

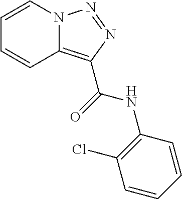 [SMILES: O=C(Nc1ccccc1Cl)c1nnn2ccccc12]

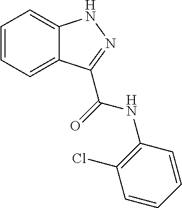 [SMILES: O=C(Nc1ccccc1Cl)c1n[nH]c2ccccc12]

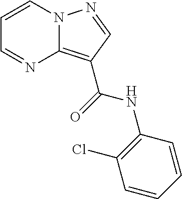 [SMILES: O=C(Nc1ccccc1Cl)c1cnn2cccnc12]

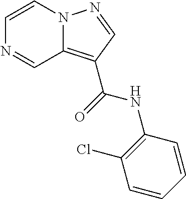 [SMILES: O=C(Nc1ccccc1Cl)c1cnn2ccncc12]

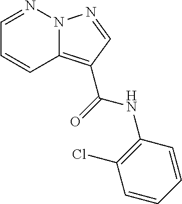 [SMILES: O=C(Nc1ccccc1Cl)c1cnn2ncccc12]

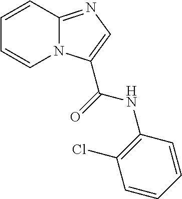 [SMILES: O=C(Nc1ccccc1Cl)c1cnc2ccccn12]

or a pharmaceutically acceptable salt thereof.

Compound of Formula (IIa)

In some embodiments, the present disclosure provides a compound of Formula

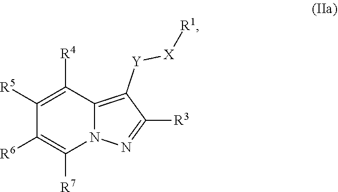

or a pharmaceutically acceptable salt thereof, wherein:
X and Y are each independently selected from C(═O) and NR²;
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from NR^{c1}R^{d1}, 4-6 membered heterocycloalkyl comprising at least one N atom, and L-R⁸; wherein said 4-6 membered heterocycloalkyl comprising at least one N atom is optionally substituted with 1, 2, or 3 substituents independently selected from R^{Cy1};
L is selected from C₁₋₆ alkylene, O—C₁₋₆ alkylene, and NH—C₁₋₆ alkylene, each of which is optionally substituted with 1 or 2 independently selected R⁹;
R⁸ is selected from NR^{c1}R^{d1}, C(O)NR^{c1}R^{d1}, and OC(O)NR^{c1}R^{d1};
each R⁹ is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
R⁴ and R⁷ are each independently selected from H, halo, CN, OR^{a1}, C₁₋₃ alkyl, and C₁₋₃ haloalkyl, wherein said C₁₋₃ alkyl is optionally substituted with 1 or 2 independently selected R^{g};
R⁶ is a reactive electrophilic warhead group;
R^{a1}, R^{b1}, R, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

In some embodiments, X is C(═O) and Y is NH.

In some embodiments, X is NH and Y is C(═O).

In some embodiments, R¹ is selected from C₆₋₁₀ aryl and 5-14 membered heteroaryl, each of which is optionally substituted with 1 or 2 substituents independently selected from halo and OR^{a1}.

In some embodiments, R³ is selected from H and C₁₋₃ alkyl.

In some embodiments, R⁵ is 4-6 membered heterocycloalkyl comprising at least one N atom, which is optionally substituted with 1 or 2 substituents independently selected from R^{Cy1}.

In some embodiments, R⁵ is and L-R⁸.

In some embodiments, L is selected from C₁₋₆ alkylene, O—C₁₋₆ alkylene, and NH—C₁₋₆ alkylene.

In some embodiments, R⁸ is NR^{c1}R^{d1}.

In some embodiments, R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

In some embodiments, R⁴ is selected from H, halo, C₁₋₃ alkyl, CN, OR^{a1}, and C₁₋₃ haloalkyl.

In some embodiments, R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

In some embodiments:
R¹ is selected from C₆₋₁₀ aryl and 5-14 membered heteroaryl, each of which is optionally substituted with 1 or 2 substituents independently selected from halo and OR^{a1};
R³ is selected from H and C₁₋₃ alkyl;
R⁵ is selected from L-R⁸ and 4-6 membered heterocycloalkyl comprising at least one N atom, which is optionally substituted with 1 or 2 substituents independently selected from R^{Cy1};
L is selected from C₁₋₆ alkylene, O—C₁₋₆ alkylene, and NH—C₁₋₆ alkylene;
R⁸ is NR^{c1}R^{d1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, C₁₋₃ alkyl, CN, OR^{a1}, and C₁₋₃ haloalkyl; and
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

In some embodiments, the compound of Formula (IIa) is selected from any one of the following compounds:

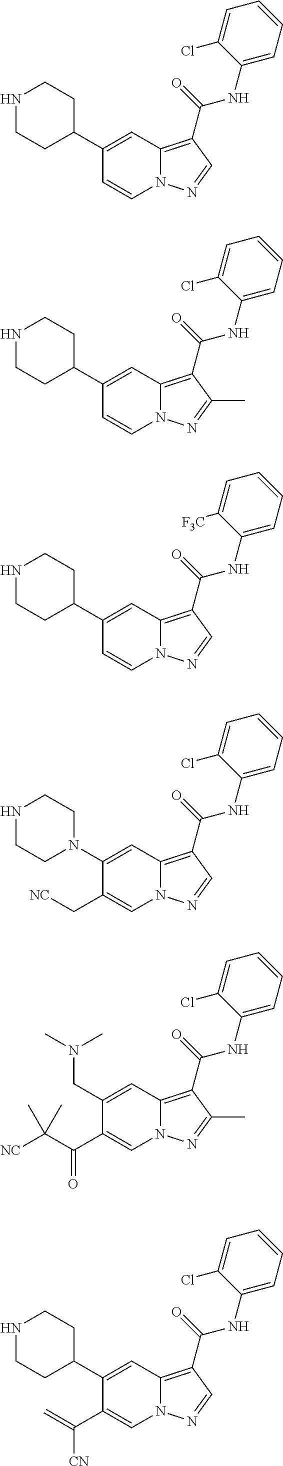 [SMILES: O=C(Nc1ccccc1Cl)c1cnn2ccc(C3CCNCC3)cc12 ; Cc1nn2ccc(C3CCNCC3)cc2c1C(=O)Nc1ccccc1Cl ; O=C(Nc1ccccc1C(F)(F)F)c1cnn2ccc(C3CCNCC3)cc12 ; N#CCc1cn2ncc(C(=O)Nc3ccccc3Cl)c2cc1N1CCNCC1 ; Cc1nn2cc(C(=O)C(C)(C)C#N)c(CN(C)C)cc2c1C(=O)Nc1ccccc1Cl ; C=C(C#N)c1cn2ncc(C(=O)Nc3ccccc3Cl)c2cc1C1CCNCC1]

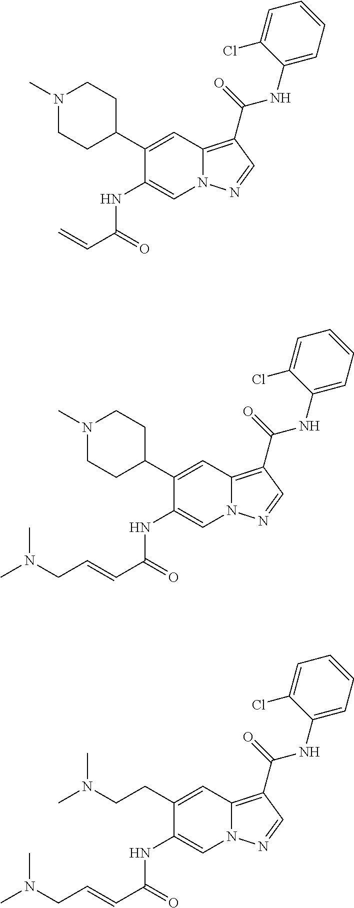 [SMILES: C=CC(=O)Nc1cn2ncc(C(=O)Nc3ccccc3Cl)c2cc1C1CCN(C)CC1 ; CN(C)C/C=C/C(=O)Nc1cn2ncc(C(=O)Nc3ccccc3Cl)c2cc1C1CCN(C)CC1 ; CN(C)C/C=C/C(=O)Nc1cn2ncc(C(=O)Nc3ccccc3Cl)c2cc1CCN(C)C]

or a pharmaceutically acceptable salt thereof.

Compound of Formula (III)

In some embodiments, the present disclosure provides a method of treating a neurodegenerative disease or condition, the method comprising administering to a subject in need thereof a therapeutically effective amount of a compound of Formula (III):

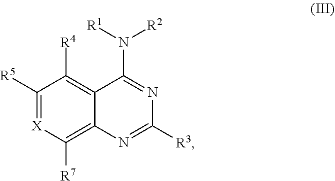

or a pharmaceutically acceptable salt thereof, wherein:
X is selected from CR⁶ and NR⁶
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, C₁₋₃ alkyl, and C₁₋₃ haloalkyl, wherein said C₁₋₃ alkyl is optionally substituted with 1 or 2 independently selected R^{g};
R⁶ is selected from H, halo, CN, OR^{a1}, C₁₋₆ alkyl, C₁₋₆ haloalkyl, and a reactive electrophilic warhead group;
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

In some embodiments, X is CR⁶.

In some embodiments, X is NR⁶.

In some embodiments, R¹ is selected from C₆₋₁₀ aryl and 5-14 membered heteroaryl, each of which is optionally substituted with 1 or 2 substituents independently selected from halo and OR^{a1}.

In some embodiments, R² is H.

In some embodiments, R³ is selected from H and C₁₋₃ alkyl.

In some embodiments, R⁵ is selected from NR^{c1}R^{d1}, OR^{a1}, C₁₋₆ alkyl, and Cy¹, wherein said C₁₋₆ alkyl is optionally substituted with NR^{c1}R^{d1}.

In some embodiments, Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

In some embodiments, R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

In some embodiments, R⁴ is selected from H, halo, CN, OR^{a1}, and C₁₋₃ haloalkyl.

In some embodiments, R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

In some embodiments:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is selected from NR^{c1}R^{d1}, OR^{a1}, C₁₋₆ alkyl, and Cy¹, wherein said C₁₋₆ alkyl is optionally substituted with NR^{c1}R^{d1};
Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl; and
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

In some embodiments, the compound of Formula (III) is selected from any one of the compounds listed in Table D:

TABLE D

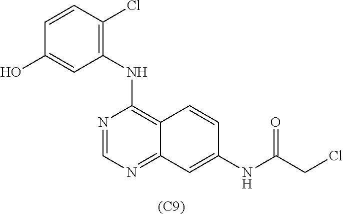 [SMILES: O=C(CCl)Nc1ccc2c(Nc3cc(O)ccc3Cl)ncnc2c1 ; "(C9)"]

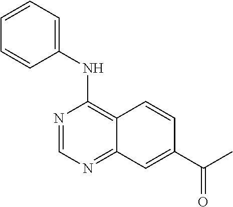 [SMILES: CC(=O)c1ccc2c(Nc3ccccc3)ncnc2c1]

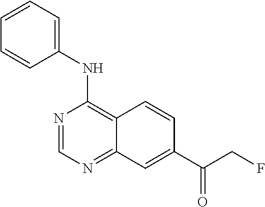 [SMILES: O=C(CF)c1ccc2c(Nc3ccccc3)ncnc2c1]

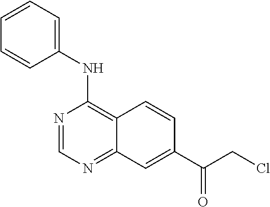 [SMILES: O=C(CCl)c1ccc2c(Nc3ccccc3)ncnc2c1]

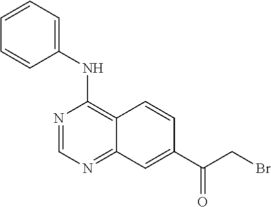 [SMILES: O=C(CBr)c1ccc2c(Nc3ccccc3)ncnc2c1]

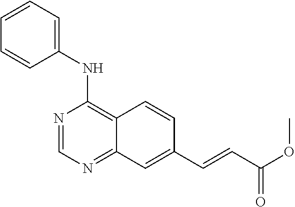 [SMILES: COC(=O)/C=C/c1ccc2c(Nc3ccccc3)ncnc2c1]

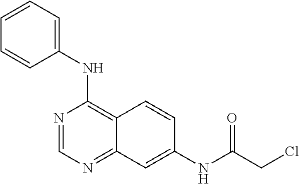 [SMILES: O=C(CCl)Nc1ccc2c(Nc3ccccc3)ncnc2c1]

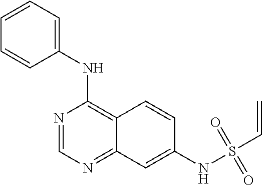 [SMILES: C=CS(=O)(=O)Nc1ccc2c(Nc3ccccc3)ncnc2c1]

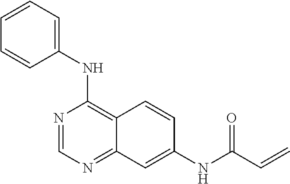 [SMILES: C=CC(=O)Nc1ccc2c(Nc3ccccc3)ncnc2c1]

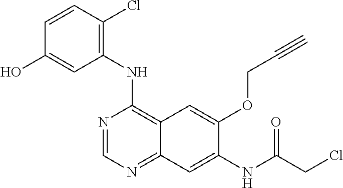 [SMILES: C#CCOc1cc2c(Nc3cc(O)ccc3Cl)ncnc2cc1NC(=O)CCl]

or a pharmaceutically acceptable salt thereof.

Compound of Formula (IIIa)

In some embodiments, the present disclosure provides a compound of Formula

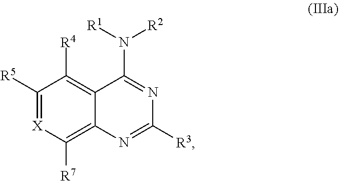

or a pharmaceutically acceptable salt thereof, wherein:
X is selected from CR⁶ and NR⁶
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from NR^{c1}R^{d1} 4-6 membered heterocycloalkyl comprising at least one N atom, and L-R⁸; wherein said 4-6 membered heterocycloalkyl comprising at least one N atom is optionally substituted with 1, 2, or 3 substituents independently selected from R^{Cy1};
L is selected from C₁₋₆ alkylene, O—C₁₋₆ alkylene, and NH—C₁₋₆ alkylene, each of which is optionally substituted with 1 or 2 independently selected R⁹;
R⁸ is selected from NR^{c1}R^{d1}, C(O)NR^{c1}R^{d1}, and OC(O)NR^{c1}R^{d1};
each R⁹ is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
R⁴ and R⁷ are each independently selected from H, halo, CN, OR^{a1}, C₁₋₃ alkyl, and C₁₋₃ haloalkyl, wherein said C₁₋₃ alkyl is optionally substituted with 1 or 2 independently selected R^{g};
R⁶ is a reactive electrophilic warhead group;
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

In some embodiments, X is CR⁶.

In some embodiments, X is NR⁶.

In some embodiments, R¹ is selected from C₆₋₁₀ aryl and 5-14 membered heteroaryl, each of which is optionally substituted with 1 or 2 substituents independently selected from halo and OR^{a1}.

In some embodiments, R² is H.

In some embodiments, R³ is selected from H and C₁₋₃ alkyl.

In some embodiments, R⁵ is 4-6 membered heterocycloalkyl comprising at least one N atom, which is optionally substituted with 1 or 2 substituents independently selected from R^{Cy1}.

In some embodiments, R⁵ is and L-R⁸.

In some embodiments, L is selected from C₁₋₆ alkylene, O—C₁₋₆ alkylene, and NH—C₁₋₆ alkylene.

In some embodiments, R⁸ is NR^{c1}R^{d1}.

In some embodiments, R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

In some embodiments, R⁴ is selected from H, halo, C₁₋₃ alkyl, CN, OR^{a1}, and C₁₋₃ haloalkyl.

In some embodiments, R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

In some embodiments:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is selected from L-R⁸ and 4-6 membered heterocycloalkyl comprising at least one N atom, which is optionally substituted with 1 or 2 substituents independently selected from R^{Cy1};
L is selected from C₁₋₆ alkylene, O—C₁₋₆ alkylene, and NH—C₁₋₆ alkylene;
R⁸ is NR^{c1}R^{d1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, C₁₋₃ alkyl, CN, OR^{a1}, and C₁₋₃ haloalkyl; and
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

In some embodiments, the compound of Formula (IIIa) is selected from any one of the following compounds:

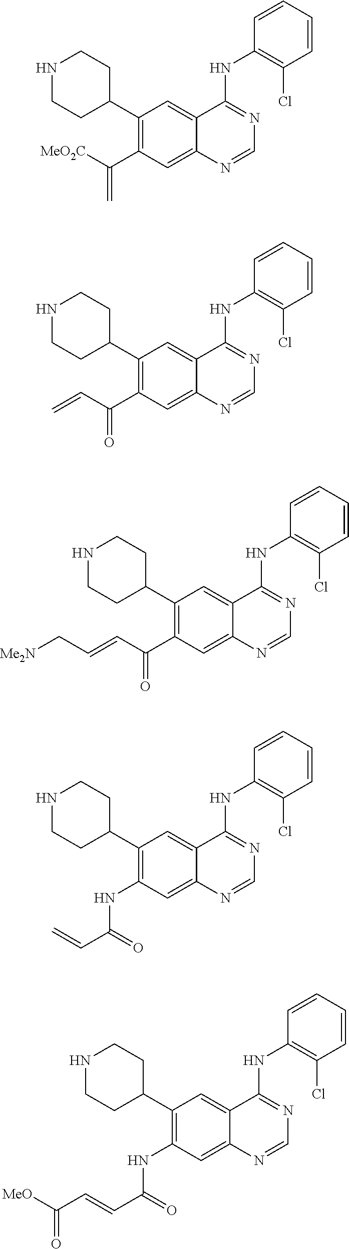 [SMILES: C=C(C(=O)OC)c1cc2ncnc(Nc3ccccc3Cl)c2cc1C1CCNCC1 ; C=CC(=O)c1cc2ncnc(Nc3ccccc3Cl)c2cc1C1CCNCC1 ; CN(C)C/C=C/C(=O)c1cc2ncnc(Nc3ccccc3Cl)c2cc1C1CCNCC1 ; C=CC(=O)Nc1cc2ncnc(Nc3ccccc3Cl)c2cc1C1CCNCC1 ; COC(=O)/C=C/C(=O)Nc1cc2ncnc(Nc3ccccc3Cl)c2cc1C1CCNCC1]

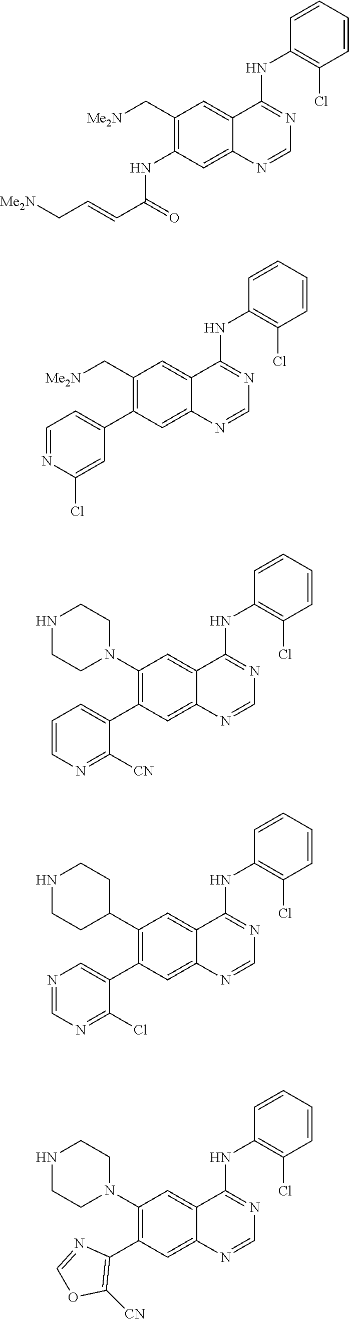 [SMILES: CN(C)C/C=C/C(=O)Nc1cc2ncnc(Nc3ccccc3Cl)c2cc1CN(C)C ; CN(C)Cc1cc2c(Nc3ccccc3Cl)ncnc2cc1-c1ccnc(Cl)c1 ; N#Cc1ncccc1-c1cc2ncnc(Nc3ccccc3Cl)c2cc1N1CCNCC1 ; Clc1ccccc1Nc1ncnc2cc(-c3cncnc3Cl)c(C3CCNCC3)cc12 ; N#Cc1ocnc1-c1cc2ncnc(Nc3ccccc3Cl)c2cc1N1CCNCC1]

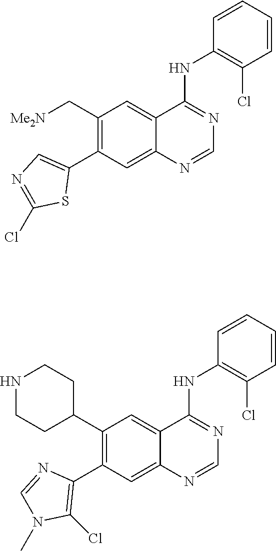 [SMILES: CN(C)Cc1cc2c(Nc3ccccc3Cl)ncnc2cc1-c1cnc(Cl)s1 ; Cn1cnc(-c2cc3ncnc(Nc4ccccc4Cl)c3cc2C2CCNCC2)c1Cl]

or a pharmaceutically acceptable salt thereof.

Pharmaceutically Acceptable Salts

In some embodiments, a salt (e.g., pharmaceutically acceptable salt) of a compound of any one of the Formulae disclosed herein is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of any one of the Formulae disclosed herein include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of any one of the Formulae disclosed herein include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH-(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the compounds of any one of the Formulae disclosed herein, or pharmaceutically acceptable salts thereof, are substantially isolated.

Pharmaceutical Compositions

The present application also provides pharmaceutical compositions comprising an effective amount of a compound of any one of the Formulae disclosed herein, or a pharmaceutically acceptable salt thereof, and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, intraventricular, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

Dosages and Regimens

In the pharmaceutical compositions of the present application, a compound of the present disclosure (e.g., a compound of any one of the Formulae disclosed herein) is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound (e.g., a compound of any one of the Formulae disclosed herein) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of any one of the Formulae disclosed herein is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

Methods of Use

In some embodiments, the present disclosure provides a method of inhibiting EphB3 receptor signaling in an astrocyte cell, the method comprising contacting the astrocyte cell with an effective amount of a compound of any of the Formulae disclosed herein, or a pharmaceutically acceptable salt thereof.

In some embodiments, the present disclosure provides a method of inhibiting EphB3 receptor signaling in an astrocyte cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting EphB3 tyrosine kinase in an astrocyte cell, the method comprising contacting the astrocyte cell with an effective amount of a compound of any of the Formulae disclosed herein, or a pharmaceutically acceptable salt thereof.

In some embodiments, the present disclosure provides a method of inhibiting EphB3 tyrosine kinase in an astrocyte cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting pro-inflammatory response in an astrocyte cell, the method comprising contacting the astrocyte cell with an effective amount of a compound of any of the Formulae disclosed herein, or a pharmaceutically acceptable salt thereof.

In some embodiments, the present disclosure provides a method of inhibiting pro-inflammatory response in an astrocyte cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting production of TNF-α in an astrocyte cell, the method comprising contacting the astrocyte cell with an effective amount of a compound of any of the Formulae disclosed herein, or a pharmaceutically acceptable salt thereof.

In some embodiments, the present disclosure provides a method of inhibiting production of TNF-α in an astrocyte cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of reducing production of IL6 in an astrocyte cell, the method comprising contacting the astrocyte cell with an effective amount of a compound of any of the Formulae disclosed herein, or a pharmaceutically acceptable salt thereof.

In some embodiments, the present disclosure provides a method of reducing production of IL6 in an astrocyte cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of reducing production of CCl2 in an astrocyte cell, the method comprising contacting the astrocyte cell with an effective amount of a compound of any of the Formulae disclosed herein, or a pharmaceutically acceptable salt thereof.

In some embodiments, the present disclosure provides a method of reducing production of CCl2 in an astrocyte cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting phosphorylation of AKT in an astrocyte cell, the method comprising contacting the astrocyte cell with an effective amount of a compound of any of the Formulae disclosed herein, or a pharmaceutically acceptable salt thereof.

In some embodiments, the present disclosure provides a method of inhibiting phosphorylation of AKT in astrocyte cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting activation of mTOR pathway in an astrocyte cell, the method comprising contacting the astrocyte cell with an effective amount of a compound of any of the Formulae disclosed herein, or a pharmaceutically acceptable salt thereof.

In some embodiments, the present disclosure provides a method of inhibiting activation of mTOR pathway in an astrocyte cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting production of mitochondrial reactive oxygen species (ROS) in an astrocyte cell, the method comprising contacting the astrocyte cell with an effective amount of a compound of any of the Formulae disclosed herein, or a pharmaceutically acceptable salt thereof.

In some embodiments, the present disclosure provides a method of inhibiting production of mitochondrial reactive oxygen species (ROS) in an astrocyte cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of restoring a homeostatic state or a normal activation state of glial cell or a neuron cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of reducing pro-inflammatory macrophages in a peripheral nervous system of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting pro-inflammatory response in a microglial cell, the method comprising contacting the microglial cell with an effective amount of a compound of any of the Formulae disclosed herein, or a pharmaceutically acceptable salt thereof.

In some embodiments, the present disclosure provides a method of inhibiting pro-inflammatory response in a microglial cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting pro-inflammatory response in a microglial cell, the method comprising contacting the microglial cell with an effective amount of a compound of any of the Formulae disclosed herein, or a pharmaceutically acceptable salt thereof.

In some embodiments, the present disclosure provides a method of inhibiting pro-inflammatory response in a microglial cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting NFkB activation in a microglial cell, the method comprising contacting the microglial cell with an effective amount of a compound of any of the Formulae disclosed herein, or a pharmaceutically acceptable salt thereof.

In some embodiments, the present disclosure provides a method of inhibiting NFkB activation in a microglial cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting production of TNF-α in a microglial cell, the method comprising contacting the microglial cell with an effective amount of a compound of any of the Formulae disclosed herein, or a pharmaceutically acceptable salt thereof.

In some embodiments, the present disclosure provides a method of inhibiting production of TNF-α in a microglial cell of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting demyelination of a neuron cell, the method comprising contacting the neuron cell with an effective amount of a compound of any of the Formulae disclosed herein, or a pharmaceutically acceptable salt thereof.

In some embodiments, the present disclosure provides a method of inhibiting demyelination of a neuron cell in a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting expression of astrocyte and microglial transcriptional modules associated with a promotion of CNS inflammation and neurodegeneration a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of reducing pro-inflammatory T cells in a central nervous system of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of reducing pro-inflammatory macrophages in a central nervous system of a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting CNS inflammation in a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of inhibiting PNS inflammation in a subject, the method comprising administering to the subject a therapeutically effective amount of a compound of any of the Formulae disclosed herein, or a pharmaceutically acceptable salt thereof, or a pharmaceutical composition comprising same. In some aspects of these embodiments, the method comprises contacting neuroglia of the PNS of the subject. Examples of such neuroglia include Schwann cells (neurolemmocytes) and satellite cells. Hence, in some embodiments the present disclosure provides a method of contacting a Schwann cell or a satellite cell of the PNS of a subject, the method comprising administering to the subject in need thereof a therapeutically effective amount of a compound of any of the Formulae disclosed herein, or a pharmaceutically acceptable salt thereof, or a pharmaceutical composition comprising same. In some embodiments, the diseases disclosed herein include conditions where PNS glial cells enter the CNS of the subject. See, e.g., DOI 10.3389/fcell.2016.00058, DOI 10.1242/dev.068411, DOI 10.1007/s00401-019-02011-1, DOI 10.1038/cddis.2015.262, and DOI 10.3389/fncel.2017.00323, which are incorporated herein by reference in their entirety.

In some embodiments, the present disclosure provides a method of inhibiting neurodegeneration in a subject, the method comprising administering to the subject a therapeutically effective amount of a compound of any of the Formulae disclosed herein, or a pharmaceutically acceptable salt thereof, or a pharmaceutical composition comprising same.

In some embodiments, the present disclosure provides a method of treating a neurodegenerative disease or condition, the method comprising administering to a subject in need thereof a therapeutically effective amount of a compound of any of the Formulae disclosed herein, or a pharmaceutically acceptable salt thereof, or a pharmaceutical composition comprising same.

Suitable examples of neurodegenerative diseases or conditions include autoimmune encephalomyelitis (AE) (including EAE), chronic inflammatory demyelinating polyneuropathy, acute disseminated encephalomyelitis (ADEM), multiple sclerosis (MS), amyotrophic lateral sclerosis, schizophrenia, Alzheimer's disease, and Parkinson's disease. In some embodiments, the AE is acute. In some embodiments, the AE is chronic. In some embodiments, the MS is a late stage MS disease. In some embodiments, the MS is in a relapsing stage.

Other examples of neurodegenerative diseases include dementia, frontotemporal lobar dementia, Huntington's disease, accessory nerve disorder, autonomic dysreflexia, peripheral neuropathy, chemotherapy-induced peripheral neuropathies, mononeuropathy, polyneuropathy, radial neuropathy, ulnar neuropathy, Villaret's syndrome, Charcot-Marie-Tooth disease, diabetic neuropathy, nerve paralysis, progressive bulbar palsy, pseudobulbar palsy, spinal bulbar muscular atrophy, myotonic dystrophy, inclusion body myositis, prion disease, seizure disorders, lysosomal storage disorders, transmissible spongiform encephalopathy, Creutzfeldt-Jacob disease (CJD), spinocerebellar ataxia, spinal muscular atrophy, and Horner's syndrome. In some embodiments, the neurodegenerative disease or condition is driven by dysregulated adaptive (T cells and B cells) and innate (monocytes, microglia and astrocytes) immune responses. Some examples of diseases or conditions include adrenoleukodystrophy, macular degeneration, glaucoma, optic neuritis, Guillain-Barre syndrome, and Lewy Body syndrome.

In some embodiments, the present disclosure provides a method of treating a disease or condition selected from autoimmune encephalomyelitis, chronic inflammatory demyelinating polyneuropathy, acute disseminated encephalomyelitis, multiple sclerosis, amyotrophic lateral sclerosis, schizophrenia, Alzheimer's disease, Parkinson's disease, acute disseminated encephalomyelitis (“ADEM”), concentric sclerosis, Charcot-Marie-Tooth disease, Guillain-Barre syndrome, HTLV-I associated myelopathy (“HAM”), neuromyelitis optica, Schilder's disease, and transverse myelitis.

In some embodiments, the present disclosure provides a method of treating a chronic autoimmune inflammatory disease, the method comprising administering to a subject in need thereof a therapeutically effective amount of a compound of any of the Formulae disclosed herein, or a pharmaceutically acceptable salt thereof, or a pharmaceutical composition comprising same.

In some embodiments, the present disclosure provides a method of treating a neuronal system injury characterized by EphB3 kinase activity, the method comprising administering to a subject in need thereof a therapeutically effective amount of a compound as disclosed herein, or a pharmaceutically acceptable salt thereof, or a pharmaceutical composition comprising same. In some embodiments, the neuronal system injury is a central nervous system injury. In some embodiments, the central nervous system injury is selected from cerebral ischemia and traumatic brain injury. In some embodiments, the present disclosure provides a method of treating a disease or condition selected from stroke, spinal cord injury, and traumatic brain injury.

In some embodiments, the present disclosure provides a method of treating a cancer, the method comprising administering to a subject in need thereof a therapeutically effective amount of a compound as disclosed herein, or a pharmaceutically acceptable salt thereof, or a pharmaceutical composition comprising same. In some embodiments, the cancer is selected from leukemia, non-small cell lung cancer, colon cancer, CNS cancer, melanoma, ovarian cancer, renal cancer, prostate cancer, and breast cancer.

Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

Combinations

The compounds of the present disclosure can be used on combination with at least one medication or therapy useful, e.g., in treating or alleviating symptoms of a neurological or neurodegenerative disease or condition.

In some embodiments, the additional therapeutic agent that could be administered in combination with the compound of the present disclosure is useful in treating AE. Suitable examples of such therapeutic agents include a steroid anti-inflammatory drug (e.g., prednisone), NSAIDs (e.g., naproxen), anti-TNF-α therapy, rituximab, and cyclophosphamide.

In some embodiments, the additional therapeutic agent that could be administered in combination with the compound of the present disclosure is useful in treating MS. Suitable examples of such therapeutic agents include fingolimod, glatiramer acetate (Copaxone), cladribine, dimethyl fumarate, monomethyl fumarate, diroximel fumarate, mitoxantrone, ozanimod, teriflunomide, ibudilast, siponimod, cyclophosphamide, alemtuzumab, interferon beta-1a, interferon beta-1b, natalizumab, ocrelizumab, and ofatumumab.

In some embodiments, the additional therapeutic agent that could be administered in combination with the compound of the present disclosure is useful in treating ALS. Suitable examples of such therapeutic agents include masitinib, riluzole, and edaravone.

In some embodiments, the additional therapeutic agent that could be administered in combination with the compound of the present disclosure is a MAO inhibitor. Suitable examples of MAO inhibitors include bifemelane, moclobemide, pirlindole, toloxatone, rasagiline, selegiline, safinamide, hydrazine, isocarboxazid, hydracarbazine, phenelzine, and tranylcypromine.

In some embodiments, the additional therapeutic agent that could be administered in combination with the compound of the present disclosure is a Ap-targeting antibody. Suitable examples of such antibodies include aducanumab, gantenerumab, solanezumab, crenezumab, CAD106, CNP520, and UB-311. In some embodiments, the additional therapeutic agent that could be administered in combination with the compound of the present disclosure is useful in treating Alzheimer's disease. Suitable examples of such therapeutic agents include donezepil, memantine, galantamine, rivastigmine, and memantine/donepezil.

In some embodiments, the additional therapeutic agent that could be administered in combination with the compound of the present disclosure is a T-cell targeting therapeutic. Suitable examples of such therapeutics include alemtuzumab, interferon beta-1a, interferon beta-1b, natalizumab, ocrelizumab, and ofatumumab.

In some embodiments, the additional therapeutic agent that could be administered in combination with the compound of the present disclosure is useful in treating Parkinson's disease. Suitable examples of such therapeutic agents include amantadine, benzatropine, entacapone, ropinirole, tolcapone, selegiline, pramipexole, levodopa, carbidopa/levodopa, and carbidopa/levodopa/entacapone.

In some embodiments, the additional therapeutic agent that could be administered in combination with the compound of the present disclosure is an enzyme replacement therapy, a gene modification therapy or a gene therapy treatment for genetic neurodegenerative diseases or disorders (e.g., spinal muscular atrophy (“SMA”), ALS, HD, PD, and lysosomal storage disorders).

The compounds of the present disclosure may be administered to the patient simultaneously with the additional therapeutic agent (in the same pharmaceutical composition or dosage form or in different compositions or dosage forms) or consecutively (the additional therapeutic agent may be administered in a separate pharmaceutical composition or dosage form before or after administration of the compound of the present disclosure).

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “aromatic” refers to a carbocycle or heterocycle having one or more polyunsaturated rings having aromatic character (i.e., having (4n+2) delocalized R (pi) electrons where n is an integer).

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cn-m” indicates a range which includes the endpoints, wherein n and m are integers and indicate the number of carbons. Examples include C₁₋₄, C₁₋₆, and the like.

As used herein, the term “Cn-m alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cn-m haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cn-m alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cn-m alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cn-m haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cn-m alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cn-m-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cn-m alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (e.g., n-propoxycarbonyl and isopropoxycarbonyl), butoxycarbonyl (e.g., n-butoxycarbonyl and tert-butoxycarbonyl), and the like.

As used herein, the term “carboxy” refers to a —C(O)OH group.

As used herein, “halo” refers to F, Cl, Br, or I. In some embodiments, a halo is F, C₁, or Br.

As used herein, the term “aryl,” employed alone or in combination with other terms, refers to an aromatic hydrocarbon group, which may be monocyclic or polycyclic (e.g., having 2, 3 or 4 fused rings). The term “Cn-m aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

As used herein, the term “oxo” refers to an oxygen atom as a divalent substituent, forming a carbonyl group when attached to a carbon (e.g., C═O), or attached to a heteroatom forming a sulfoxide or sulfone group.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the Eph kinase with a compound of the invention includes the administration of a compound of the present invention to an individual or patient, such as a human, having Eph kinase, as well as, for example, introducing a compound of the invention into a sample containing a cellular or purified preparation containing the Eph kinase.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### Examples

Materials and Methods

Mice. Experimental animals were kept in the pathogen-free facility at the Hale Building for Transformative Medicine at Brigham and Women's Hospital and all in vivo experiments were carried out in accordance with the Institutional Animal Care and Use Committee (IACUC) guidelines. Adult C57BL/6J (#000664) and NOD/ShiLtJ (NOD mice) (#001976) were obtained from the Jackson Laboratory. Postnatal pups were bred in the facility and used between PO-P3 to establish astrocyte and microglia cultures. B6.Cg-Tg(Gfap-cre) 73.12Mvs/J hemizygous mice (The Jackson Laboratory, #012886) were crossed to homozygous B6; 129P2 Gt(ROSA)26Sortm1(CAG-RABVgp4,-TVA)Arenk/J mice (The Jackson Laboratory, #024708).

Mouse primary astrocyte cultures. Cerebral cortices from neonatal mice (1-3 days) were dissected, carefully stripped of their meninges, homogenized on 0.25% trypsin-EDTA (#25200-072, Thermo Fisher Scientific), incubated at 37° C. for 15 min and dispersed to single-cell level by passing through a cell strainer. The cell suspension was then cultured at 37° C. in humidified 5% CO₂ on poly-L-Lysine (#P4707, Sigma-Aldrich) pre-coated cell culture flasks. Medium was replaced every 3-5 days. The cells reached confluence after 7-10 days. Microglia were separated by shaking the glia culture at 225 rpm at 37° C. for at least 3 h and washing extensively with 1×PBS. Astrocytes were detached by mild trypsinization with Trypsin-EDTA (0.05%) at 37° C. and plated. The complete astrocyte culture medium is DMEM/F12 medium (#10565018, GIBCO) supplied with 10% FBS and 100 unit/mL penicillin/streptomycin. For qPCR, cytokine and signaling studies, primary mouse astrocyte cultures were pre-treated with the stated concentration of A38, 10 μM ZSTK474 (#S1072 Selleck Chemicals) or 100 nM rapamycin (#R-5000 LC Laboratories) for 30 min and then, when indicated, stimulated with 50 ng/mL TNFα (#410-MT010, R&D Systems) and 100 ng/mL IL-1β (#401-ML-025, R&D Systems) for 18 h unless otherwise indicated. Mouse astrocytes primary cultures were additionally depleted of microglia by magnetic separation using anti CD11b and antiCD45 magnetic beads combined (#130-049-301 and #130-052-301, Miltenyi Biotec) and separated through LS columns (#130-042-401, Miltenyi Biotec) following manufacturers instruction. The negative fraction collected corresponded to astrocytes that were 98% pure, while the positive fraction was enriched in microglia 95% pure. Both cell types were then cultured separately for downstream procedures. When stated, after siRNA knockdown and stimulation, astrocytes were washed and cocultured 1:1 with microglia for 18 h. Finally, microglia and astrocytes of each condition where magnetically separated as before and lysed for downstream gene expression analysis. Recombinant Human Ephrin-B3 Fc Chimera Protein (R&D #7924-EB-050), recombinant Mouse Ephrin-B3 Fc Chimera Protein, (R&D #7655-EB-050) and Recombinant Mouse EphB3 Fc Chimera Protein (R&D Systems #432-B3-200) were incubated overnight (ON) at a concentration of 2.5 pgr/cm² on Poly-L-Lysine pre-coated plates, and after 1×PBS rinse, cells were seeded.

Isolation, culture and stimulation of human primary astrocytes. Human fetal astrocytes were isolated from human CNS tissue (cerebral hemispheres) from fetuses at 17-23 weeks of gestation obtained from the Laboratory of Developmental Biology (Eunice Kennedy Shriver National Institute of Child Health and Human Development, project number: 5R24HD000836) following Canadian Institutes of Health Research—approved guidelines. The sex of the human astrocytes used was unidentified. Astrocyte cultures were obtained by dissociation of the fetal CNS with 0.25% trypsin (#25200-072, Thermo Fisher Scientific) and 50 μg/mL DNase I (#10104159001, Roche) followed by mechanical dissociation. After washing, the cell suspension was plated at a concentration of 3-5×10⁶ cells mL⁻¹ on poly-1-lysine (#P4707, Sigma-Aldrich) pre-coated 75 cm² flasks in DMEM supplemented with 10% FCS (#SH3007303, Fisher Scientific) and penicillin/streptomycin.

To obtain pure astrocytes, the mixed CNS cell culture (containing astrocyte, microglia and neuron) was passaged upon confluency, starting at 2 weeks post-isolation, using 0.25% trypsin-EDTA (#25200-072, Thermo Fisher Scientific). Human fetal astrocytes were used between passages 2 and 4 and cultures, which corresponds to a time frame of 2 weeks to 3 months post-isolation, and purity (>90%) was determined by immunostaining using anti-glial fibrillary acidic protein (GFAP) rabbit mAb (#05269784001, Roche, 1:100) followed by goat anti-rabbit IgG conjugated with Texas Red (#T-2767, Thermo Fisher Scientific, 1:100). Human astrocytes were stimulated, when indicated, with 10 ng mL⁻¹ of human IL-1β. Treatments with A38 and human Ephrin Fc Chimera were performed similarly to what stated above for murine astrocytes. The cells were harvested for RNA isolation and qPCR, or alternatively washed and cultured in fresh media, which after 2 days of incubation was used for cytokines measurement by ELISA.

Microglia stimulation. Microglia was treated with A38, plated on Ephb3 Fc-chimera coated plates (see above) or incubated with LPS (100 ng/ml Invivogen #tlrl-3pelps) as stated.

Generation of astrocyte-conditioned medium (ACM). To collect ACM murine astrocytes were stimulated as stated above with the indicated pretreatment of 1 hour of the inhibitors A38 and C9. After 24 h, cells were extensively washed with 1×PBS, and incubated with fresh astrocyte complete medium for 48 h. Supernatants were spun down and kept at −80° C. until tested in migration and neurotoxicity assays.

Monocyte Migration Assay. Monocytes were purified from spleens from C57BL/6J mice using CD11b Microbeads (#130-049-601, Miltenyi) following the manufacturer's instructions. 100,000 monocytes were seeded in the upper chamber of a 24-well cell culture transwell, with a 5 μm pore size (#3421, Thermo Fisher Scientific), containing 300 μL of astrocyte-conditioned medium (as detailed earlier) in the bottom chamber. After 3 h incubation at 37° C. in 5% CO₂, cells were collected from the lower chamber after shaking and accutase solution incubation (#A6964, Sigma-Aldrich) and quantified by FACS.

Neurotoxicity Assay. N2A neuronal cells (#CCL-131, ATCC) were plated and pre-activated with 100 ng/mL mouse IFNγ (#485-MI-100; R&D Systems) for 24 h.

Thereafter, after extensive washing with 1×PBS, medium was replaced with ACM and after 24 h the supernatant was harvested for cytotoxicity evaluation by measuring LDH release with CytoTox 96 nonradioactive cytotoxicity assay kit (#G1780, Promega) following the manufacturer's protocol.

In vitro knockdown with siRNA. Smart pools of ON-TARGETplus siRNA of EphB3 (L-043340-00-0005 Dharmacon) or non-targeting control (D-001810-10-05 Dharmacon) were mixed with INTERFERin (#409, Polyplus transfection) in Opti-MEM (#51985-034, GIBCO) and incubated at room temperature for 10 minutes, and then added to primary astrocytes in complete medium following the manufacturer's instructions with a working concentration of siRNA of 1 nM. After 48 h incubation, media was removed and downstream experiments were performed. Knockdown efficiency was confirmed by qPCR.

Analysis of human brain tissue by immunofluorescence. Human brain tissue was obtained from patients with clinical and neuropathological MS diagnosis according to the revised 2010 McDonald's criteria. Tissue samples were collected from healthy donors and MS patients with full ethical approval (BH07.001) and informed consent as approved by the local ethics committee. Autopsy samples were preserved and lesions classified using Luxol Fast Blue/Haematoxylin & Eosin staining and Oil Red O staining as described. Frozen brain tissue from 4 MS patients and 4 healthy controls was cut into 7 μm thick sections, air dried and fixed in ice-cold acetone for 10 minutes. Sections were delipidised in 70% ethanol for 5 minutes, followed by blocking endogenous avidin/biotin using an avidin-biotin blocking kit (Life Technologies, 004303) and non-specific binding of antibodies was blocked with either 10% goat serum or 10% donkey serum (Sigma). Anti EphB3 (rabbit anti-human, Abcam ab133742, 1:25) or a combination of anti Ephrin-B3 (goat anti-human, R&D, AF395, 1:50) and anti TMEM119 (mouse anti-human, Novus Biologicals, NBP2-76985, 1:200) were incubated in blocking buffer ON at 4° C. The next day slides were washed with 0.05% PBS-Tween and either incubated with goat anti-rabbit biotin (DAKO, E0432, 1:500) or a mixture of donkey anti-mouse-Cy3 (Jackson ImmunoResearch, 715-166-147, 1:200) and donkey anti-goat AF488 (Jackson ImmunoResearch, 705-546-147, 1:350) for 40 min at room temperature. EphB3 sections were washed and incubated with streptavidin-AF488 (Jackson ImmunoResearch, 016-540-084, 1:250) for 40 minutes at room temperature and incubated with anti GFAP-Cy3 (mouse anti-human, Sigma C9205, 1:500) for 1 hr at RT. After extensive washing, sections were counterstained with 4′,6-diamidino-2-phenylindole (DAPI) (Sigma, D9542, 1:500) and mounted in Mowiol containing pro-long gold (Life Technologies, P36934). Primary antibodies were omitted to control for non-specific binding. Images (z-stacks) were acquired using a Leica SP5 confocal microscope with Leica LAS AF software and processed using Fiji and LAS X. All settings were kept the same. EphB3 intensity was quantified in GFAP⁺ cells, whereas Ephrin-B3 intensity was quantified in TMEM119⁺ cells.

EAE. EAE was induced in 8-10 week old female C57BL/6 or NOD by subcutaneous immunization with 150 μg MOG35-55 (#110582, Genemed Synthesis) emulsified in 200 μL of complete Freund's adjuvant per mouse, followed by administration of 200 ng pertussis toxin (#180, List biological Laboratories) on days 0 and 2 as described. A38 20 mg kg¹ was administrated i.p. twice a day starting at day 16 after EAE induction for C57BL/6 (at peak of the disease) or at day 30 for NOD (at the beginning of the progressive phase). EAE was assessed as follows: 0, no signs of disease; 1, loss of tone in the tail; 2, hind limb paresis; 3, hind limb paralysis; 4, tetraplegia; 5, moribund.

Compound A38. A38 has desirable physicochemical properties (MW=355, cLogP=2.4 and PSA=57) for a CNS drug, and it was profiled in a number of drug-like property assays. A38 displayed excellent mouse liver microsome stability (Tu2>5 h, Clint=4 μL/min/mg protein), and its oxalate salt had very high aqueous solubility (>10 mg/mL at pH 7.4). A preliminary in vivo pharmacokinetic evaluation, using the mono-oxalate salt of A38, was conducted.

Following IP (intraperitoneal) administration (3 mg/kg in 100% saline solution) to male Sprague-Dawley rats, both plasma and brain concentrations were determined at 0.25, 0.5, 1, 2, and 8 h. In the plasma, the compound reached promising levels (Cₘₐₓ=221 ng/mL) with a plasma half-life (T_{1/2}=2.25 h). Brain levels of A38 were higher (Cₘₐₓ=331 ng/mL), and a brain to plasma ratio of 1.5 to 1 indicated little resistance to brain penetration and that A38 was not a Pgp substrate.

Mononuclear cells. Mononuclear cells, including astrocytes, monocytes, and microglia, were isolated from the CNS as described. Briefly, mice were perfused with 1×PBS and the isolated brain and spinal cords were homogenized with a razor blade, digested in 0.66 mg/mL papain (#P4762, Sigma-Aldrich) for 15 minutes at 37° C. and subsequently 15 minutes with 0.66 mg/mL Collagenase D (#11088858001, Roche) and 8 U/mL DNase I (#90083, Thermo Fisher Scientific). Samples where then homogenized and filtered through a 70 m cell strainer and resuspended in 30% Percoll TM solution (#17-5445-01, GE Healthcare) overlayed by 1×PBS for myelin removal. Isolated mononuclear CNS cells were washed with 1×PBS and stained with fluorochrome-conjugated antibody to FITC anti-CD11b (Cat #11-0112-85, eBioscience), APC anti-CD45 (Cat #17-0451-83, eBioscience), BV421 anti-Ly-6C (Cat #128031, Biolegend), PE anti-mouse CD45R/B220 (Cat #553089, BD Biosciences), PE anti-CD140a (Cat #12-1401-81, eBioscience), PE anti-CD105 (Cat #12-1051-82, eBioscience), PE anti-04 (Cat #FAB1326P, R&D), PE anti-Ly-6G (Cat #127608, BioLegend) and PE anti-mouse TER-119 (Cat #116207, BioLegend). Microglia were sorted as CD11b⁺CD45^{lo} Ly6C1^{lo}, inflammatory monocytes were considered as CD45^{hi}CD11b⁺Ly6C1^{hi}. Astrocytes were sorted as CD11b^{lo}CD45^{lo} Ly6C^{lo}CD105^{lo} CD140a^{lo} CD11b^{lo}Ter119^{lo}CD19^{lo} after the exclusion of erythrocytes, lymphocytes, microglia, oligodendrocytes, and monocytes. FACSAria IIU (BD Biosciences) was used for cell sorting.

Analysis of T cells. For T-cell flow cytometry analysis, CNS single-cell suspensions or splenocytes were stimulated with 50 ng/mL phorbol 12-myristate 13-acetate (PMA, #P1585, Sigma-Aldrich), 1 μM ionomycin (#I9657, Sigma-Aldrich), GolgiSTOP and (#554724, BD Biosciences, 1:1500) GolgiPLUG (#555029, BD Biosciences, 1:1500) in RPMI 1640 medium (#11875119, Life Technologies) containing 10% FBS (#10438026, GIBCO), penicillin/streptomycin (#15140122, GIBCO, 1:100) for 4 h at 37° C. in a 5% CO₂ incubator. Following stimulation, T cells were stained with antibodies against surface markers, and thereafter fixed and stained with antibody against intracellular target protein with an intracellular antibody labeling kit following its instructions (#00-5523, eBioscience).

Antibodies used were: BV421 anti-CD3 (#100227, BioLegend, 1:100), BV605 anti-CD4 (#100547, BioLegend, 1:50), 405 Aqua LIVE/DEAD cell stain kit (#L34966, Thermo Fisher Scientific, 1:400), FITC anti-IFT (#505806, BioLegend, 1:100), PE anti-IL-17a (#12-7177-81, eBioscience, 1:100), APC anti-IL-1β (#505010, BioLegend, 1:100), PerCP-Cy5.5 anti-FoxP3 (#45-5773-82, eBioscience, 1:100). Samples were acquired on an LSRFortessa (BD Biosciences). Astrocytes were stained with anti-EphB3APC Rabbit MAb (Sino Biological #50581-R001-A-100). For intracellular staining the intracellular antibody labeling kit following (#00-5523, eBioscience) was used with V450 Mouse anti-S6 (pS235/pS236) (BD #561457).

For recall responses, splenocytes were cultured in complete RPMI medium for 72 h at a density of 4×10⁵ cell/well in 96 well plates in the presence of MOG35-55 peptide (#110582, Genemed Synthesis). During the final 16 h, cells were pulsed with 1 μCi [³H]thymidine (#NET027A005MC PerkinElmer) followed by collection on glass fiber filters (#1450-421, PerkinElmer) and analysis of incorporated [³H]thymidine in a beta-counter (1450 MicroBeta TriLux; PerkinElmer). Alternatively, supernatants were collected after 72 h of culture for cytokine measurement by enzyme-linked immunosorbent assay (ELISA).

ELISA. Cytokines were measured in murine or human astrocytes supernatants following manufacturer's instructions using the following kits: Mouse TNFα ELISA Ready SET Go (eBioscience #50-173-31), Mouse IL-6 ELISA Ready SET Go (eBioscience #50-112-8808), Mouse CCL2 ELISA Ready SET Go (eBioscience #88-7391-86), Human IL-6 DuoSet ELISA (R&D Systems #DY206-05), Human TNFα DuoSet ELISA (R&D Systems #DY210-05), Human CCL2/MCP-1 DuoSet ELISA (R&D Systems #DY279-05). Cytokines in supernatants of MOG re-stimulated splenocytes were measured using the following kits: BD Mouse IFN-7 ELISA Set (BD #555138), BD Mouse IL-1β ELISA Set (BD #555252) and IL-17 Mouse ELISA Kit (Invitrogen #BMS6001).

In vivo knockdown with shRNA lentivirus. To produce lentiviral plasmids containing shRNA targeting EphB3, Ephrin-B3, the following validated shRNA sequences, based on the mission shRNA (SIGMA), were cloned into pLenti-GFAP-EGFP-mir30-shRNA or pLenti-CD11b-EGFP-mir30-shRNA: Ephb3 (5′-CCGGCCATAGCCTATCGGAAGTTTACTCGAGTAAACTTCCGATAGGCTATGG TTTTT-3′), and Efnb3 (5′-CCGGCCTGTGTACATTGTGCAGGATCTCGAGATCCTGCACAATGTACACAG GTTTTTG-3′) as described. For RabΔG-mCherry cloning, a cDNA encoding the mCherry ORF was inserted into the vector pSADdeltaG-GFP-F2 (Addgene, #32635), by PCR of a template plasmid encoding mCherry (Addgene, #80139) using SbfI and SacII restriction enzyme sites and the primers forward: 5′-AAACCTGCAGGGCCACCATGGTGAGCAAGGG-3′ and reverse: 5′-TTTCCGCGGTTTTTTGCTAGCTTACTTGTACAGCTCGTCCATGC-3′, also inserting a unique NheI site.

A 156 bp dsDNA fragment (Genewiz) was cloned into the NheI/SacII site viaXbaI/SacII, which introduced unique NheI/AscI sites flanking a unique PacI site. Enzymes used in this study are: XbaI (NEB, #R0145S), SacII (NEB, #R0157S), NheI-HF (NEB, #R3131M), AscI (NEB, #R0558S), SbfI-HF (NEB, #R3642L), DpnI (NEB, #R0176L), and PacI (NEB, #R0547L).

Lentivirus particles were generated by transfecting HEK293FT cells (#R70007, Invitrogen) with Lipofectamine 2000 Transfection Reagent (#11668019, Thermo Fisher Scientific) containing the corresponding viral vector and ViraPower Packaging mix (helper plasmids pLP1, pLP2, pLP/VSV-G, Invitrogen) according to manufacturer's protocol. Supernatants were filtered through a 0.45 m PVDF filter (#SLHVM33RS, Millipore), and lentiviral particles precipitated using Lenti-X concentrator (#631231, Clontech) following manufacturer's instructions. Lentiviruses were resuspended in 100th of original volume of PBS aliquoted, and stored at −80° C.

Viral titer was determined using the Lenti-X qRT-PCR titration kit (#LV900, Applied Biological Materials). Lentiviruses were delivered via intracerebroventricular (ICV) injection 7 days after EAE induction or twice at progressive stage for NOD (37 and 44 days after EAE induction). Mice were anesthetized with isoflurane and positioned in a Kopf Stereotaxic Alignment System for bilateral injection of 10 μL containing 107 IU of lentivirus (+/−1.0 (lateral), −0.44 (posterior), −2.2 (ventral) relative to Bregma; #1900, Kopf) was performed very slowly avoiding any spill overs. Both sides were injected using Hamilton syringe (#20787, Sigma-Aldrich), skin incisions were closed carefully by surgical sutures and 1 mg/kg of buprenorphine SR (#1Z-74000-192703, ZooPharm) was administered subcutaneously at the end of the procedure.

Rabies virus production. All plasmids used for subsequent rabies virus production were prepared using an endotoxin-free plasmid Giga kit (Qiagen, #12391). Pseudotyped G-deficient rabies virus was produced largely as previously described. Briefly, one day prior to transfection, baby hamster kidney (BHK) cells expressing T7 RNA polymerase, rabies glycoprotein G, and GFP (hereafter: B7GG cells) were seeded into ten 10-cm plates (Thermo Fisher Scientific, #08-772E) at a density of 2.2e6 cells/plate. Cells were grown in DMEM with high glucose, L-glutamine, and sodium pyruvate (Life Technologies, #11995073) supplemented with 10% FBS (Life Technologies, #10438026) (hereafter: B7GG media). The next day, they were transfected with the following helper plasmids: 150 μg pcDNA-SADB19N (Addgene, #32630), 75 μg pcDNA-SADB19P (Addgene, #32631), 75 μg pcDNA-SADB19L (Addgene, #32632), 50 μg pcDNA-SADB19G (Addgene, #32633), all gifts from Edward Callaway, and 300 μg RabΔG-mCherry using Lipofectamine 2000 (Thermo Fisher Scientific, #11668019) according to the standard protocol. Cells were transfected at 37° C. and 5% CO₂ for 18 hours. The next day, cells were split into 15-cm plates (Fisher Scientific, #08-772-6) and grown at 35° C. and 3% CO₂.

The following day, the supernatant was aspirated, and 24 mL fresh media was added to each plate. Thereafter, every 2 days, 10 mL of B7GG media was added to each plate. Then 2 days later, all viral supernatant was harvested, vacuum filtered through 0.45 μm pores (Fisher Scientific, #SCHVU11RE) and frozen. Altogether, 6 batches of viral supernatant were collected. For viral pseudotyping, 10 15-cm plates of BHK cells expressing the rabies virus envelope protein, EnvA, (hereafter BHK-EnvA) were seeded at 60% confluency at 35° C. and 3% CO₂.

The next day, unpseudotyped virus was applied to the BHK-EnvA cells for 48 hours. Each dish was washed 10× with 1×PBS to remove unpseudotyped virus. The next day, viral supernatant was aspirated and 24 mL of fresh B7GG media was added. Thereafter, viral supernatant was collected every 2 days. Altogether, 5 pseudotyped viral preparations were collected. To concentrate pseudotyped and unpseudotyped RabΔG-mCherry viruses, 35 mL of the collected supernatant was ultracentrifuged using a Beckman SW28 rotor at 70,000 g for 2 hours at 4° C. Afterwards, pellets were resuspended in HBSS, gently vortexed, and added to 2.5 mL of 20% sucrose in HBSS. Virus was centrifuged at 50,000 g for 2 hours at 4° C. using an SW55 rotor. Each viral pellet was resuspended in 100 μL ice cold HBSS, briefly vortexed, and chilled at 4° C. for >1 hour. Viral titration and pseudotyping specificity were performed using HEK293-TVA cells and HEK293 cells. The B7GG, BHK-EnvA, and HEK293-TVA cell lines were obtained from the GT3 Core Facility of the Salk Institute (NIH-NCI CCSG: P30 014195, an NINDS R24 Core Grant and funding from NEI). Gfap-CreTVAG/+ mice were transduced with 5 μL of 10¹⁰ IU/mL RabΔG mCherry virus bilaterally using coordinates: +/−1.0 (lateral), +0.6 (anterior), −3.0 (ventral) relative to Bregma.

AlphaLISA. Astrocyte-microglia co-cultures were seeded in 384 well plates pre coated with Poly-L-Lysine at a density of 10⁴ cells/well. After 3 days, cells were preincubated with the corresponding compounds 30 minutes before LPS stimulation (0.5 ng/mL Invivogen #tlrl-3pelps) and supernatants were collected after 18 h of incubation. mTNFalpha kit (Perkin Elmer #AL505) was used to measure TNFα in the supernatants following the high sensitivity protocol according to manufacturer's instructions. Briefly, 2 μl of the supernatants or given standards were subsequently mixed with 5 μl of anti-TNFα acceptor beads, 30 minutes after biotinylated antibody anti-TNFα was added after 1 h SA-Donor beads were added and incubated 30 minutes. All incubations were performed at 23° C. in the dark. Fluorescence at 615 nm was then measured using an EnVision-Apha Reader (Perkin Elmer #2105-0010).

EphB3 radiometric kinase activity assay. To study the EphB3 kinase activity, a radioactive filter binding assay using 33P ATP was performed as described at the MRU PPU International Centre for Kinase profiling. Briefly, different concentrations of A38 were combined with a mixture of EphB3 and substrate that was subsequently provided 33P ATP and halted by orthophosphoric acid addition. The mix was harvested onto P81 filter plates and counts were read on a Topcount NXT.

Cytotoxicity and apoptosis assay. Compound cytotoxicity was inferred by quantifying Lactate Deshidrogenase (LDH) release to the cells supernatants after 24 h using the CytoTox 96© Non-Radioactive Cytotoxicity Assay, (Promega #G1780) following by manufacturer's protocol. Apoptosis was evaluated in the cells after A38 treatment by the analysis of caspase-3/7 activation. Briefly cells were washed with 0.5% BSA, 2 mM EDTAin 1×PBS and incubated with surface antibodies and a live/dead cell marker on ice for 30 minutes. After, cells were washed and following staining for Caspase-3/7 according the manufacturer's instructions (Invitrogen, #C10427).

RNA-sequencing. Sorted astrocytes or microglia were re-suspended in extraction buffer of Picopure RNA isolation kit (KIT0204, Thermo Fisher) and after 30 min incubation at 42° C. samples were extracted following manufacturer's instructions with on-column DNase I digestion (QIAGEN, #79254). RNA was suspended in 10 μl of nuclease free water and 5 μl were sent for SMARTseq sequencing by the Broad Technology Labs and the Broad Genomics Platform. Processed RNA-Seq data was filtered, removing genes with low read counts. Read counts were normalized using TMM normalization and CPM (counts per million) were calculated to create a matrix of normalized expression values.

The fastq files of each RNA-seq data sample were aligned to Mus musculus GRCm38 transcriptome using Kallisto (v0.46.1), and the same software was used to quantify the alignment results. The differential expression analysis was used to conduct using DESeq2, and the log 2 fold change was adjusted using apeGLM for downstream analysis. The Benjamini-Hochberg method was used for multiple hypothesis testing correction. The GSEA analysis was performed using the apeGLM adjusted differential expression analysis results. Genes that were differentially expressed with adjusted p values <0.05 were analyzed with the Ingenuity© Pathway Analysis (IPA) tool to determine significantly regulated pathways. P-values of canonical signaling networks were obtained using the NetworkAnalyst tool with the input of the differentially decreased genes in astrocytes from A38-treated mice belonging to Eph pathway.

RNA isolation, cDNA synthesis and qPCR. RNA was extracted from in vitro generated samples with RNeasy Mini kit (#74106, QIAGEN) and cDNA was prepared by using the High-Capacity cDNA Reverse Transcription Kit (#4368813, Life Technologies), both according to the manufacturer's protocol. Gene expression was measured by qPCR with Taqman Fast Universal PCR Master Mix (#4367846, Life Technologies). Data were analyzed by the ddCt method by normalizing the expression of each gene to GAPDH and HPRT for mouse and GAPDH for human and then to the control group. All probes are listed below.
Mouse: EphB3 Mm00802553_m1, Ccl2 Mm00441242_m1, Csf2 Mm01290062_m1, Nos2 Mm00440502_m1, Tnf Mm00443258 m1, Il1b Mm00434228_m1, 116 Mm00446190_m1, GAPDH Mm99999915_g1.
Human: CCL2 Hs00234140_m1, CSF2 Hs00929873 ml, NOS2 Hs01075529_m1, TNF
Hs00174128_m1, IL6 Hs00985639_m1, IL1b Hs01555410_m1, GAPDH Hs02758991_g1.

Oxygen consumption. Respiration was measured using the XFe24 or XFe96 analyzers (Agilent Technologies) with 70,000 or 30,000 cells per well respectively, starved for 24 h and stimulated for 16 h with 50 ng/mL TNFα (#410-MT-010, R&D Systems) and 100 ng/mL IL-1β (R&D Systems, #401-ML-025) after 30 min pretreatment with 50 μM A38 or 100 nM rapamycin. Mito Stress assay was then performed with Seahorse XF Cell Mito Stress Test Kit (Agilent Technologies, #103015-100) following the manufacturer's manual. Oxygen consumption rate (OCR) was then quantified after sequential addition of 2 μM oligomycin, 1 μM FCCP and 5 μM rotenone/antimycin A. The assay medium (DEMEM with 10 mM Glucose, 1 mM pyruvate and 2 mM glutamine) was used during the assay. The OCR rate was normalized to cell number estimated using CyQUANT cell proliferation assay kit (Invitrogen, #C7026).

Mitochondrial ROS measurement. 50,000 astrocytes were seeded per well in 96-well black plates and rested for 2 days in complete medium, followed by activation or inhibitor pretreatment for 24 h. MitoSOX red (Thermo Fisher Scientific, #M36008) was used to stain for mitochondrial ROS for 10 min following the manual and the signal was detected on Infinite M1000 PRO Microplate Reader (Tecan). The fluorescent signal was normalized to cell number estimated using CyQUANT cell proliferation assay kit (Invitrogen, #C7026) first and then compared to control group for data analysis.

Immunofluorescence analysis of EAE samples. Immunostaining was performed largely as described previously. Mice were intracardially perfused with ice cold 1×PBS followed by ice cold 4% PFA. Brains and spinal cords were harvested, post-fixed in 4% PFA overnight at 4° C., followed by dehydration in 30% sucrose for 2 days at 4° C. Brains were then frozen in OCT (Sakura, #4583) and 30 μm tissue slices were sectioned by cryostat on SuperFrost Plus slides (Fisher Scientific, #22-037-246). A hydrophobic barrier was drawn (Vector Laboratories, #H-4000) and sections were washed 3× for 5 minutes with 0.1% Triton X-100 in PBS (PBS-T).

Sections were permeabilized with 0.3% PBS-T for 20 minutes, then washed 3× with 0.1% PBS-T. Sections were blocked with 5% donkey serum (Sigma-Aldrich, #D9663) in 0.1% PBS-T at RT for 30 minutes. Sections were then incubated with primary antibodies diluted in blocking buffer ON at 4° C. Following primary antibody incubation, sections were washed 3× with 0.1% PBS-T and incubated with secondary antibodies diluted in blocking buffer for 2 hours at room temperature (RT).

Following secondary incubation, sections were washed 3× with 0.1% PBS-T, dried, and coverslips were mounted using Fluoromount-G with DAPI (SouthernBiotech, #0100-20) or without DAPI (SouthernBiotech, #00-4958-02).

Primary antibodies used in this study were: mouse anti-GFAP (Millipore, 1:500, #MAB360), rabbit anti-Iba1 (Abcam, 1:100, ab178846), rabbit anti-Ephrin-B3 (Abcam, 1:100, ab101699), rabbit anti-Eph Receptor B3 (Abcam, 1:100, ab133742), rabbit anti-acetyl NF-κB p65 (Lys-310) (Sigma-Aldrich, 1:100, SAB4502616-100UG), and rabbit anti-mCherry (Abcam, 1:500, ab167453).

Secondary antibodies used in this study were: Alexa Fluor 647 donkey anti-mouse (Abcam, #ab150107), donkey anti-Rabbit IgG (H+L) Highly Cross-Adsorbed, Alexa Fluor 568 (Life Technologies, #A10042), Rhodamine Red-X-AffiniPure Fab Fragment Donkey Anti-Rabbit IgG (H+L) (Jackson Immunoresearch, #711-297-003), Alexa Fluor 647 AffiniPure Fab Fragment Donkey Anti-Rabbit IgG (H+L) (Jackson Immunoresearch, #711-607-003), Goat anti-Rabbit IgG (H+L) Cross-Adsorbed Secondary Antibody, Alexa Fluor 405 (Thermo Fisher, #A-31556), Alexa Fluor 488 AffiniPure Fab Fragment Donkey Anti-Rabbit IgG (H+L) (Jackson Immunoresearch, #711-547-003), all at 1:500 working dilution.

Iterative labeling using rabbit primary antibodies was accomplished by incubating with a single primary antibody on Day 1, staining with the anti-rabbit Fab fragment on Day 2, washing 6× with PBS-T, followed by incubation with primary and secondary antibodies as described above. Sections were imaged on an LSM710 Zeiss confocal. acetyl-P65hi and GFAPhi or Iba1hi cells were scored for colocalization. Similarly, GFAP⁺ or Iba1⁺ cells were scored followed by analysis of EphB3 Receptor or Ephrin-B3 expression, respectively. For quantification in rabies tracing experiments, mCherry⁺ cells were scored followed by analysis of GFAP⁺ or Iba1⁺ immunoreactivity.

SDS-PAGE and Western blot. Protein lysates were prepared with 1× Lysis Buffer (Cell Signaling Technology #9803S) containing 1× Halt Protease and Phosphatase Inhibitor Cocktail (Thermo Fisher Scientific #78441) following manufacturer's instructions, or using the Cell Fractionation Kit (Cell Signaling Technology #9038S) following the manufacturer's instructions for cytoplasmic and nuclear subcellular fractions. When required, protein content of each sample was normalized to 100 μg/mL after quantification with Micro BCA Protein Assay Kit (Thermo Fisher Scientific #23235). 1× Laemmli buffer (#BP-111R, Boston BioProducts) was added followed by boiling at 95° C. for 5 minutes to protein lysates before loading. SDS-PAGE was performed in Bolt 4%-12% Bis-Tris Plus gradient gels (#NWO4125BOX, Invitrogen). Western blotting was performed by transferring proteins onto a Nitrocellulose membrane (Thermo Scientific #88018) in 1× Bolt MES SDS Running Buffer (Life Technologies #B000202). Membranes were blocked in 10% skimmed milk (#M0841, Lab Scientific) in TBS-T (#IBB-180-2L, Boston BioProducts). Primary antibodies were incubated ON at 4° C. and secondary 30 minutes at RT.

HRP-conjugated blots were developed using the KwikQuant imaging system and KwikQuant western blot detection kit (#R1004 Kindle Biosciences). Primary antibodies used in this study were from Cell Signaling Technology: rabbit anti-Phospho-P13 Kinase p85(Tyr458)/p55(Tyr199) (#4228), mouse anti-PI3 Kinase p85a (6G10) (#13666), rabbit anti-Phospho-Akt (Ser473) (D9E) (#4060), rabbit anti-Phospho-Akt (Thr308) (D25E6) (#13038S), mouse anti-Akt (40D4) (#2920), rabbit anti-Phospho-S6 Ribosomal Protein (Ser235/236) (D57.2.2E) (#4858), rabbit anti-S6 Ribosomal Protein (5G10) (#2217), rabbit anti-P-p65 (Ser536) (#3033) and rabbit anti-GAPDH (#2118). Secondary antibodies used in this study are: anti-mouse IgG-HRP conjugate (#7076S, Cell Signaling Technology, 1:2000), anti-rabbit IgG-HRP conjugate (#7074S, Cell Signaling Technology, 1:5000).

H&E, LFB and silver staining. Transverse sections from lumbar spinal cords of EAE mice at the end of the experiment were fixed in 4% paraformaldehyde, pH 7.4, embedded in paraffin and cut at 10-μm thickness. Sections were stained with Hematoxylin and Eosin, Luxol fast blue, or silver nitrate. Images were acquired by using a Zeiss Axioskop2 plus microscope with AxioCam HRC and AxioVision software and analyzed by using ImageJ software.

Electron microscopy. Electron microscopy was conducted as previously described. After transcardial perfusion with phosphate-buffered saline (PBS), spinal cords were fixed in 3% glutaraldehyde solution. The glutaraldehyde solution was prepared as follows: Sorensen buffer, pH 7.45, was used for the dilution of 25% glutaraldehyde solution (Merck-Millipore, #1.042.390.250) and prepared by titration of disodium phosphate solution (17.8 g Na₂HPO₄×2H₂O in 1000 ml ddH₂O, Sigma-Aldrich, #S9763) and monopotassium phosphate solution (4.08 g KH₂PO₄ in 300 ml ddH₂O).

Next, the tissue was washed in Sorensen buffer and incubated in 1% osmium tetroxide solution (1:1 mix of 2% Osmium (Science Service, #E19172) and 0.365 g K₄Fe(CN)₆ in 10 ml ddH₂O (Merck, P3289)) for two hours. After incubation in ascending alcohol series starting from 30% ethanol to 100% ethanol, tissue was placed in 100% 1,2-propylenoxide (Sigma-Aldrich, #8.07027.1000) for 30 minutes.

Resin was prepared by mixing 30 g glycid ether (Serva, #21045.01), 56 g 2-dodecenylsuccinic acid (Serva, #20755.01), 16 g Renlam (Serva, #13825) and 2 ml phthalic acid dibutylester (Serva, #32805) in a glass stirrer for one hour. Afterwards, 2 ml 2,4,6 tris(dimethylaminomethyl)phenol (Serva, #36975) were added and the solution was mixed for another 5 minutes. Tissue was incubated in a 2:1 propylenoxide/resin mix for 1 hour, followed by the ON incubation in a 1:2 mix. After supernatant removal and liquid evaporation, 100% resin was added for 4 hours at room temperature. The block was fully polymerized after incubation for 24 hours in resin at 75° C. 70 nm ultra-thin sections were cut with a microtome (Reichert Ultracut S) and Histo Diamant Knives (Diatome). Sections were transferred to a 200 square mesh grid (Science Service, #T200-Cu), treated with 1% uranyl acetate and 3% lead citrate (Leica, #16707235) and analyzed using an electron microscope (Philips, CM-100).

Statistical Analysis. Statistical analyses were performed with Prism 8 software (GraphPad), using the indicated test. Regression slope test was applied for EAE clinical scores of NOD model only. No samples were excluded. At least 3 biological repeats for each condition were included and 3 independent experiments were performed for all in vitro assays, and displayed figures are representative. No statistical methods were used to pre-determine sample sizes and no blinding was performed in this study. All statistical tests, comparisons, and sample sizes are included in the figures and figure legends. All data are shown as mean±SEM. In all cases, ****p<0.0001, ***p<0.001, **p<0.01, *p<0.05, ns=not statistically significant, p>0.05.

Data and Code Availability. All RNA-seq data have been deposited in the GEO database as GEO SuperSeries GSE150389.

### Example 1—Role of the EphB3 Receptor in Microglia-Astrocyte Communication

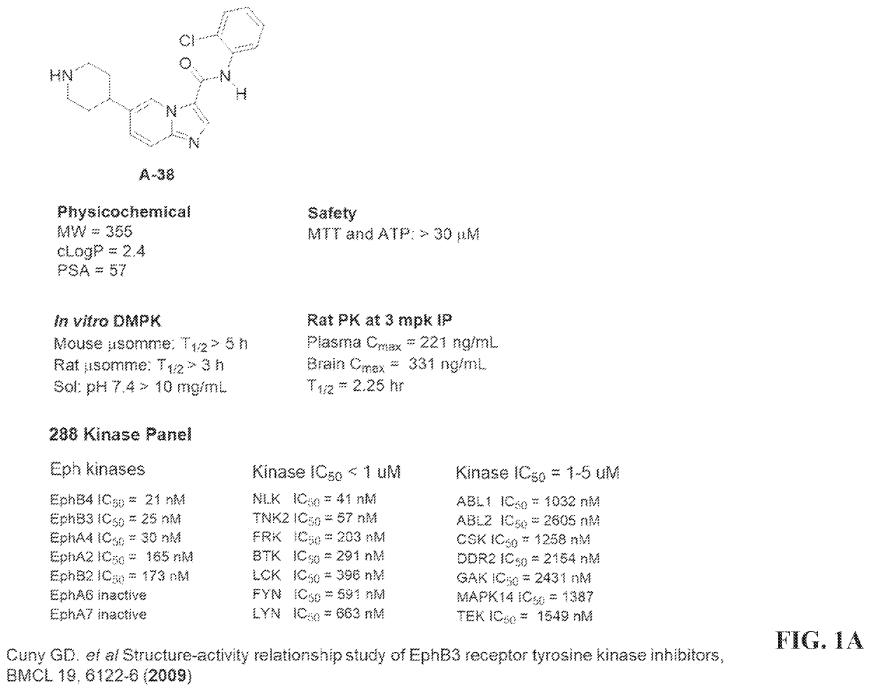

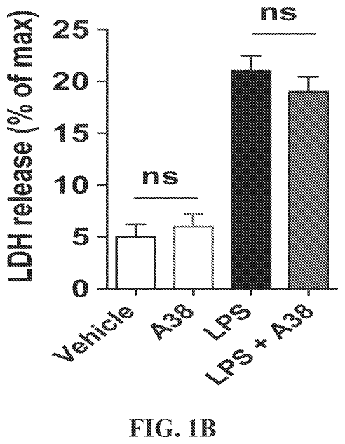

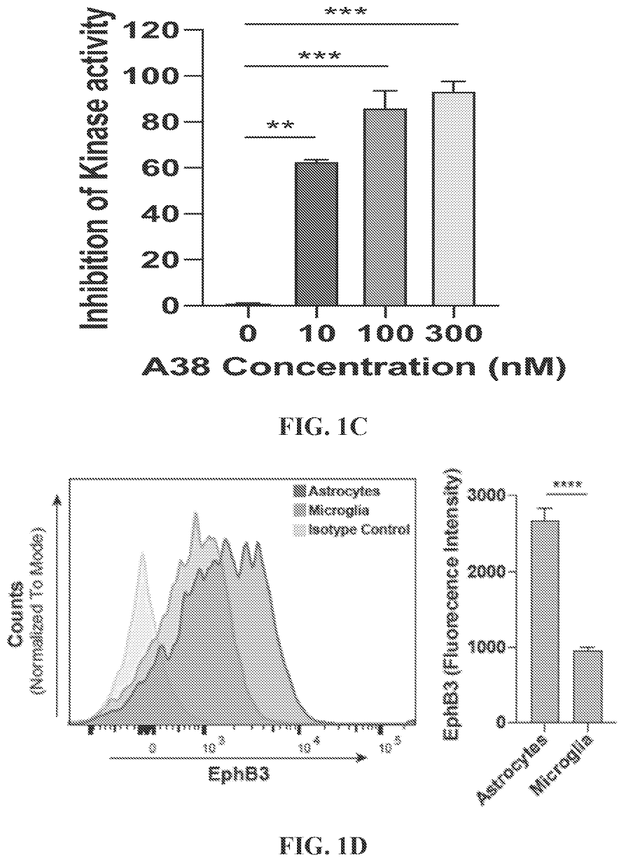

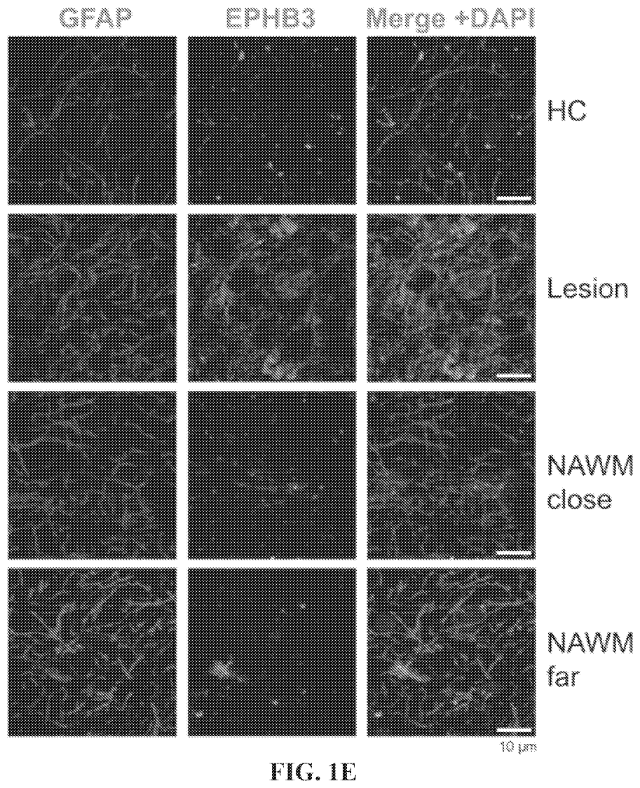

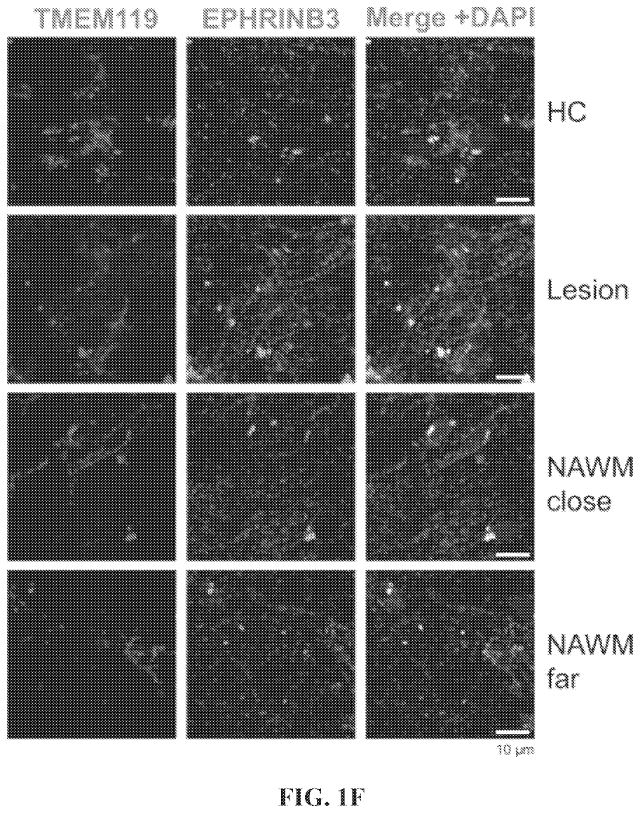

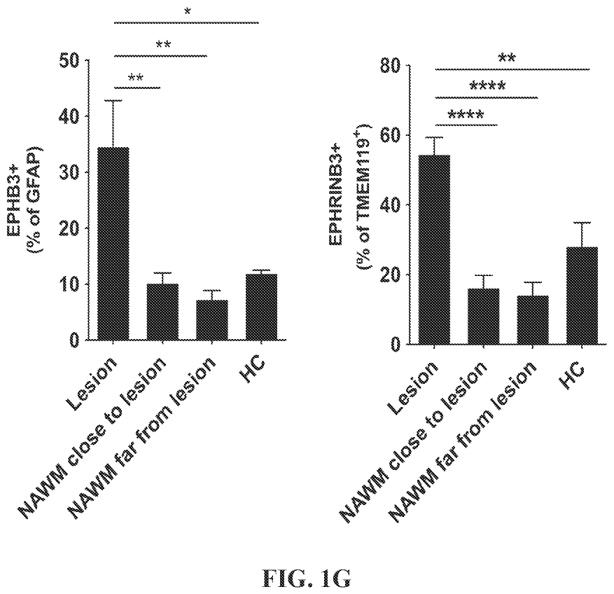

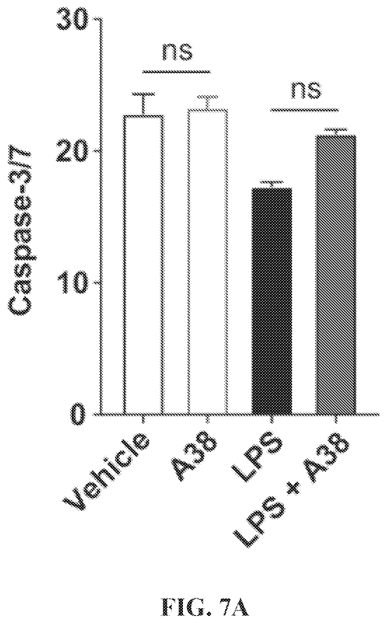

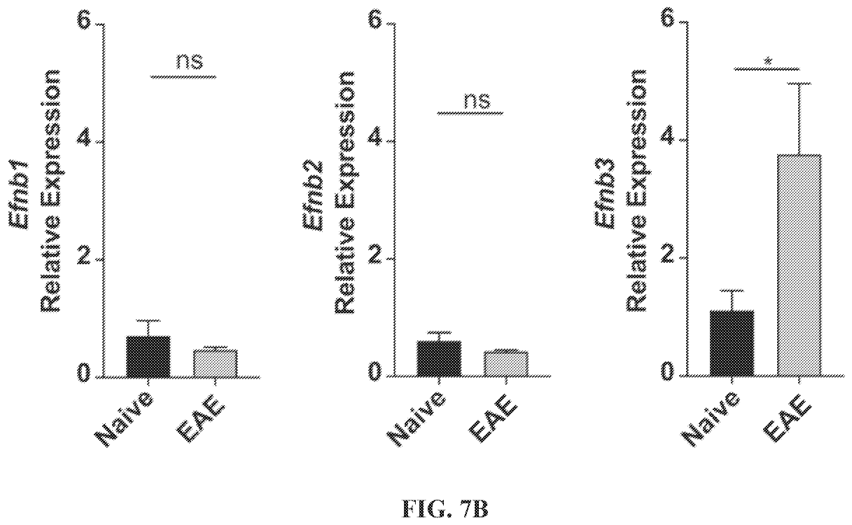

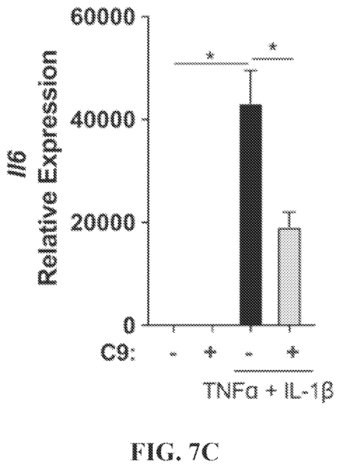

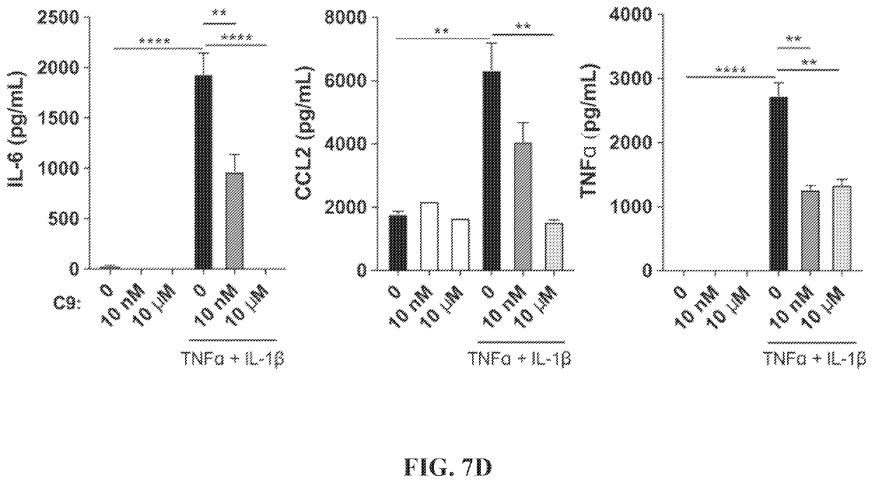

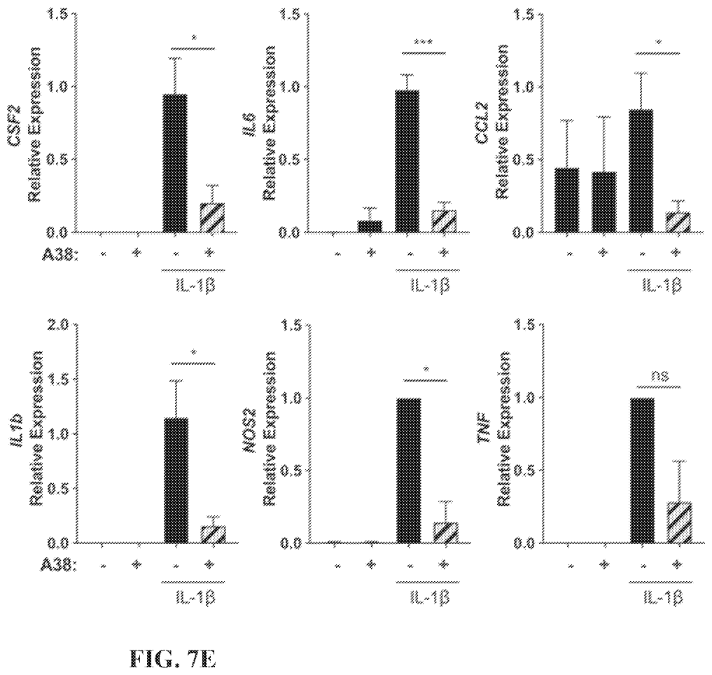

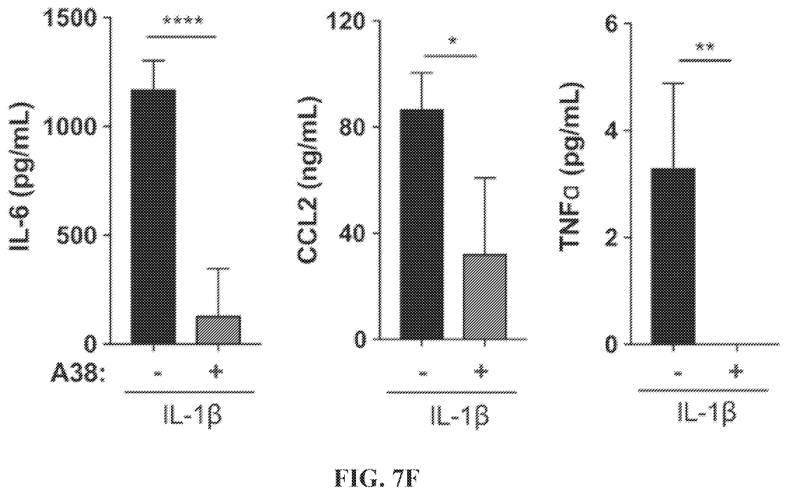

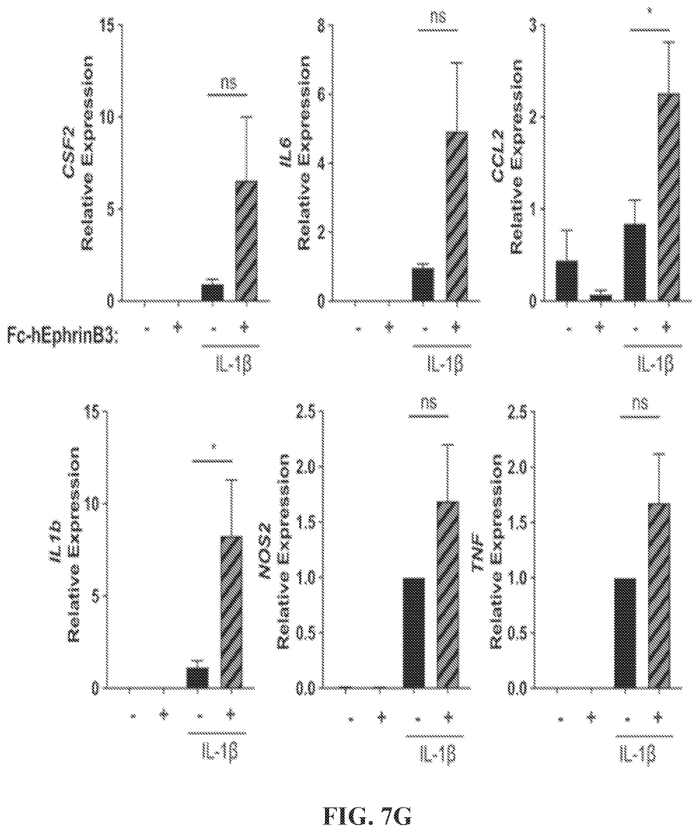

Compound A38 was identified (See FIG. 1A). This compound suppressed TNFα production in astrocytes, but did not induce cytotoxicity or apoptosis (FIG. 1B, FIG. 7A).

A38 is an inhibitor of kinase activity of the erythropoietin-producing human hepatocellular B3 receptor (EphB3), a member of a receptor tyrosine kinase family with important roles in axon guidance among other biological processes. Indeed, A38 inhibited EphB3 tyrosine kinase activity in a dose-dependent manner in a cell-free assay (FIG. 1C).

The analysis of neonatal mouse astrocyte and microglia cultures detected higher EphB3 expression in astrocytes than in microglia as shown in FIG. 1D. More importantly, increased EPHB3 expression was detected in astrocytes located in MS CNS lesions (FIG. 1E, FIG. 1G).

EphB3 receptor has been shown to be activated by interactions with its membrane-bound ligands Ephrin-B1, Ephrin-B2 and Ephrin-B3, encoded by Efnb1, Efnb2 and Efnb3, respectively, and Ephrin-B3 has been mostly expressed in the CNS, while Ephrin-B1 and Ephrin-B2 showed a broader expression pattern.

In this example, increased expression of Efnb3, but not Efnb1 or Efnb2, was detected in microglia during EAE (FIG. 7B). Moreover, increased expression of EPHRIN-B3 was detected in microglia in MS CNS lesions (FIG. 1F, FIG. 1G). Taken together, these results show a potential role in CNS inflammation for the interaction between Ephrin-B3 expressed in microglia and EphB3 receptor expressed in astrocytes.

### Example 2—EphB3 Signaling Boosts Astrocyte Pro-Inflammatory Activities

Pro-inflammatory cytokines TNFα and IL-1β have been associated with the pathogenesis of MS and EAE, and induce astrocyte transcriptional responses similar to those detected during EAE.

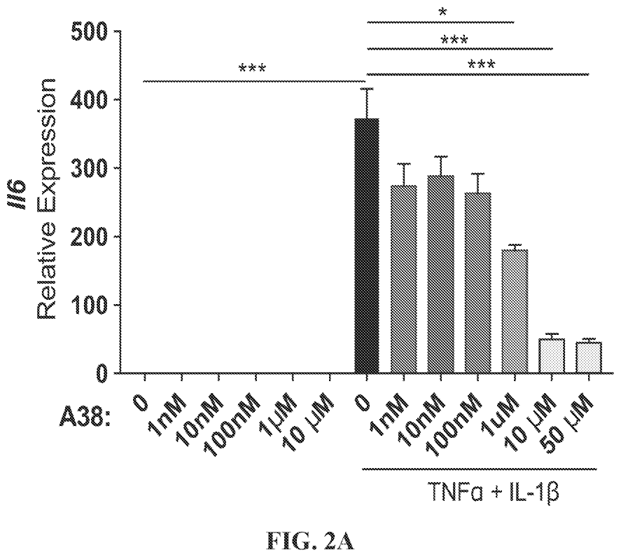

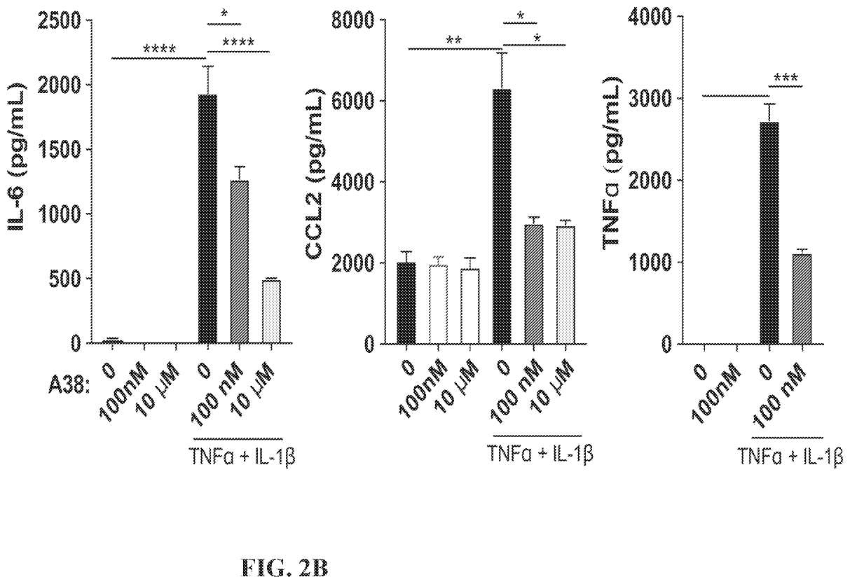

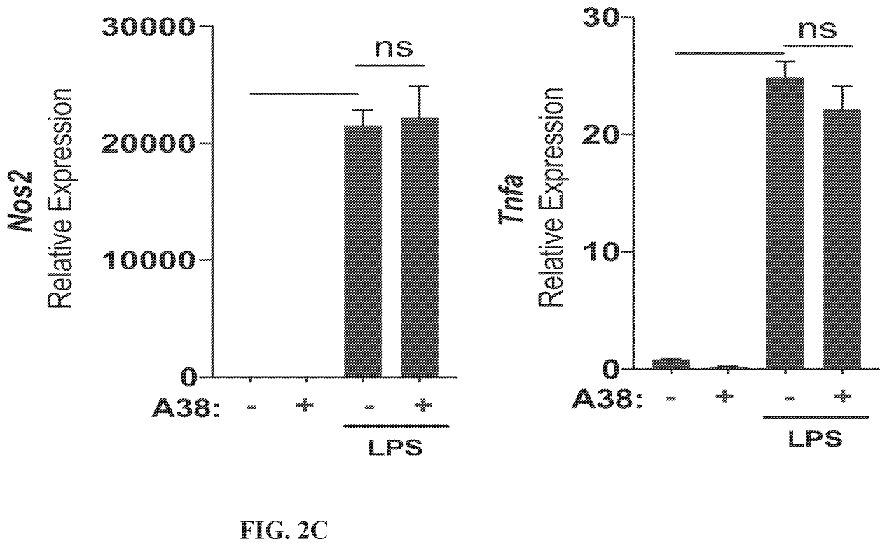

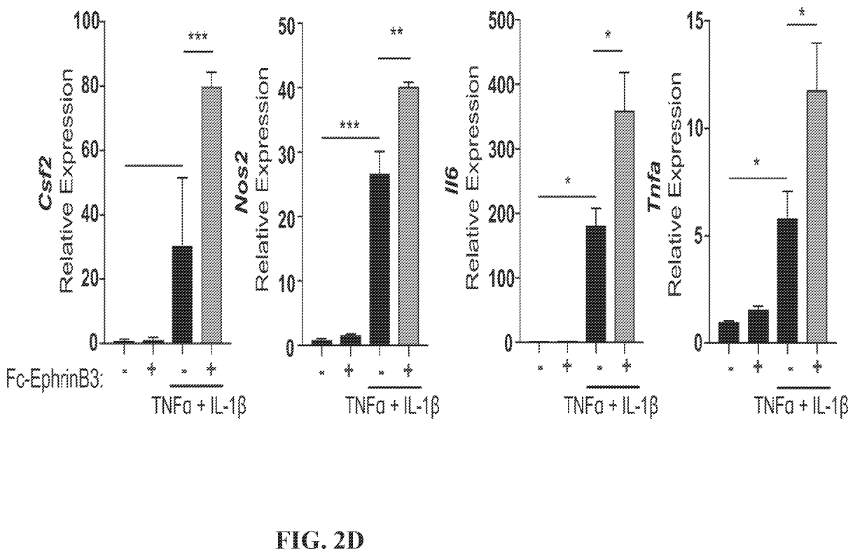

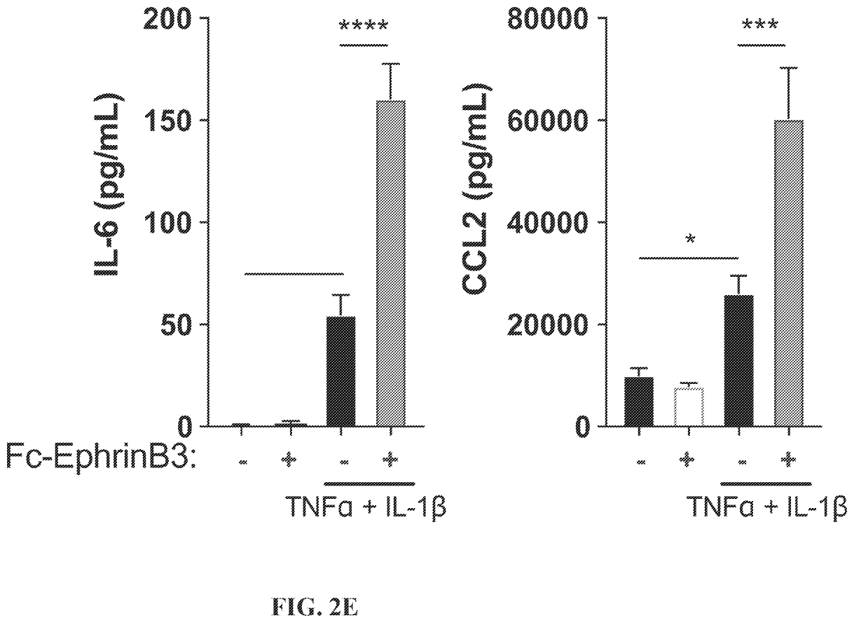

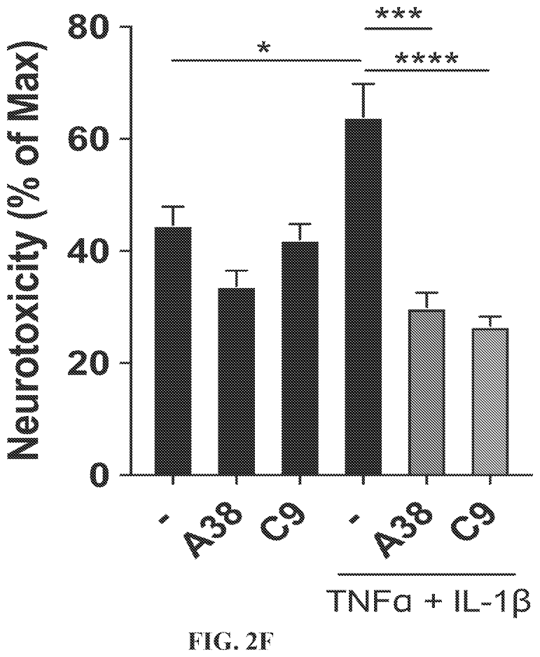

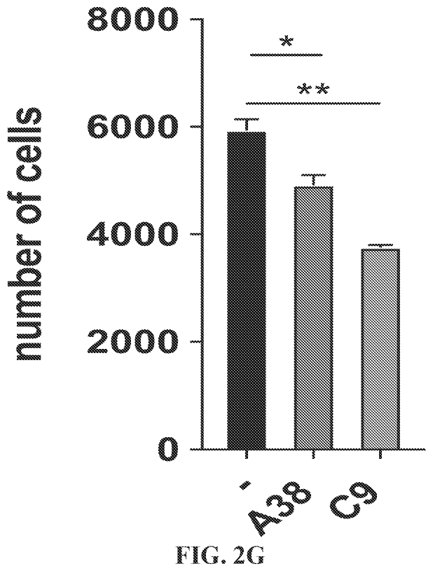

To study the effects of EphB3 signaling in astrocytes, neonatal murine astrocytes in culture activated with TNFα and IL-1β were treated with A38. As shown in FIG. 2A, A38 reduced 116 expression in astrocytes in a dose-dependent manner.

A38 also decreased the production of IL-6, TNFα and CCL2 (FIG. 2B). Similar results were obtained with compound C₉, a previously identified inhibitor of EphB3 kinase activity (FIG. 7C-D):

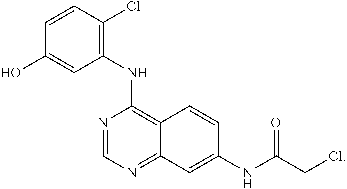 [SMILES: O=C(CCl)Nc1ccc2c(Nc3cc(O)ccc3Cl)ncnc2c1]

In agreement with the higher expression of EphB3 detected in astrocytes compared to microglia (FIG. 1F-I), A38 showed no effect on the microglial expression of the pro-inflammatory genes Nos2 and Tnfa induced by LPS stimulation (FIG. 2C), suggesting that EphB3 intracellular signaling played important role in the regulation of astrocyte, but not microglial responses.

To further study the effects of EphB3 signaling, murine astrocytes were activated in vitro with TNFα, IL-1β and plate-bound Ephrin-B3-Fc chimera. As shown in (FIG. 2D), EphB3 activation boosted the expression of genes associated with astrocyte pro-inflammatory activities such as 116, Nos2, Csf2 and Tnfa; EphB3 activation also boosted IL-6 and CCL2 production (FIG. 2E).

Furthermore, as shown in FIG. 7E-G, similar results were obtained when the effects of EphB3 inhibition with A38 or EphB3 activation with plate-bound Ephrin-B3-Fc chimera on primary human astrocytes in culture were investigated. TNFα and nitric oxide have been linked to astrocyte neurotoxic activities.

Moreover, CCL2 produced by astrocytes have been shown to promote monocyte recruitment to the CNS. Thus, based on the observed effects of A38 and C9 on TNFα, Nos2 and CCL2 expression, neurotoxic and chemotactic activities of astrocyte-conditioned medium (ACM) collected from astrocytes pre-treated with A38 or C9 were evaluated. As shown in FIG. 2F-G, pharmacological inhibition of EphB3 in astrocytes reduced ACM neurotoxic and chemoattractant activity. The experimental data presented in this example shows that EphB3 signaling boosts pro-inflammatory activities in astrocyte cells.

### Example 3—Astrocytes and Microglia Establish Cell Contacts During EAE

Microglia-astrocyte interactions mediated by soluble factors are known to participate in the control of CNS inflammation. Since EphB3 receptor signaling is triggered by interactions with membrane-bound ephrins, the findings from the present disclosure suggest the existence of cell contacts that regulate astrocyte and microglial responses. To investigate astrocyte-microglia interactions in vivo, recombinant G-deficient pseudorabies virus (RabΔG) was utilized. RabΔG has been used to trace neural circuits, but can also infect astrocytes when used in combination with proper pseudotyping and transgenic mice.

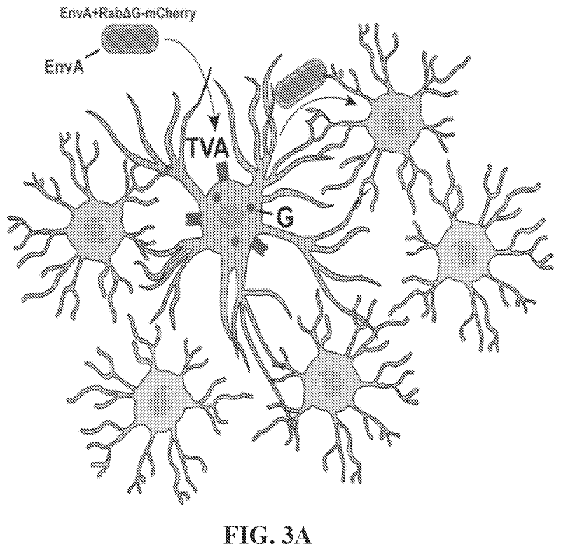

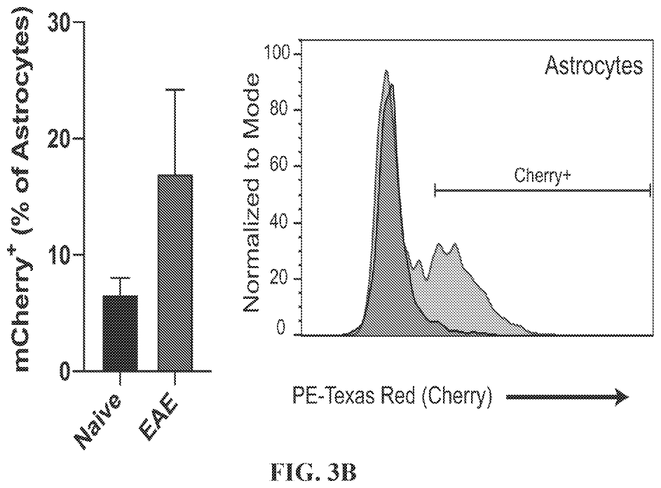

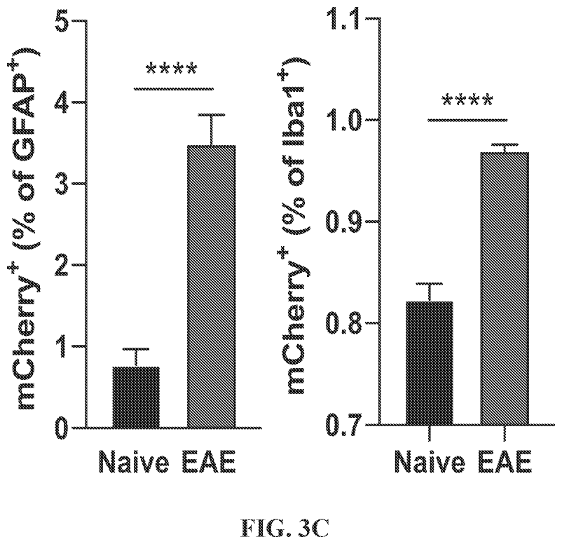

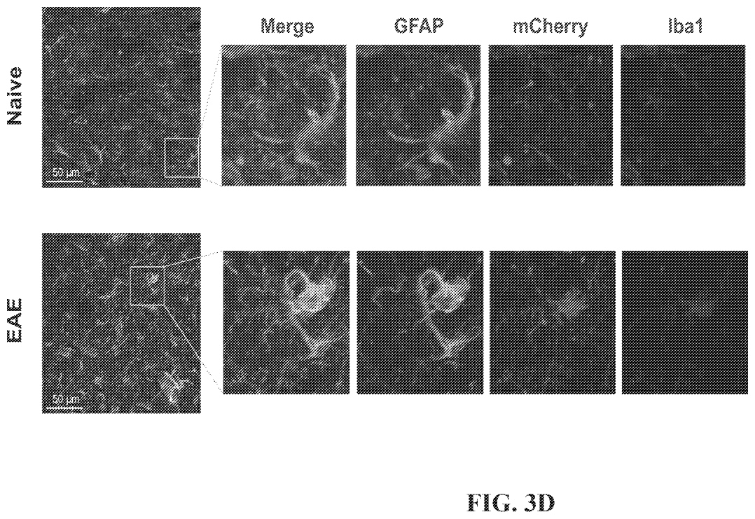

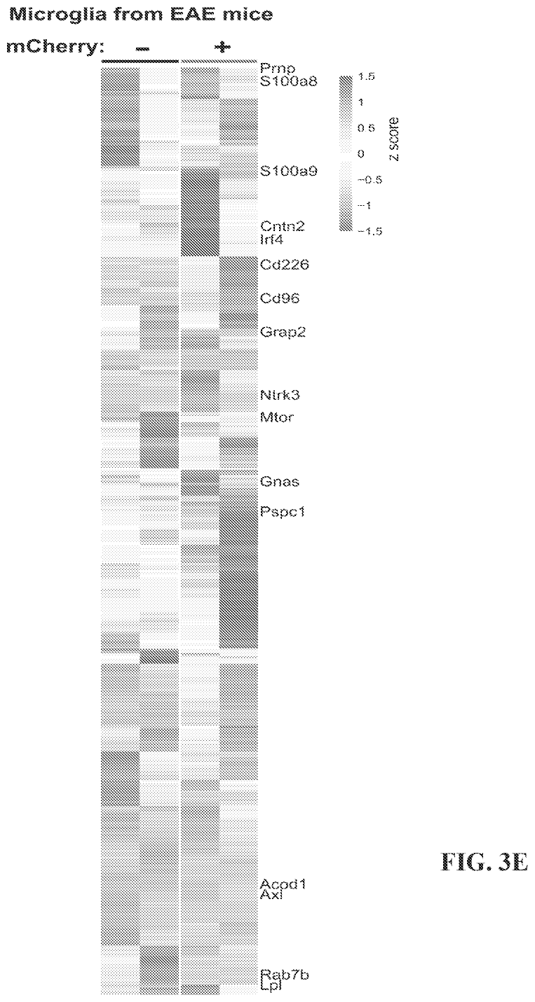

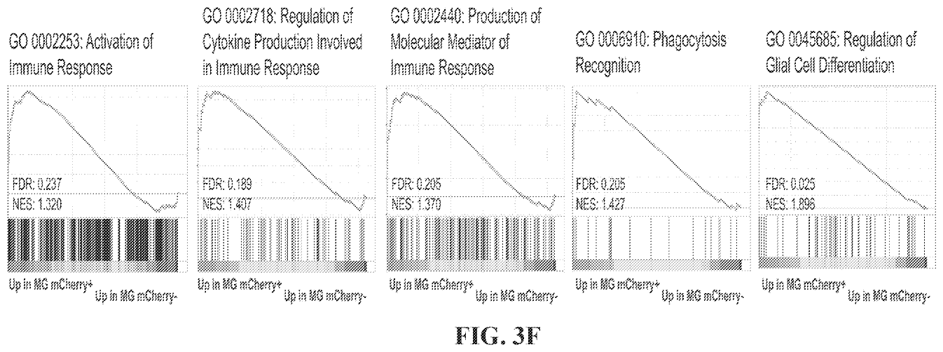

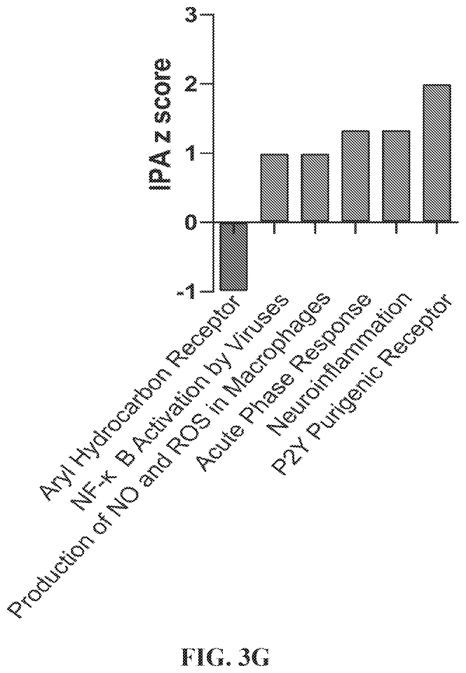

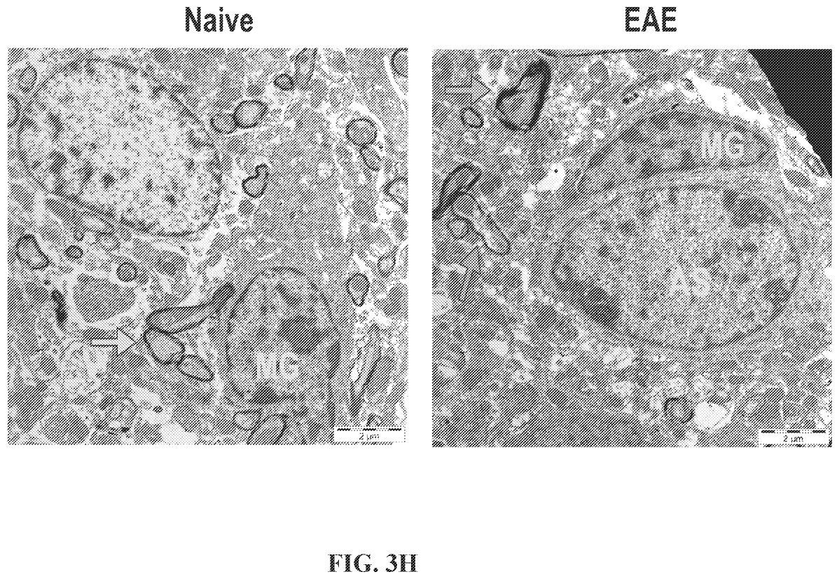

In this investigation, RabΔG expressing the fluorescent protein mCherry and pseudotyped with EnvA was used in combination with transgenic mice that express the viral glycoprotein G and the EnvA receptor TVA in astrocytes under the control of the Gfap promoter (Gfap^{TVA/G} mice) (FIG. 3A).

The pseudotyping of RabΔG with EnvA has been shown to restrict the initial viral infection to astrocytes expressing the transgenic EnvA receptor TVA. Since pseudotyped RabΔG lacks glycoprotein G, it only replicates in GFAP⁺ astrocytes, which express transgenic glycoprotein G.

Thus, following injection into Gfap^{TVA/G} mice, RabΔG initially infects and replicates in TVA⁺ astrocytes that express rabies glycoprotein G, which is incorporated to the surface of new virions thereby allowing the infection of cells in contact with astrocytes as previously shown in oligodendrocyte viral tracing studies. The expression of mCherry allows the detection and isolation of infected cells in contact with astrocytes.

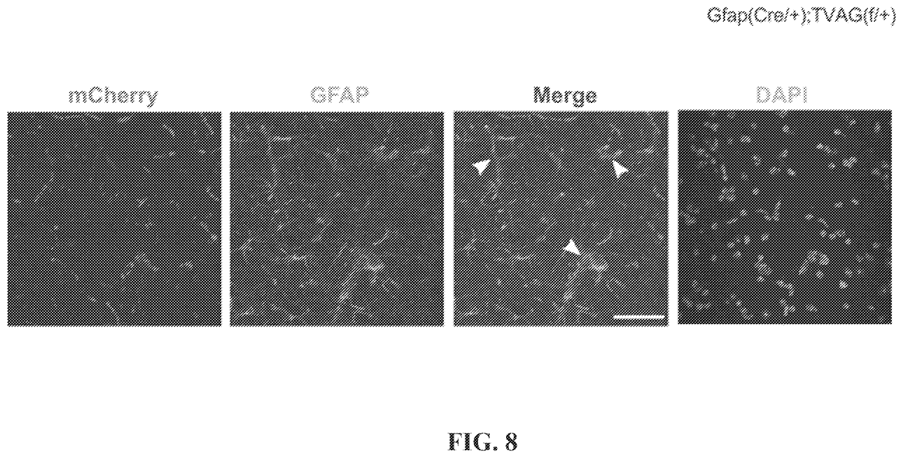

Thus, RabΔG was injected into Gfap^{TVA/G} mice, and mCherry expression in astrocytes was detected by flow cytometry and immunofluorescence (See FIG. 3B-C, and FIG. 8). At 3-7 days post-injection, higher microglial mCherry expression in EAE was also detected, suggesting increased astrocyte-microglia interactions (FIG. 3C-D). As shown in (FIG. 3E-G), RNA-seq analysis of microglia isolated by flow cytometry from RabΔG-injected Gfap^{TVA/G} EAE mice detected higher expression of pro-inflammatory transcripts in mCherry*than in mCherry-microglia from the same mice, suggesting that microglial activation boosted astrocyte-microglia interactions. The results also suggested that some astrocyte-microglia interactions were associated with microglial pro-inflammatory transcriptional responses.

Finally, further support for the existence of astrocyte-microglia contacts during, e.g., EAE was provided by transmission electron microscopy studies in which direct contacts between astrocytes and microglia during EAE were detected (FIG. 3H). Collectively, these data suggest that astrocytes and microglia establish cell contacts during CNS inflammation.

### Example 4—Microglial Ephrin-B3 and EphB3 Expressed in Astrocytes Promote EAE CNS Pathology

To study the role of Ephrin-B3 and EphB3 on the regulation of astrocyte and microglial responses in the context of CNS inflammation, knock-down experiments of Ephb3 in astrocytes and Efnb3 in microglia during EAE using lentivirus-delivered small hairpin RNAs (shRNAs) expressed under the control of Gfap or Itgam promoters, respectively were performed.

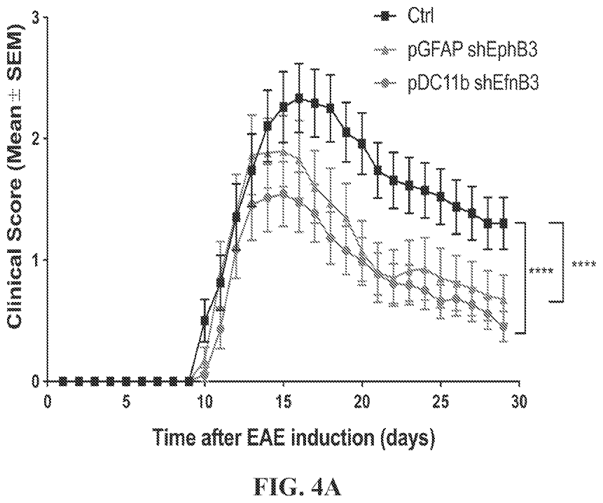

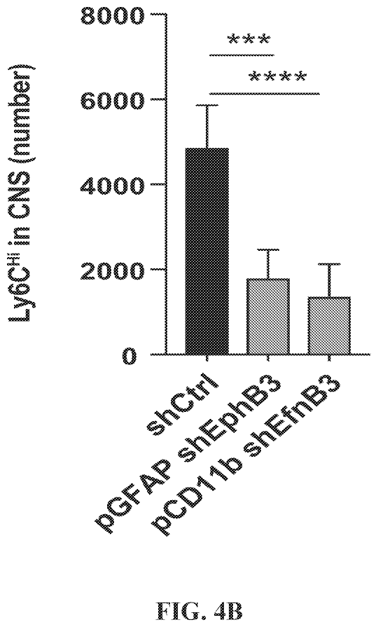

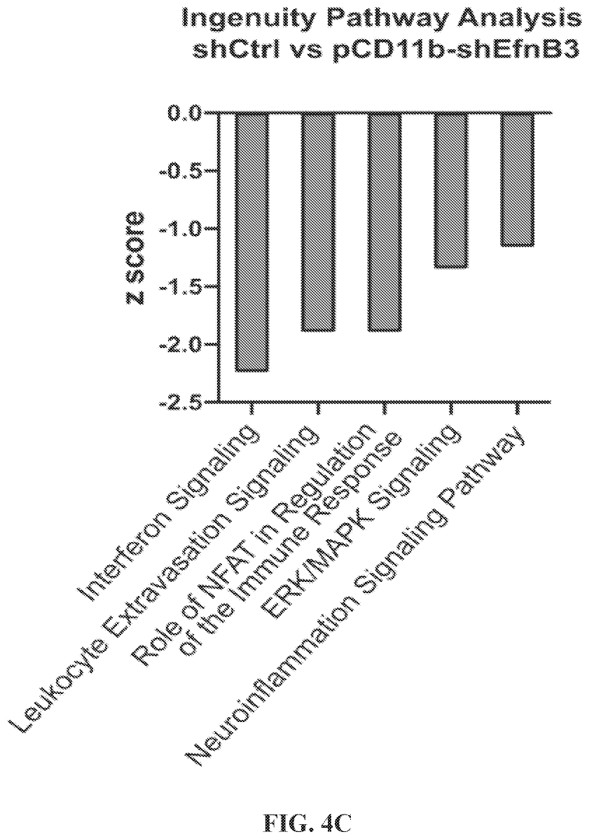

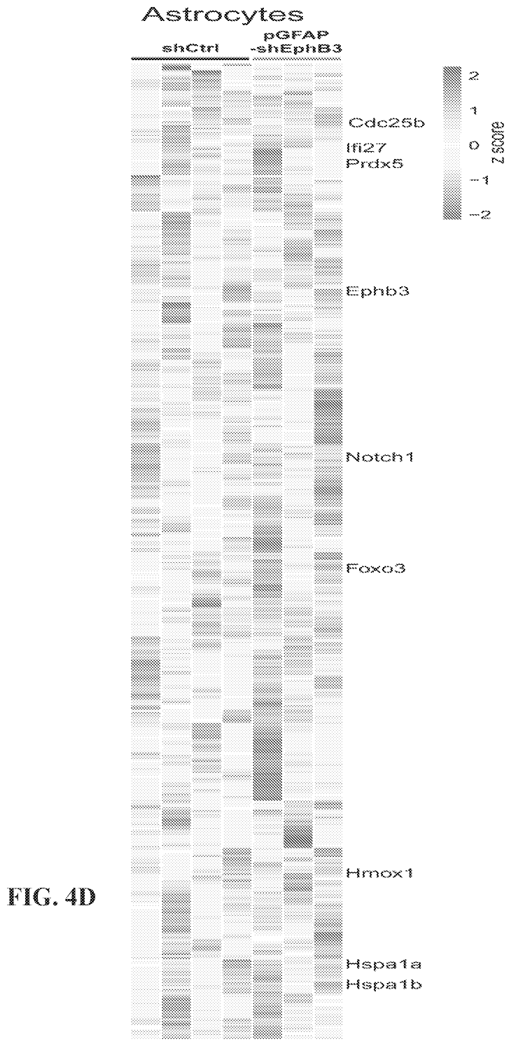

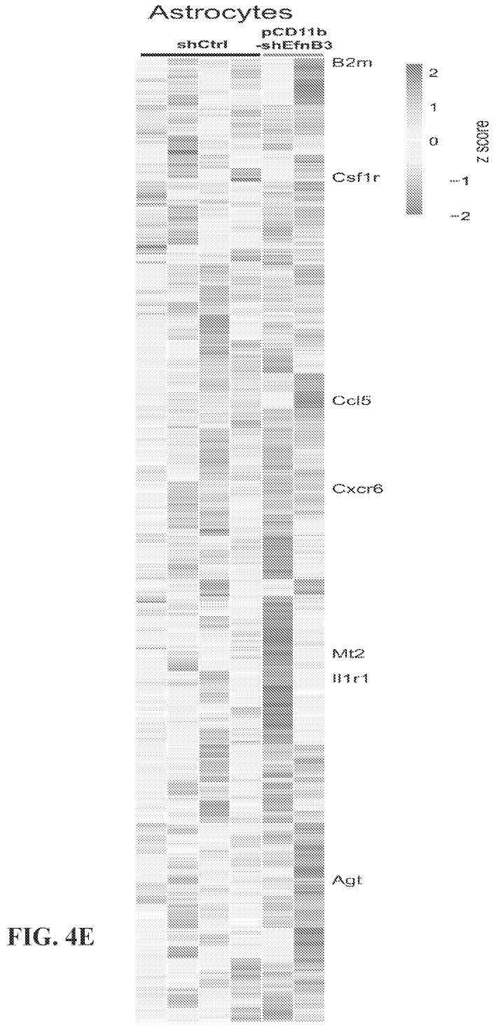

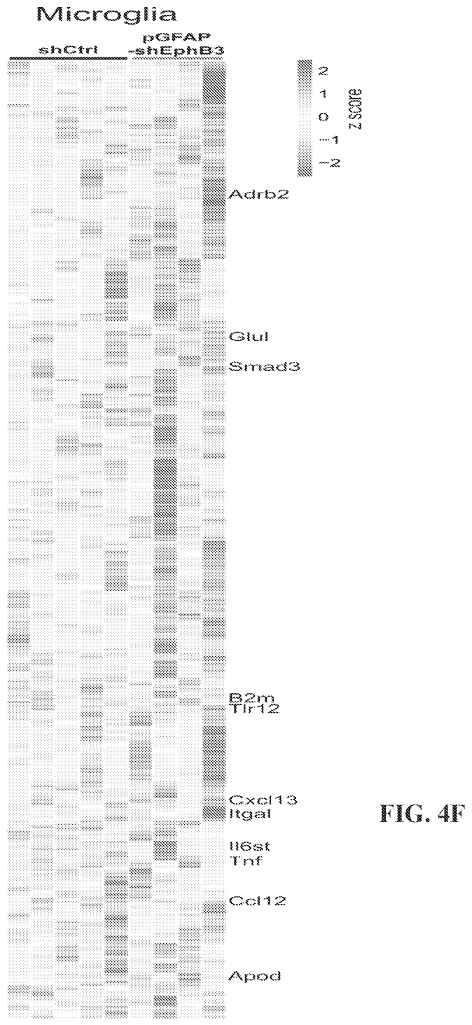

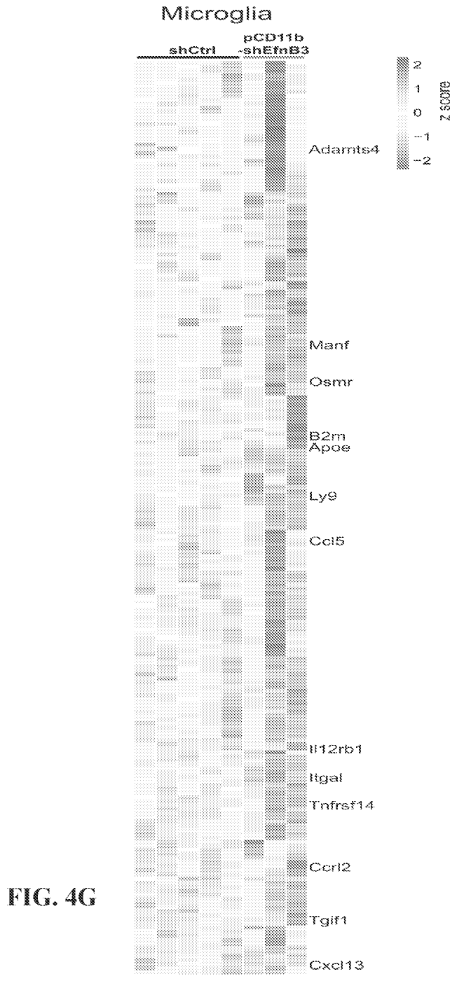

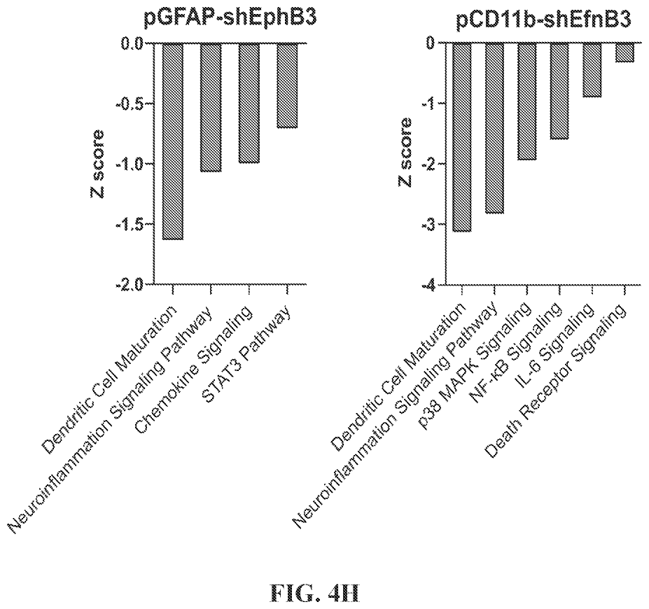

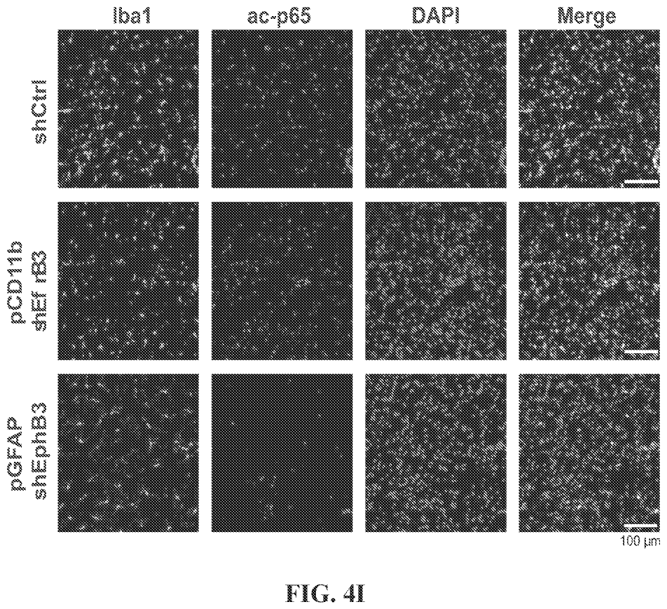

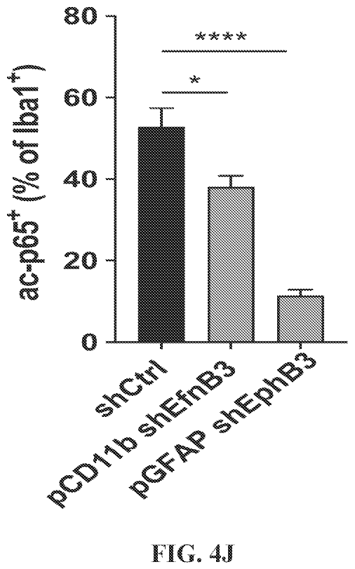

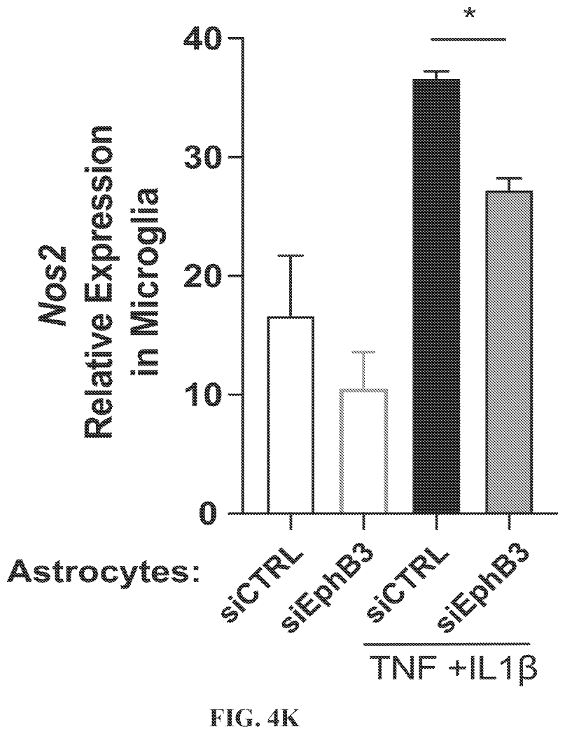

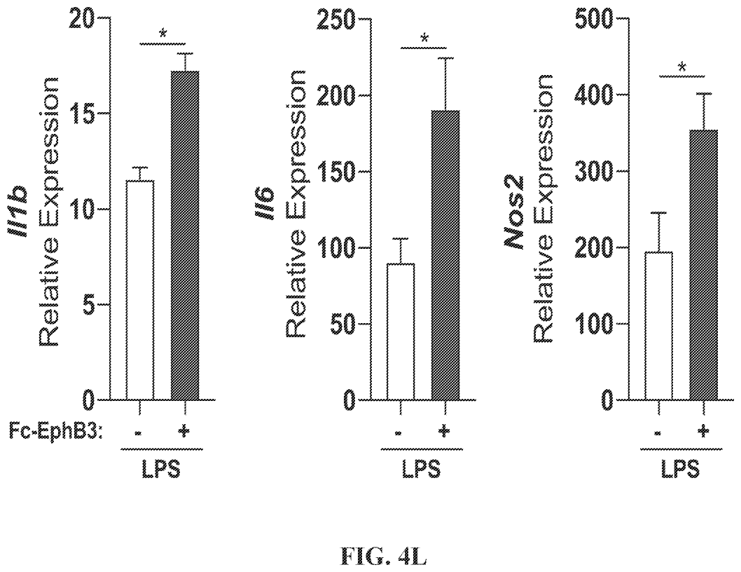

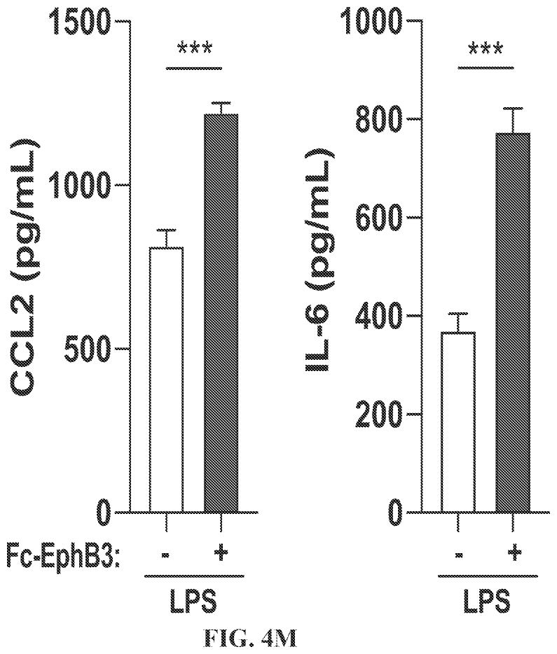

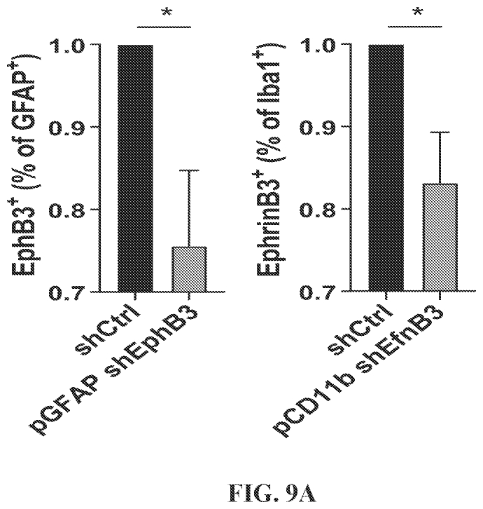

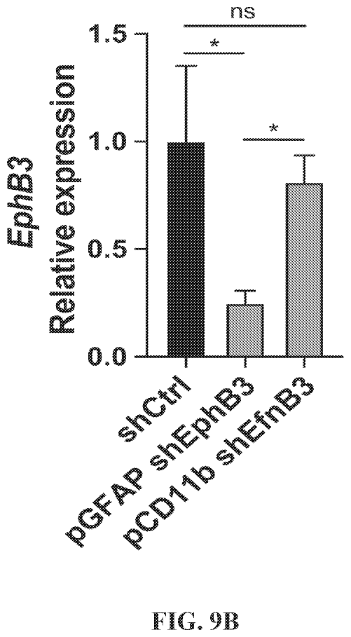

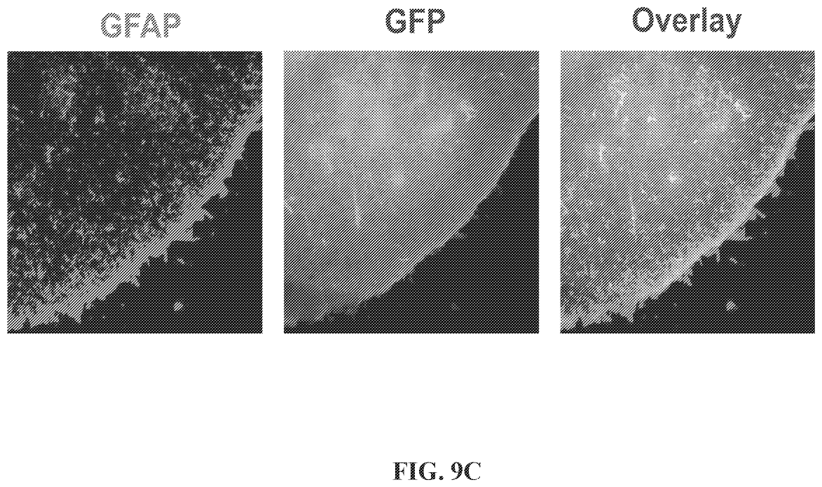

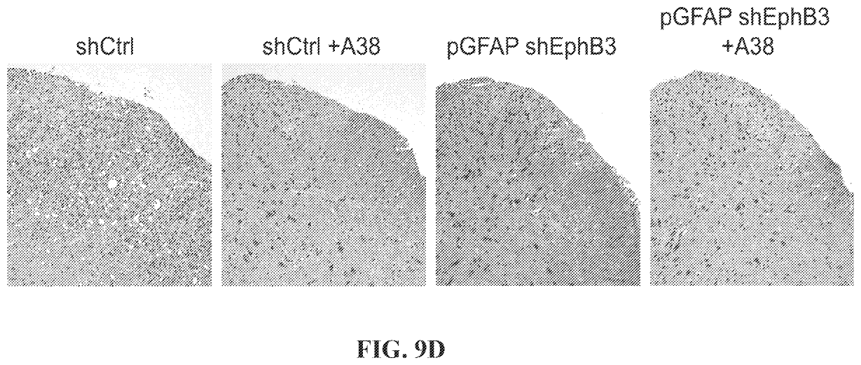

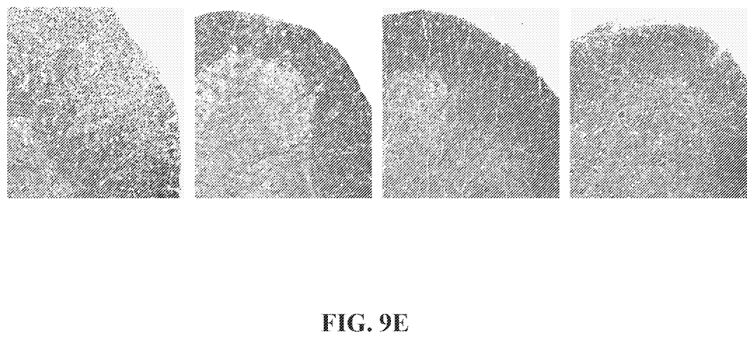

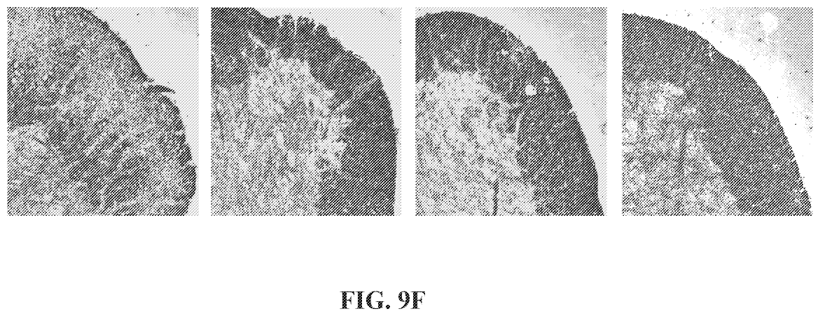

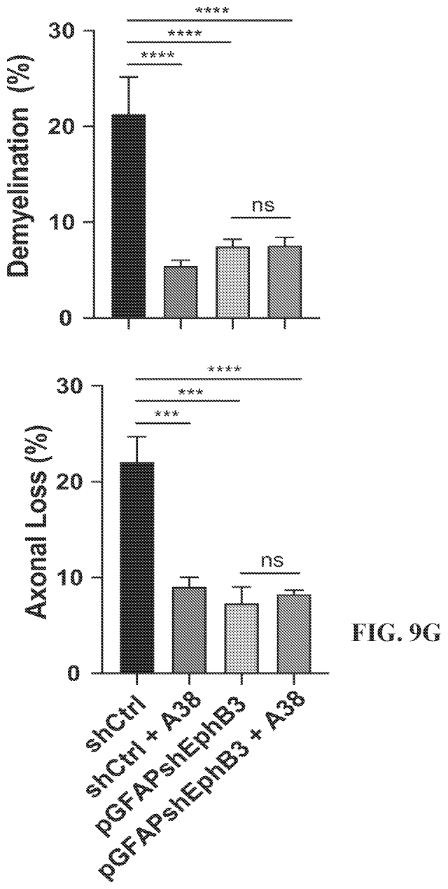

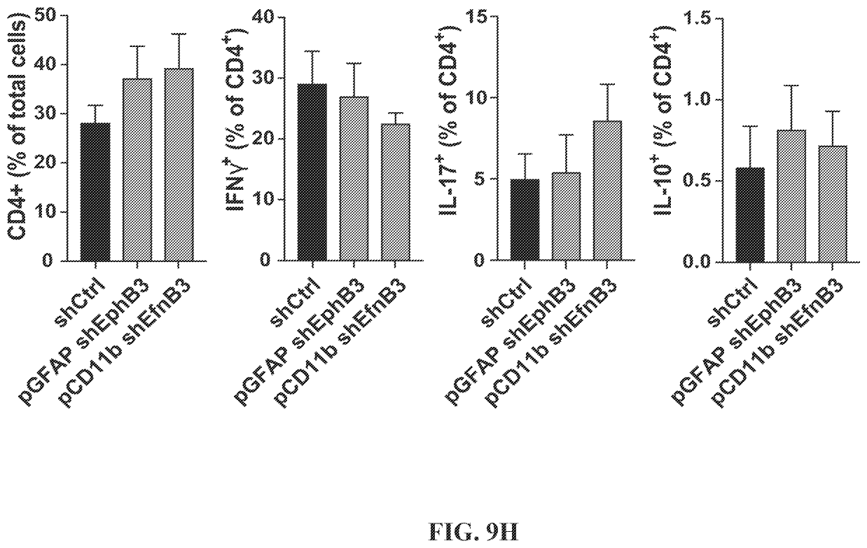

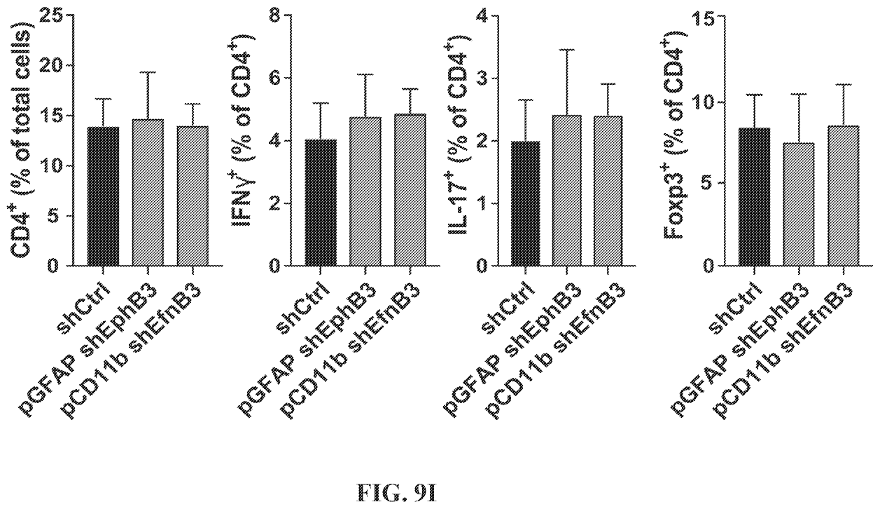

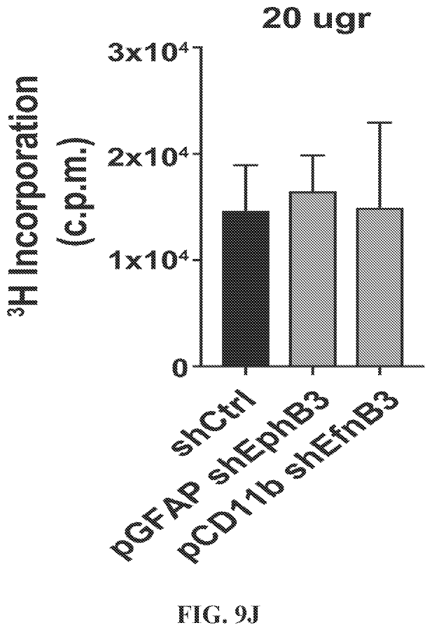

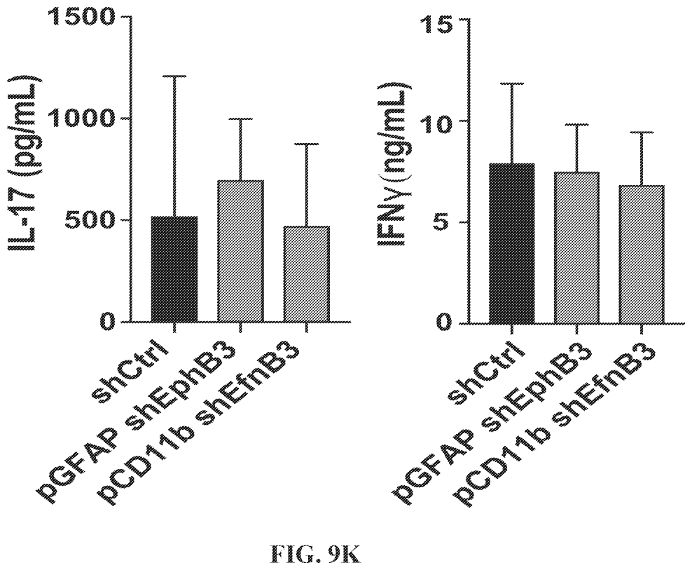

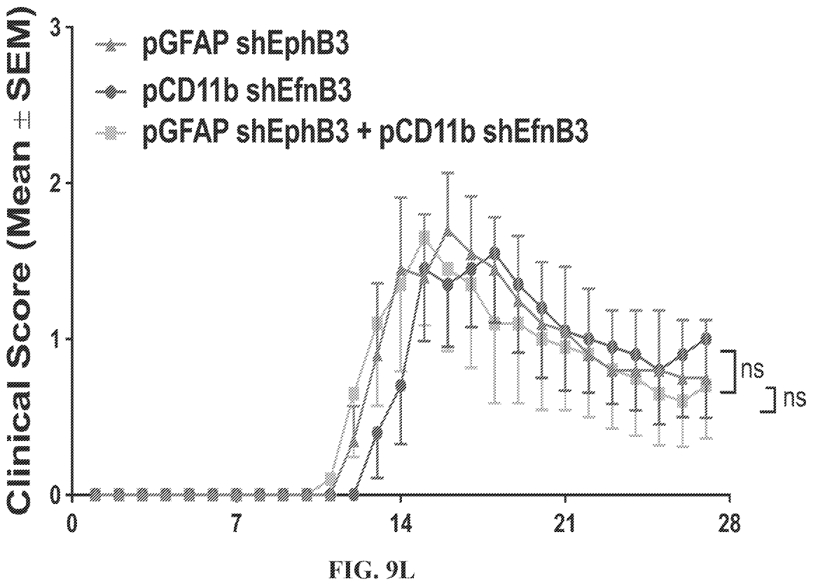

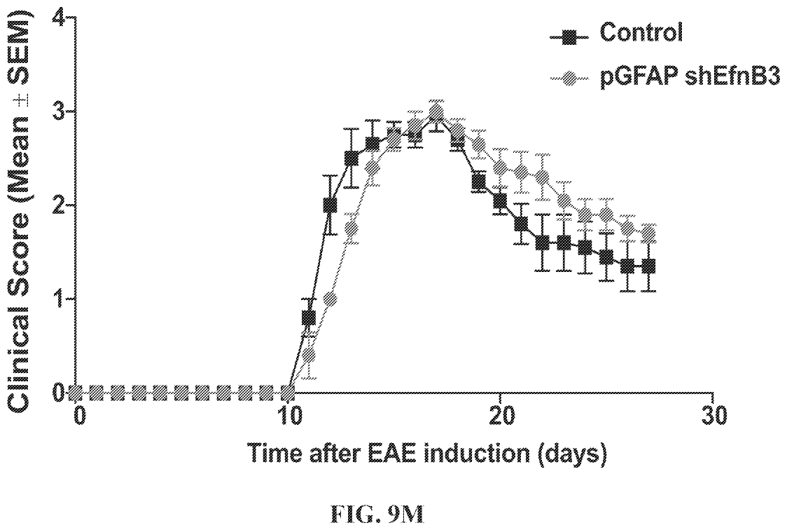

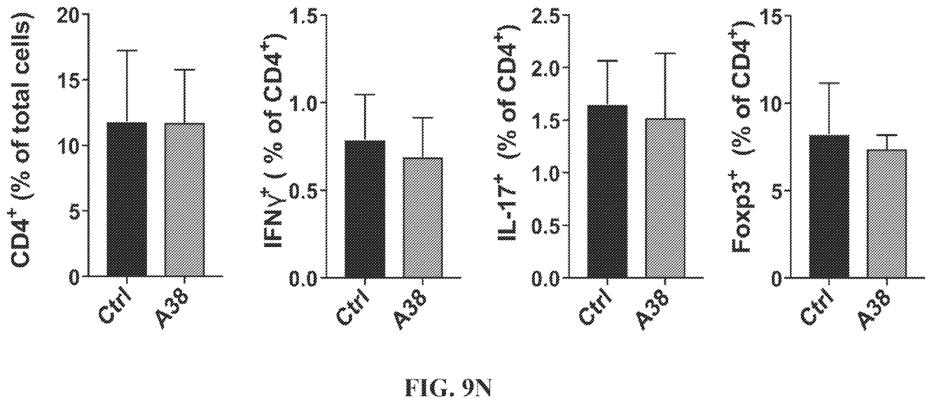

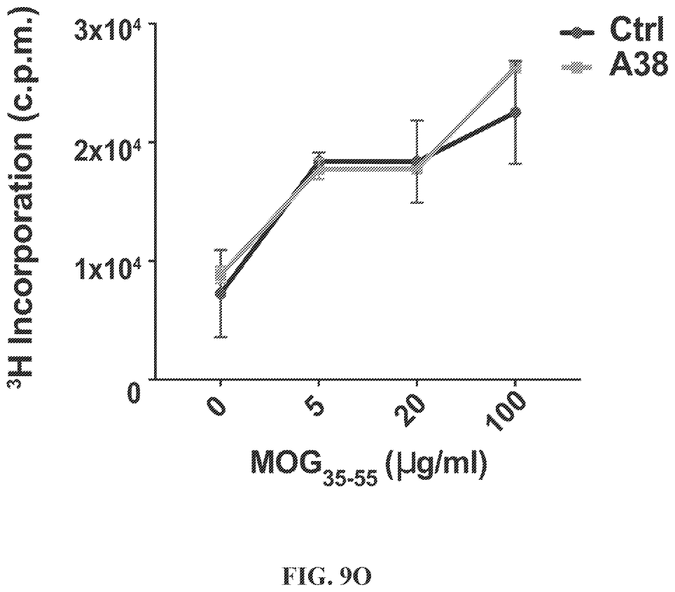

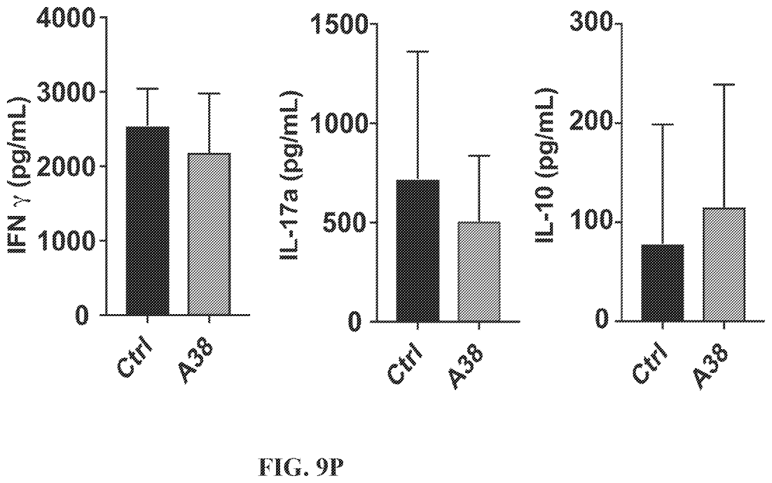

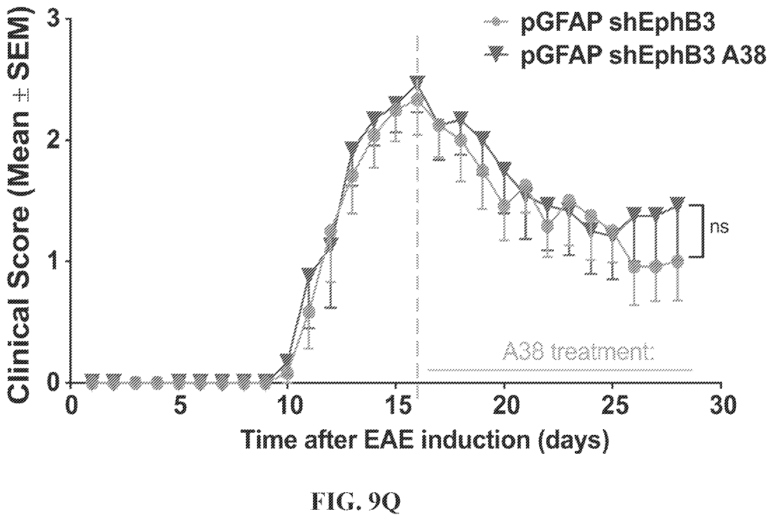

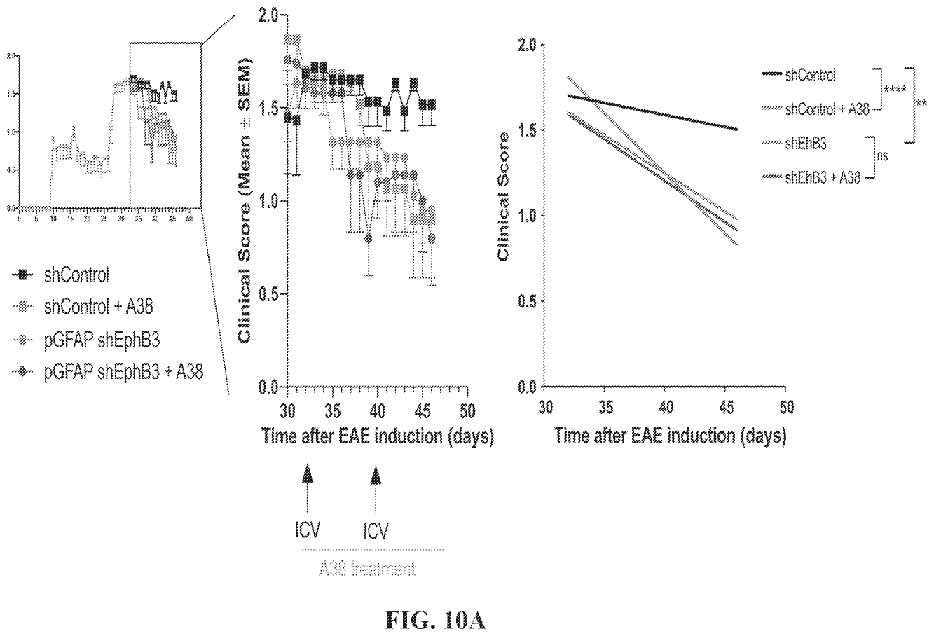

EAE mice were injected ICV at day 7 after immunization, before disease onset, to target CNS resident cells as described. The lentiviruses reached the spinal cord, and the knockdown of Ephb3 in astrocytes or Efnb3 in microglia resulted in a comparable amelioration of EAE (FIG. 4A, FIG. 9A-G), but did not affect T cell responses (FIG. 9H-K). Ephb3 and Efnb3 knockdown led to a reduction in pro-inflammatory Ly6C^{Hi} monocytes recruited to the CNS during EAE (FIG. 4B). Of note, the simultaneous knockdown of Ephb3 in astrocytes and Efnb3 in microglia did not further ameliorate the disease as compared to single knockdowns (FIG. 9L). Moreover, no EAE amelioration was detected following Efnb3 knockdown in astrocytes (FIG. 9M). These results suggest directionality of Ephrin-B3/EphB3 signaling from microglia to astrocytes, ruling out significant effects of autocrine or paracrine astrocyte-astrocyte Ephrin-B3/EphB3 interactions in vivo.

The analysis of the transcriptional response of astrocytes from EAE mice following the knockdown of Ephb3 in astrocytes or Efnb3 in microglia revealed decreased expression of genes associated with inflammation and neurodegeneration (FIG. 4C-E). Taken together, these data demonstrate that microglial Ephrin-B3 and astrocyte EphB3 receptor participate in the control of astrocyte pro-inflammatory activities during a neurodegenerative disease such as an EAE.

### Example 5—Reverse Ephrin-B3 Signaling Boosts NF-κB Driven Responses in Microglia During EAE

The interaction between EphB receptors and their membrane-bound ligands, ephrins of the B family, triggers reverse signaling in Ephrin-B-expressing cells. In support for a role of reverse Ephrin-B3 signaling in the control of microglial responses during EAE, the knockdown of Efnb3 in microglia or Ephb3 in astrocytes decreased pro-inflammatory gene expression in microglia as shown in FIG. 4F-H.

Moreover, in agreement with the diminished microglial pro-inflammatory transcriptional response detected by RNA-seq, decreased NF-κB activation was detected following the knockdown of Efnb3 in microglia or EphB3 in astrocytes, suggesting that NF-κB-driven pro-inflammatory transcriptional programs in microglia was boosted by Ephrin-B3 signaling (FIG. 4I-J).

Ephrin-B3/EphB3 interactions may modulate microglial responses via reverse signaling through Ephrin-B3 expressed in microglia, and indirectly via EphB3-controlled astrocyte secreted factors.

To study the role of reverse Ephrin-B3 signaling in the control of microglial responses, mouse neonatal microglia were co-cultured with mouse neonatal astrocytes pre-stimulated with TNFα and IL-1β under in vitro conditions. The co-culture with pre-stimulated astrocytes increased microglial Nos2 expression, and this increase was diminished by Ephb3 knockdown in astrocytes (See FIG. 4K), suggesting that EphB3-induced reverse signaling contributed to the control of microglia activation by astrocytes.

Indeed, plate bound EphB3-Fc chimera boosted Il1b, Il6 and Nos2 expression, and IL-6 and CCL2 secretion by mouse primary microglia activated in culture with LPS (FIG. 4L-M). Thus, the data show that reverse Ephrin-B3 signaling boosted microglial pro-inflammatory activities during a neurodegenerative disease such as EAE or MS.

### Example 6—Pharmacologic Inhibition of EphB3 Receptor Kinase Ameliorates EAE

Multiple signaling events are triggered by EphB3 receptor activation; one of these signaling mechanisms is EphB3 kinase activity.

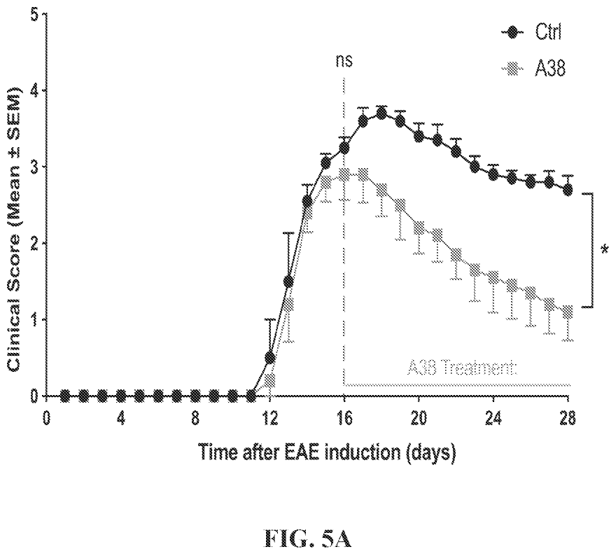

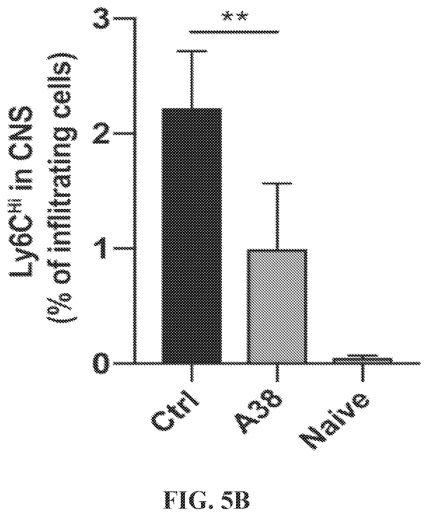

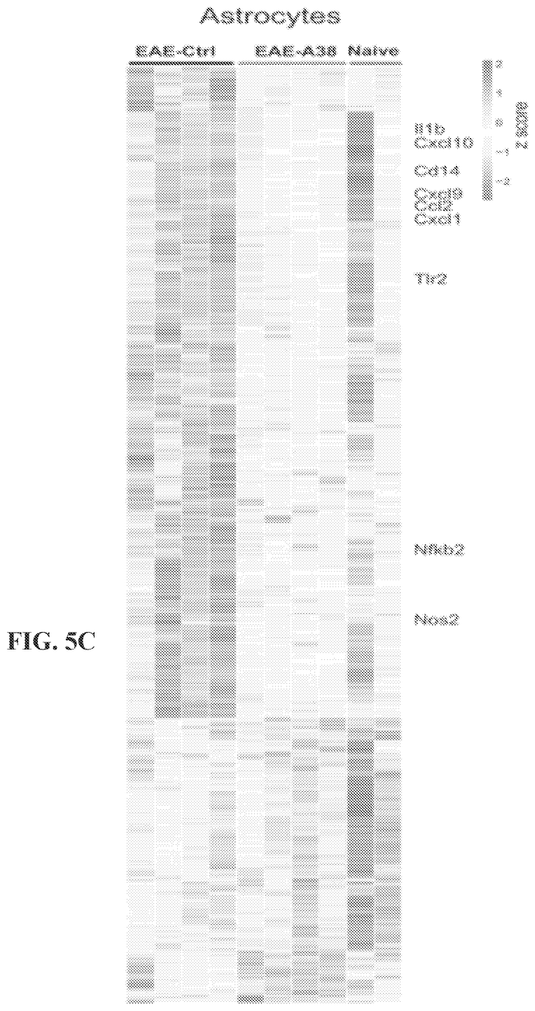

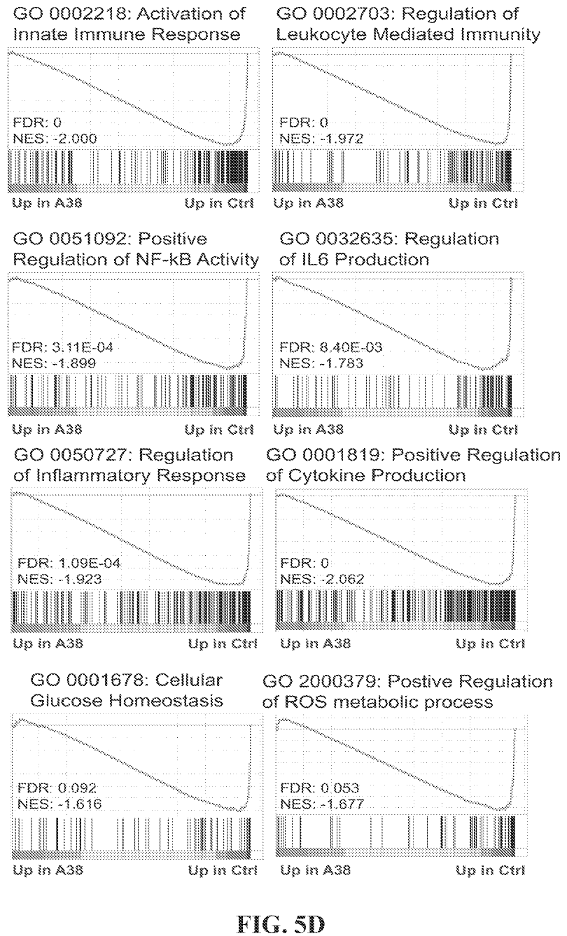

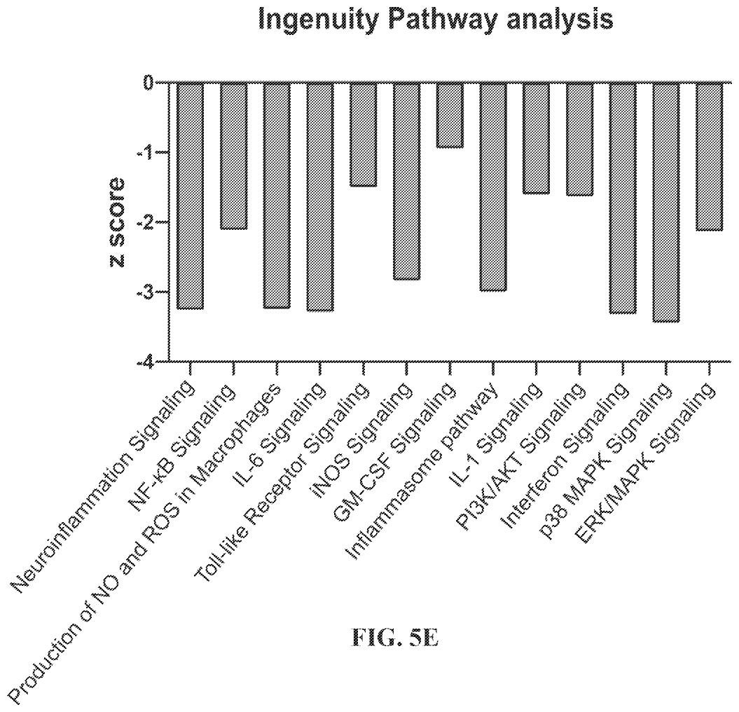

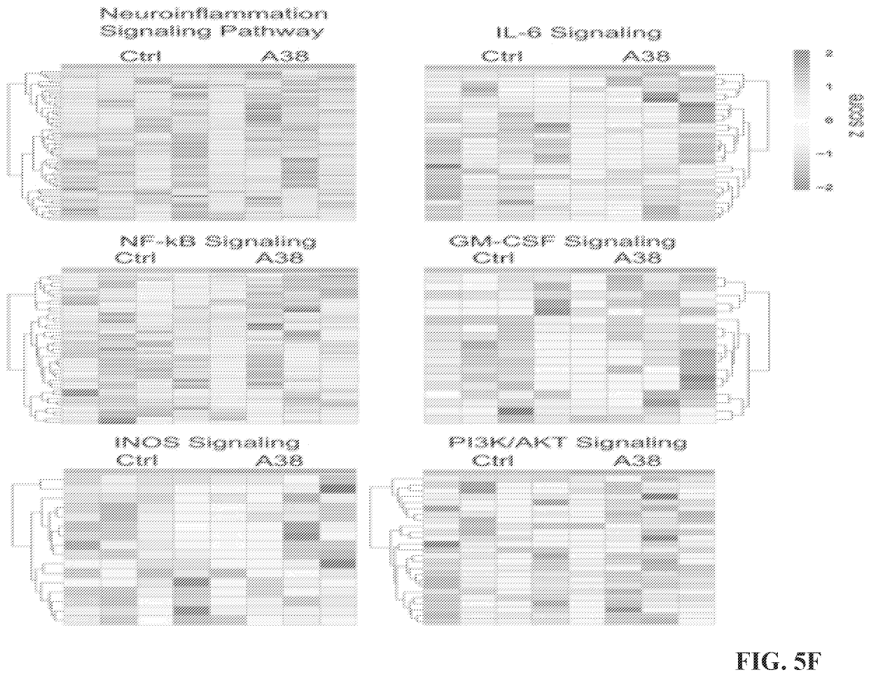

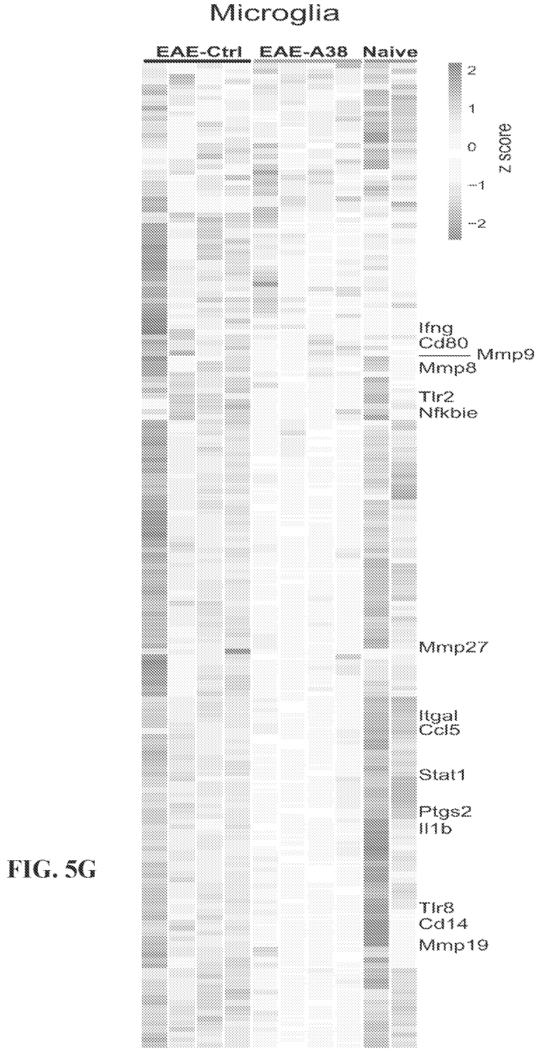

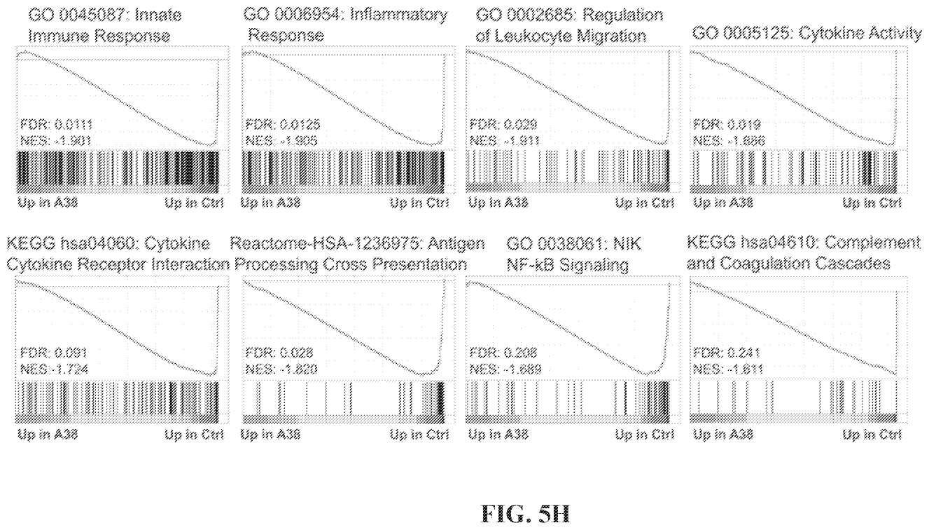

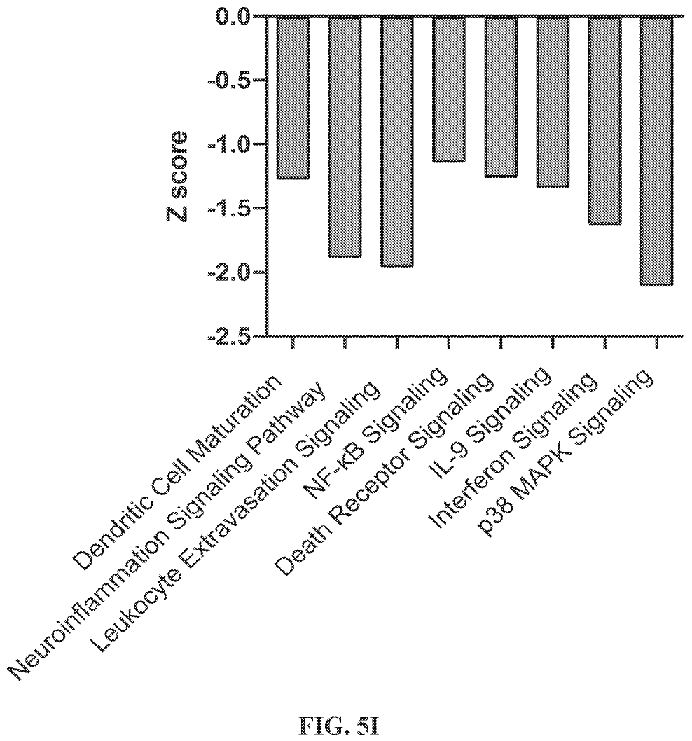

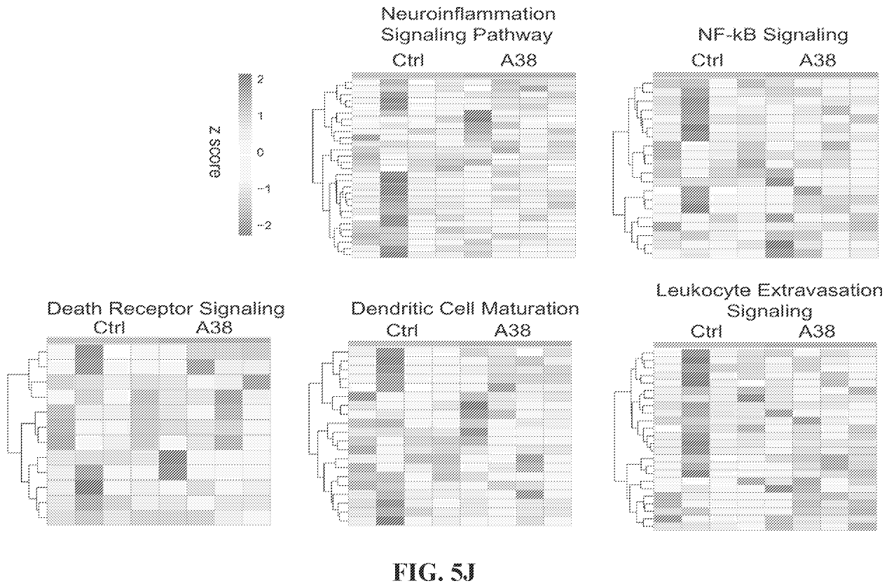

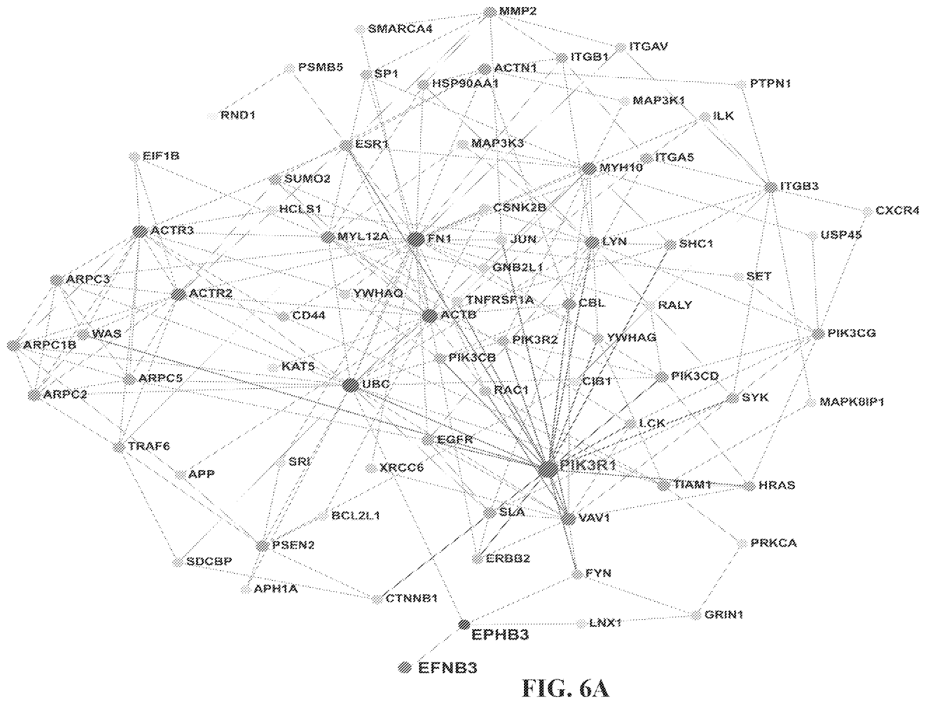

To evaluate the potential of the EphB3 kinase as a therapeutic target during CNS inflammation, EAE was induced in B6 WT mice by immunization with MOG₃₅₋₅₅. Treatment was initiated with A38 (20 mg/kg body weight) at the peak of the disease; vehicle was used as a control. A38 administration ameliorated EAE, as indicated by the reduction in clinical scores, reduced demyelination, and axonal loss detected in histopathological analyses (FIG. 5A, FIG. 9D-G). A38 also reduced the recruitment of Ly6C^{Hi} pro-inflammatory monocytes to the CNS (See FIG. 5B), but did not affect T-cell response (FIG. 9N-P).

Moreover, A38 administration decreased astrocyte and microglial expression of transcriptional modules associated with the promotion of CNS inflammation and neurodegeneration as determined by RNA-seq (FIG. 5C-J).

Of note, A38 administration concomitant with EphB3 knockdown in astrocytes did not further increase the therapeutic effects achieved by these interventions alone (FIG. 9D-G, FIG. 9Q), suggesting that the amelioration of EAE by A38 involved the inhibition of EphB3 kinase activity in astrocytes.

To further validate the potential of EphB3 kinase inhibition for the therapeutic modulation of CNS inflammation and neurodegeneration, a study was performed using non-obese diabetic (NOD) mouse model of chronic progressive EAE induced by immunization with MOG₃₅₋₅₅, which recapitulated aspects of secondary progressive MS, including the progressive and irreversible accumulation of neurologic disability.

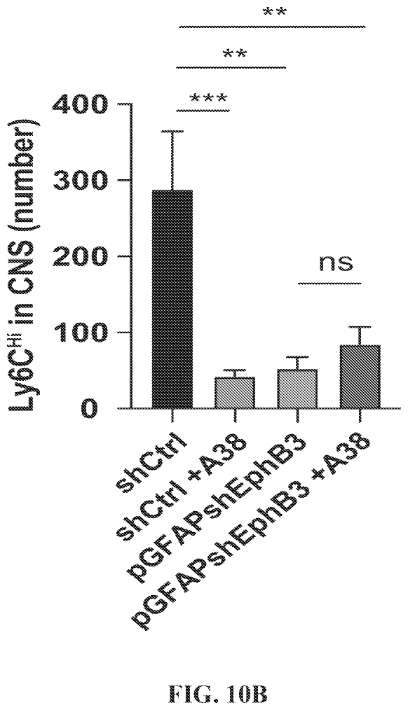

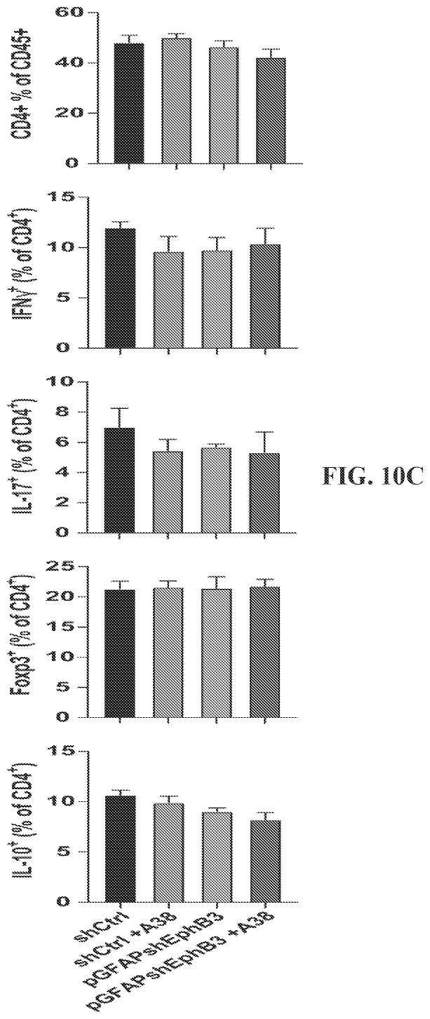

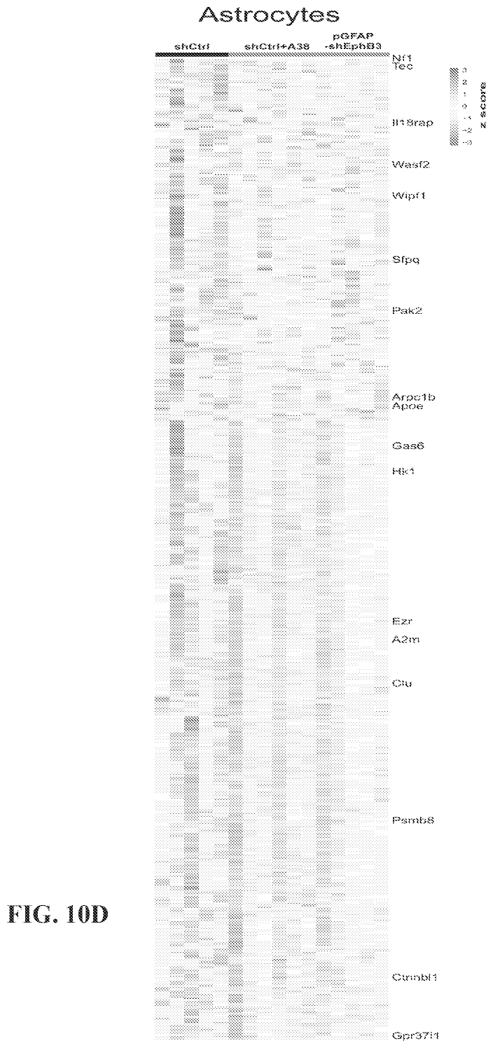

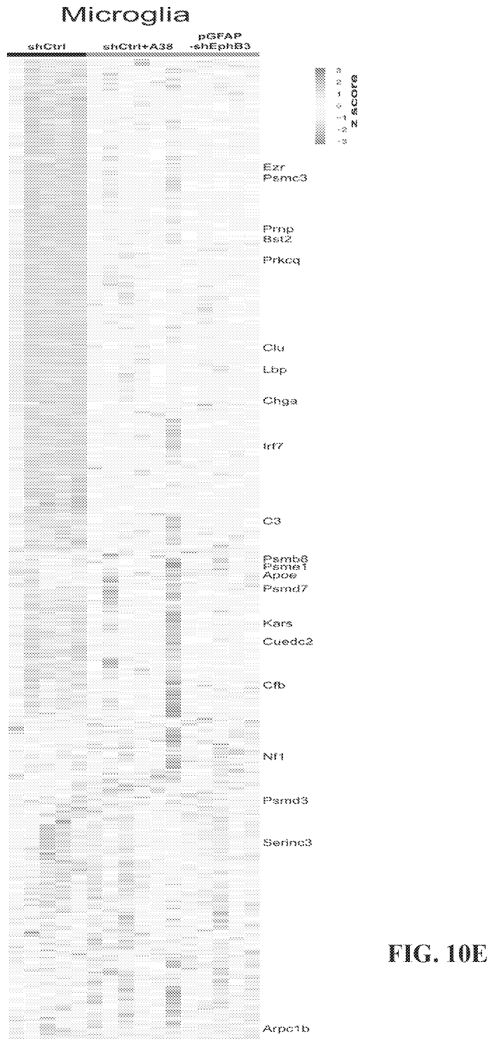

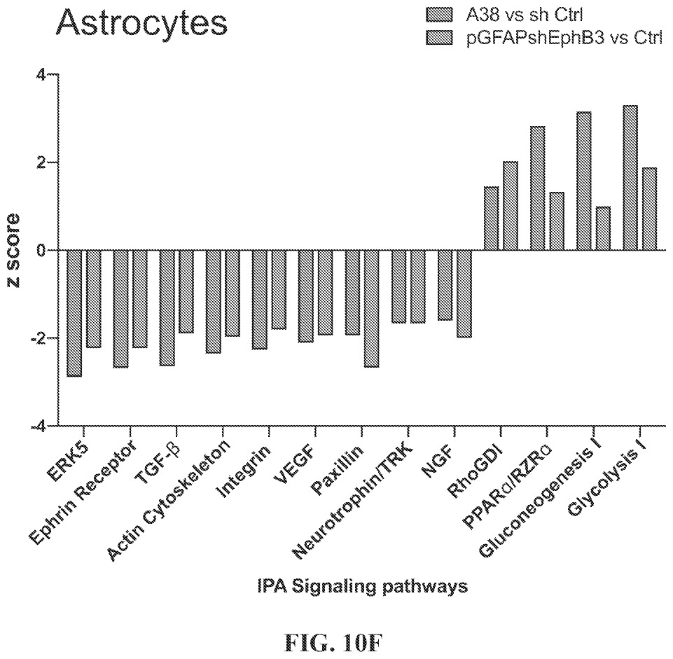

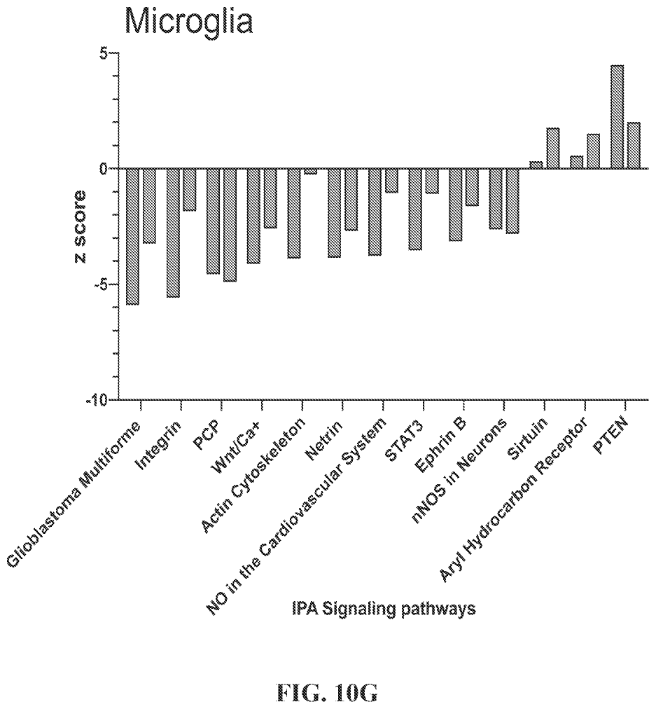

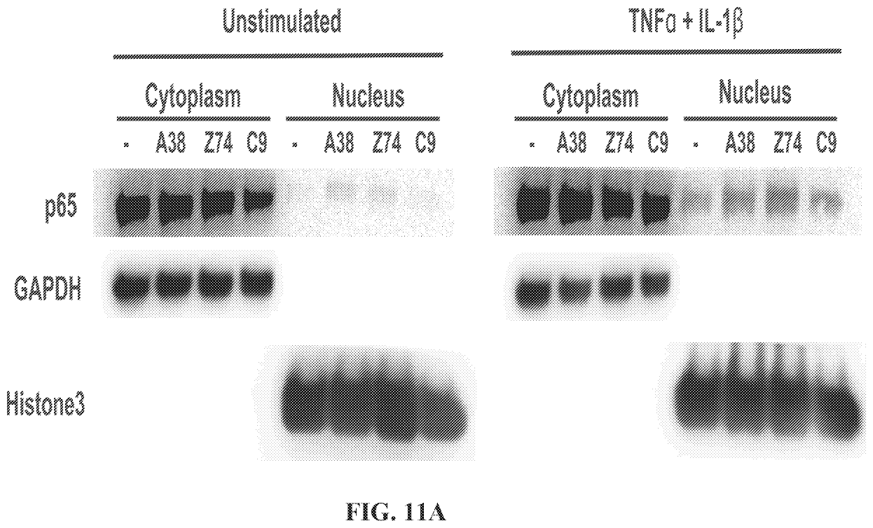

Specifically, the effects of A38 administration or EphB3 knockdown in astrocytes during the progressive phase of NOD EAE were evaluated (FIG. 10A). Pharmacological inactivation of EphB3 kinase activity with A38 and the knockdown of Ephb3 in astrocytes by lentivirus-delivered shRNAs ameliorated NOD EAE, as indicated by the reduced clinical scores and the decreased recruitment of pro-inflammatory monocytes to the CNS; no changes in the peripheral T-cell response were detected (FIG. 10A-C). Additionally, A38 administration and EphB3 knockdown in astrocytes suppressed the astrocyte and microglial expression of transcriptional modules associated with CNS inflammation and neurodegeneration (FIG. 10D-G). Thus, EphB3 signaling in astrocytes was shown to promote CNS inflammation. Inhibition of that signaling reduced or eliminated the inflammatory response.

### Example 7—EphB3 Kinase Activates mTOR and Boosts Mitochondrial ROS Production in Astrocytes

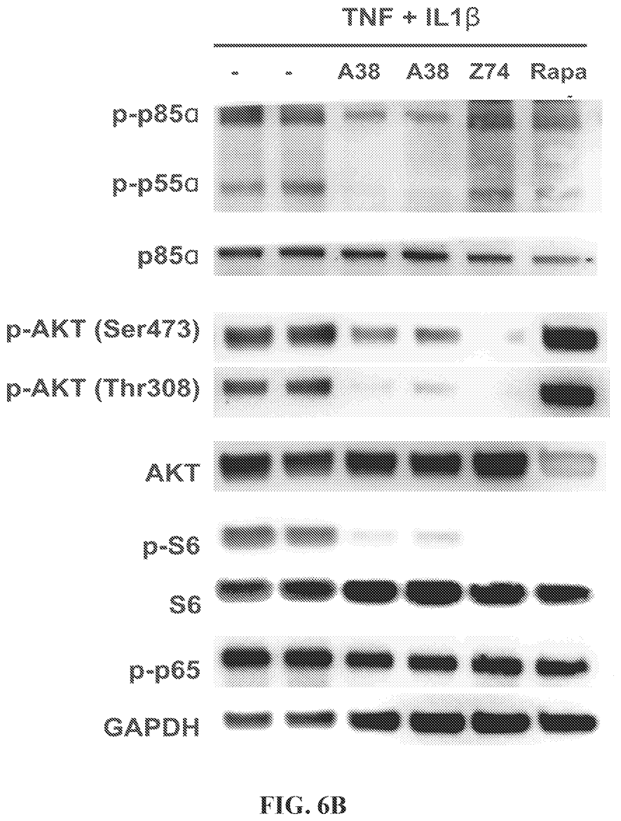

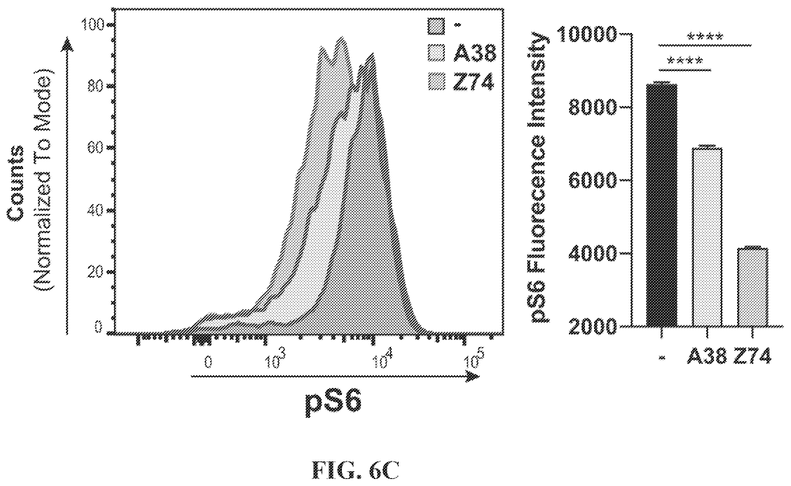

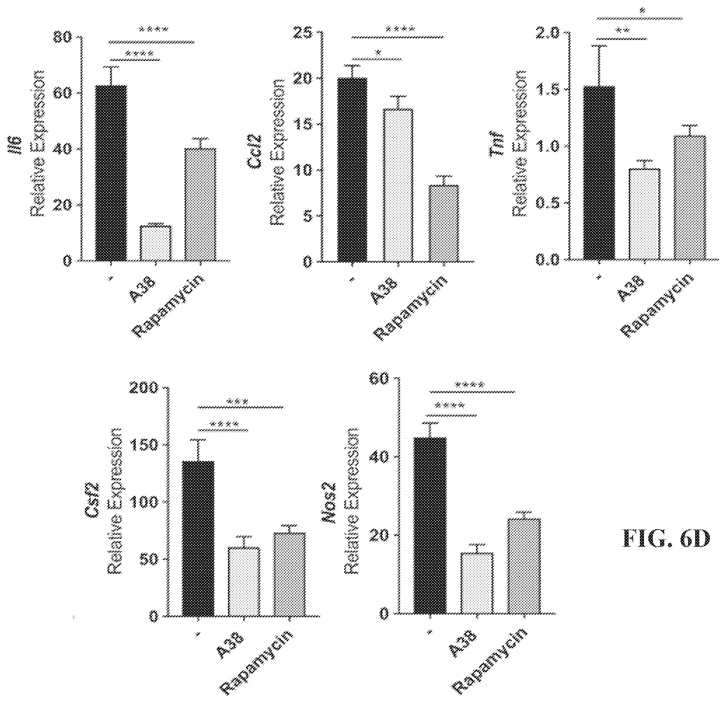

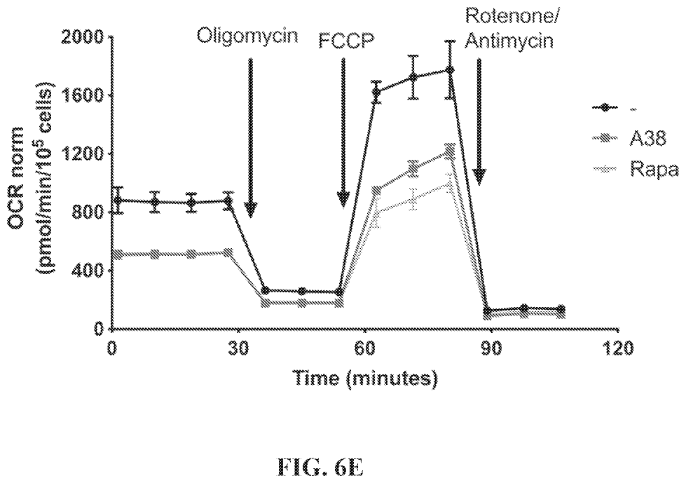

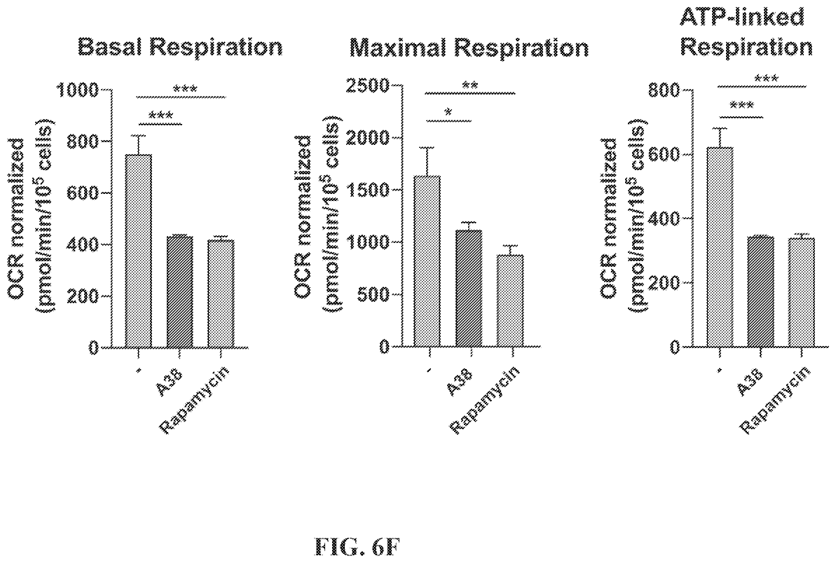

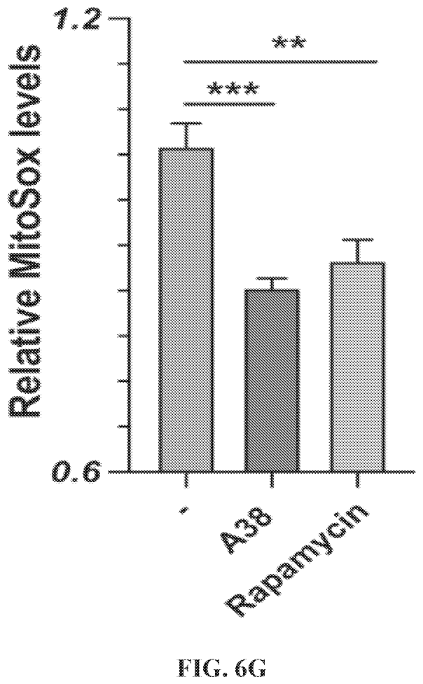

Finally, the mechanisms involved in the control of astrocyte pro-inflammatory activities by EphB3 signaling were investigated. Bioinformatic analysis of the transcriptional response of astrocytes following EphB3 inactivation during EAE, identified PIK3R1 as a candidate mediator of the effects of EphB3 signaling (FIG. 6A). PIK3R1 encodes the regulatory subunits (p85α, p55α and p50α) of Class I PI3Kα, which is associated to the control of innate immunity. Supporting the role of PIK3R1 in EphB3 signaling in astrocytes, A38 suppressed the phosphorylation of p85a, p55a and their downstream signaling molecule AKT in primary murine astrocytes in culture (FIG. 6B). Similarly, the pharmacological inhibition of Class I PI3Kα by ZSTK474 suppressed the phosphorylation of its target AKT (FIG. 6B).

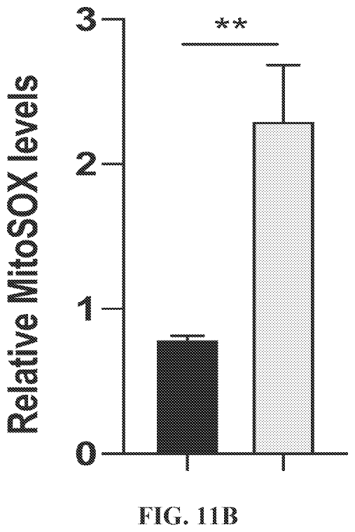

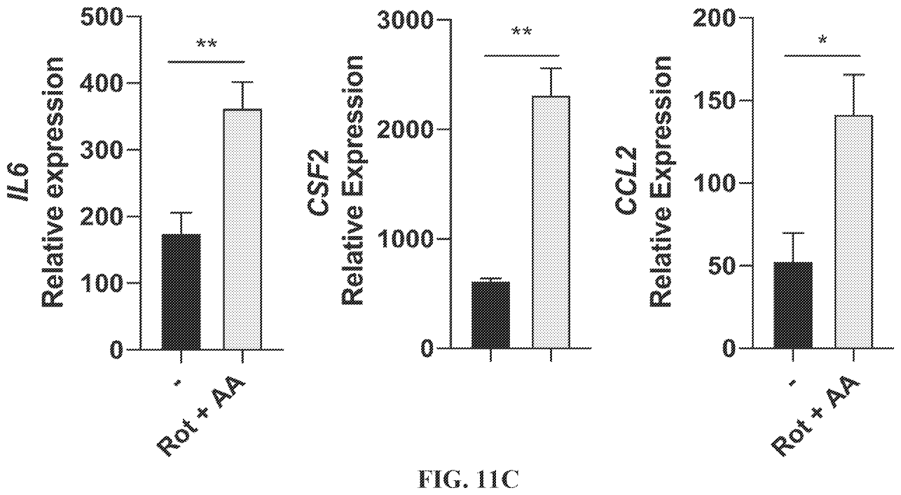

AKT has been reported to activate the transcription factor NF-κB and the mammalian target of rapamycin (mTOR). The inhibition of AKT phosphorylation by A38 did not suppress NF-κB activation as determined by the analysis of its phosphorylation and nuclear translocation in primary astrocytes stimulated with TNFα and IL-1β (FIG. 6B, FIG. 11A). However, A38 suppressed S6 phosphorylation, downstream of mTOR^{c1}, suggesting that A38 interferes with mTOR activation (FIG. 6B). Indeed, reduced S6 phosphorylation was detected when the effects of the pharmacological inhibition of EphB3 kinase or PI3K in primary astrocytes activated in vitro with TNFα and IL-1 was analyzed by flow cytometry (FIG. 6C). Collectively, these data suggest that, through its effects on the PI3K/AKT axis, EphB3 kinase activity promoted mTOR activation in astrocytes.

To investigate the role of mTOR on astrocyte responses the mTOR inhibitor rapamycin was used. Rapamycin treatment suppressed the expression of pro-inflammatory genes in astrocytes stimulated in vitro with TNFα and IL-1β (FIG. 6D), and recapitulated previous observations presented in this disclosure on the effects of A38 on astrocytes. In agreement with its inhibitory effects on mTOR, rapamycin suppressed S6 phosphorylation but did not suppress the phosphorylation of ATK, p85 or NF-κB subunit p65 (FIG. 6B).

mTOR controls mitochondrial function, which has been linked to pathogenic activities of astrocytes and microglia in neurologic disorders. Indeed, mTOR-driven mitochondrial respiration produces reactive oxygen species (ROS), which promote pro-inflammatory gene expression and contribute to neurodegeneration. Thus, the effects of A38 on mitochondrial function and ROS production was investigated. The inhibition of EphB3 kinase activity by A38 decreased basal and maximal mitochondrial respiration, as well as ATP-linked respiration in primary astrocytes (FIG. 6E-F); similar results were obtained with rapamycin. In agreement with its suppressive effects on mitochondrial respiration, A38 and rapamycin also decreased the mitochondrial production of ROS induced in astrocytes by stimulation with TNFα and IL-1β (FIG. 6G). Accordingly, a decrease in the expression of genes related to ROS metabolic processes in astrocytes isolated from A38-treated EAE mice was detected (FIG. 5D). Moreover, treatment with Rotenone and Antimycin A, which increases mitochondrial ROS levels and FIG. 11B), boosted the expression of proinflammatory genes in astrocytes (FIG. 11C). Taken together, these results show that the production of mitochondrial ROS driven by PI3K/AKT/mTOR signaling contributed to the pro-inflammatory effects of EphB3 kinase activation in astrocytes, and that inhibitors of EphB3 kinase (such as A38) reduced or eliminated these inflammatory responses.

### Example 8—Targeting of Ephrin Signaling for Astrocyte Modulation by Compound A38

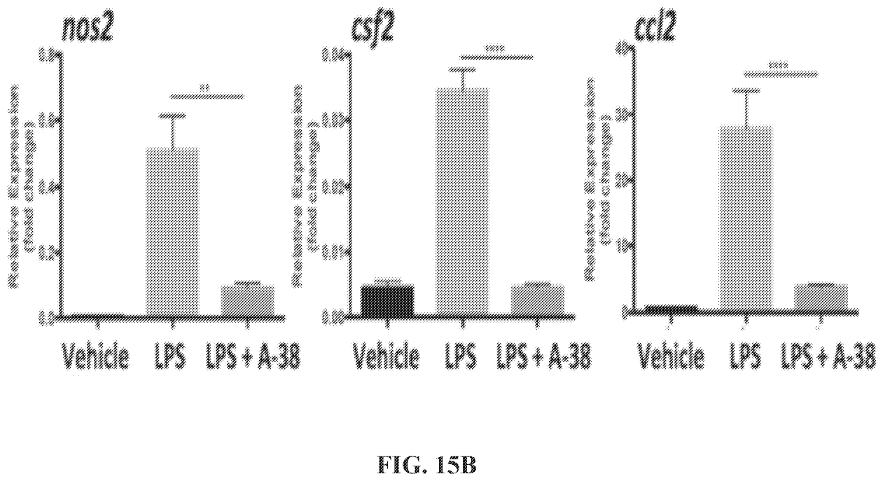

As discussed above, A38 is a modulator of Eph/Ephrin signaling and is highly efficient in down-modulating TNF-α production from primary astrocytes (See FIG. 15A and FIG. 15B). A38 has desirable physicochemical properties for a CNS drug: MW=355, cLogP=2.4 and PSA=57. Compound A-38 displayed excellent mouse liver microsome stability (T_{1/2}>5 h, Clᵢₙₜ=4 μL/min/mg protein) and had very high aqueous solubility (>10 mg/mL at pH 7.4) due to the basic piperidine group. In addition, compound A-38 demonstrated no cytotoxicity at 30 μM in the both MTT and ATP-viability assays.

Based on the excellent in vitro drug-like properties, a preliminary in vivo pharmacokinetic evaluation, using the mono-oxalate salt of A-38, was conducted. Following IP (intraperitoneal) administration (3 mg/kg in 100% saline solution) to male Sprague-Dawley rats, both plasma and brain concentrations were determined at 0.25, 0.5, 1, 2, and 8 h. In the plasma, the compound reached encouraging levels (Cₘₐₓ=221 ng/mL) with a moderate plasma half-life (T_{1/2}=2.25 h). Brain exposure of A-38 was good (Cₘₐₓ=331 ng/mL), and a brain to plasma ratio of 1.5 to 1 indicated little resistance to brain penetration and that A-38 was not a Pgp substrate. In sum, compound A-38 demonstrated desirable properties for use in treatment of EAE and MS.

The compound A38 was assessed for its neuroprotective effects in vivo in an animal model of MS. In the model of experimental autoimmune encephalomyelitis (EAE), a susceptible mouse strain (C57Bl/6) is immunized with a myelin peptide (MOG35-55) to induce pathogenic T cell activation and additional pro-inflammatory cascades that ultimately lead to the formation of inflammatory lesions in the CNS. This animal model recapitulates several aspects of MS and has proven instrumental for the preclinical evaluation of novel therapeutic strategies for MS.

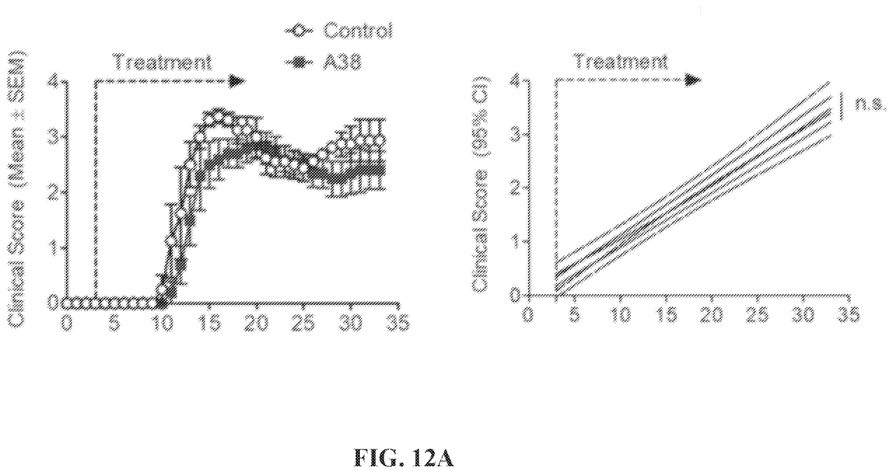

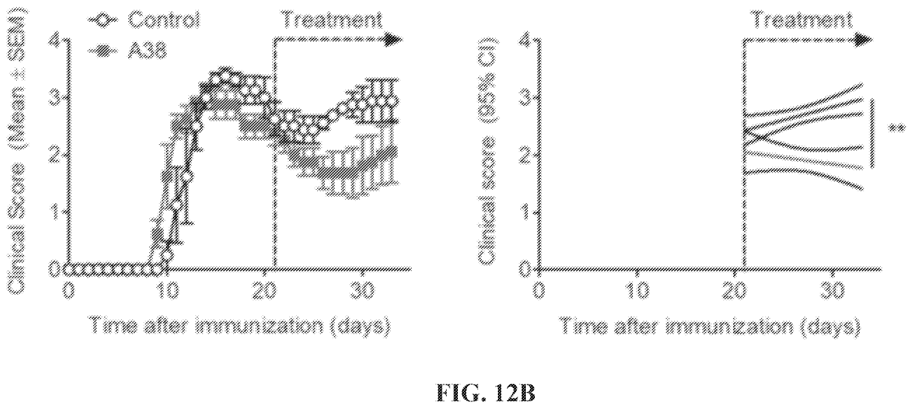

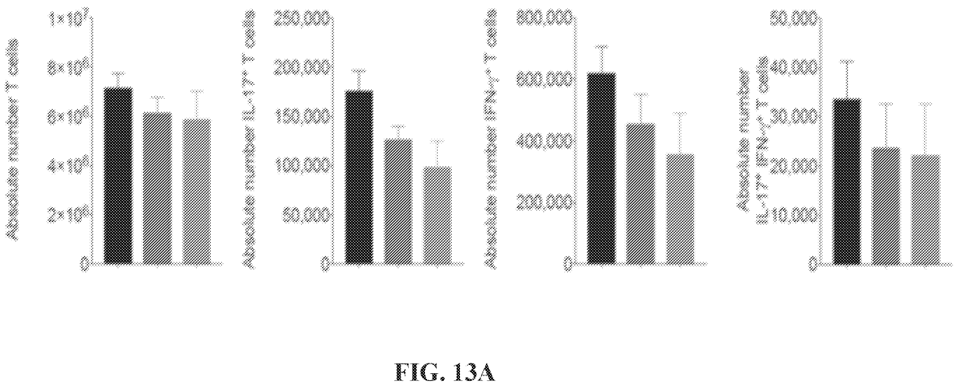

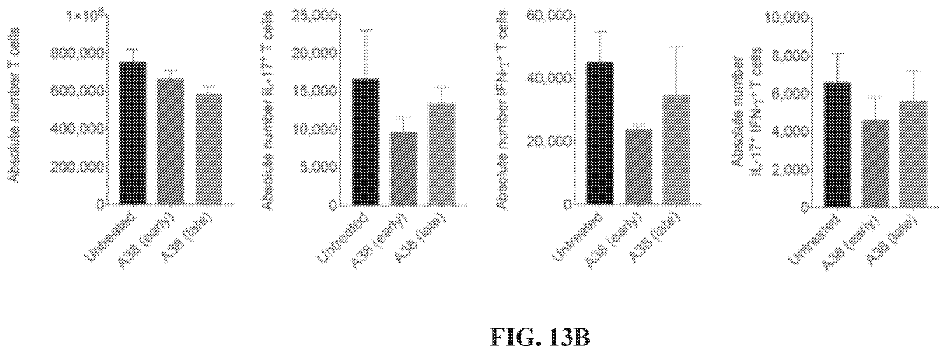

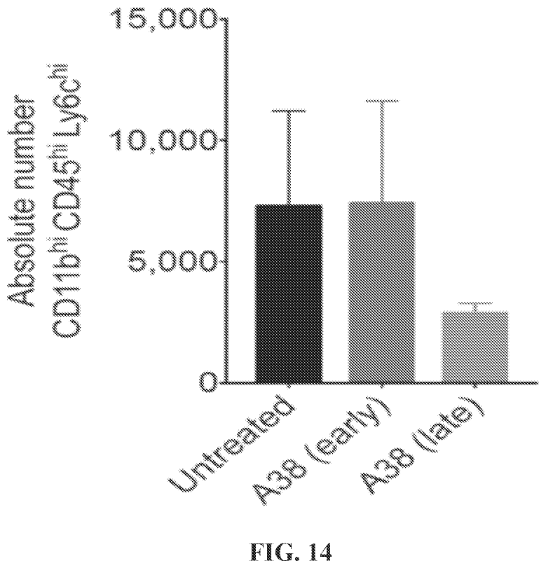

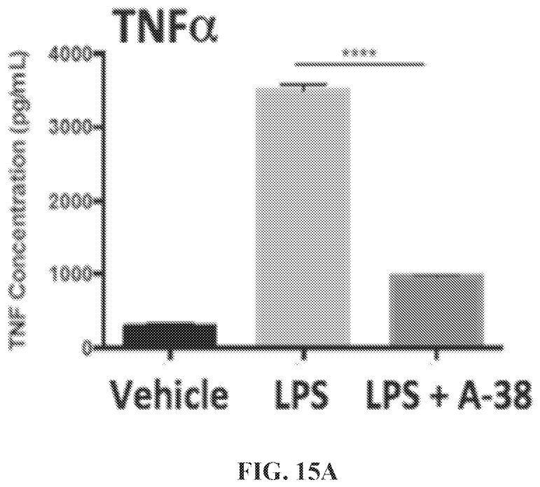

The effects of A38 on EAE were tested. After immunization, mice were treated with daily intraperitoneal injections of A38 or Control starting from day 3 (priming phase, FIG. 12A), or day 21 after disease induction (chronic phase, FIG. 12B). While the preventive treatment from day 3 onwards did not alter the course of EAE, treatment during the chronic phase ameliorated the clinical course significantly and led to a lower level of impairment at the end of the experimental period (FIG. 12B). This observation was reflected in a significant decrease in IFN-γ and IL-17 producing T cells in the CNS. T cell counts in the spleen, however, were unchanged (FIG. 13A and FIG. 13B). Pro-inflammatory macrophages in the CNS have been described as mediators of disease progression in chronic stages of MS. Accordingly, a decrease of pro-inflammatory monocytes was detected in the CNS of A38 treated mice (FIG. 14). This immunological profile shows the role of A38 in the down-modulation of pro-inflammatory and neurotoxic pathways. Hence, A38 and other compounds of this disclosure are useful in treatment of AE and MS (including late stage MS) and other neurodegenerative diseases driven by dysregulated adaptive (T cells and B cells) and innate (monocytes, microglia and astrocytes) immune responses.

Discussion of Examples 1-8

Communication between glial cells plays a role in CNS physiology. Microglia modulate astrocyte responses through the secretion of cytokines and other soluble factors. Conversely, astrocytes produce factors that modulate microglial responses. Experimental results presented in this disclosure show that Eph receptor signaling, classically associated with axon guidance, participated in microglial-astrocyte bi-directional communication, promoted CNS inflammation and neurodegeneration.

Eph receptor signaling plays important roles in development, and in multiple homeostatic processes including immune-regulation, maintenance of epithelial architecture, control of neural progenitor proliferation, axon guidance and synapse formation. Eph receptors participate in astrocyte-neuron communication, and EphA receptor upregulation in astrocytes has been reported in MS patients, but the functional relevance of this observation was never investigated. In addition, increased EphB2 and EphB3 expression is detected in astrocytes following spinal cord injury, and Eph receptor signaling has been linked to the pathology of Alzheimer's disease, Parkinson's disease, amyotrophic lateral sclerosis and schizophrenia. However, a distinguishing feature of Ephrin-Eph receptor interactions is the induction of reverse signaling in Ephrin-expressing cells, which amplifies NF-κB-driven pro-inflammatory responses in microglia (as shown by experimental results presented in this disclosure). Thus, Eph receptor signaling provides a cell-contact dependent mechanism to co-regulate microglial and astrocyte responses.

The experimental results presented in the present disclosure also show that EphB3 kinase activity in astrocytes modulates mTOR activation and mitochondrial ROS production. ROS-producing myeloid cells in the CNS were recently characterized (see Akassoglou et al., Nat Immunol. 2020, 21(5), 513-524) and it was found that the genetic suppression of ROS production decreases pro-inflammatory gene expression. ROS triggers the production of pro-inflammatory cytokines via the regulation of NLRP3 and MAPK activity. In addition, EphB3 and EphA4 signaling in astrocytes via PICK1 was reported to induce the production of D-serine which acts as a co-agonist of NMDA receptors to promote synaptic damage. Thus, EphB3 signaling provides a mechanism for the microglial control of astrocyte metabolism and its multiple effects on CNS inflammation.

The investigations conducted in this disclosure showed that the therapeutic blockade of Ephrin-B3/EphB3 signaling interferes not only with disease-promoting responses in astrocytes and microglia, but also with additional mechanisms associated with neurodegenerative disease pathology and linked to this pathway, including the disruption of the blood-brain barrier and the inhibition of remyelination.

### Example 9—Synthesis of N-(2-chlorophenyl)-6-(piperidin-4-yl)imidazo[1,2-a]pyridine-3-carboxamide (compound A-38)

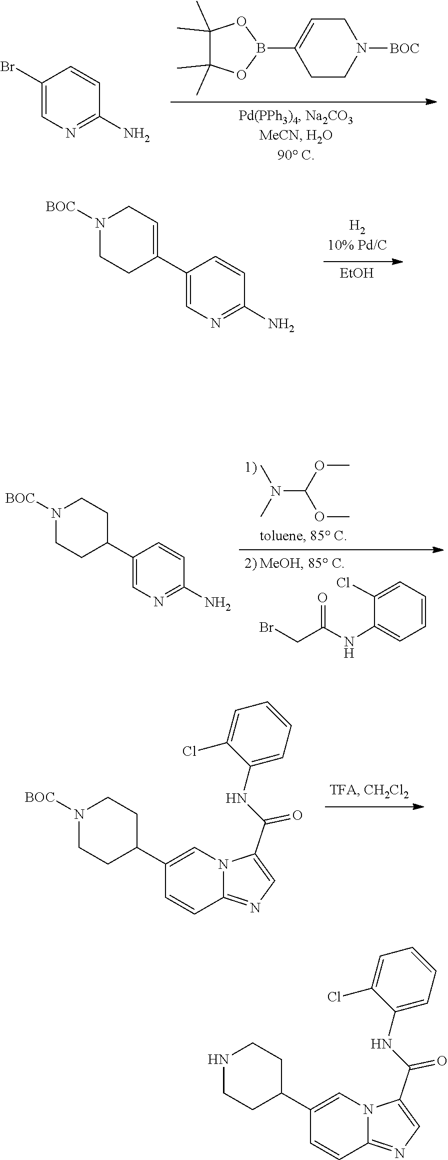

### Step 1—tert-butyl 6-amino-3′,6′-dihydro-[3,4′-bipyridine]-1′(2′H)-carboxylate

2-Amino-5-bromopyridine (1.97 g, 0.0114 mol), 3,6-dihydro-2,4-pyridine-N-Boc-4,4-boronic acid pinacol ester (5.28 g, 0.0171 mol), sodium carbonate (3.62 g, 0.0342 mol), acetonitrile (100 mL) and water (20 mL) were degassed with argon. Tetrakis(triphenylphospine)palladium(0) (65.9 mg, 0.057 mmol) was added and the reaction mixture was heated under an argon atmosphere at 90° C. for 16 hrs. The reaction was cooled to RT and diluted with brine (40 mL). The mixture was extracted with ethyl acetate (3×15 mL), the combined organic layers were collected, dried over anhydrous sodium sulphate, filtered and concentrated. The crude mixture was purified by silica column chromatograph (eluent 95:5 dichloromethane:1% ammonia in methanol) to yield the title compound as thick oil (2.02 g, 64%).

### Step 2—tert-butyl 4-(6-aminopyridin-3-yl)piperidine-1-carboxylate

tert-butyl 6-amino-3′,6′-dihydro-[3,4′-bipyridine]-1′(2′H)-carboxylate (2.02 g, 7.33 mmol), ethanol (60 mL) and palladium on carbon (220 mg) were first degassed with argon and then hydrogen and stirred at RT overnight. The resulting mixture was degassed with argon, filtered over celite, and concentrated. The crude mixture was purified by silica column chromatograph (eluent 95:5 dichloromethane:1% ammonia in methanol) to yield the title compound (976 mg, 48%).

### Step 3—tert-Butyl 4-(3-((2-chlorophenyl)carbamoyl)imidazo[1,2-a]pyridin-6-yl)piperidine-1-carboxylate

A mixture of—tert-butyl 4-(6-aminopyridin-3-yl)piperidine-1-carboxylate (976 mg, 3.52 mmol) and N,N-dimethylformamide-dimethyl acetal (0.608 mL, 4.58 mmol) in anhydrous toluene (30 mL) was heated at 85° C. for 8 h. After the mixture was allowed to cool, 2-bromo-N-(2-chlorophenyl)acetamide (1.1 g, 4.5 mmol) and anhydrous MeOH (5 mL) were added. The reaction mixture was reheated at 85° C. for 18 h, cooled to RT and diluted with water (˜50 mL). The mixture was extracted with ethyl acetate (3×30 mL), the combined organic layers were collected, dried over anhydrous sodium sulphate, filtered and concentrated. The crude mixture was purified by flash column chromatography on silica gel (40% to 70% of EtOAc in hexane) to give the title compound (620 mg, 39%) as a waxy solid.

### Step 4—N-(2-Chlorophenyl)-6-(piperidin-4-yl)imidazo[1,2-a]pyridine-3-carboxamide

To tert-butyl 4-(3-((2-chlorophenyl)carbamoyl)imidazo[1,2-a]pyridin-6-yl)piperidine-1-carboxylate (226 mg, 0.5 mmol) in DCM (6 mL) was treated with TFA (3 mL) at 0° C. and then stirred at room temperature for 1 h. The excess TFA was evaporated, 1N NaOH (15 mL) was added and the mixture extracted with ethyl acetate (4×25 mL). The combined organic layers were washed with sat. NaHCO₃, dried over anhydrous Na₂SO₄, filtered, and concentrated. The residue was purified purified by silica column chromatograph (eluent 95:5 dichloromethane:1% ammonia in methanol) to yield the title compound (136 mg, 78%). ¹H NMR (CD30D, 400 MHz) δ 9.39 (s, 1H), 8.45 (s, 1H), 7.76 (dd, 1H), 7.70 (d, 1H), 7.52-7.58 (m, 2H), 7.38 (td, 1H), 7.28 (td, 1H), 3.20-3.22 (m, 2H), 2.81-2.90 (m, 3H), 1.97-1.99 (m, 2H), 1.74 (qd, 2H).

### Step 5—Oxalate Salt

The oxalate salt of N-(2-chlorophenyl)-6-(piperidin-4-yl)imidazo[1,2-a]pyridine-3-carboxamide was prepared for some in vivo studies. To a stirring solution of N-(2-chlorophenyl)-6-(piperidin-4-yl)imidazo[1,2-a]pyridine-3-carboxamide free base (0.41 mmol) in EtOAc (100 mL) and MeOH (20 mL) was added 74 mg (2 equiv.) of oxalic acid in EtOAc (5 mL). The resulting mixture was stirred at room temperature for 1 h. The solvent was removed under reduced pressure and then the residue was washed sequentially with Et2O and EtOAc then filtered and dried. ¹H NMR (CD30D, 400 MHz) δ 9.46 (s, 1H), 8.51 (s, 1H), 7.76 (d, 1H), 7.73 (dd, 1H), 7.61 (dd, 1H), 7.54 (dd, 1H), 7.39 (td, 1H), 7.29 (td, 1H), 3.54 (br d, 2H), 3.18 (td, 2H), 3.09 (tt, 1H), 2.19 (br d, 2H), 1.97 (qd, 2H).

### Example 10—Synthesis of Exemplified Compounds 101-119

### Intermediate 1-6-Bromo-N-(2-chlorophenyl)imidazo[1,2-a]pyridine-3-carboxamide

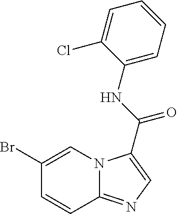 [SMILES: O=C(Nc1ccccc1Cl)c1cnc2ccc(Br)cn12]

To a 250 mL round bottom flask was added, 6-bromoimidazo[1,2-a]pyridine-3-carboxylic acid (2 g, 8.26 mmol), 2-chloroaniline (2.17 mL, 0.0207 mol), 1-ethyl-3-(3-dimethylaminopropyl)carbodiimide-HCL (2.38 g, 0.0124 mol), HATU (4.17 g, 0.0124 mol) and N,N-dimethylformamide (25 mL). The mixture was stirred overnight at 60° C., cooled to RT, water (about 30 mL) added and stirred for 30 minutes. The resulting white solid was collected by filtration and air-dried. Recrystallization from ethyl acetate and hexanes afforded to give the title compound (2.25 g, 78%). ¹H NMR (400 MHz, DMSO) δ 10.18 (s, 1H), 9.58 (dd, 1H), 8.59 (s, 1H), 7.77 (dd, 1H), 7.65 (dd, 1H), 7.58 (dt, 2H), 7.40 (dt, 1H), 7.31 (dt, 1H).

### Intermediate 2-N-(2-Chlorophenyl)-6-(4,4,5,5-tetramethyl-1,3,2-dioxaborolan-2-yl)imidazo[1,2-a]pyridine-3-carboxamide

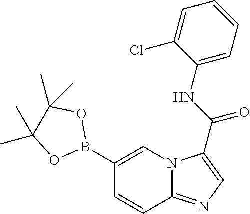 [SMILES: CC1(C)OB(c2ccc3ncc(C(=O)Nc4ccccc4Cl)n3c2)OC1(C)C]

Intermediate 1 (351 mg, 1.00 mmol), potassium acetate (400 mg, 4.00 mmol) and bis(pinacolato)diboron (305 mg, 1.20 mmol) were combined in 1,4-dioxane and degassed with argon. [1,1′-Bis(diphenylphosphino)ferrocene]dichloropalladium(II) (44 mg, 0.06 mmol) was added and the mixture was heated at 80° C. for 24 hours. The reaction was cooled and the solvent removed by rotary evaporation. The resulting solid was dissolved in ethyl acetate, washed with brine, dried with magnesium sulfate, filtered and concentrated. The brown solid (183 mg, 46%) was used for further reactions. ¹H NMR (400 MHz, CDCl₃): δ 9.84 (s, 1H), 8.48 (dd, 1H), 8.23 (m, 2H), 7.71 (d, 2H), 7.44 (dt, 1H), 7.34 (m, 1H), 7.10 (td, 1H), 1.36 (s, 12H).

### Synthesis of compound 101—N-(2-Chlorophenyl)-6-(2-methoxyphenyl)imidazo[1,2-a]pyridine-3-carboxamide

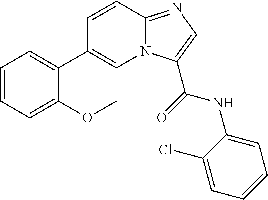 [SMILES: COc1ccccc1-c1ccc2ncc(C(=O)Nc3ccccc3Cl)n2c1]

A mixture of Intermediate 1 (100 mg, 0.285 mmol), 2-methoxyphenylboronic acid (64.9 mg, 0.428 mmol), sodium carbonate (90.6 mg, 0.855 mmol), acetonitrile (3 mL) and water (1.2 mL) was degassed with argon.

Tetrakis(triphenylphospine)palladium(O) (65.9 mg, 0.057 mmol) was added and the mixture heated under an argon atmosphere overnight at 90° C. The mixture was cooled to RT, quenched with brine (15 mL), extracted with ethyl acetate (3×10 mL) and the organic layers collected. The combined organic extracts were dried over anhydrous sodium sulphate, filtered and concentrated. The crude mixture was purified by silica column chromatograph (eluent 95:5 dichloromethane:1% ammonia in methanol) to yield the title compound as a white solid (98 mg, 91%). ¹H NMR (400 MHz, DMSO) δ 10.08 (s, 1H), 9.55 (s, 1H), 8.60 (s, 1H), 7.79 (d, 1H), 7.65 (dd, 1H), 7.60 (i, 2H), 7.40 (q, 3H), 7.30 (dt, 1H), 7.16 (d, 1H), 7.06 (t, 1H), 3.79 (s, 3H).

Compounds 102-111 are prepared according to the methods and procedures similar to those described for compound 101 using commercially available starting materials.),

| No. | Structure | Chemical name | Analytical data |
|---|---|---|---|
| 102 | 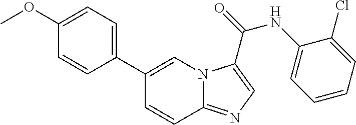 [SMILES: COc1ccc(-c2ccc3ncc(C(=O)Nc4ccccc4Cl)n3c2)cc1] | N-(2- Chlorophenyl)-6- (4- methoxyphenyl) imidazo[1,2- a]pyridine-3- carboxamide | ¹H NMR (400 MHz, DMSO) δ 10.10 (s, 1H), 9.63 (s, 1H), 8.59 (s, 1H), 7.82 (s, 2H), 7.62 (dd, J = 8.03 Hz, 3H), 7.57 (dd, 1H), 7.40 (dt, 1H), 7.31 (dt, 1H), 7.06 (t, 2H), 3.79 (s, 3H). |
| 103 | 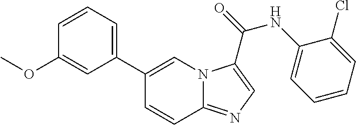 [SMILES: COc1cccc(-c2ccc3ncc(C(=O)Nc4ccccc4Cl)n3c2)c1] | N-(2- Chlorophenyl)-6- (3- methoxyphenyl) imidazo[1,2- a]pyridine-3- carboxamide | ¹H NMR (400 MHz, DMSO) δ 10.13 (s, 1H), 9.68 (s, 1H), 8.62 (s, 1H), 7.86 (s, 2H), 7.61 (dd, 1H), 7.57 (dd, 1H), 7.41 (q, H- 1), 7.32 (q, 1H), 7.24 (d, 1H), 7.21 (s, 1H), 7.00 (dd, 1H), 3.82 (s, 3H). |
| 104 | 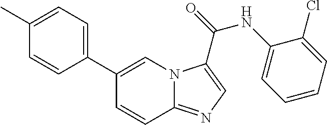 [SMILES: Cc1ccc(-c2ccc3ncc(C(=O)Nc4ccccc4Cl)n3c2)cc1] | N-(2- Chlorophenyl)-6- (4- methylphenyl) imidazo[1,2- a]pyridine-3- carboxamide | ¹H NMR (400 MHz, DMSO) δ 10.11 (s, 1H), 9.67 (s, 1H), 8.61 (s, 1H), 7.84 (s, 2H), 7.66 (d, 1H), 7.59 (m, 3H), 7.40 (dt, 1H), 7.31 (d, 2H), 7.12 (d, 1H), 2.34 (s, 3H). |
| 105 | 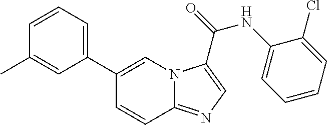 [SMILES: Cc1cccc(-c2ccc3ncc(C(=O)Nc4ccccc4Cl)n3c2)c1] | N-(2- Chlorophenyl)-6- (3- methylphenyl) imidazo[1,2- a]pyridine-3- carboxamide | ¹H NMR (400 MHz, DMSO) δ 10.13 (s, 1H), 9.68 (s, 1H), 8.61 (s, 1H), 7.85 (s, 1H), 7.61 (dd, 1H), 7.57 (dd, 1H), 7.48 (d, 2H), 7.39 (m, 3H), 7.31 (dt, 1H), 7.23 (d, 1H), 2.38 (s, 3H). |
| 106 | 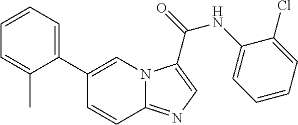 [SMILES: Cc1ccccc1-c1ccc2ncc(C(=O)Nc3ccccc3Cl)n2c1] | N-(2- Chlorophenyl)-6- (2- methylphenyl) imidazo[1,2- a]pyridine-3- carboxamide | ¹H NMR (400 MHz, DMSO) δ 10.10 (s, 1H), 9.36 (s, 1H), 8.62 (s, 1H), 7.82 (dd, 1H), 7.57 (dt, 3H), 7.38 (dt, 1H), 7.34 (q, 2H), 7.29 (m, 3H), 2.26 (s, 3H). ¹H NMR (400 MHz, DMSO-d6) δ 10.18 (s, 1H), 9.30 (s, 1H), 8.75-8.69 (m, 1H), 7.89-7.87 |
| | | | (m, 1H), 7.61-7.56 (m, 2H), |
| | | | 7.52-7.27 (m, 7H). LC/MS m/z |
| | | | 362.1 [M + H]⁺ |
| 107 | 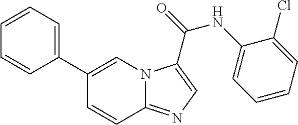 [SMILES: O=C(Nc1ccccc1Cl)c1cnc2ccc(-c3ccccc3)cn12] | N-(2- Chlorophenyl)-6- phenylimidazo[1,2- a]pyridine-3- carboxamide | ¹H NMR (400 MHz, CDCl₃): δ 9.77 (s, 1H), 8.46 (dd, 1H), 8.26 (s, 2H), 7.82 (d, 1H), 7.72 (dd, 1H), 7.65 (m, 2H), 7.46 (m, 4H), 7.34 (m, 1H), 7.10 (td, 1H). |
| 108 | 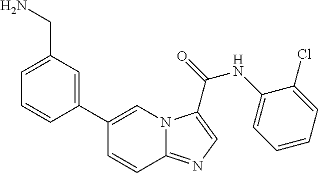 [SMILES: NCc1cccc(-c2ccc3ncc(C(=O)Nc4ccccc4Cl)n3c2)c1] | 6-(3- (Aminomethyl) phenyl)-N-(2- chlorophenyl) imidazo[1,2- a]pyridine-3- carboxamide | ¹H NMR (400 MHz, CDCl₃): δ 9.76 (s, 1H), 8.45 (dd, 1H), 8.27 (s, 1H), 8.25 (s, 1H), 7.81 (d, 1H), 7.72 (m, 1H), 7.59 (s, 1H), 7.52 (d, 1H), 7.45 (s, 1H), 7.44 (d, 1H), 7.38 (d, 1H), 7.34 (m, 1H), 7.1 (td, 1H), 3.97 (s, 2H), 1.74 (br s, 2H). |
| 109 | 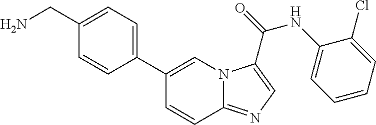 [SMILES: NCc1ccc(-c2ccc3ncc(C(=O)Nc4ccccc4Cl)n3c2)cc1] | 6-(4- (Aminomethyl) phenyl)-N-(2- chlorophenyl) imidazo[1,2- a]pyridine-3- carboxamide | ¹H NMR (400 MHz, CDCl₃): δ 9.76 (s, 1H), 8.45 (dd, 1H), 8.27 (s, 1H), 8.26 (s, 1H), 7.81 (m, 1H), 7.71 (dd, 1H), 7.62 (d, 2H), 7.44 (dd, 3H), 7.34 (m, 1H), 7.10 (td, 1H), 3.95 (s, 2H), 1.73 (br s, 2H). |
| 110 | 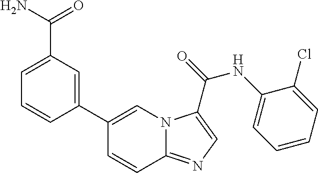 [SMILES: NC(=O)c1cccc(-c2ccc3ncc(C(=O)Nc4ccccc4Cl)n3c2)c1] | 6-(3- Carbamoylphenyl)- N-(2- chlorophenyl) imidazo[1,2- a]pyridine-3- carboxamide | ¹H NMR (400 MHz, DMSO- D₆): δ 10.14 (s, 1H), 9.74 (s, 1H), 8.62 (s, 1H), 8.18 (dt, 1H), 8.12 (s, 1H), 7.90 (m, 2H), 7.84 (m, 1H), 7.63 (m, 1H), 7.57 (m, 2H), 7.45 (s, 1H), 7.40 (m, 1H), 7.30 (m, 1H). |
| 111 | 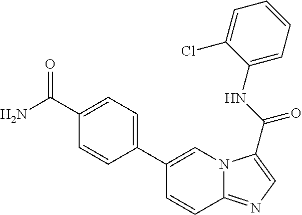 [SMILES: NC(=O)c1ccc(-c2ccc3ncc(C(=O)Nc4ccccc4Cl)n3c2)cc1] | 6-(4- carbamoylphenyl)- N-(2- chlorophenyl) imidazo[1,2- a]pyridine-3- carboxamide | ¹H NMR (400 MHz, DMSO- D₆): δ 10.15 (s, 2H), 9.75 (s, 1H), 8.63 (s, 1H), 8.04 (s, 1H), 7.99 (m, 2H), 7.91 (d, 1H), 7.89 (m, 1H), 7.78 (d, 2H), 7.59 (qd, 2H), 7.40 (m, 2H), 7.31 (td, 1H). |

### Synthesis of compound 112-N-(2-chlorophenyl)-6-(pyridin-2-yl)imidazo[1,2-a]pyridine-3-carboxamide

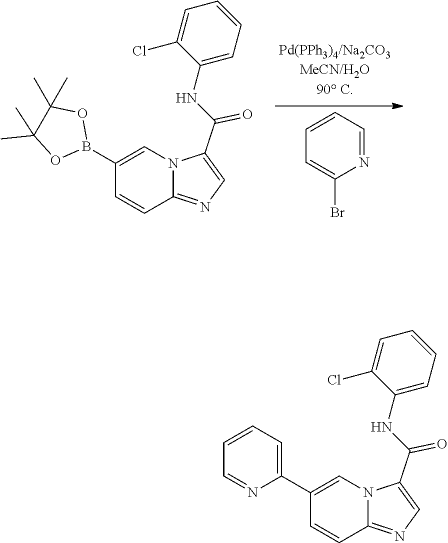 [SMILES: CC1(C)OB(c2ccc3ncc(C(=O)Nc4ccccc4Cl)n3c2)OC1(C)C ; Brc1ccccn1 ; O=C(Nc1ccccc1Cl)c1cnc2ccc(-c3ccccn3)cn12]

A mixture of intermediate 2 (159 mg, 0.4 mmol), 2-bromopyridine (79 mg, 0.5 mmol) and sodium carbonate (159 mg, 1.5 mmol) were dissolved in water (6 mL) and acetonitrile (2 mL). The mixture was stirred, degassed and tetrakis(triphenylphosphine)palladium(O) (66 mg, 0.05 mmol) added. The reaction mixture was heated at 90° C. for 5 hours. Then, the reaction solvent was cooled and the solvents removed by rotary evaporation. The resulting solid was taken up in ethyl acetate (25 mL), washed with saturated sodium chloride solution (20 mL), dried with magnesium sulfate, filtered and concentrated. The product was isolated by flash column chromatography and gave the title compound as a white solid (50 mg, 36%). ¹H NMR (400 MHz, CDCl₃): δ 10.15 (s, 1H), 8.73 (d, 1H), 8.48 (dd, 1H), 8.23 (br s, 2H), 8.20 (dd, 1H), 7.81 (m, 3H), 7.45 (dd, 1H), 7.35 (m, 1H), 7.30 (ddd, 1H), 7.10 (td, 1H).

Compounds 113-115 are prepared according to the methods and procedures similar to those described for compound 112 using commercially available starting materials:

| No. | Structure | Chemical name | Analytical data |
|---|---|---|---|
| 113 | 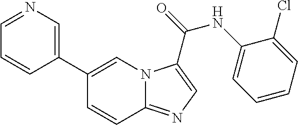 [SMILES: O=C(Nc1ccccc1Cl)c1cnc2ccc(-c3cccnc3)cn12] | N-(2- Chlorophenyl)-6- (pyridin-3- yl)imidazo[1,2- a]pyridine-3- carboxamide | ¹H NMR (400 MHz, CDCl₃): δ 9.82 (s, 1H), 8.92 (d, 1H), 8.68 (dd, 1H), 8.45 (m, 1H), 8.29 (s, 1H), 8.27 (s, 1H), 7.96 (dt, 1H), 7.88 (m, 1H), 7.69 (dd, 1H), 7.45 (dd, 1H), 7.42 (t, 1H), 7.35 (m, 1H), 7.11 (m, 1H). |
| 114 | 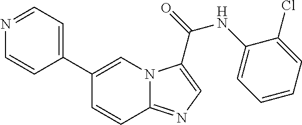 [SMILES: O=C(Nc1ccccc1Cl)c1cnc2ccc(-c3ccncc3)cn12] | N-(2- Chlorophenyl)-6- (pyridin-4- yl)imidazo[1,2- a]pyridine-3- carboxamide | ¹H NMR (400 MHz, CDCl₃): δ 9.90 (s, 1H), 8.73 (dd, 2H), 8.44 (dd, 1H), 8.29 (s, 1H), 8.28 (s, 1H), 7.88 (d, 1H), 7.72 (dd, 1H), 7.58 (dd, 2H), 7.46 (dd, 1H), 7.35 (m, 1H), 7.12 (td, 1H). |
| 115 | 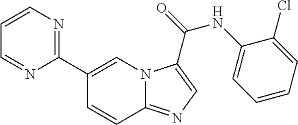 [SMILES: O=C(Nc1ccccc1Cl)c1cnc2ccc(-c3ncccn3)cn12] | N-(2- Chlorophenyl)-6- (pyrimidin-2- yl)imidazo[1,2- a]pyridine-3- carboxamide | ¹H NMR (400 MHz, CDCl₃): δ 10.63 (s, 1H), 8.83 (d, 2H), 8.28 (s, 2H), 7.67 (m, 2H), 7.54 (m, 1H), 7.47 (dd, 2H), 7.34 (m, 1H), 7.10 (td, 1H). |

### Synthesis of Compound 116-N-(2-chlorophenyl)-6-(2-hydroxyphenyl)imidazo[1,2-a]pyridine-3-carboxamide

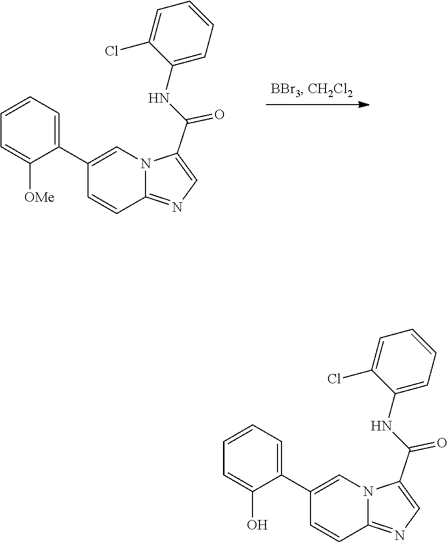 [SMILES: COc1ccccc1-c1ccc2ncc(C(=O)Nc3ccccc3Cl)n2c1 ; O=C(Nc1ccccc1Cl)c1cnc2ccc(-c3ccccc3O)cn12]

N-(2-Chlorophenyl)-6-(2-methoxyphenyl)imidazo[1,2-a]pyridine-3-carboxamide (56 mg, 0.148 mmol) and dichloromethane (11 mL) were degassed with argon, cooled to 0° C. and boron tribromide (0.506 mL, 2.96 mmol) was added dropwise. The solution was allowed to return to RT and stirred overnight. Saturated sodium bicarbonate solution (11 mL) was added, the mixture was extracted with dichloromethane (2×10 mL) and the combined extracts washed with brine (20 mL). The organics were collected, dried with anhydrous sodium sulphate, filtered and concentrated. The crude mixture was purified by silica column chromatograph (eluent 95:5 dichloromethane:1% ammonia in methanol) to yield the title compound as a white-yellow solid (28 mg, 527). ¹H NMR (400 MHz, DM 7) δ 10.06 (s, 1H), 9.80 (s, 1H), 9.66 (s, 1H), 8.59 (s, 1H), 7.78 (d, 1H), 7.71 (dd, 1H), 7.60 (dd, 1H), 7.58 (dd, 1H), 7.36 (i, 2H), 7.29 (dt, 1H), 7.21 (dt, 1H), 6.97 (d, 1H), 6.91 (t, 1H).

Compounds 117-119 are prepared according to the methods and procedures similar to those described for compound A-38 (Example 9) using commercially available starting materials:

| No. | Structure | Chemical name | Analytical data |
|---|---|---|---|
| 117 | 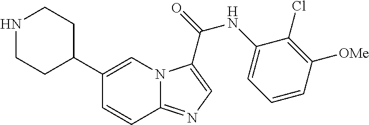 [SMILES: COc1cccc(NC(=O)c2cnc3ccc(C4CCNCC4)cn23)c1Cl] | N-(2-Chloro-3- methoxyphenyl)-6- (piperidin-4- yl)imidazo[1,2- a]pyridine-3- carboxamide | |
| 118 | 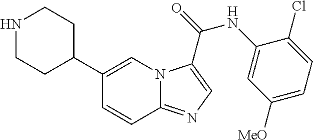 [SMILES: COc1ccc(Cl)c(NC(=O)c2cnc3ccc(C4CCNCC4)cn23)c1] | N-(2-Chloro-5- methoxyphenyl)-6- (piperidin-4- yl)imidazo[1,2- a]pyridine-3- carboxamide | 1H NMR (400 MHz, DMSO) δ 10.00 (s, 1H), 9.32 (s, 1H), 8.50 (s, 1H), 7.76 (d, 1H), 7.44 (d, 2H), 7.20 (d, 1H), 6.88 (dd, 1H), 3.77 (s, 3H), 3.37 (m, 2H), 3.00 (m, 3H), 2.00 (m, 2H), 1.83 (m, 2H). |
| 119 | 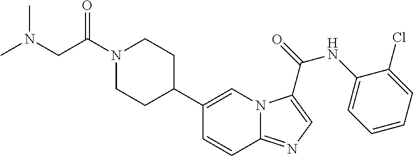 [SMILES: CN(C)CC(=O)N1CCC(c2ccc3ncc(C(=O)Nc4ccccc4Cl)n3c2)CC1] | N-(2-Chlorophenyl)-6- (1- (dimethylglycyl) piperidin- 4-yl)imidazo[1,2- a]pyridine-3- carboxamide | 1H NMR (400 MHz, DMSO) δ 10.08 (s, 1H), 9.31 (s, 1H), 8.59 (s, 1H), 7.74 (d, 1H), 7.50 (dd, 2H), 7.47 (m, 2H), 7.37 (q, 3H), 7.30 (dt, 1H), 4.52 (m, 1H), 4.24 (m, 1H), 3.71 (br d, 2H), 3.17 (t, 2H), 2.96 (t, 1H), 2.77 (s, 6H), 1.97 (m, 2H), 1.68 (m, 1H), 1.45 (m, 1H). |

Compounds 120-124 are prepared according to the methods and procedures similar to those described for compounds A38 and 101-119 using commercially available starting materials:

| No. | Structure | Chemical name | Analytical data |
|---|---|---|---|
| 120 | 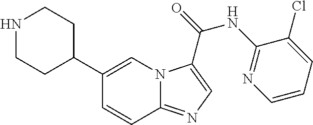 [SMILES: O=C(Nc1ncccc1Cl)c1cnc2ccc(C3CCNCC3)cn12] | N-(3-chloropyridin-2- yl)-6-(piperidin-4- yl)imidazo[1,2- a]pyridine-3- carboxamide | ¹H NMR (400 MHz, MeOD) δ 9.69 (s, 1H), 8.87 (s, 1H), 8.46 (dd, J = 4.8, 1.4 Hz, 1H), 8.17 (dd, J = 9.3, 1.3 Hz, 1H), 8.11- 8.04 (m, 2H), 7.43 (dd, J = 8.1, 4.8 Hz, 1H), 3.59- 3.52 (m, 2H), 3.29-3.15 |
| | | | (m, 3H), 2.28-2.18 (m, |
| | | | 2H), 2.10-1.95 (m, 2H). |
| | | | LC/MS: Observed m/z |
| | | | 356.1 [M + H]+ |
| 121 | 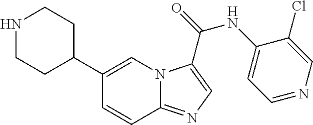 [SMILES: O=C(Nc1ccncc1Cl)c1cnc2ccc(C3CCNCC3)cn12] | N-(3-chloropyridin-4- yl)-6-(piperidin-4- yl)imidazo[1,2- a]pyridine-3- carboxamide | ¹H NMR (400 MHz, MeOD) δ 9.60 (s, 1H), 9.20 (s, 1H), 9.15 (s, 1H), 8.97 (d, J = 6.7 Hz, 1H), 8.81 (d, J = 6.7 Hz, 1H), 8.28 (d, J = 9.2 Hz, 1H), 8.16 (d, J = 9.3 Hz, 1H), 3.61-3.56 (m, 2H), 3.39- |
| | | | 3.32 (m, 1H), 3.29-3.20 |
| | | | (m, 2H), 2.30-2.21 (m, |
| | | | 2H), 2.17-2.05 (m, 2H); |
| | | | LC/MS: Observed m/z |
| | | | 356.1 [M + H]+ |
| 122 | 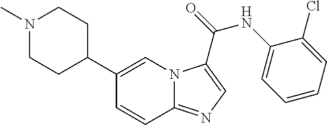 [SMILES: CN1CCC(c2ccc3ncc(C(=O)Nc4ccccc4Cl)n3c2)CC1] | N-(2-chlorophenyl)-6- (1-methylpiperidin-4- yl)imidazo[1,2- a]pyridine-3- carboxamide | ¹H NMR (400 MHz, MeOH-d4) δ 9.44 (s, 1H), 8.48 (s, 1H), 7.77-7.72 (m, 2H), 7.57 (ddd, J = 9.4, 8.7, 1.5 Hz, 2H), 7.40 (td, J = 7.7, 1.4 Hz, 1H), 7.32- 7.29 (m, 1H), 3.38-3.35 (m, 2H), 2.94-2.87 (m, |
| | | | 1H), 2.76-2.70 (m, 2H), |
| | | | 2.68-2.64 (m, 3H), 2.14- |
| | | | 2.11 (m, 2H), 1.95-1.92 |
| | | | (m, 2H); LC/MS: Observed |
| | | | m/z 369.1 [M + H]+ |
| 123 | 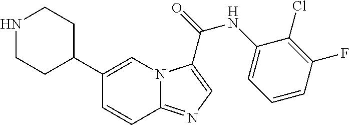 [SMILES: O=C(Nc1cccc(F)c1Cl)c1cnc2ccc(C3CCNCC3)cn12] | N-(2-chloro-3- fluorophenyl)-6- (piperidin-4- yl)imidazo[1,2- a]pyridine-3- carboxamide | ¹H NMR (400 MHz, MeOD-d4) δ 9.72 (s, 1H), 8.99 (s, 1H), 8.19 (d, J = 7.2 Hz, 1H), 8.11 (d, J = 8.3 Hz, 1H), 7.59 (dt, J = 8.1, 1.2 Hz, 1H), 7.43 (td, J = 8.3, 5.8 Hz, 1H), 7.25 (td, J = 8.7, 1.3 Hz, 1H), |
| | | | 3.62-3.52 (m, 2H), 3.25- |
| | | | 3.19 (m, 3H), 2.25-2.22 |
| | | | (m, 2H), 2.04 (qd, J = 13.6, |
| | | | 3.9 Hz, 2H); LC/MS: |
| | | | Observed m/z 373.1 |
| | | | [M + H]+ |
| 124 | 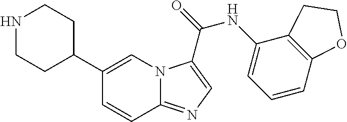 [SMILES: O=C(Nc1cccc2c1CCO2)c1cnc2ccc(C3CCNCC3)cn12] | N-(2,3- dihydrobenzofuran-4- yl)-6-(piperidin-4- yl)imidazo[1,2- a]pyridine-3- carboxamide | ¹H NMR (400 MHz, MeOD-d4) δ 9.70 (s, 1H), 8.83 (s, 1H), 8.15 (dd, J = 9.2, 0.8 Hz, 1H), 8.07 (d, J = 9.3 Hz, 1H), 7.15 (t, J = 8.0 Hz, 1H), 6.97 (dd, J = 8.0, 0.8 Hz, 1H), 6.69 (d, J = 7.9 Hz, 1H), 4.58 (t, J = |
| | | | 8.7 Hz, 2H), 3.59-3.56 |
| | | | (m, 2H), 3.28-3.17 (m, |
| | | | 5H), 2.25-2.22 (m, 2H), |
| | | | 2.08-2.00 (m, 2H); |
| | | | LC/MS: Observed m/z |
| | | | 363.2 [M + H]+ |

### Example 11—Eph Receptor Kinase Inhibitory Activity by the Exemplified Compounds

The assays were carried out are previously described, see Hastie et al., Nat Protoc., 2006, 1(2), 968-71; and Bain et al., Biochem J, 2007, 408 (3), 297-315.

TABLE 1

| | EphA2 | | | | EphA4 | | | | EphB3 | | | | EphB4 | | | |
|---|---|---|---|---|---|---|---|---|---|---|---|---|---|---|---|---|
| | % | % | % | % | % | % | % | % | % | % | % | % | % | % | % | % |
| | act | act | act | act | act | act | act | act | act | act | act | act | act | act | act | act |
| Compd. | 300 | 100 | 30 | 10 | 300 | 100 | 30 | 10 | 300 | 100 | 30 | 10 | 300 | 100 | 30 | 10 |
| No. | nM | nM | nM | nM | nM | nM | nM | nM | nM | nM | nM | nM | nM | nM | nM | nM |
| A-38 | 14 | 21 | | 47 | 9 | 14 | | 32 | 7 | 14 | | 37 | 3 | 9 | | 68 |
| 101 | | 77.0 | 65.4 | 67.7 | | 74.5 | 80.2 | 100.4 | | 52.7 | 62.0 | 80.9 | | 82.0 | 100.8 | 98.8 |
| 102 | | 59.6 | 69.4 | 79.9 | | 83.9 | 74.1 | 80.4 | | 88.6 | 92.7 | 92.4 | | 98.0 | 102.7 | 104.2 |
| 103 | | 70.5 | 75.3 | 76.0 | | 85.6 | 85.1 | 101.5 | | 41.0 | 86.2 | 103.2 | | 131.5 | 105.9 | 94.7 |
| 104 | | 55.9 | 86.0 | 78.1 | | 57.8 | 62.8 | 83.8 | | 55.9 | 60.5 | 109.9 | | 98.4 | 95.5 | 93.1 |
| 105 | | 65.3 | 88.1 | 104.6 | | 35.3 | 40.9 | 53.9 | | 63.7 | 81.1 | 120.9 | | 93.8 | 108.6 | 103.4 |
| 106 | | 72.7 | 78.9 | 109.6 | | 55.1 | 58.3 | 83.8 | | 63.5 | 93.8 | 131.5 | | 110.5 | 106.5 | 98.9 |
| 107 | 18 | 32 | | 68 | 13 | 26 | | 55 | 8 | 16 | | 56 | 3 | 8 | | 55 |
| 108 | 11 | 25 | | 55 | 6 | 12 | | 36 | 4 | 7 | | 46 | 2 | 12 | | 62 |
| 109 | 0 | 11 | | 77 | 5 | 8 | | 24 | 4 | 6 | | 22 | 3 | 6 | | 22 |
| 110 | 21 | 46 | | 71 | 12 | 36 | | 65 | 7 | 39 | | 58 | 3 | 48 | | 75 |
| 112 | 14 | 33 | | 53 | 12 | 25 | | 50 | 7 | 14 | | 50 | 2 | 7 | | 37 |
| 113 | 17 | 29 | | 52 | 19 | 26 | | 53 | 11 | 16 | | 49 | 4 | 7 | | 30 |
| 114 | 14 | 32 | | 51 | 10 | 21 | | 49 | 5 | 13 | | 52 | 4 | 4 | | 39 |
| 115 | 40 | 67 | | 115 | 34 | 54 | | 104 | 28 | 55 | | 108 | 9 | 32 | | 89 |
| 116 | | 61.3 | 84.0 | 76.4 | | 82.4 | 77.0 | 99.5 | | 42.9 | 65.5 | 111.7 | | 102.9 | 102.6 | 96.5 |
| 117 | | 80.8 | 83.8 | 95.8 | | 89.8 | 75.9 | 74.7 | | 102.1 | 110.0 | 109.7 | | 86.7 | 95.6 | 104.1 |
| 118 | | 82.1 | 83.5 | 104.4 | | 47.3 | 55.9 | 87.0 | | 91.4 | 73.9 | 116.5 | | 66.5 | 97.5 | 97.4 |
| 119 | | 45.1 | 61.2 | 71.3 | | 72.8 | 74.2 | 80.3 | | 78.3 | 90.3 | 116.0 | | 76.3 | 75.9 | 87.2 |

### Example 12—IC₅₀ Values of Eph Receptor Kinase Activity of Exemplified Compounds

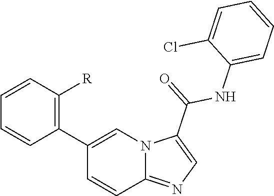

TABLE 2

| | | | | |
|---|---|---|---|---|
| | EphB3 | EphB4 | EphA4 | EphA2 |
| Compound No. | (nM) | (nM) | (nM) | (nM) |

| | | | | | |
|---|---|---|---|---|---|
| 116 | (R = OH) | 60 | 687 | 248 | 345 |
| 101 | (R = OMe) | 90 | 675 | 297 | 315 |
| 106 | (R = Me) | 182 | 675 | 110 | 245 |

### Example 13—IC₅₀ Values of Eph Receptor Kinase Activity of Exemplified Compounds

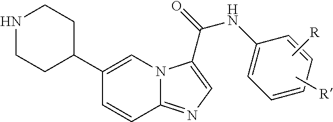

TABLE 3

| Compound No. | EphB3 (nM) | EphB4 (nM) | EphA4 (nM) | EphA2 (nM) |
|---|---|---|---|---|
| 118 (R = o-Cl, | 630 | 407 | 57 | 288 |
| R′ = p-OMe) | | | | |
| 117 (R = o-C1, | <10 | <10 | <10 | <10 |
| R′ = m-OMe) | | | | |

### Example 14—ICso Values for EphB3 Receptor Kinase Inhibitory Activity of Exemplified Compounds

| Compd. No. | IC₅₀ (μM) |
|---|---|
| 122 | 0.089 |
| 123 | 6 |
| 124 | 84 |
| 121 | 8.1 |
| 120 | 2.9 |
| 106 | 0.13 |

### REFERENCES

1. L. Ben Haim, D. H. Rowitch, Functional diversity of astrocytes in neural circuit regulation. Nat Rev Neurosci 18, 31-41 (2017).
2. R. Daneman, A. Prat, The blood-brain barrier. Cold Spring Harb Perspect Biol 7, a020412 (2015).
3. A. D. Greenhalgh, S. David, F. C. Bennett, Immune cell regulation of glia during CNS injury and disease. Nat Rev Neurosci 21, 139-152 (2020).
4. S. A. Liddelow et al., Neurotoxic reactive astrocytes are induced by activated microglia. Nature 541, 481-487 (2017).
5. L. Mayo et al., Regulation of astrocyte activation by glycolipids drives chronic CNS inflammation. Nat Med 20, 1147-1156 (2014).
6. V. Rothhammer et al., Type I interferons and microbial metabolites of tryptophan modulate astrocyte activity and central nervous system inflammation via the aryl hydrocarbon receptor. Nat Med 22, 586-597 (2016).
7. M. V. Sofroniew, Astrocyte barriers to neurotoxic inflammation. Nat Rev Neurosci 16, 249-263 (2015).
8. M. A. Wheeler, F. J. Quintana, Regulation of Astrocyte Functions in Multiple Sclerosis. Cold Spring Harbor perspectives in medicine 9, (2019).
9. C. C. Chao et al., Metabolic Control of Astrocyte Pathogenic Activity via cPLA2-MAVS. Cell 179, 1483-1498.e1422 (2019).
10. V. Rothhammer et al., Microglial control of astrocytes in response to microbial metabolites. Nature, (2018).
11. I. D. Vainchtein et al., Astrocyte-derived interleukin-33 promotes microglial synapse engulfment and neural circuit development. Science 359, 1269-1273 (2018).
12. M. A. Burguillos et al., Caspase signalling controls microglia activation and neurotoxicity. Nature 472, 319-324 (2011).
13. L. Qiao et al., Structure-activity relationship study of EphB3 receptor tyrosine kinase inhibitors. Bioorg Med Chem Lett 19, 6122-6126 (2009).
14. A. Kania, R. Klein, Mechanisms of ephrin-Eph signalling in development, physiology and disease. Nat Rev Mol Cell Biol 17, 240-256 (2016).
15. R. Imondi, C. Wideman, Z. Kaprielian, Complementary expression of transmembrane ephrins and their receptors in the mouse spinal cord: a possible role in constraining the orientation of longitudinally projecting axons. Development 127, 1397-1410 (2000).
16. C. C. Lin, B. T. Edelson, New Insights into the Role of IL-1beta in Experimental Autoimmune Encephalomyelitis and Multiple Sclerosis. J Immunol 198, 4553-4560 (2017).
17. A. Valentin-Torres et al., Sustained TNF production by central nervous system infiltrating macrophages promotes progressive autoimmune encephalomyelitis. J Neuroinflammation 13, 46 (2016).
18. M. A. Wheeler et al., MAFG-driven astrocytes promote CNS inflammation. Nature 578, 593-599 (2020).
19. M. A. Wheeler et al., MAFG-driven astrocytes promote CNS inflammation. Nature, (2020).
20. A. Kung et al., Development of Specific, Irreversible Inhibitors for a Receptor Tyrosine Kinase EphB3. J Am Chem Soc 138, 10554-10560 (2016).
21. C. Lock et al., Gene-microarray analysis of multiple sclerosis lesions yields new targets validated in autoimmune encephalomyelitis. Nat Med 8, 500-508 (2002).
22. L. Ye et al., IL-1beta and TNF-alpha induce neurotoxicity through glutamate production: a potential role for neuronal glutaminase. J Neurochem 125, 897-908 (2013).
23. S. J. Hewett, C. A. Csernansky, D. W. Choi, Selective potentiation of NMDA-induced neuronal injury following induction of astrocytic iNOS. Neuron 13, 487-494 (1994).
24. M. A. Wheeler et al., Environmental Control of Astrocyte Pathogenic Activities in CNS Inflammation. Cell 176, 581-596 e518 (2019).
25. J. M. Weiss, J. W. Berman, Astrocyte expression of monocyte chemoattractant protein-1 is differentially regulated by transforming growth factor beta. J Neuroimmunol 91, 190-197 (1998).
26. M. Linnerbauer, M. A. Wheeler, F. J. Quintana, Astrocyte Crosstalk in CNS Inflammation. Neuron, (2020).
27. E. M. Callaway, L. Luo, Monosynaptic Circuit Tracing with Glycoprotein-Deleted Rabies Viruses. J Neurosci 35, 8979-8985 (2015).
28. I. R. Wickersham et al., Monosynaptic restriction of transsynaptic tracing from single, genetically targeted neurons. Neuron 53, 639-647 (2007).
29. E. Motori et al., Inflammation-induced alteration of astrocyte mitochondrial dynamics requires autophagy for mitochondrial network maintenance. Cell Metab 18, 844-859 (2013).
30. C. W. Mount, B. Yalcin, K. Cunliffe-Koehler, S. Sundaresh, M. Monje, Monosynaptic tracing maps brain-wide afferent oligodendrocyte precursor cell connectivity. Elife 8, (2019).
31. F. Osakada, E. M. Callaway, Design and generation of recombinant rabies virus vectors. Nat Protoc 8, 1583-1601 (2013).
32. K. W. Huang, B. L. Sabatini, Single-Cell Analysis of Neuroinflammatory Responses Following Intracranial Injection of G-Deleted Rabies Viruses. Front Cell Neurosci 14, 65 (2020).
33. A. Mildner et al., CCR2+Ly-6Chi monocytes are crucial for the effector phase of autoimmunity in the central nervous system. Brain 132, 2487-2500 (2009).
34. K. Bruckner, E. B. Pasquale, R. Klein, Tyrosine phosphorylation of transmembrane ligands for Eph receptors. Science 275, 1640-1643 (1997).
35. C. A. Cowan, M. Henkemeyer, The SH2/SH3 adaptor Grb4 transduces B-ephrin reverse signals. Nature 413, 174-179 (2001).
36. M. Henkemeyer et al., Nuk controls pathfinding of commissural axons in the mammalian central nervous system. Cell 86, 35-46 (1996).
37. S. J. Holland et al., Bidirectional signalling through the EPH-family receptor Nuk and its transmembrane ligands. Nature 383, 722-725 (1996).
38. R. Klein, Bidirectional modulation of synaptic functions by Eph/ephrin signaling. Nat Neurosci 12, 15-20 (2009).
39. C. C. Chao et al., Metabolic Control of Astrocyte Pathogenic Activity via cPLA2-MAVS. Cell 179, 1483-1498 e1422 (2019).
40. V. Rothhammer et al., Sphingosine 1-phosphate receptor modulation suppresses pathogenic astrocyte activation and chronic progressive CNS inflammation. Proc Natl Acad Sci USA 114, 2012-2017 (2017).
41. S. B. Simmons, E. R. Pierson, S. Y. Lee, J. M. Goverman, Modeling the heterogeneity of multiple sclerosis in animals. Trends in immunology 34, 410-422 (2013).
42. D. A. Fruman et al., The PI3K Pathway in Human Disease. Cell 170, 605-635 (2017).
43. K. Inoki, Y. Li, T. Zhu, J. Wu, K. L. Guan, TSC2 is phosphorylated and inhibited by Akt and suppresses mTOR signalling. Nat Cell Biol 4, 648-657 (2002).
44. D. M. Sabatini, H. Erdjument-Bromage, M. Lui, P. Tempst, S. H. Snyder, RAF T1: a mammalian protein that binds to FKBP12 in a rapamycin-dependent fashion and is homologous to yeast TORs. Cell 78, 35-43 (1994).
45. J. T. Cunningham et al., mTOR controls mitochondrial oxidative function through a YY1-PGC-1alpha transcriptional complex. Nature 450, 736-740 (2007).
46. T. K. Ulland et al., TREM2 Maintains Microglial Metabolic Fitness in Alzheimer's Disease. Cell 170, 649-663 e613 (2017).
47. A. C. Bulua et al., Mitochondrial reactive oxygen species promote production of proinflammatory cytokines and are elevated in TNFR1-associated periodic syndrome (TRAPS). J Exp Med 208, 519-533 (2011).
48. W. K. E. Ip, N. Hoshi, D. S. Shouval, S. Snapper, R. Medzhitov, Anti-inflammatory effect of IL-1β mediated by metabolic reprogramming of macrophages. Science 356, 513-519 (2017).
49. A. S. Mendiola et al., Transcriptional profiling and therapeutic targeting of oxidative stress in neuroinflammation. Nat Immunol, (2020).
50. N. Li et al., Mitochondrial complex I inhibitor rotenone induces apoptosis through enhancing mitochondrial reactive oxygen species production. J Biol Chem 278, 8516-8525 (2003).
51. T. A. Sarafian et al., Disruption of astrocyte STAT3 signaling decreases mitochondrial function and increases oxidative stress in vitro. PLoS One 5, e9532 (2010).
52. L. Wang et al., Mitochondrial Respiratory Chain Inhibitors Involved in ROS Production Induced by Acute High Concentrations of Iodide and the Effects of SOD as a Protective Factor. Oxid Med Cell Longev 2015, 217670 (2015).
53. I. D. Vainchtein, A. V. Molofsky, Astrocytes and Microglia: In Sickness and in Health. Trends Neurosci 43, 144-154 (2020).
54. V. Mirakaj, P. Rosenberger, Immunomodulatory Functions of Neuronal Guidance Proteins. Trends in immunology 38, 444-456 (2017).
55. R. Muramatsu et al., RGMa modulates T cell responses and is involved in autoimmune encephalomyelitis. Nat Med 17, 488-494 (2011).
56. K. Suzuki et al., Semaphorin 7A initiates T-cell-mediated inflammatory responses through alpha1beta1 integrin. Nature 446, 680-684 (2007).
57. H. Takamatsu et al., Semaphorins guide the entry of dendritic cells into the lymphatics by activating myosin II. Nat Immunol 11, 594-600 (2010).
58. R. Tordjman et al., A neuronal receptor, neuropilin-1, is essential for the initiation of the primary immune response. Nat Immunol 3, 477-482 (2002).
59. P. Lu, C. Shih, H. Qi, Ephrin B1-mediated repulsion and signaling control germinal center T cell territoriality and function. Science 356, (2017).
60. E. Batlle et al., EphB receptor activity suppresses colorectal cancer progression. Nature 435, 1126-1130 (2005).
61. A. Contractor et al., Trans-synaptic Eph receptor-ephrin signaling in hippocampal mossy fiber LTP. Science 296, 1864-1869 (2002).
62. B. K. Lim, N. Matsuda, M. M. Poo, Ephrin-B reverse signaling promotes structural and functional synaptic maturation in vivo. Nat Neurosci 11, 160-169 (2008).
63. A. C. McClelland, S. I. Sheffler-Collins, M. S. Kayser, M. B. Dalva, Ephrin-B1 and ephrin-B2 mediate EphB-dependent presynaptic development via syntenin-1. Proc Natl Acad Sci USA 106, 20487-20492 (2009).
64. M. A. Carmona, K. K. Murai, L. Wang, A. J. Roberts, E. B. Pasquale, Glial ephrin-A3 regulates hippocampal dendritic spine morphology and glutamate transport. Proc Natl Acad Sci USA 106, 12524-12529 (2009).
65. A. Filosa et al., Neuron-glia communication via EphA4/ephrin-A3 modulates LTP through glial glutamate transport. Nat Neurosci 12, 1285-1292 (2009).
66. K. K. Murai, L. N. Nguyen, F. Irie, Y. Yamaguchi, E. B. Pasquale, Control of hippocampal dendritic spine morphology through ephrin-A3/EphA4 signaling.

Nat Neurosci 6, 153-160 (2003).
67. R. A. Sobel, Ephrin A receptors and ligands in lesions and normal-appearing white matter in multiple sclerosis. Brain Pathol 15, 35-45 (2005).
68. M. A. Anderson et al., Astrocyte scar formation aids central nervous system axon regeneration. Nature 532, 195-200 (2016).
69. J. D. Miranda et al., Induction of Eph B3 after spinal cord injury. Exp Neurol 156, 218-222 (1999).
70. M. Cisse et al., Reversing EphB2 depletion rescues cognitive functions in Alzheimer model. Nature 469, 47-52 (2011).
71. A. Nkiliza et al., RNA-binding disturbances as a continuum from spinocerebellar ataxia type 2 to Parkinson disease. Neurobiol Dis 96, 312-322 (2016).
72. A. Van Hoecke et al., EPHA4 is a disease modifier of amyotrophic lateral sclerosis in animal models and in humans. Nat Med 18, 1418-1422 (2012).
73. S. Parrinello et al., EphB signaling directs peripheral nerve regeneration through Sox2-dependent Schwann cell sorting. Cell 143, 145-155 (2010).
74. S. P. Gadani, J. T. Walsh, I. Smirnov, J. Zheng, J. Kipnis, The glia-derived alarmin IL-33 orchestrates the immune response and promotes recovery following CNS injury. Neuron 85, 703-709 (2015).
75. S. Hong et al., Complement and microglia mediate early synapse loss in Alzheimer mouse models. Science 352, 712-716 (2016).
76. A. Sekar et al., Schizophrenia risk from complex variation of complement component 4. Nature 530, 177-183 (2016).
77. B. Stevens et al., The classical complement cascade mediates CNS synapse elimination. Cell 131, 1164-1178 (2007).
78. S. Werneburg et al., Targeted Complement Inhibition at Synapses Prevents Microglial Synaptic Engulfment and Synapse Loss in Demyelinating Disease. Immunity 52, 167-182.e167 (2020).
79. Z. Zhuang et al., EphrinBs regulate D-serine synthesis and release in astrocytes. J Neurosci 30, 16015-16024 (2010).
80. E. J. Perez et al., Enhanced astrocytic d-serine underlies synaptic damage after traumatic brain injury. J Clin Invest 127, 3114-3125 (2017).
81. P. Ayata et al., Epigenetic regulation of brain region-specific microglia clearance activity. Nat Neurosci 21, 1049-1060 (2018).
82. A. C. Wendeln et al., Innate immune memory in the brain shapes neurological disease hallmarks. Nature 556, 332-338 (2018).
83. P. Assis-Nascimento, Y. Tsenkina, D. J. Liebl, EphB3 signaling induces cortical endothelial cell death and disrupts the blood-brain barrier after traumatic brain injury. Cell Death Dis 9, 7 (2018).
84. Y. A. Syed et al., Antibody-mediated neutralization of myelin-associated EphrinB3 accelerates CNS remyelination. Acta Neuropathol 131, 281-298 (2016).
85. A. W. Boyd, P. F. Bartlett, M. Lackmann, Therapeutic targeting of EPH receptors and their ligands. Nat Rev Drug Discov 13, 39-62 (2014).
86. Y. J. Zuchero et al., Discovery of Novel Blood-Brain Barrier Targets to Enhance Brain Uptake of Therapeutic Antibodies. Neuron 89, 70-82 (2016).
87. M. B. Dalva et al., EphB receptors interact with NMDA receptors and regulate excitatory synapse formation. Cell 103, 945-956 (2000).
88. M. A. Takasu, M. B. Dalva, R. E. Zigmond, M. E. Greenberg, Modulation of NMDA receptor-dependent calcium influx and gene expression through EphB receptors. Science 295, 491-495 (2002).
89. C. H. Polman et al., Diagnostic criteria for multiple sclerosis: 2010 revisions to the McDonald criteria. Ann Neurol 69, 292-302 (2011).
90. T. Dhaeze et al., CD70 defines a subset of proinflammatory and CNS-pathogenic TH1/TH17 lymphocytes and is overexpressed in multiple sclerosis. Cell Mol Immunol 16, 652-665 (2019).
91. T. Kuhlmann et al., An updated histological classification system for multiple sclerosis lesions. Acta neuropathologica 133, 13-24 (2017).
92. V. Rothhammer et al., Microglial control of astrocytes in response to microbial metabolites. Nature, (2018).
93. L. Mayo et al., Regulation of astrocyte activation by glycolipids drives chronic CNS inflammation. Nat Med 20, 1147-1156 (2014).
94. V. Rothhammer et al., Type I interferons and microbial metabolites of tryptophan modulate astrocyte activity and central nervous system inflammation via the aryl hydrocarbon receptor. Nat Med 22, 586-597 (2016).
95. F. Osakada et al., New rabies virus variants for monitoring and manipulating activity and gene expression in defined neural circuits. Neuron 71, 617-631 (2011).
96. H. Y. Kueh, A. Champhekar, S. L. Nutt, M. B. Elowitz, E. V. Rothenberg, Positive feedback between PU.1 and the cell cycle controls myeloid differentiation. Science 341, 670-673 (2013).
97. F. Osakada, E. M. Callaway, Design and generation of recombinant rabies virus vectors. Nat Protoc 8, 1583-1601 (2013).
98. C. J. Hastie, H. J. McLauchlan, P. Cohen, Assay of protein kinases using radiolabeled ATP: a protocol. Nat Protoc 1, 968-971 (2006).
99. G. Zhou et al., NetworkAnalyst 3.0: a visual analytics platform for comprehensive gene expression profiling and meta-analysis. Nucleic Acids Res 47, W234-W241 (2019).
100. M. A. Wheeler et al., Environmental Control of Astrocyte Pathogenic Activities in CNS Inflammation. Cell 176, 581-596 e518 (2019).
101. C. A. Schneider, W. S. Rasband, K. W. Eliceiri, NIH Image to ImageJ: 25 years of image analysis. Nat Methods 9, 671-675 (2012).
102. T. Goldmann et al., Origin, fate and dynamics of macrophages at central nervous system interfaces. Nat Immunol 17, 797-805 (2016).

### Numbered Paragraphs

Paragraph 1. A method of treating a neurodegenerative or a demyelinating disease or condition, the method comprising administering to a subject in need thereof a therapeutically effective amount of a compound of Formula (I):

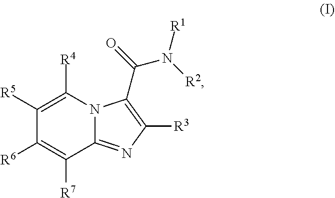

or a pharmaceutically acceptable salt thereof, wherein:
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is optionally substituted with 1, 2, or 3 substituents independently selected from R^{Cy2};
each R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}, wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₃ alkyl, and C₁₋₃ haloalkyl, wherein said C₁₋₃ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

Paragraph 2. The method of paragraph 1, wherein R¹ is selected from C₆₋₁₀ aryl and 5-14 membered heteroaryl, each of which is optionally substituted with 1 or 2 substituents independently selected from halo and OR^{a1}.

Paragraph 3. The method of paragraph 1 or 2, wherein R² is H.

Paragraph 4. The method of any one of paragraphs 1-3, wherein R³ is selected from H and C₁₋₃ alkyl.

Paragraph 5. The method of any one of paragraphs 1-4, wherein R⁵ is selected from NR^{c1}R^{d1}, OR^{a1}, C₁₋₆ alkyl, and Cy¹, wherein said C₁₋₆ alkyl is optionally substituted with NR^{c1}R^{d1}.

Paragraph 6. The method of any one of paragraphs 1-5, wherein Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

Paragraph 7. The method of any one of paragraphs 1-6, wherein R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

Paragraph 8. The method of any one of paragraphs 1-7, wherein R⁴ is selected from H, halo, CN, OR^{a1}, and C₁₋₃ haloalkyl.

Paragraph 9. The method of paragraph 8, wherein R⁴ is H.

Paragraph 10. The method of any one of paragraphs 1-9, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

Paragraph 11. The method of paragraph 10, wherein R⁷ is H.

Paragraph 12. The method of any one of paragraphs 1-11, wherein R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 13. The method of paragraph 12, wherein R⁶ is H.

Paragraph 14. The method of paragraph 1, wherein:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is selected from NR^{c1}R^{d1}, OR^{a1}, C₁₋₆ alkyl, and Cy¹, wherein said C₁₋₆ alkyl is optionally substituted with NR^{c1}R^{d1};
Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 15. The method of paragraph 14, wherein R⁴, R⁶, and R⁷ are each H.

Paragraph 16. The method of paragraph 1, wherein the compound of Formula (I) is selected from any one of the compounds listed in Table A, or a pharmaceutically acceptable salt thereof.

Paragraph 17. The method of paragraph 1, wherein the compound of Formula (I) is selected from any one of the compounds 101-119 disclosed herein, or a pharmaceutically acceptable salt thereof.

Paragraph 18. A compound of Formula (Ia):

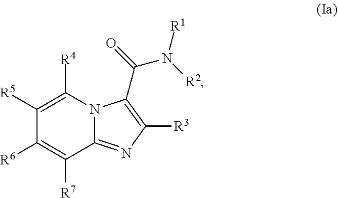

or a pharmaceutically acceptable salt thereof, wherein:
R⁵ is 5-14 membered heteroaryl, optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, C₁₋₆ alkyl, and C₁₋₆ haloalkyl, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, and C₁₋₃ haloalkyl, wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

Paragraph 19. The compound of paragraph 18, wherein R¹ is selected from C₆₋₁₀ aryl and 5-14 membered heteroaryl, each of which is optionally substituted with 1 or 2 substituents independently selected from halo and OR^{a1}.

Paragraph 20. The compound of paragraph 18 or 19, wherein R² is H.

Paragraph The compound of any one of paragraphs 18-20, wherein R³ is selected from H and C₁₋₃ alkyl.

Paragraph 22. The compound of any one of paragraphs 18-21, wherein R⁵ is 5-6 membered heteroaryl, optionally substituted with 1, 2, or 3 substituents independently selected from OR^{a1}, C(O)R^{b1}, C(O)NR^{c1}R^{d1}, and C₁₋₆ alkyl, wherein said C₁₋₆ alkyl is optionally substituted with OR^{a1} or NR^{c1}R^{d1}.

Paragraph 23. The compound of any one of paragraphs 18-22, wherein R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 24. The compound of paragraph 23, wherein R⁶ is H.

Paragraph 25. The compound of any one of paragraphs 18-24, wherein R⁴ is selected from H, halo, CN, OR^{a1}, and C₁₋₃ haloalkyl.

Paragraph The compound of paragraph 25, wherein R⁴ is H.

Paragraph 27. The compound of any one of paragraphs 18-26, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, and C₁₋₃ haloalkyl, wherein said C₁₋₃ alkyl is optionally substituted with 1 or 2 independently selected R⁹.

Paragraph 28. The compound of paragraph 27, wherein R⁷ is H.

Paragraph 29. The compound of paragraph 18, wherein:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is 5-6 membered heteroaryl, optionally substituted with 1, 2, or 3 substituents independently selected from OR^{a1}, C(O)R^{b1}, C(O)NR^{c1}R^{d1}, and C₁₋₆ alkyl, wherein said C₁₋₆ alkyl is optionally substituted with OR^{a1} or NR^{c1}R^{d1};
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1};
R⁴ is selected from H, halo, CN, OR^{a1}, and C₁₋₃ haloalkyl; and
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, and C₁₋₃ haloalkyl, wherein said C₁₋₃ alkyl is optionally substituted with 1 or 2 independently selected R⁹.

Paragraph 30. The compound of paragraph 29, wherein R⁴, R⁶, and R⁷ are each H.

Paragraph 31. The compound of paragraph 18, wherein the compound of Formula (Ia) is selected from any one of the following compounds:

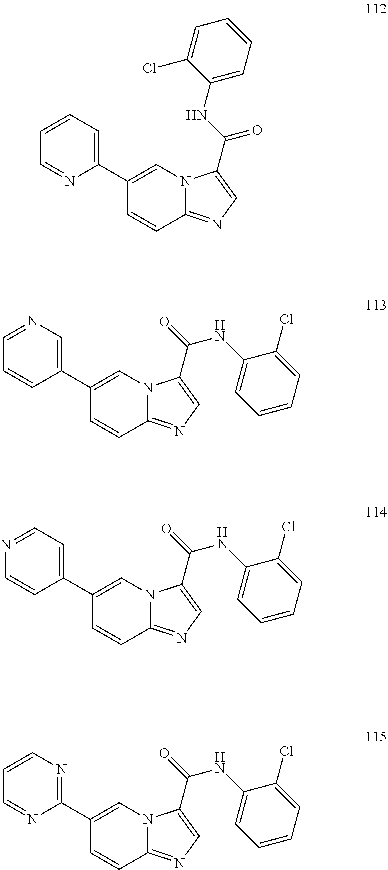 [SMILES: O=C(Nc1ccccc1Cl)c1cnc2ccc(-c3ccccn3)cn12 ; O=C(Nc1ccccc1Cl)c1cnc2ccc(-c3cccnc3)cn12 ; O=C(Nc1ccccc1Cl)c1cnc2ccc(-c3ccncc3)cn12 ; O=C(Nc1ccccc1Cl)c1cnc2ccc(-c3ncccn3)cn12]

or a pharmaceutically acceptable salt thereof.

Paragraph 32. The compound of paragraph 18, wherein the compound of Formula (Ia) is selected from any one of the following compounds:

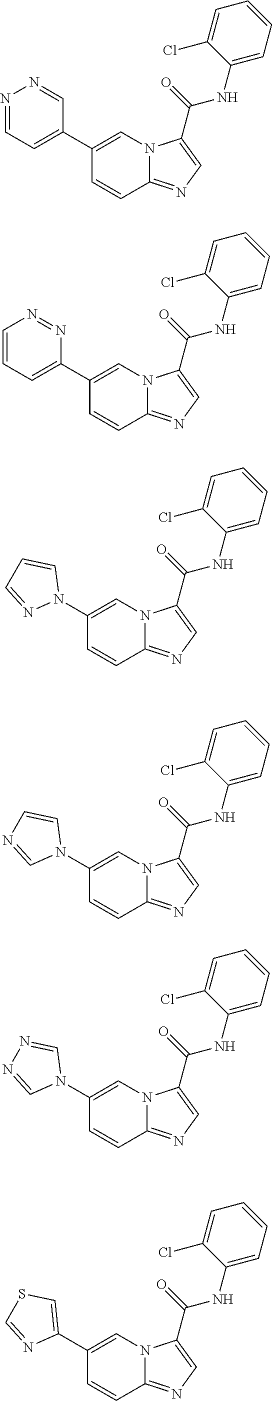 [SMILES: O=C(Nc1ccccc1Cl)c1cnc2ccc(-c3ccnnc3)cn12 ; O=C(Nc1ccccc1Cl)c1cnc2ccc(-c3cccnn3)cn12 ; O=C(Nc1ccccc1Cl)c1cnc2ccc(-n3cccn3)cn12 ; O=C(Nc1ccccc1Cl)c1cnc2ccc(-n3ccnc3)cn12 ; O=C(Nc1ccccc1Cl)c1cnc2ccc(-n3cnnc3)cn12 ; O=C(Nc1ccccc1Cl)c1cnc2ccc(-c3cscn3)cn12]

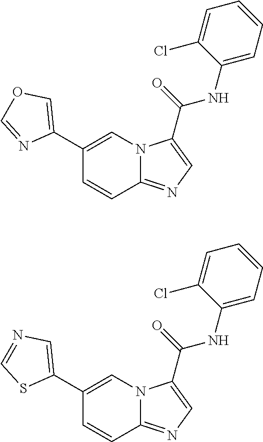 [SMILES: O=C(Nc1ccccc1Cl)c1cnc2ccc(-c3cocn3)cn12 ; O=C(Nc1ccccc1Cl)c1cnc2ccc(-c3cncs3)cn12]

or a pharmaceutically acceptable salt thereof.

Paragraph 33. A compound of Formula (Ib):

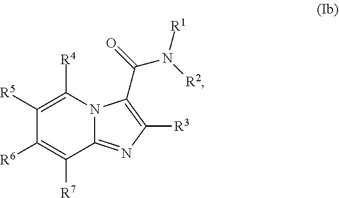

or a pharmaceutically acceptable salt thereof, wherein:
R¹ is selected from 5-14 membered heteroaryl and 4-10 membered heterocycloalkyl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

Paragraph 34. The compound of paragraph 33, wherein R¹ is 5-6 membered heteroaryl, optionally substituted with 1, 2, or 3 substituents independently selected from halo, CN, C₁₋₃ alkoxy, C₁₋₃ alkyl, and C₁₋₃ haloalkyl.

Paragraph 35. The compound of paragraph 33 or 34, wherein R² is H.

Paragraph 36. The compound of any one of paragraphs 33-35, wherein R³ is selected from H and C₁₋₃ alkyl.

Paragraph 37. The compound of any one of paragraphs 33-36, wherein R⁵ is selected from NR^{c1}R^{d1}, OR^{a1}, C₁₋₆ alkyl, and Cy¹, wherein said C₁₋₆ alkyl is optionally substituted with NR^{c1}R^{d1}.

Paragraph 38. The compound of any one of paragraphs 33-37, wherein Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

Paragraph 39. The compound of any one of paragraphs 33-38, wherein R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

Paragraph The compound of any one of paragraphs 33-39, wherein R⁴ is selected from H, halo, CN, OR^{a1}, and C₁₋₃ haloalkyl.

Paragraph 41. The compound of paragraph 40, wherein R⁴ is H.

Paragraph 42. The compound of any one of paragraphs 33-41, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

Paragraph 43. The compound of paragraph 42, wherein R⁷ is H.

Paragraph 44. The compound of any one of paragraphs 33-43, wherein R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 45. The compound of paragraph 44, wherein R⁶ is H.

Paragraph 46. The compound of paragraph 33, wherein:
R¹ is 5-6 membered heteroaryl, optionally substituted with 1, 2, or 3 substituents independently selected from halo, CN, C₁₋₃ alkoxy, C₁₋₃ alkyl, and C₁₋₃ haloalkyl;
R² is H;
R³ is selected from H and C₁₋₃ alkyl;
R⁵ is selected from NR^{c1}R^{d1} OR^{a1}, C₁₋₆ alkyl, and Cy¹, wherein said C₁₋₆ alkyl is optionally substituted with NR^{c1}R^{d1}; Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}; R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 47. The compound of paragraph 46, wherein R⁴, R⁶, and R⁷ are each H.

Paragraph 48. The compound of paragraph 33, wherein the compound of Formula (Ib) is selected from any one of the following compounds:

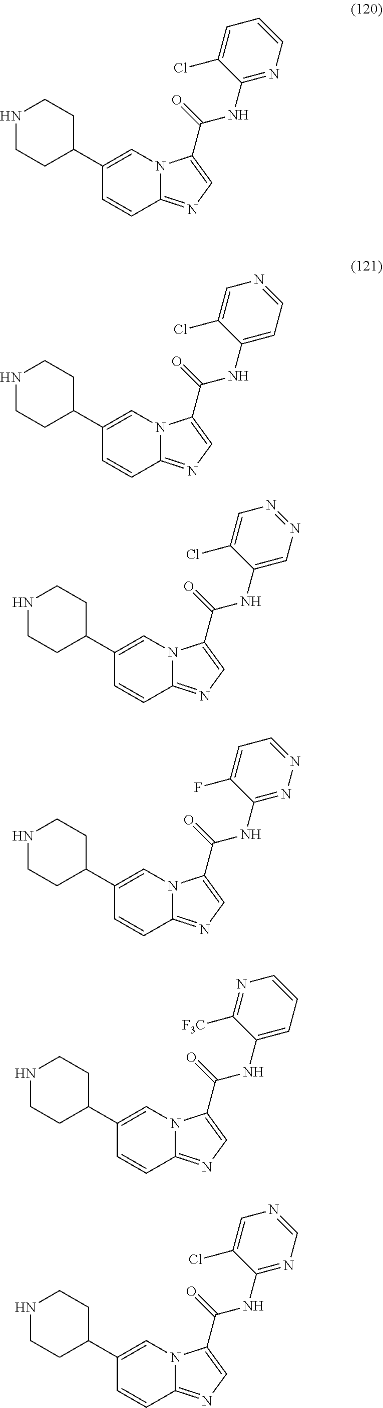 [SMILES: O=C(Nc1ncccc1Cl)c1cnc2ccc(C3CCNCC3)cn12 ; O=C(Nc1ccncc1Cl)c1cnc2ccc(C3CCNCC3)cn12 ; O=C(Nc1cnncc1Cl)c1cnc2ccc(C3CCNCC3)cn12 ; O=C(Nc1nnccc1F)c1cnc2ccc(C3CCNCC3)cn12 ; O=C(Nc1cccnc1C(F)(F)F)c1cnc2ccc(C3CCNCC3)cn12 ; O=C(Nc1ncncc1Cl)c1cnc2ccc(C3CCNCC3)cn12]

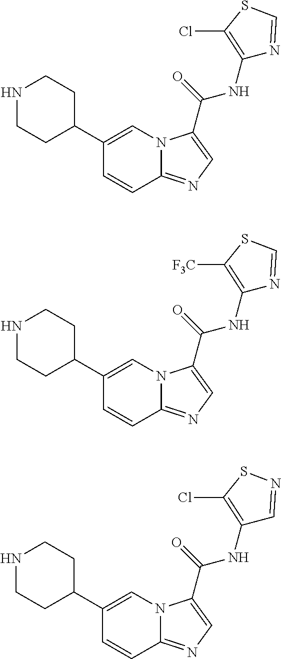 [SMILES: O=C(Nc1ncsc1Cl)c1cnc2ccc(C3CCNCC3)cn12 ; O=C(Nc1ncsc1C(F)(F)F)c1cnc2ccc(C3CCNCC3)cn12 ; O=C(Nc1cnsc1Cl)c1cnc2ccc(C3CCNCC3)cn12]

or a pharmaceutically acceptable salt thereof.

Paragraph 49. A compound of Formula (Ic):

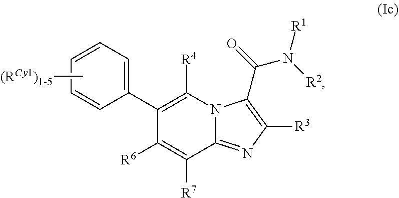

or a pharmaceutically acceptable salt thereof, wherein:
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino, provided that the compound is not:

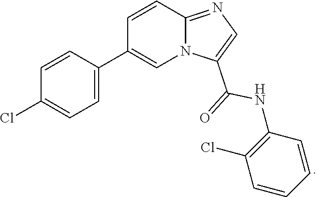 [SMILES: O=C(Nc1ccccc1Cl)c1cnc2ccc(-c3ccc(Cl)cc3)cn12]

Paragraph 50. The compound of paragraph 49, wherein R¹ is selected from C₆₋₁₀ aryl and 5-14 membered heteroaryl, each of which is optionally substituted with 1 or 2 substituents independently selected from halo and OR^{a1}.

Paragraph 51. The compound of paragraph 49 or 50, wherein R² is H.

Paragraph 52. The compound of any one of paragraphs 49-51, wherein R³ is selected from H and C₁₋₃ alkyl.

Paragraph 53. The compound of any one paragraphs 49-52, wherein R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

Paragraph 54. The compound of any one of paragraphs 49-53, wherein R⁴ is selected from H, halo, CN, OR^{a1}, and C₁₋₃ haloalkyl.

Paragraph 55. The compound of paragraph 54, wherein R⁴ is H.

Paragraph 56. The compound of any one of paragraphs 49-55, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

Paragraph 57. The compound of paragraph 56, wherein R⁷ is H.

Paragraph 58. The compound of any one of paragraphs 49-57, wherein R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 59. The compound of paragraph 58, wherein R⁶ is H.

Paragraph 60. The compound of paragraph 49, wherein:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 61. The compound of paragraph 60, wherein R⁴, R⁶, and R⁷ are each H.

Paragraph 62. The compound of paragraph 49, wherein the compound is selected from any one of the following compounds:

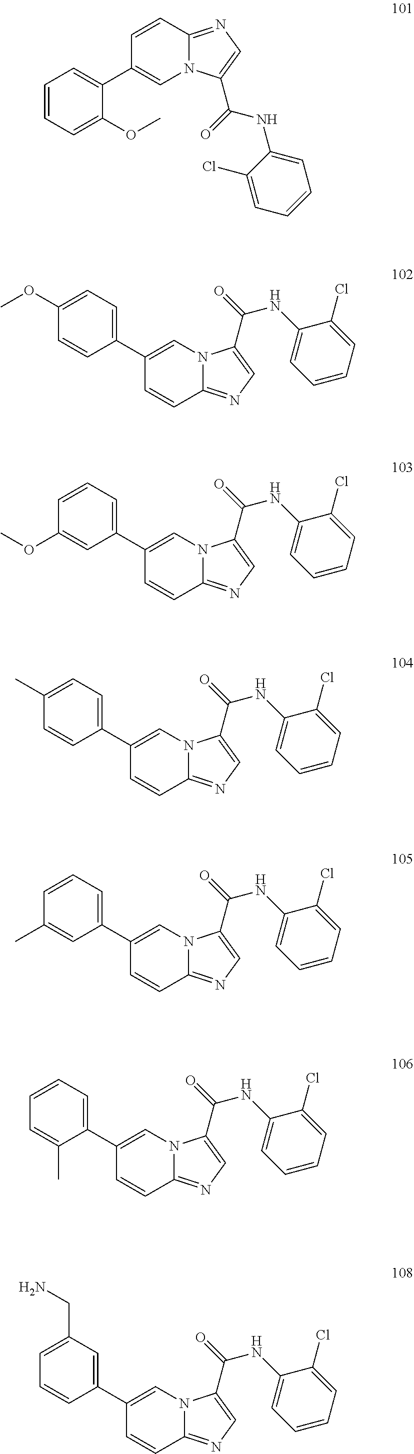

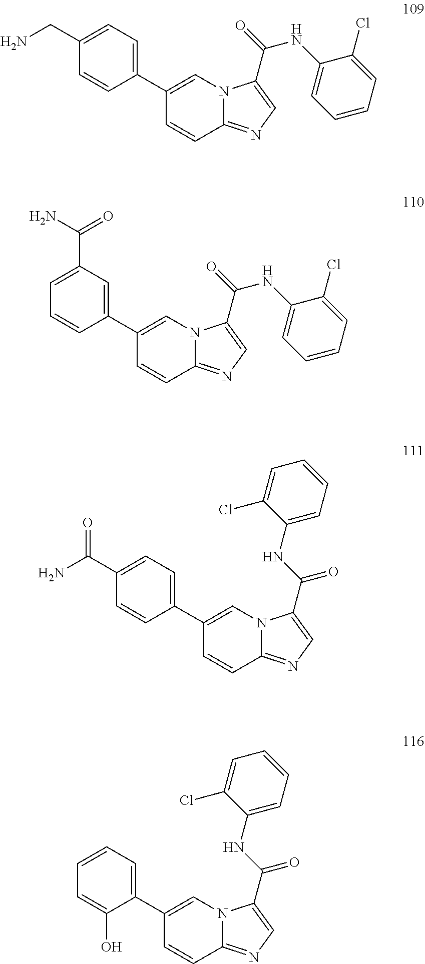 [SMILES: NCc1ccc(-c2ccc3ncc(C(=O)Nc4ccccc4Cl)n3c2)cc1 ; NC(=O)c1cccc(-c2ccc3ncc(C(=O)Nc4ccccc4Cl)n3c2)c1 ; NC(=O)c1ccc(-c2ccc3ncc(C(=O)Nc4ccccc4Cl)n3c2)cc1 ; O=C(Nc1ccccc1Cl)c1cnc2ccc(-c3ccccc3O)cn12]

or a pharmaceutically acceptable salt thereof.

Paragraph 63. The compound of paragraph 49, wherein the compound is selected from any one of the following compounds:

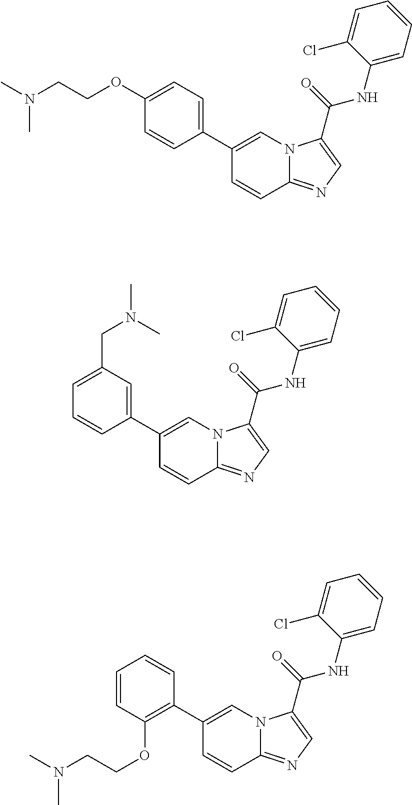 [SMILES: CN(C)CCOc1ccc(-c2ccc3ncc(C(=O)Nc4ccccc4Cl)n3c2)cc1 ; CN(C)Cc1cccc(-c2ccc3ncc(C(=O)Nc4ccccc4Cl)n3c2)c1 ; CN(C)CCOc1ccccc1-c1ccc2ncc(C(=O)Nc3ccccc3Cl)n2c1]

or a pharmaceutically acceptable salt thereof.

Paragraph 64. A compound of Formula (Id):

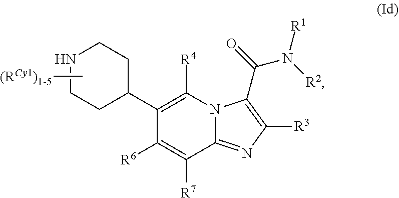

or a pharmaceutically acceptable salt thereof, wherein:
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}, wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino, provided that the compound is not:

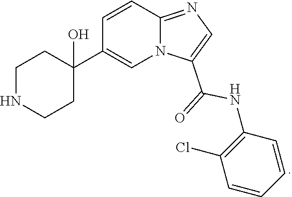 [SMILES: O=C(Nc1ccccc1Cl)c1cnc2ccc(C3(O)CCNCC3)cn12]

Paragraph 65. The compound of paragraph 64, wherein R¹ is selected from C₆₋₁₀ aryl and 5-14 membered heteroaryl, each of which is optionally substituted with 1 or 2 substituents independently selected from halo and OR^{a1}.

Paragraph 66. The compound of paragraph 64 or 65, wherein R² is H.

Paragraph 67. The compound of any one of paragraphs 64-66, wherein R³ is selected from H and C₁₋₃ alkyl.

Paragraph 68. The compound of any one of paragraphs 64-67, wherein R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

Paragraph 69. The compound of any one of paragraphs 64-68, wherein R⁴ is selected from H, halo, CN, OR^{a1}, and C₁₋₃ haloalkyl.

Paragraph 70. The compound of paragraph 69, wherein R⁴ is H.

Paragraph 71. The compound of any one of paragraphs 64-70, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

Paragraph 72. The compound of paragraph 71, wherein R⁷ is H.

Paragraph 73. The compound of any one of paragraphs 64-72, wherein R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 74. The compound of paragraph 73, wherein R⁶ is H.

Paragraph 75. The compound of paragraph 64, wherein:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 76. The compound of paragraph 75, wherein R⁴, R⁶, and R⁷ are each H.

Paragraph 77. The compound of paragraph 64, wherein the compound of Formula (Id) is:

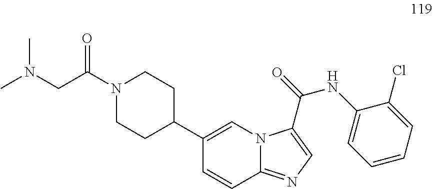 [SMILES: CN(C)CC(=O)N1CCC(c2ccc3ncc(C(=O)Nc4ccccc4Cl)n3c2)CC1]

or a pharmaceutically acceptable salt thereof.

Paragraph 78. The compound of paragraph 64, wherein the compound of Formula (Id) is selected from any one of the following compounds:

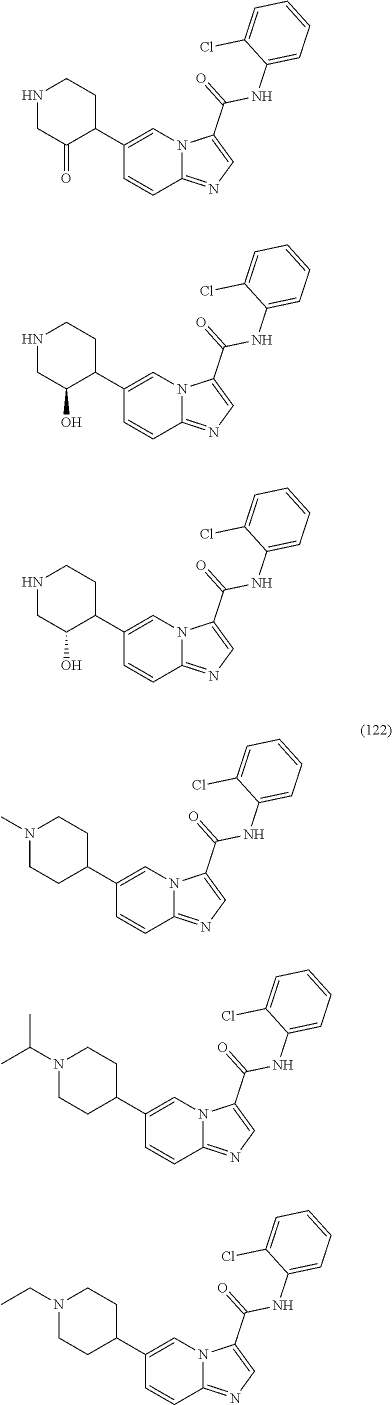 [SMILES: O=C(Nc1ccccc1Cl)c1cnc2ccc(C3CCNCC3=O)cn12 ; O=C(Nc1ccccc1Cl)c1cnc2ccc(C3CCNC[C@@H]3O)cn12 ; O=C(Nc1ccccc1Cl)c1cnc2ccc(C3CCNC[C@H]3O)cn12 ; CN1CCC(c2ccc3ncc(C(=O)Nc4ccccc4Cl)n3c2)CC1 ; CC(C)N1CCC(c2ccc3ncc(C(=O)Nc4ccccc4Cl)n3c2)CC1 ; CCN1CCC(c2ccc3ncc(C(=O)Nc4ccccc4Cl)n3c2)CC1]

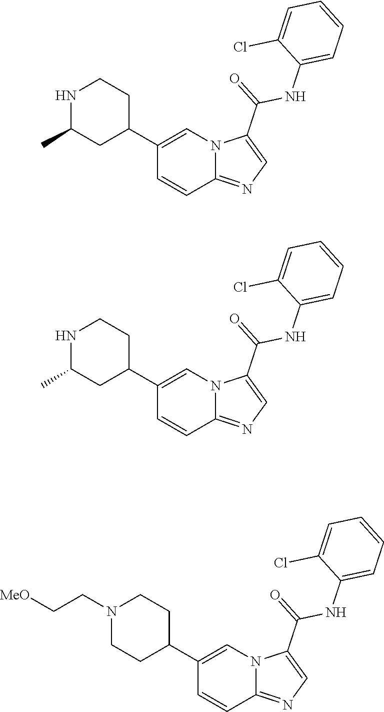 [SMILES: C[C@@H]1CC(c2ccc3ncc(C(=O)Nc4ccccc4Cl)n3c2)CCN1 ; C[C@H]1CC(c2ccc3ncc(C(=O)Nc4ccccc4Cl)n3c2)CCN1 ; COCCN1CCC(c2ccc3ncc(C(=O)Nc4ccccc4Cl)n3c2)CC1]

or a pharmaceutically acceptable salt thereof.

Paragraph 79. A compound of Formula (Ie):

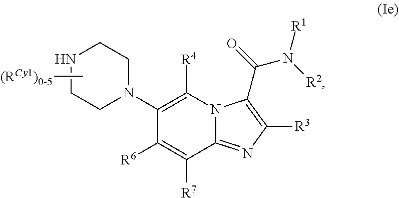

or a pharmaceutically acceptable salt thereof, wherein:
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1}; R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

Paragraph 80. The compound of paragraph 79, wherein R¹ is selected from C₆₋₁₀ aryl and 5-14 membered heteroaryl, each of which is optionally substituted with 1 or 2 substituents independently selected from halo and OR^{a1}.

Paragraph 81. The compound of paragraph 79 or 80, wherein R² is H.

Paragraph 82. The compound of any one of paragraphs 79-81, wherein R³ is selected from H and C₁₋₃ alkyl.

Paragraph 83. The compound of any one of paragraphs 79-82, wherein R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

Paragraph 84. The compound of any one of paragraphs 79-83, wherein R⁴ is selected from H, halo, CN, OR^{a1}, and C₁₋₃ haloalkyl.

Paragraph 85. The compound paragraph 84, wherein R⁴ is H.

Paragraph 86. The compound of any one of paragraphs 79-85, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

Paragraph 87. The compound of paragraph 86, wherein R⁷ is H.

Paragraph 88. The compound of any one of paragraphs 79-87, wherein R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 89. The compound of paragraph 88, wherein R⁶ is H.

Paragraph 90. The compound of paragraph 79, wherein:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R^{CY1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 91. The compound of paragraph 90, wherein R⁴, R⁶, and R⁷ are each H.

Paragraph 92. The compound of paragraph 79, wherein the compound of Formula (Ie) is selected from any one of the following compounds:

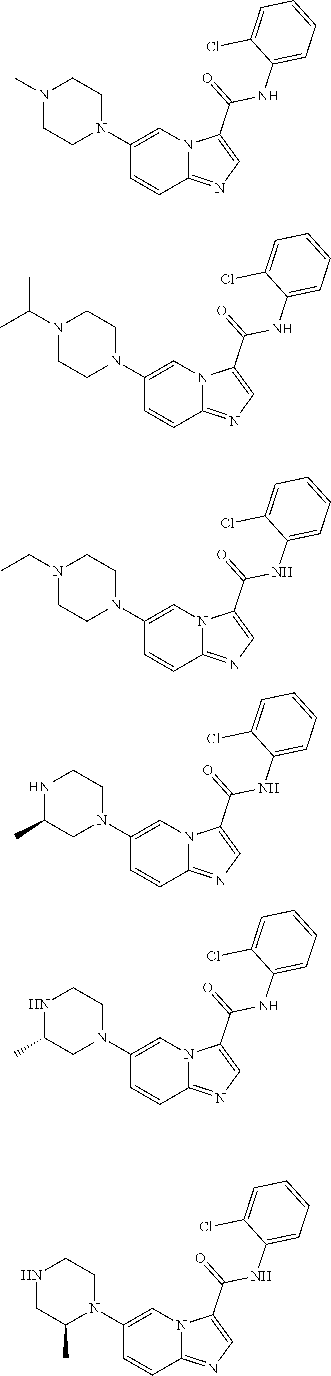

or a pharmaceutically acceptable salt thereof.

Paragraph 93. A compound of Formula (If):

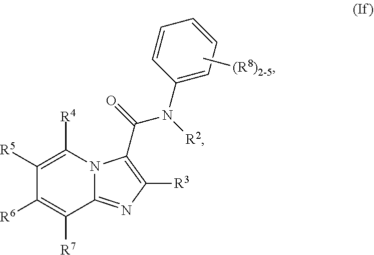

or a pharmaceutically acceptable salt thereof, wherein:
each R⁸ is independently selected from halo, CN, OR^{a1}, C₁₋₃ alkyl, and C₁₋₃ haloalkyl, wherein said C₁₋₃ alkyl is optionally substituted with 1 or 2 independently selected R^{g};
or any two adjacent R⁸ groups together with the carbon atoms to which they are attached from a ring selected from 5-6 membered heteroaryl and 4-6 membered heterocycloalkyl, each of which is optionally substituted with 1, 2, or 3 substituents independently selected from R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

Paragraph 94. The compound of paragraph 93, wherein each R⁸ is independently selected from halo, CN, C₁₋₃ alkoxy, C₁₋₃ alkyl, and C₁₋₃ haloalkyl, wherein said C₁₋₃ alkyl is optionally substituted with R^{g}.

Paragraph 95. The compound of paragraph 93, wherein any two adjacent R⁸ groups together with the carbon atoms to which they are attached form a 5-6 membered heteroaryl ring, optionally substituted with 1 or 2 substituents independently selected from R⁹.

Paragraph 96. The compound of paragraph 93, wherein any two adjacent R⁸ groups together with the carbon atoms to which they are attached form a 4-6 membered heterocycloalkyl, optionally substituted with 1 or 2 substituents independently selected from R⁹.

Paragraph 97. The compound of any one of paragraphs 93-96, wherein R² is H.

Paragraph 98. The compound of any one of paragraphs 93-97, wherein R³ is selected from H and C₁₋₃ alkyl.

Paragraph 99. The compound of any one of paragraphs 93-98, wherein R⁵ is selected from NR^{c1}R^{d1} OR^{a1}, C₁₋₆ alkyl, and Cy¹, wherein said C₁₋₆ alkyl is optionally substituted with NR^{c1}R^{d1}.

Paragraph 100. The compound of any one of paragraphs 93-99, wherein Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

Paragraph 101. The compound of any one of paragraphs 93-100, wherein R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

Paragraph 102. The compound of any one of paragraphs 93-101, wherein R⁴ is selected from H, halo, CN, OR^{a1}, and C₁₋₃ haloalkyl.

Paragraph 103. The compound of paragraph 102, wherein R⁴ is H.

Paragraph 104. The compound of any one of paragraphs 93-103, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

Paragraph 105. The compound of paragraph 104, wherein R⁷ is H.

Paragraph 106. The compound of any one of paragraphs 93-105, wherein R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 107. The compound of paragraph 106, wherein R⁶ is H.

Paragraph 108. The compound of any one of paragraphs 93-96, wherein:
R² is H;
R³ is selected from H and C₁₋₃ alkyl;
R⁵ is selected from NR^{c1}R^{d1} OR^{a1}, C₁₋₆ alkyl, and Cy¹, wherein said C₁₋₆ alkyl is optionally substituted with NR^{c1}R^{d1}; Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 109. The compound of paragraph 108, wherein R⁴, R⁶, and
R⁷ are each H.

Paragraph 110. The compound of the above paragraph, wherein the compound of Formula (If) is selected from any one of the following compounds:

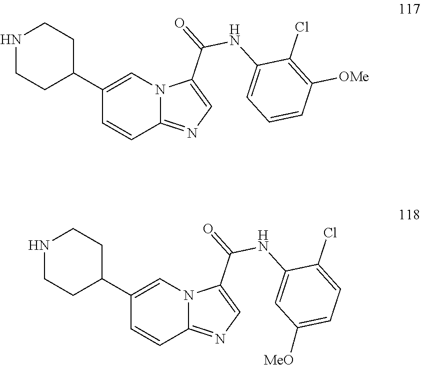 [SMILES: COc1cccc(NC(=O)c2cnc3ccc(C4CCNCC4)cn23)c1Cl ; COc1ccc(Cl)c(NC(=O)c2cnc3ccc(C4CCNCC4)cn23)c1]

or a pharmaceutically acceptable salt thereof.

Paragraph 111. The compound of the above paragraph, wherein the compound of Formula (If) is selected from any one of the following compounds:

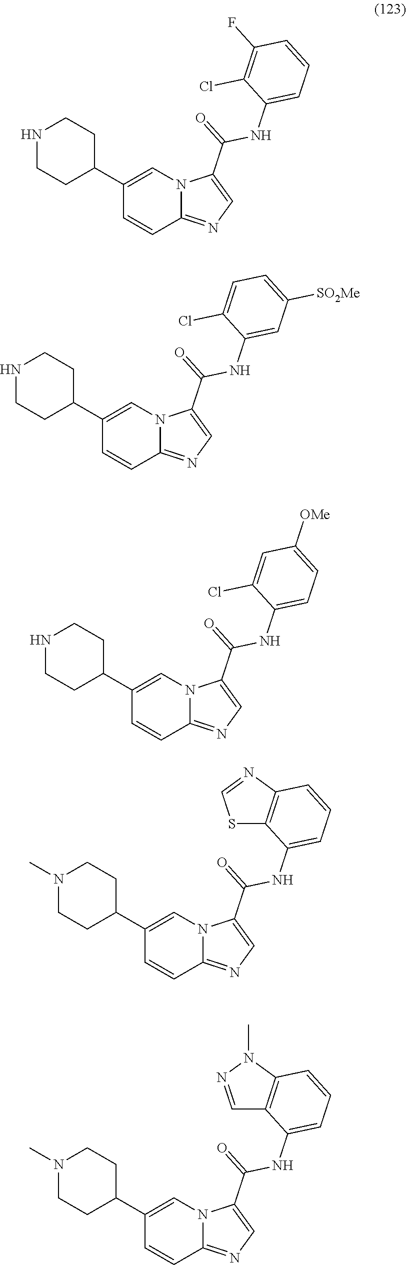 [SMILES: O=C(Nc1cccc(F)c1Cl)c1cnc2ccc(C3CCNCC3)cn12 ; CS(=O)(=O)c1ccc(Cl)c(NC(=O)c2cnc3ccc(C4CCNCC4)cn23)c1 ; COc1ccc(NC(=O)c2cnc3ccc(C4CCNCC4)cn23)c(Cl)c1 ; CN1CCC(c2ccc3ncc(C(=O)Nc4cccc5ncsc45)n3c2)CC1 ; CN1CCC(c2ccc3ncc(C(=O)Nc4cccc5c4cnn5C)n3c2)CC1]

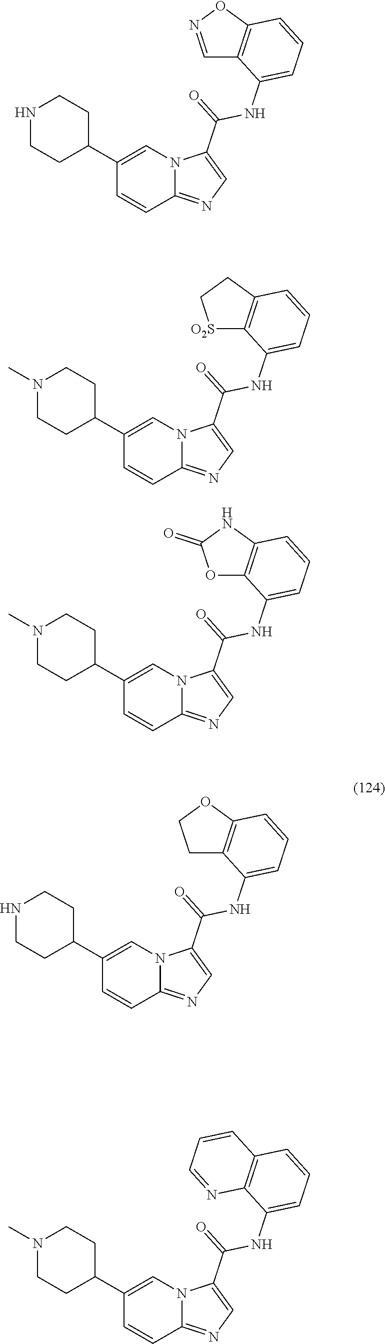 [SMILES: O=C(Nc1cccc2oncc12)c1cnc2ccc(C3CCNCC3)cn12 ; CN1CCC(c2ccc3ncc(C(=O)Nc4cccc5c4S(=O)(=O)CC5)n3c2)CC1 ; CN1CCC(c2ccc3ncc(C(=O)Nc4cccc5[nH]c(=O)oc45)n3c2)CC1 ; O=C(Nc1cccc2c1CCO2)c1cnc2ccc(C3CCNCC3)cn12 ; CN1CCC(c2ccc3ncc(C(=O)Nc4cccc5cccnc45)n3c2)CC1]

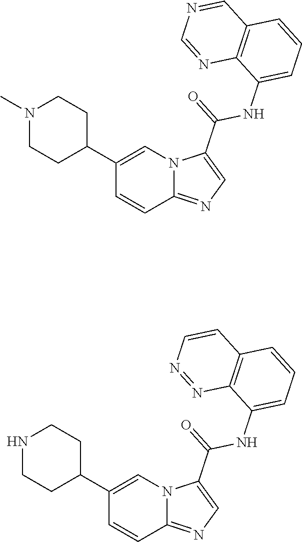 [SMILES: CN1CCC(c2ccc3ncc(C(=O)Nc4cccc5cncnc45)n3c2)CC1 ; O=C(Nc1cccc2ccnnc12)c1cnc2ccc(C3CCNCC3)cn12]

or a pharmaceutically acceptable salt thereof.

Paragraph 112. A compound of Formula (Ig):

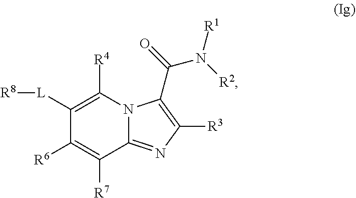

or a pharmaceutically acceptable salt thereof, wherein:
L is selected from C₁₋₆ alkylene, O—C₁₋₆ alkylene, and NH—C₁₋₆ alkylene, each of which is optionally substituted with 1 or 2 independently selected R⁹;
R⁸ is selected from NR^{c1}R^{d1}, C(O)NR^{c1}R^{d1}, and OC(O)NR^{c1}R^{d1};
each R⁹ is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1} and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

Paragraph 113. The compound of paragraph 112, wherein L is selected from C₁₋₆ alkylene, O—C₁₋₆ alkylene, and NH—C₁₋₆ alkylene.

Paragraph 114. The compound of paragraph 112 or 113, wherein R⁸ is NR^{c1}R^{d1}.

Paragraph 115. The compound of any one of paragraphs 112-114, wherein R¹ is selected from C₆₋₁₀ aryl and 5-14 membered heteroaryl, each of which is optionally substituted with 1 or 2 substituents independently selected from halo and OR^{a1}.

Paragraph 116. The compound of any one of paragraphs 112-115, wherein R² is H.

Paragraph 117. The compound of any one of paragraphs 112-116, wherein R³ is selected from H and C₁₋₃ alkyl.

Paragraph 118. The compound of any one of paragraphs 112-117, wherein R⁴ is selected from H, halo, CN, OR^{a1}, and C₁₋₃ haloalkyl.

Paragraph 119. The compound of paragraph 118, wherein R⁴ is H.

Paragraph 120. The compound of any one of paragraphs 112-119, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}.

Paragraph 121. The compound of paragraph 120, wherein R⁷ is H.

Paragraph 122. The compound of any one of paragraphs 112-121, wherein R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 123. The compound of paragraph 122, wherein R⁶ is H.

Paragraph 124. The compound of paragraph 112, wherein: L is selected from C₁₋₆ alkylene, O—C₁₋₆ alkylene, and NH—C₁₋₆ alkylene;
R⁸ is NR^{c1}R^{d1};
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

Paragraph 125. The compound of paragraph 124, wherein R⁴, R⁶, and
R⁷ are each H.

Paragraph 126. The compound of paragraph 112, wherein the compound of Formula (Ig) is selected from any one of the following compounds:

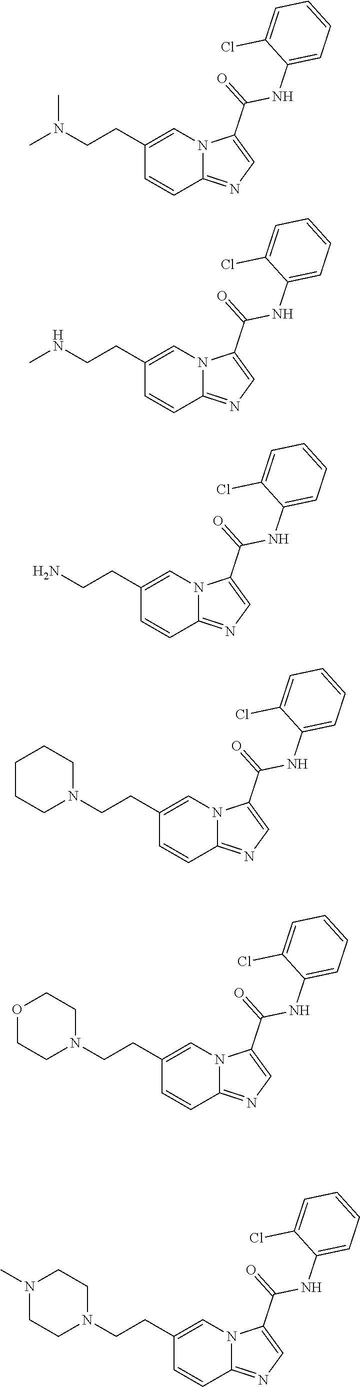 [SMILES: CN(C)CCc1ccc2ncc(C(=O)Nc3ccccc3Cl)n2c1 ; CNCCc1ccc2ncc(C(=O)Nc3ccccc3Cl)n2c1 ; NCCc1ccc2ncc(C(=O)Nc3ccccc3Cl)n2c1 ; O=C(Nc1ccccc1Cl)c1cnc2ccc(CCN3CCCCC3)cn12 ; O=C(Nc1ccccc1Cl)c1cnc2ccc(CCN3CCOCC3)cn12 ; CN1CCN(CCc2ccc3ncc(C(=O)Nc4ccccc4Cl)n3c2)CC1]

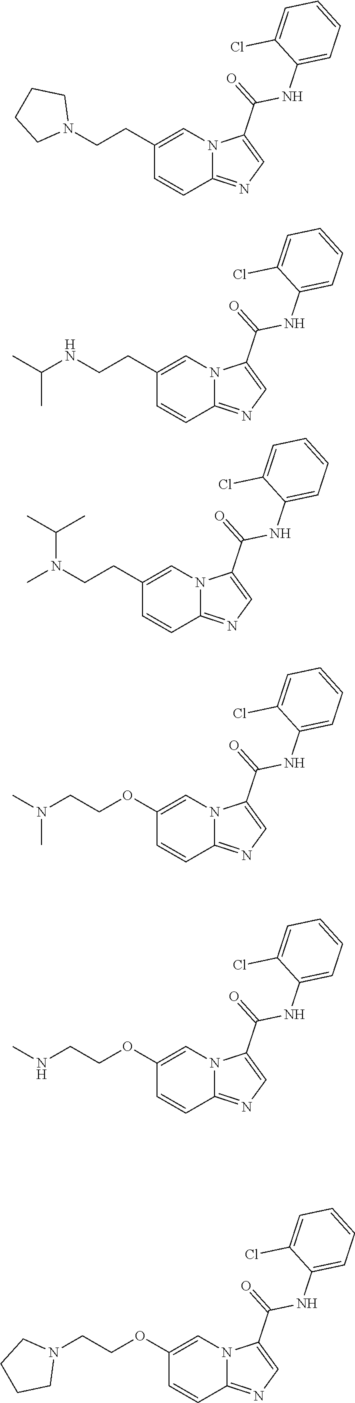

or a pharmaceutically acceptable salt thereof.

Paragraph 127. A compound of Formula (Ih):

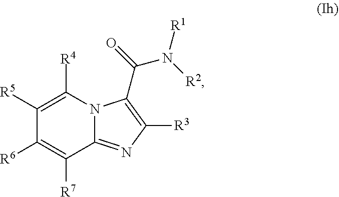

or a pharmaceutically acceptable salt thereof, wherein:
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴, R⁶, and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
provided that at least one of R⁴, R⁶, and R⁷ is selected from halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

Paragraph 128. The compound of paragraph 127, wherein R¹ is selected from C₆₋₁₀ aryl and 5-14 membered heteroaryl, each of which is optionally substituted with 1 or 2 substituents independently selected from halo and OR^{a1}.

Paragraph 129. The compound of paragraph 127 or 128, wherein R² is H.

Paragraph 130. The compound of any one of paragraphs 127-129, wherein R³ is selected from H and C₁₋₃ alkyl.

Paragraph 131. The compound of any one of paragraphs 127-130, wherein R⁵ is selected from NR^{c1}R^{d1} OR^{a1}, C₁₋₆ alkyl, and Cy¹, wherein said C₁₋₆ alkyl is optionally substituted with NR^{c1}R^{d1}.

Paragraph 132. The compound of any one of paragraphs 127-131, wherein Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

Paragraph 133. The compound of any one of paragraphs 127-132, wherein R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

Paragraph 134. The compound of any one of paragraphs 127-135, wherein R⁴, R⁶, and R⁷ are each independently selected from H, halo, CN, C₁₋₃ alkoxy, S(O)₂R^{b1}, S(O)R^{b1}, C(O)NR^{c1}R^{d1}, C(O)OR^{a1}, C₁₋₃ alkyl, C₁₋₃ haloalkyl, and 5-6 membered heteroaryl, wherein said C₁₋₃ alkyl is optionally substituted with R⁹.

Paragraph 135. The compound of paragraph 127, wherein:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is selected from NR^{c1}R^{d1}, OR^{a1}, C₁₋₆ alkyl, and Cy¹, wherein said C₁₋₆ alkyl is optionally substituted with NR^{c1}R^{d1}; Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴, R⁶, and R⁷ are each independently selected from H, halo, CN, OH, C₁₋₃ alkoxy, S(O)₂R^{b1}, S(O)R^{b1}, C(O)NR^{c1}R^{d1}, C(O)OR^{a1}, C₁₋₃ alkyl, C₁₋₃ haloalkyl, and 5-6 membered heteroaryl, wherein said C₁₋₃ alkyl is optionally substituted with R9.

Paragraph 136. The compound of paragraph 127, wherein the compound of Formula (Ih) is selected from any one of the following compounds:

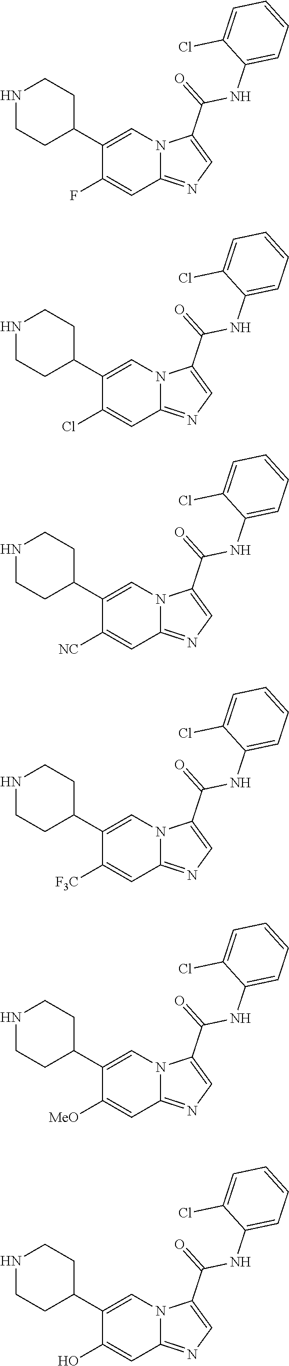 [SMILES: O=C(Nc1ccccc1Cl)c1cnc2cc(F)c(C3CCNCC3)cn12 ; O=C(Nc1ccccc1Cl)c1cnc2cc(Cl)c(C3CCNCC3)cn12 ; N#Cc1cc2ncc(C(=O)Nc3ccccc3Cl)n2cc1C1CCNCC1 ; O=C(Nc1ccccc1Cl)c1cnc2cc(C(F)(F)F)c(C3CCNCC3)cn12 ; COc1cc2ncc(C(=O)Nc3ccccc3Cl)n2cc1C1CCNCC1 ; O=C(Nc1ccccc1Cl)c1cnc2cc(O)c(C3CCNCC3)cn12]

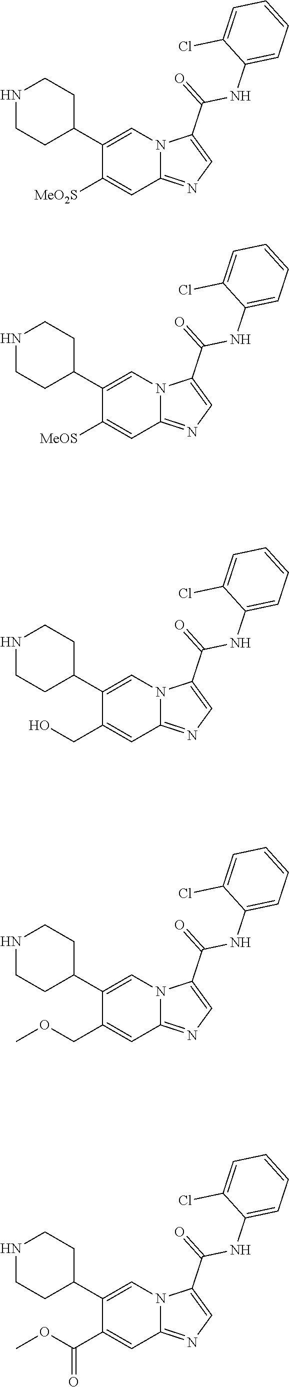

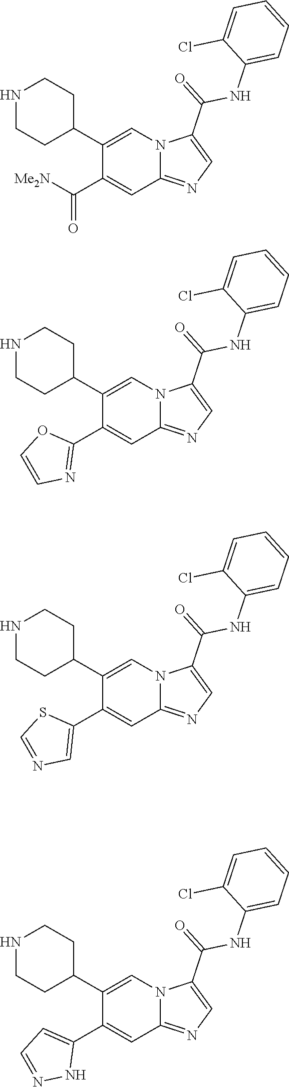 [SMILES: CN(C)C(=O)c1cc2ncc(C(=O)Nc3ccccc3Cl)n2cc1C1CCNCC1 ; O=C(Nc1ccccc1Cl)c1cnc2cc(-c3ncco3)c(C3CCNCC3)cn12 ; O=C(Nc1ccccc1Cl)c1cnc2cc(-c3cncs3)c(C3CCNCC3)cn12 ; O=C(Nc1ccccc1Cl)c1cnc2cc(-c3ccn[nH]3)c(C3CCNCC3)cn12]

or a pharmaceutically acceptable salt thereof.

Paragraph 137. A compound of Formula (Ii):

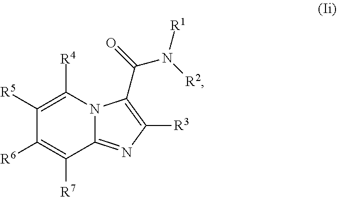

or a pharmaceutically acceptable salt thereof, wherein:
R⁶ is a reactive electrophilic warhead group;
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; or any two R^{c1} and R^{d1}, together with the N atom to which they are attached, for a 4-7 membered heterocycloalkyl ring, which is optionally substituted with 1, 2, or 3 substituents independently selected from R^{g};
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

Paragraph 138. The compound of paragraph 137, wherein R⁶ is a warhead group of formula:

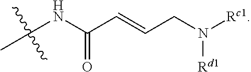

Paragraph 139. The compound of paragraph 138, wherein R⁶ is a warhead group selected from:

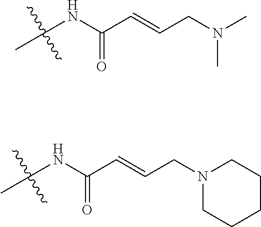

Paragraph 140. The compound of paragraph 137, wherein R⁶ is a

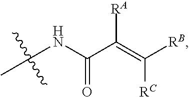

wherein:
R^{A}, R^{B}, and R^{C} are each independently selected from H, halo, CN, and C₁₋₃ alkyl.

Paragraph 141. The compound of paragraph 140, wherein R⁶ is a warhead selected from:

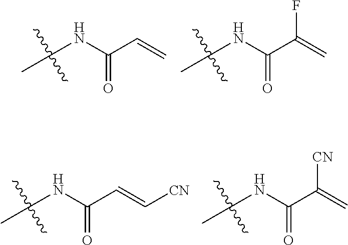

Paragraph 142. The compound of paragraph 137, wherein R⁶ is a warhead group of formula:

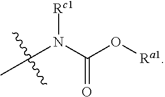

Paragraph 143. The compound of paragraph 142, wherein R⁶ is a warhead selected from:

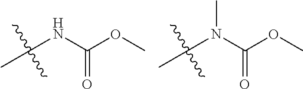

Paragraph 144. The compound of paragraph 137, wherein R⁶ is a

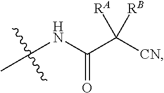

wherein:
R^{A} and R^{B} are each independently selected from H and C₁₋₃ alkyl, or
R^{A} and R^{B}, together with the carbon atom to which they are attached, for a C₃₋₅ cycloalkyl ring.

Paragraph 145. The compound of paragraph 144, wherein R⁶ is a warhead selected from:

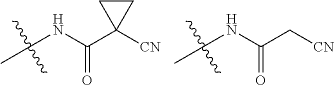 [SMILES: *NC(=O)C1(C#N)CC1 ; *NC(=O)CC#N]

Paragraph 146. The compound of paragraph 137, wherein R⁶ is a warhead group of formula:

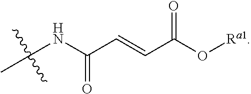

Paragraph 147. The compound of paragraph 137, wherein R⁶ is a warhead group of formula:

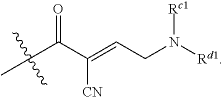

Paragraph 148. The compound of paragraph 137, wherein R⁶ is a warhead group of formula:

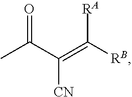

wherein:
R^{A} and R^{B} are each independently selected from H, C(O)NR^{c1}R^{d1}, and C(O)OR^{a1}.

Paragraph 149. The compound of paragraph 148, herein R⁶ is a warhead selected from:

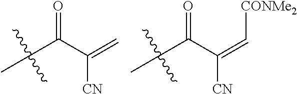

Paragraph 150. The compound of paragraph 137, wherein R⁶ is a warhead of formula:

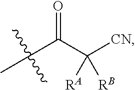

wherein:
R^{A} and R^{B} are each independently selected from H and C₁₋₃ alkyl, or
R^{A} and R^{B}, together with the carbon atom to which they are attached, for a C₃₋₅ cycloalkyl ring.

Paragraph 151. The compound of paragraph 150, wherein R⁶ is a warhead selected from:

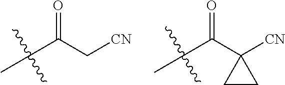 [SMILES: *C(=O)CC#N ; *C(=O)C1(C#N)CC1]

Paragraph 152. The compound of paragraph 137, wherein R⁶ is a warhead of formula:

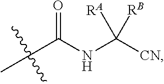

wherein:
R^{A} and R^{B} are each independently selected from H and C₁₋₃ alkyl, or
R^{A} and R^{B}, together with the carbon atom to which they are attached, for a C₃₋₅ cycloalkyl ring.

Paragraph 153. The compound of paragraph 152, wherein R⁶ is a warhead selected from:

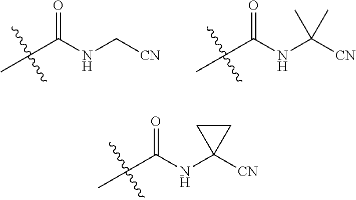

Paragraph 154. The compound of paragraph 137, wherein R⁶ is a

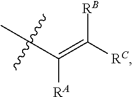

wherein:
R^{A}, R^{B}, and R^{C} are each independently selected from H, CN, C(O)NR^{c1}R^{d1}, and C(O)OR^{a1}, provided that at least one of R^{A}, R^{B}, and R^{C} is selected from CN, C(O)NR^{c1}R^{d1}, and C(O)OR^{a1}.

Paragraph 155. The compound of paragraph 154, wherein R⁶ is a warhead selected from:

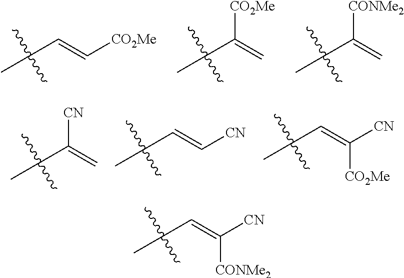

Paragraph 156. The compound of paragraph 137, wherein R⁶ is a warhead selected from a moiety of any one of the following formulae:

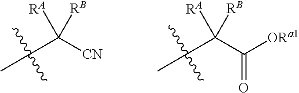

wherein R^{A} and R^{B} are each independently selected from H and C₁₋₃ alkyl.

Paragraph 157. The compound of paragraph 156, wherein R⁶ is a warhead selected from:

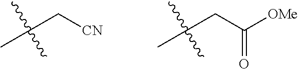

Paragraph 158. The compound of paragraph 137, wherein R⁶ is a warhead of formula:

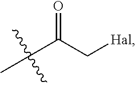

wherein Hal is a halo.

Paragraph 159. The compound of paragraph 158, wherein R⁶ is a warhead selected from:

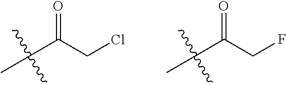 [SMILES: *C(=O)CCl ; *C(=O)CF]

Paragraph 160. The compound of paragraph 137, wherein R⁶ is a warhead of formula:

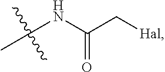

wherein Hal is a halo.

Paragraph 161. The compound of paragraph 160, wherein R⁶ is a warhead selected from:

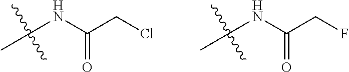 [SMILES: *NC(=O)CCl ; *NC(=O)CF]

Paragraph 162. The compound of paragraph 137, wherein R⁶ is a warhead of formula:

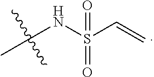 [SMILES: *NS(=O)(=O)C=C]

Paragraph 163. The compound of paragraph 137, wherein R⁶ is a warhead which is a 5-6 membered heteroaryl, which is substituted with a reactive group selected from halo, CN, ethynyl, and vinyl.

Paragraph 164. The compound of paragraph 163, wherein R⁶ is a warhead selected from:

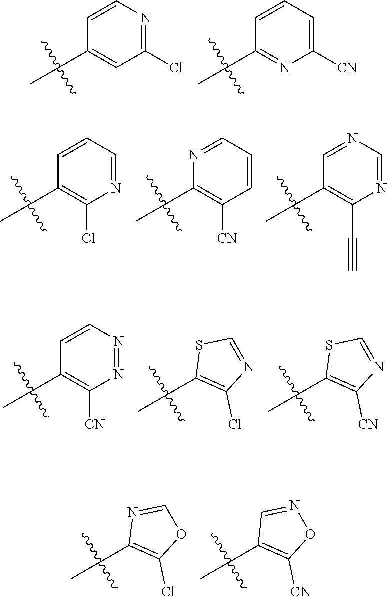

Paragraph 165. The compound of any one of paragraphs 137-164, wherein R¹ is selected from C₆₋₁₀ aryl and 5-14 membered heteroaryl, each of which is optionally substituted with 1 or 2 substituents independently selected from halo and OR^{a1}.

Paragraph 166. The compound of any one of paragraphs 137-165, wherein R² is H.

Paragraph 167. The compound of any one of paragraphs 137-166, wherein R³ is selected from H and C₁₋₃ alkyl.

Paragraph 168. The compound of any one of paragraphs 137-167, wherein R⁵ is selected from NR^{c1}R^{d1}, OR^{a1}, C₁₋₆ alkyl, and Cy¹, wherein said C₁₋₆ alkyl is optionally substituted with NR^{c1}R^{d1}.

Paragraph 169. The compound of any one of paragraphs 137-168, wherein Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

Paragraph 170. The compound of any one of paragraphs 137-169, wherein R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

Paragraph 171. The compound of any one of paragraphs 137-170, wherein R⁴ is selected from H, halo, CN, OR^{a1}, and C₁₋₃ haloalkyl.

Paragraph 172. The compound of paragraph 171, wherein R⁴ is H.

Paragraph 173. The compound of any one of paragraphs 137-172, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

Paragraph 174. The compound of paragraph 173, wherein R⁷ is H.

Paragraph 175. The compound of any one of paragraphs 137-164, wherein:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is selected from NR^{c1}R^{d1} OR^{a1}, C₁₋₆ alkyl, and Cy¹, wherein said C₁₋₆ alkyl is optionally substituted with NR^{c1}R^{d1}; Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl; and
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

Paragraph 176. The compound of paragraph 137, wherein the compound of Formula (Ii) is selected from any one of the following compounds:

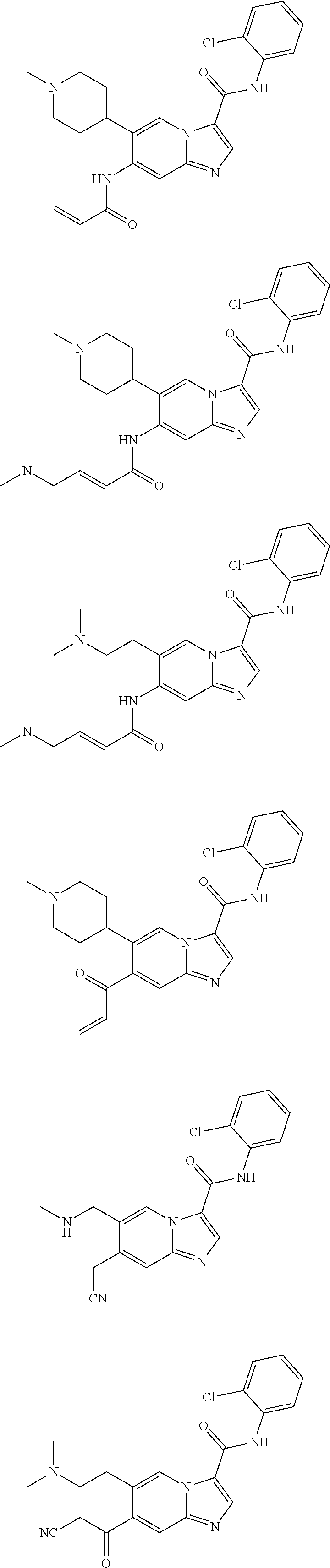 [SMILES: C=CC(=O)Nc1cc2ncc(C(=O)Nc3ccccc3Cl)n2cc1C1CCN(C)CC1 ; CN(C)C/C=C/C(=O)Nc1cc2ncc(C(=O)Nc3ccccc3Cl)n2cc1C1CCN(C)CC1 ; CN(C)C/C=C/C(=O)Nc1cc2ncc(C(=O)Nc3ccccc3Cl)n2cc1CCN(C)C ; C=CC(=O)c1cc2ncc(C(=O)Nc3ccccc3Cl)n2cc1C1CCN(C)CC1 ; CNCc1cn2c(C(=O)Nc3ccccc3Cl)cnc2cc1CC#N ; CN(C)CCc1cn2c(C(=O)Nc3ccccc3Cl)cnc2cc1C(=O)CC#N]

or a pharmaceutically acceptable salt thereof.

Paragraph 177. A method of treating a neurodegenerative or a demyelinating disease or condition, the method comprising administering to a subject in need thereof a therapeutically effective amount of a compound of Formula (II):

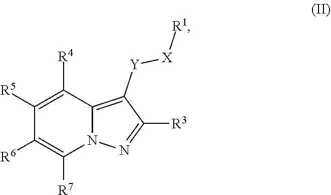

or a pharmaceutically acceptable salt thereof, wherein:
X and Y are each independently selected from C(═O) and NR²;
R¹ is selected from C₆₋₁₀ aryl, C₆₋₁₀ aryl-C₁₋₃ alkyl, 4-10 membered heterocycloalkyl, and 5-14 membered heteroaryl, each of which is optionally substituted with 1, 2, or 3 substituents independently selected from halo, CN, OR^{a1}, S(O)₂R^{b1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}, wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, phenyl, carboxy, C₁₋₆ alkoxycarbonyl, amino, C₁₋₆ alkylamino, and di(C₁₋₆ alkyl)amino.

Paragraph 178. The method of paragraph 177, wherein X is C(═O) and Y is NH.

Paragraph 179. The method of paragraph 177, wherein X is NH and Y is C(═O).

Paragraph 180. The method of any one of paragraphs 177-179, wherein R¹ is selected from C₆₋₁₀ aryl and C₆₋₁₀ aryl-C₁₋₃ alkyl, each of which is optionally substituted with 1 or 2 substituents independently selected from halo, CN, OR^{a1}, S(O)₂R^{b1}, C₁₋₃ alkyl, and C₁₋₃ haloalkyl.

Paragraph 181. The method of any one of paragraphs 177-180, wherein R³ is selected from H and C₁₋₃ alkyl.

Paragraph 182. The method of any one of paragraphs 177-181, wherein R⁵ is selected from halo, NR^{c1}R^{d1}, OR^{a1}, C₁₋₆ alkyl, and Cy¹, wherein said C₁₋₆ alkyl is optionally substituted with NR^{c1}R^{d1}.

Paragraph 183. The method of any one of paragraphs 177-182, wherein Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

Paragraph 184. The method of any one of paragraphs 177-183, wherein R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

Paragraph 185. The method of any one of paragraphs 177-184, wherein R⁴ is selected from H, halo, C₁₋₃ alkyl, CN, OR^{a1}, and C₁₋₃ haloalkyl.

Paragraph 186. The method of any one of paragraphs 177-185, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

Paragraph 187. The method of any one of paragraphs 177-186, wherein R⁶ is selected from H, halo, CN, OR^{a1}, and C₁₋₆ alkyl.

Paragraph 188. The method of any one of paragraphs 177-179, wherein:
R¹ is selected from C₆₋₁₀ aryl and C₆₋₁₀ aryl-C₁₋₃ alkyl, each of which is optionally substituted with 1 or 2 substituents independently selected from halo, CN, OR^{a1}, S(O)₂R^{b1}, C₁₋₃ alkyl, and C₁₋₃ haloalkyl;
R³ is selected from H and C₁₋₃ alkyl;
R⁵ is selected from halo, NR^{c1}R^{d1}, OR^{a1}, C₁₋₆ alkyl, and Cy¹, wherein said C₁₋₆ alkyl is optionally substituted with NR^{c1}R^{d1};
Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, C₁₋₃ alkyl, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, and C₁₋₆ alkyl.

Paragraph 189. The method of paragraph 177, wherein the compound of Formula (II) is selected from any one of the compounds listed in Table C, or a pharmaceutically acceptable salt thereof.

Paragraph 190. A method of treating a neurodegenerative disease or condition, the method comprising administering to a subject in need thereof a therapeutically effective amount of any one of the compounds listed in Table B, or a pharmaceutically acceptable salt thereof.

Paragraph 191. A compound of Formula (IIa):

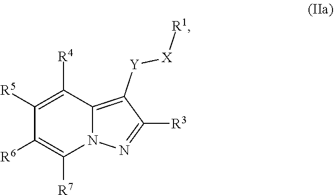

or a pharmaceutically acceptable salt thereof, wherein:
X and Y are each independently selected from C(═O) and NR²;
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from NR^{c1}R^{d1}, 4-6 membered heterocycloalkyl comprising at least one N atom, and L-R⁸; wherein said 4-6 membered heterocycloalkyl comprising at least one N atom is optionally substituted with 1, 2, or 3 substituents independently selected from R^{Cy1};
L is selected from C₁₋₆ alkylene, O—C₁₋₆ alkylene, and NH—C₁₋₆ alkylene, each of which is optionally substituted with 1 or 2 independently selected R⁹;
R⁸ is selected from NR^{c1}R^{d1}, C(O)NR^{c1}R^{d1}, and OC(O)NR^{c1}R^{d1};
each R⁹ is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
R⁴ and R⁷ are each independently selected from H, halo, CN, OR^{a1}, C₁₋₃ alkyl, and C₁₋₃ haloalkyl, wherein said C₁₋₃ alkyl is optionally substituted with 1 or 2 independently selected R^{g};
R⁶ is a reactive electrophilic warhead group;
R^{a1}, R^{b1}, R, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

Paragraph 192. The compound of paragraph 191, wherein X is C(═O) and Y is NH.

Paragraph 193. The compound of paragraph 191, wherein X is NH and Y is C(═O).

Paragraph 194. The compound of any one of paragraphs 191-193, wherein R¹ is selected from C₆₋₁₀ aryl and 5-14 membered heteroaryl, each of which is optionally substituted with 1 or 2 substituents independently selected from halo and OR^{a1}.

Paragraph 195. The compound of any one of paragraphs 191-194, wherein R³ is selected from H and C₁₋₃ alkyl.

Paragraph 196. The compound of any one of paragraphs 191-195, wherein R⁵ is 4-6 membered heterocycloalkyl comprising at least one N atom, which is optionally substituted with 1 or 2 substituents independently selected from R^{Cy1}.

Paragraph 197. The compound of any one of paragraphs 191-195, wherein R⁵ is and L-R⁸.

Paragraph 198. The compound of paragraph 197, wherein L is selected from C₁₋₆ alkylene, O—C₁₋₆ alkylene, and NH—C₁₋₆ alkylene.

Paragraph 199. The compound of paragraph 198, wherein R⁸ is NR^{c1}R^{d1}.

Paragraph 200. The compound of any one of paragraphs 191-196, wherein R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

Paragraph 201. The compound of any one of paragraphs 191-200, wherein R⁴ is selected from H, halo, C₁₋₃ alkyl, CN, OR^{a1}, and C₁₋₃ haloalkyl.

Paragraph 202. The compound of any one of paragraphs 191-201, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

Paragraph 203. The compound of any one of paragraphs 191-193, wherein:
R¹ is selected from C₆₋₁₀ aryl and 5-14 membered heteroaryl, each of which is optionally substituted with 1 or 2 substituents independently selected from halo and OR^{a1};
R³ is selected from H and C₁₋₃ alkyl;
R⁵ is selected from L-R⁸ and 4-6 membered heterocycloalkyl comprising at least one N atom, which is optionally substituted with 1 or 2 substituents independently selected from R^{Cy1};
L is selected from C₁₋₆ alkylene, O—C₁₋₆ alkylene, and NH—C₁₋₆ alkylene;
R⁸ is NR^{c1}R^{d1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, C₁₋₃ alkyl, CN, OR^{a1}, and C₁₋₃ haloalkyl; and
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

Paragraph 204. The compound of paragraph 191, wherein the compound of Formula (IIa) is selected from any one of the following compounds:

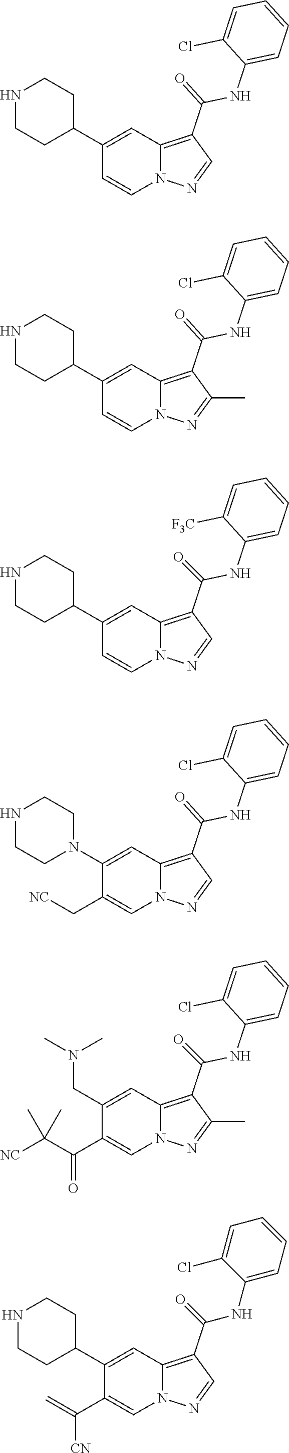 [SMILES: O=C(Nc1ccccc1Cl)c1cnn2ccc(C3CCNCC3)cc12 ; Cc1nn2ccc(C3CCNCC3)cc2c1C(=O)Nc1ccccc1Cl ; O=C(Nc1ccccc1C(F)(F)F)c1cnn2ccc(C3CCNCC3)cc12 ; N#CCc1cn2ncc(C(=O)Nc3ccccc3Cl)c2cc1N1CCNCC1 ; Cc1nn2cc(C(=O)C(C)(C)C#N)c(CN(C)C)cc2c1C(=O)Nc1ccccc1Cl ; C=C(C#N)c1cn2ncc(C(=O)Nc3ccccc3Cl)c2cc1C1CCNCC1]

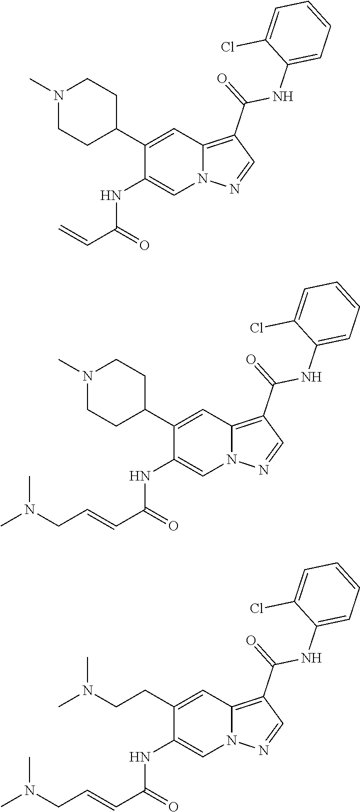 [SMILES: C=CC(=O)Nc1cn2ncc(C(=O)Nc3ccccc3Cl)c2cc1C1CCN(C)CC1 ; CN(C)C/C=C/C(=O)Nc1cn2ncc(C(=O)Nc3ccccc3Cl)c2cc1C1CCN(C)CC1 ; CN(C)C/C=C/C(=O)Nc1cn2ncc(C(=O)Nc3ccccc3Cl)c2cc1CCN(C)C]

or a pharmaceutically acceptable salt thereof.

Paragraph 205. A method of treating a neurodegenerative or a demyelinating disease or condition, the method comprising administering to a subject in need thereof a therapeutically effective amount of a compound of Formula (III):

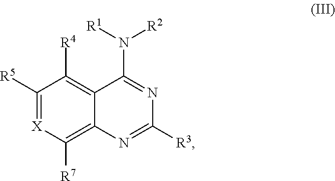

or a pharmaceutically acceptable salt thereof, wherein:
X is selected from CR⁶ and NR⁶;
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}, wherein said C₁₋₆ alkyl is optionally substituted with 1, 2, or 3 independently selected R^{g};
R⁴ and R⁷ are each independently selected from H, halo, CN, OR^{a1}, C₁₋₃ alkyl, and C₁₋₃ haloalkyl, wherein said C₁₋₃ alkyl is optionally substituted with 1 or 2 independently selected R^{g};
R⁶ is selected from H, halo, CN, OR^{a1}, C₁₋₆ alkyl, C₁₋₆ haloalkyl, and a reactive electrophilic warhead group;
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

Paragraph 206. The method of paragraph 205, wherein X is CR⁶.

Paragraph 207. The method of paragraph 205, wherein X is NR⁶.

Paragraph 208. The method of any one of paragraphs 205-207, wherein R¹ is selected from C₆₋₁₀ aryl and 5-14 membered heteroaryl, each of which is optionally substituted with 1 or 2 substituents independently selected from halo and OR^{a1}.

Paragraph 209. The method of any one of paragraphs 205-208, wherein R² is H.

Paragraph 210. The method of any one of paragraphs 205-209, wherein R³ is selected from H and C₁₋₃ alkyl.

Paragraph 211. The method of any one of paragraphs 205-210, wherein R⁵ is selected from NR^{c1}R^{d1}, OR^{a1}, C₁₋₆ alkyl, and Cy¹, wherein said C₁₋₆ alkyl is optionally substituted with NR^{c1}R^{d1}.

Paragraph 212. The method of any one of paragraphs 205-211, wherein Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

Paragraph 213. The method of any one of paragraphs 205-212, wherein R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

Paragraph 214. The method of any one of paragraphs 205-213, wherein R⁴ is selected from H, halo, CN, OR^{a1}, and C₁₋₃ haloalkyl.

Paragraph 215. The method of any one of paragraphs 205-214, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

Paragraph 216. The method of any one of paragraphs 205-207, wherein:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is selected from NR^{c1}R^{d1}, OR^{a1}, C₁₋₆ alkyl, and Cy¹, wherein said C₁₋₆ alkyl is optionally substituted with NR^{c1}R^{d1};
Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl; and
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

Paragraph 217. The method of paragraph 205, wherein the compound of Formula (III) is selected from any one of the compounds listed in Table D, or a pharmaceutically acceptable salt thereof.

Paragraph 218. A compound of Formula (IIIa):

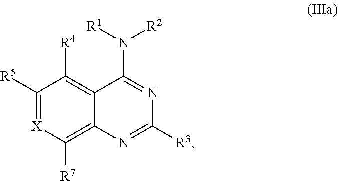

or a pharmaceutically acceptable salt thereof, wherein:
X is selected from CR⁶ and NR⁶
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from NR^{c1}R^{d1} 4-6 membered heterocycloalkyl comprising at least one N atom, and L-R⁸; wherein said 4-6 membered heterocycloalkyl comprising at least one N atom is optionally substituted with 1, 2, or 3 substituents independently selected from R^{Cy1};
L is selected from C₁₋₆ alkylene, O—C₁₋₆ alkylene, and NH—C₁₋₆ alkylene, each of which is optionally substituted with 1 or 2 independently selected R⁹;
R⁸ is selected from NR^{c1}R^{d1}, C(O)NR^{c1}R^{d1}, and OC(O)NR^{c1}R^{d1};
each R⁹ is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
R⁴ and R⁷ are each independently selected from H, halo, CN, OR^{a1}, C₁₋₃ alkyl, and C₁₋₃ haloalkyl, wherein said C₁₋₃ alkyl is optionally substituted with 1 or 2 independently selected R^{g};
R⁶ is a reactive electrophilic warhead group;
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

Paragraph 219. The compound of paragraph 218, wherein X is CR⁶.

Paragraph 220. The compound of paragraph 218, wherein X is NR⁶.

Paragraph 221. The compound of any one of paragraphs 218-220, wherein R¹ is selected from C₆₋₁₀ aryl and 5-14 membered heteroaryl, each of which is optionally substituted with 1 or 2 substituents independently selected from halo and OR^{a1}.

Paragraph 222. The compound of any one of paragraphs 218-221, wherein R² is H.

Paragraph 223. The compound of any one of paragraphs 218-222, wherein R³ is selected from H and C₁₋₃ alkyl.

Paragraph 224. The compound of any one of paragraphs 218-223, wherein R⁵ is 4-6 membered heterocycloalkyl comprising at least one N atom, which is optionally substituted with 1 or 2 substituents independently selected from R^{Cy1}.

Paragraph 225. The compound of any one of paragraphs 218-223, wherein R⁵ is and L-R⁸.

Paragraph 226. The compound of paragraph 225, wherein L is selected from C₁₋₆ alkylene, O—C₁₋₆ alkylene, and NH—C₁₋₆ alkylene.

Paragraph 227. The compound of paragraph 226, wherein R⁸ is NR^{c1}R^{d1}.

Paragraph 228. The compound of any one of paragraphs 218-224, wherein R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

Paragraph 229. The compound of any one of paragraphs 218-228, wherein R⁴ is selected from H, halo, C₁₋₃ alkyl, CN, OR^{a1}, and C₁₋₃ haloalkyl.

Paragraph 230. The compound of any one of paragraphs 218-229, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

Paragraph 231. The compound of any one of paragraphs 218-220, wherein:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is selected from L-R⁸ and 4-6 membered heterocycloalkyl comprising at least one N atom, which is optionally substituted with 1 or 2 substituents independently selected from R^{Cy1};
L is selected from C₁₋₆ alkylene, O—C₁₋₆ alkylene, and NH—C₁₋₆ alkylene;
R⁸ is NR^{c1}R^{d1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, C₁₋₃ alkyl, CN, OR^{a1}, and C₁₋₃ haloalkyl; and
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

Paragraph 232. The compound of paragraph 218, wherein the compound of Formula (IIIa) is selected from any one of the following compounds:

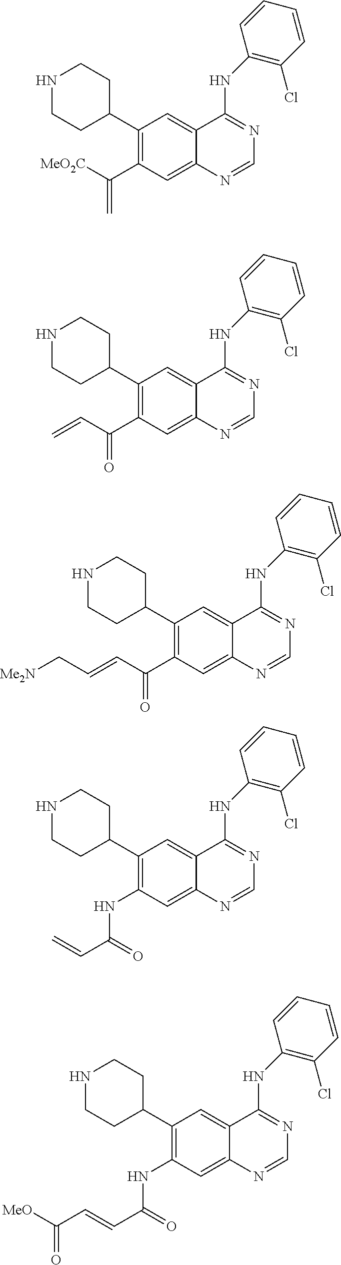 [SMILES: C=C(C(=O)OC)c1cc2ncnc(Nc3ccccc3Cl)c2cc1C1CCNCC1 ; C=CC(=O)c1cc2ncnc(Nc3ccccc3Cl)c2cc1C1CCNCC1 ; CN(C)C/C=C/C(=O)c1cc2ncnc(Nc3ccccc3Cl)c2cc1C1CCNCC1 ; C=CC(=O)Nc1cc2ncnc(Nc3ccccc3Cl)c2cc1C1CCNCC1 ; COC(=O)/C=C/C(=O)Nc1cc2ncnc(Nc3ccccc3Cl)c2cc1C1CCNCC1]

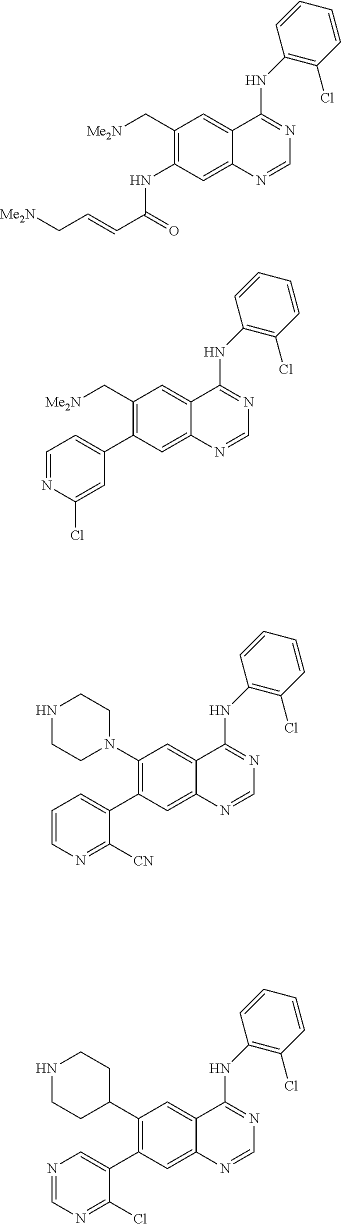 [SMILES: CN(C)C/C=C/C(=O)Nc1cc2ncnc(Nc3ccccc3Cl)c2cc1CN(C)C ; CN(C)Cc1cc2c(Nc3ccccc3Cl)ncnc2cc1-c1ccnc(Cl)c1 ; N#Cc1ncccc1-c1cc2ncnc(Nc3ccccc3Cl)c2cc1N1CCNCC1 ; Clc1ccccc1Nc1ncnc2cc(-c3cncnc3Cl)c(C3CCNCC3)cc12]

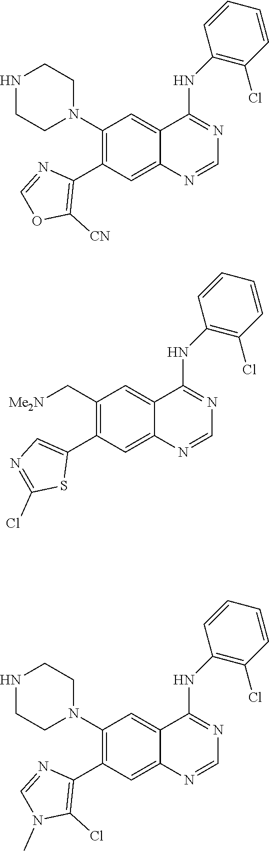 [SMILES: N#Cc1ocnc1-c1cc2ncnc(Nc3ccccc3Cl)c2cc1N1CCNCC1 ; CN(C)Cc1cc2c(Nc3ccccc3Cl)ncnc2cc1-c1cnc(Cl)s1 ; Cn1cnc(-c2cc3ncnc(Nc4ccccc4Cl)c3cc2N2CCNCC2)c1Cl]

or a pharmaceutically acceptable salt thereof.

Paragraph 233. The method of any one of paragraphs 1-17, 177-190, or 205-217, wherein the neurodegenerative or a demyelinating disease or condition is selected from autoimmune encephalomyelitis, chronic inflammatory demyelinating polyneuropathy, multiple sclerosis, amyotrophic lateral sclerosis, schizophrenia, Alzheimer's disease, Parkinson's disease, acute disseminated encephalomyelitis (“ADEM”), concentric sclerosis, Charcot-Marie-Tooth disease, Guillain-Barre syndrome, HTLV-J associated myelopathy (“HAM”), neuromyelitis optica, Schilder's disease, transverse myelitis, dementia, frontotemporal lobar dementia, Huntington's disease, accessory nerve disorder, autonomic dysreflexia, peripheral neuropathy, chemotherapy-induced peripheral neuropathies, mononeuropathy, polyneuropathy, radial neuropathy, ulnar neuropathy, Villaret's syndrome, diabetic neuropathy, nerve paralysis, progressive bulbar palsy, pseudobulbar palsy, spinal bulbar muscular atrophy, myotonic dystrophy, inclusion body myositis, prion disease, seizure disorders, lysosomal storage disorders, transmissible spongiform encephalopathy, Creutzfeldt-Jacob disease (CJD), spinocerebellar ataxia, spinal muscular atrophy, Horner's syndrome, adrenoleukodystrophy, macular degeneration, glaucoma, optic neuritis, and Lewy Body syndrome.

Paragraph 234. The method of paragraph 233, wherein the disease or condition is autoimmune encephalomyelitis.

Paragraph 235. The method of paragraph 233, wherein the disease or condition is multiple sclerosis.

Paragraph 236. A pharmaceutical composition comprising a compounds of any one of paragraphs 18-176, 191-204, or 218-232, or a pharmaceutically acceptable salt thereof, and a pharmaceutically acceptable carrier.

Paragraph 237. A method of treating a neuronal system injury characterized by EphB3 kinase activity, the method comprising administering to a subject in need thereof a therapeutically effective amount of a compound of any one of paragraphs 18-176, 191-204, or 218-232, or a pharmaceutically acceptable salt thereof, or a pharmaceutical composition of paragraph 236.

Paragraph 238. The method of paragraph 237, wherein the neuronal system injury is a central nervous system injury.

Paragraph 239. The method of paragraph 238, wherein the central nervous system injury is selected from cerebral ischemia and traumatic brain injury.

Paragraph 240. A method of treating a cancer, the method comprising administering to a subject in need thereof a therapeutically effective amount of a compound of any one of paragraphs 18-176, 191-204, or 218-232, or a pharmaceutically acceptable salt thereof, or a pharmaceutical composition of paragraph 236.

Paragraph 241. The method of paragraph 240, wherein the cancer is selected from leukemia, non-small cell lung cancer, colon cancer, CNS cancer, melanoma, ovarian cancer, renal cancer, prostate cancer, and breast cancer.

Paragraph 242. A method selected from:
inhibiting EphB3 receptor signaling in an astrocyte cell of a subject; and/or
inhibiting EphB3 tyrosine kinase in an astrocyte cell of a subject; and/or
inhibiting pro-inflammatory response in an astrocyte cell of a subject; and/or
inhibiting production of TNF-α in an astrocyte cell of a subject; and/or
reducing production of IL6 in an astrocyte cell of a subject; and/or
reducing production of CCl2 in an astrocyte cell of a subject; and/or
inhibiting phosphorylation of AKT in astrocyte cell of a subject; and/or
inhibiting activation of mTOR pathway in an astrocyte cell of a subject; and/or
inhibiting production of mitochondrial reactive oxygen species (ROS) in an astrocyte cell of a subject; and/or
restoring a homeostatic state or a normal activation state of glial cell or a neuron cell of a subject; and/or
inhibiting pro-inflammatory response in a microglial cell of a subject; and/or
inhibiting NFkB activation in a microglial cell of a subject; and/or
inhibiting production of TNF-α in a microglial cell of a subject; and/or
inhibiting demyelination of a neuron cell in a subject; and/or
reducing pro-inflammatory T cells in a central nervous system of a subject; and/or
reducing pro-inflammatory macrophages in a central nervous system of a subject; and/or
reducing pro-inflammatory macrophages in a peripheral nervous system of a subject; and/or
inhibiting CNS inflammation in a subject;
inhibiting PNS inflammation in a subject; and/or
inhibiting neurodegeneration in a subject,
the method comprising administering to the subject a therapeutically effective amount of a compound of any one of paragraphs 18-176, 191-204, or 218-232, or a pharmaceutically acceptable salt thereof, or a pharmaceutical composition of paragraph 236.

Paragraph 243. A method of treating a neurodegenerative or a demyelinating disease or condition, the method comprising administering to a subject in need thereof a therapeutically effective amount of a compound of any one of paragraphs 18-176, 191-204, or 218-232, or a pharmaceutically acceptable salt thereof, or a pharmaceutical composition of paragraph 236.

Paragraph 244. The method of paragraph 243, wherein the disease or condition is selected from autoimmune encephalomyelitis, chronic inflammatory demyelinating polyneuropathy, acute disseminated encephalomyelitis, multiple sclerosis, amyotrophic lateral sclerosis, schizophrenia, Alzheimer's disease, Parkinson's disease, acute disseminated encephalomyelitis (“ADEM”), concentric sclerosis, Charcot-Marie-Tooth disease, Guillain-Barre syndrome, HTLV-I associated myelopathy (“HAM”), neuromyelitis optica, Schilder's disease, and transverse myelitis.

Paragraph 245. The method of paragraph 244, wherein the disease or condition is autoimmune encephalomyelitis.

Paragraph 246. The method of paragraph 244, wherein the disease or condition is multiple sclerosis.

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A method of treating a neurodegenerative or a demyelinating disease or condition, the method comprising administering to a subject in need thereof a therapeutically effective amount of a compound of Formula (I): 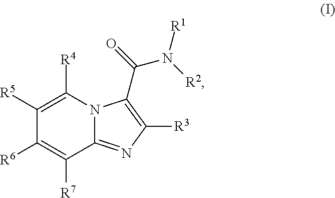
or a pharmaceutically acceptable salt thereof, wherein:
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is optionally substituted with 1, 2, or 3 substituents independently selected from R^{Cy2};
each R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}, wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₃ alkyl, and C₁₋₃ haloalkyl, wherein said C₁₋₃ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

2. The method of claim 1, wherein R¹ is selected from C₆₋₁₀ aryl and 5-14 membered heteroaryl, each of which is optionally substituted with 1 or 2 substituents independently selected from halo and OR^{a1}.

3. The method of claim 1 or 2, wherein R³ is selected from H and C₁₋₃ alkyl.

4. The method of any one of claims 1-3, wherein R⁵ is selected from NR^{c1}R^{d1}, OR^{a1}, C₁₋₆ alkyl, and Cy¹, wherein said C₁₋₆ alkyl is optionally substituted with NR^{c1}R^{d1}.

5. The method of any one of claims 1-4, wherein Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1}.

6. The method of any one of claims 1-5, wherein R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1}.

7. The method of any one of claims 1-6, wherein R⁴ is selected from H, halo, CN, OR^{a1}, and C₁₋₃ haloalkyl.

8. The method of any one of claims 1-7, wherein R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R⁹.

9. The method of any one of claims 1-8, wherein R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

10. The method of claim 1, wherein:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is selected from NR^{c1}R^{d1} OR^{a1}, C₁₋₆ alkyl, and Cy¹, wherein said C₁₋₆ alkyl is optionally substituted with NR^{c1}R^{d1};
Cy¹ is selected from C₆₋₁₀ aryl, 5-14 membered heteroaryl, and 4-10 membered heterocycloalkyl, each of which is optionally substituted with 1, 2, or 3 substituents independently selected R^{Cy1};
R^{Cy1} is selected from halo, OR^{a1}, C(O)R^{b1}, C(O)NR^{c1}R^{d1}, and C₁₋₆ alkyl, wherein said C₁₋₆ alkyl is optionally substituted with 1, 2, or 3 substituents independently selected from OR^{a1} and NR^{c1}R^{d1};
R⁴ is selected from H, halo, CN, OR^{a1}, and C₁₋₃ haloalkyl;
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and 5-14 membered heteroaryl, wherein said C₁₋₃ alkyl is optionally substituted with 1 or 2 independently selected R^{g}; and
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1}.

11. The method of claim 10, wherein R⁴, R⁶, and R⁷ are each H.

12. The method of claim 1, wherein the compound of Formula (I) is selected from any one of the following compounds 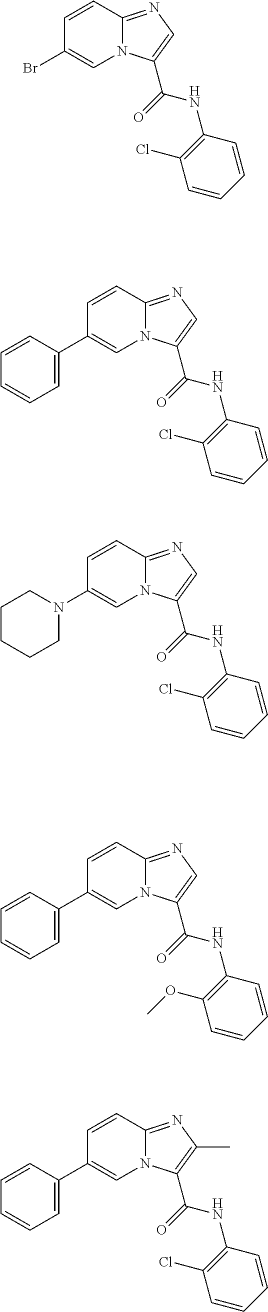 [SMILES: O=C(Nc1ccccc1Cl)c1cnc2ccc(Br)cn12 ; O=C(Nc1ccccc1Cl)c1cnc2ccc(-c3ccccc3)cn12 ; O=C(Nc1ccccc1Cl)c1cnc2ccc(N3CCCCC3)cn12 ; COc1ccccc1NC(=O)c1cnc2ccc(-c3ccccc3)cn12 ; Cc1nc2ccc(-c3ccccc3)cn2c1C(=O)Nc1ccccc1Cl] 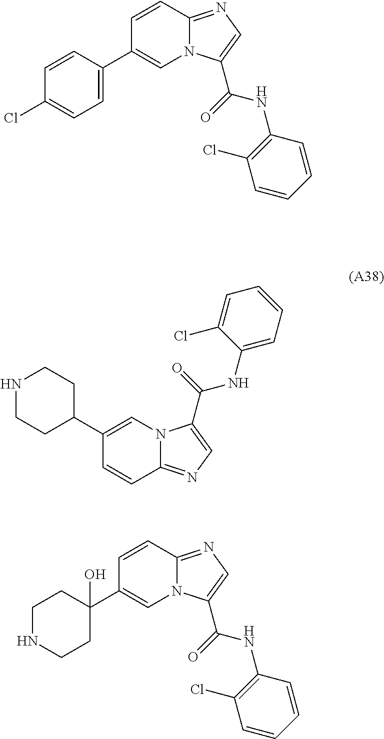 [SMILES: O=C(Nc1ccccc1Cl)c1cnc2ccc(-c3ccc(Cl)cc3)cn12 ; O=C(Nc1ccccc1Cl)c1cnc2ccc(C3CCNCC3)cn12 ; O=C(Nc1ccccc1Cl)c1cnc2ccc(C3(O)CCNCC3)cn12]
or a pharmaceutically acceptable salt thereof.

13. The method of claim 1, wherein the compound of Formula (I) is selected from any one of the following compounds:

| No. | Structure |
|---|---|
| 101 | 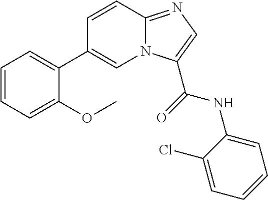 [SMILES: COc1ccccc1-c1ccc2ncc(C(=O)Nc3ccccc3Cl)n2c1] |
| 102 | 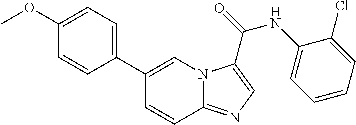 [SMILES: COc1ccc(-c2ccc3ncc(C(=O)Nc4ccccc4Cl)n3c2)cc1] |
| 103 | 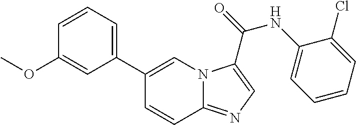 [SMILES: COc1cccc(-c2ccc3ncc(C(=O)Nc4ccccc4Cl)n3c2)c1] |
| 104 | 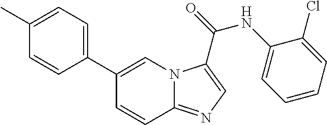 [SMILES: Cc1ccc(-c2ccc3ncc(C(=O)Nc4ccccc4Cl)n3c2)cc1] |
| 105 | 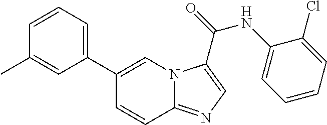 [SMILES: Cc1cccc(-c2ccc3ncc(C(=O)Nc4ccccc4Cl)n3c2)c1] |
| 106 | 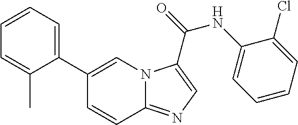 [SMILES: Cc1ccccc1-c1ccc2ncc(C(=O)Nc3ccccc3Cl)n2c1] |
| 107 | 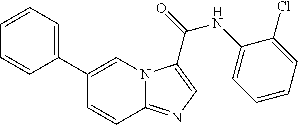 [SMILES: O=C(Nc1ccccc1Cl)c1cnc2ccc(-c3ccccc3)cn12] |
| 108 | 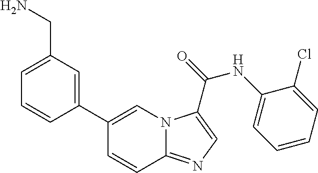 [SMILES: NCc1cccc(-c2ccc3ncc(C(=O)Nc4ccccc4Cl)n3c2)c1] |
| 109 | 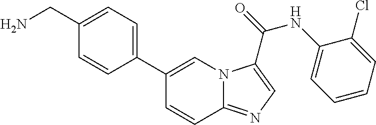 [SMILES: NCc1ccc(-c2ccc3ncc(C(=O)Nc4ccccc4Cl)n3c2)cc1] |
| 110 | 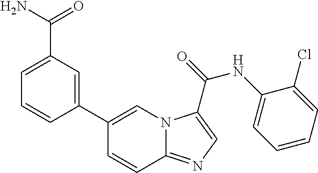 [SMILES: NC(=O)c1cccc(-c2ccc3ncc(C(=O)Nc4ccccc4Cl)n3c2)c1] |
| 111 | 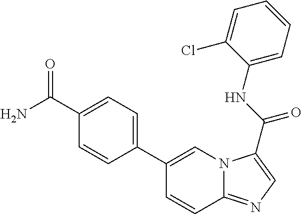 [SMILES: NC(=O)c1ccc(-c2ccc3ncc(C(=O)Nc4ccccc4Cl)n3c2)cc1] |
| 112 | 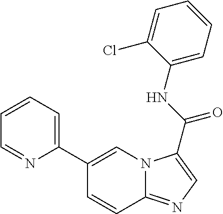 [SMILES: O=C(Nc1ccccc1Cl)c1cnc2ccc(-c3ccccn3)cn12] |
| 113 | 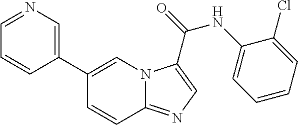 [SMILES: O=C(Nc1ccccc1Cl)c1cnc2ccc(-c3cccnc3)cn12] |
| 114 | 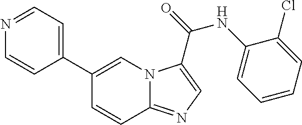 [SMILES: O=C(Nc1ccccc1Cl)c1cnc2ccc(-c3ccncc3)cn12] |
| 115 | 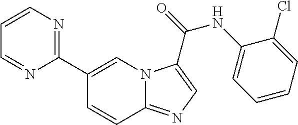 [SMILES: O=C(Nc1ccccc1Cl)c1cnc2ccc(-c3ncccn3)cn12] |
| 116 | 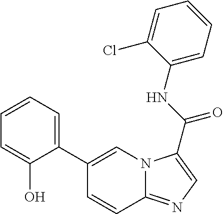 [SMILES: O=C(Nc1ccccc1Cl)c1cnc2ccc(-c3ccccc3O)cn12] |
| 117 | 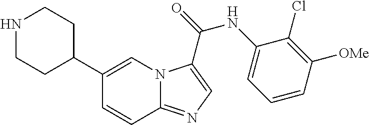 [SMILES: COc1cccc(NC(=O)c2cnc3ccc(C4CCNCC4)cn23)c1Cl] |
| 118 | 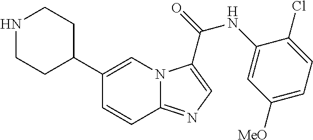 [SMILES: COc1ccc(Cl)c(NC(=O)c2cnc3ccc(C4CCNCC4)cn23)c1] |
| 119 | 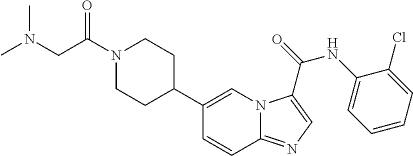 [SMILES: CN(C)CC(=O)N1CCC(c2ccc3ncc(C(=O)Nc4ccccc4Cl)n3c2)CC1] |
| 120 | 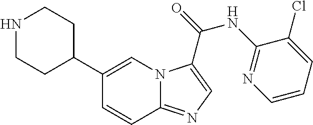 [SMILES: O=C(Nc1ncccc1Cl)c1cnc2ccc(C3CCNCC3)cn12] |
| 121 | 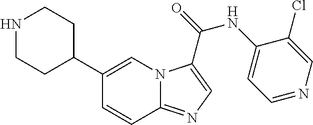 [SMILES: O=C(Nc1ccncc1Cl)c1cnc2ccc(C3CCNCC3)cn12] |
| 122 | 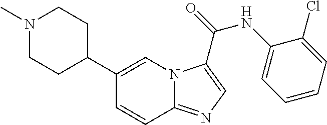 [SMILES: CN1CCC(c2ccc3ncc(C(=O)Nc4ccccc4Cl)n3c2)CC1] |
| 123 | 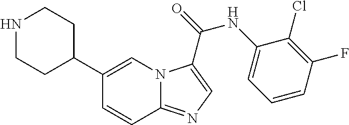 [SMILES: O=C(Nc1cccc(F)c1Cl)c1cnc2ccc(C3CCNCC3)cn12] |
| 124 | 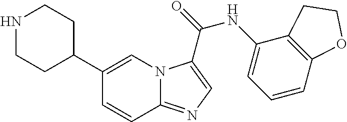 [SMILES: O=C(Nc1cccc2c1CCO2)c1cnc2ccc(C3CCNCC3)cn12] |

pharmaceutically acceptable salt thereof.

14. The method of any one of claims 1-13, wherein the neurodegenerative or a demyelinating disease or condition is selected from autoimmune encephalomyelitis, chronic inflammatory demyelinating polyneuropathy, multiple sclerosis, amyotrophic lateral sclerosis, schizophrenia, Alzheimer's disease, Parkinson's disease, acute disseminated encephalomyelitis (“ADEM”), concentric sclerosis, Charcot-Marie-Tooth disease, Guillain-Barre syndrome, HTLV-I associated myelopathy (“HAM”), neuromyelitis optica, Schilder's disease, transverse myelitis, dementia, frontotemporal lobar dementia, Huntington's disease, accessory nerve disorder, autonomic dysreflexia, peripheral neuropathy, chemotherapy-induced peripheral neuropathies, mononeuropathy, polyneuropathy, radial neuropathy, ulnar neuropathy, Villaret's syndrome, diabetic neuropathy, nerve paralysis, progressive bulbar palsy, pseudobulbar palsy, spinal bulbar muscular atrophy, myotonic dystrophy, inclusion body myositis, prion disease, seizure disorders, lysosomal storage disorders, transmissible spongiform encephalopathy, Creutzfeldt-Jacob disease (CJD), spinocerebellar ataxia, spinal muscular atrophy, Horner's syndrome, adrenoleukodystrophy, macular degeneration, glaucoma, optic neuritis, and Lewy Body syndrome.

15. The method of claim 14, wherein the disease or condition is autoimmune encephalomyelitis.

16. The method of claim 14, wherein the disease or condition is multiple sclerosis.

17. A compound of Formula (Ia): 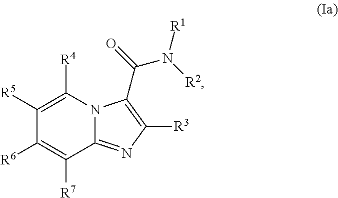
or a pharmaceutically acceptable salt thereof, wherein:
R⁵ is 5-14 membered heteroaryl, optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, C₁₋₆ alkyl, and C₁₋₆ haloalkyl, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, and C₁₋₃ haloalkyl, wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

18. The compound of claim 17, wherein:
R¹ is selected from C₆₋₁₀ aryl and 5-14 membered heteroaryl, each of which is optionally substituted with 1 or 2 substituents independently selected from halo and OR^{a1};
R² is H;
R³ is selected from H and C₁₋₃ alkyl;
R⁵ is 5-6 membered heteroaryl, optionally substituted with 1, 2, or 3 substituents independently selected from OR^{a1}, C(O)R^{b1}, C(O)NR^{c1}R^{d1}, and C₁₋₆ alkyl, wherein said C₁₋₆ alkyl is optionally substituted with OR^{a1} or NR^{c1}R^{d1};
R⁶ is selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, 5-14 membered heteroaryl, and a reactive electrophilic warhead group, wherein said C₁₋₃ alkyl is optionally substituted with OR^{a1} or NR^{c1}R^{d1};
R⁴ is selected from H, halo, CN, OR^{a1}, and C₁₋₃ haloalkyl; and
R⁷ is selected from H, halo, CN, OR^{a1}, S(O)₂R^{b1}, S(O)R^{b1}, C(O)NR^{c1}R^{d1}, C(O)OR^{a1}, C₁₋₃ alkyl, and C₁₋₃ haloalkyl, wherein said C₁₋₃ alkyl is optionally substituted with 1 or 2 independently selected R^{g}.

19. The compound of claim 17, wherein the compound of Formula (Ia) is selected from any one of the following compounds: 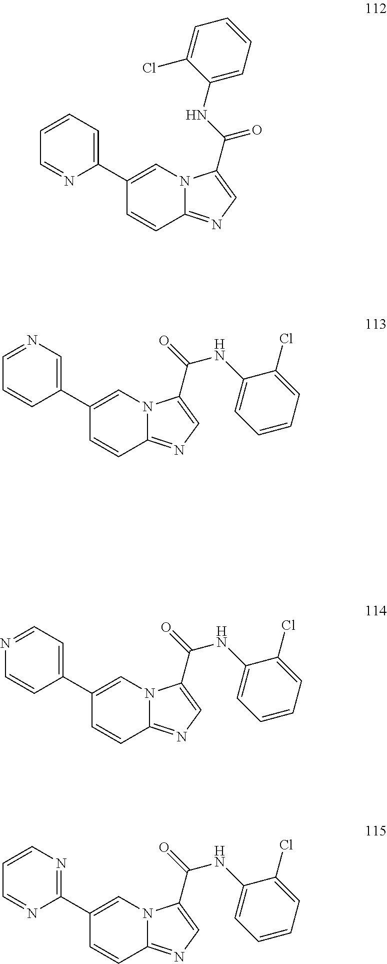 [SMILES: O=C(Nc1ccccc1Cl)c1cnc2ccc(-c3ccccn3)cn12 ; O=C(Nc1ccccc1Cl)c1cnc2ccc(-c3cccnc3)cn12 ; O=C(Nc1ccccc1Cl)c1cnc2ccc(-c3ccncc3)cn12 ; O=C(Nc1ccccc1Cl)c1cnc2ccc(-c3ncccn3)cn12]
or a pharmaceutically acceptable salt thereof.

20. The compound of claim 17, wherein the compound of Formula (Ia) is selected from any one of the following compounds: 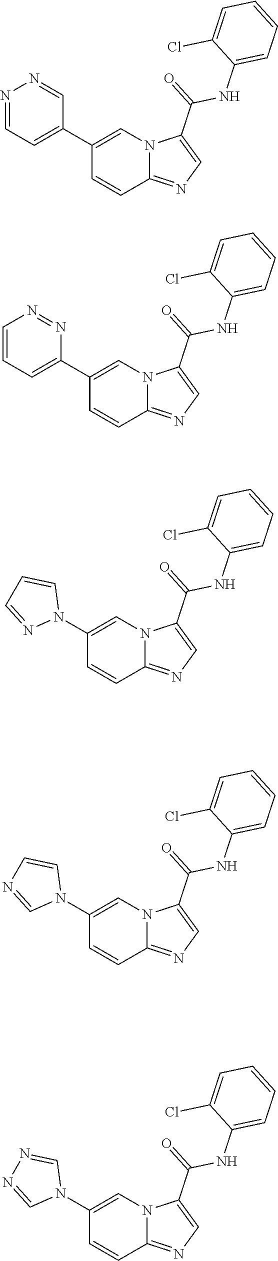 [SMILES: O=C(Nc1ccccc1Cl)c1cnc2ccc(-c3ccnnc3)cn12 ; O=C(Nc1ccccc1Cl)c1cnc2ccc(-c3cccnn3)cn12 ; O=C(Nc1ccccc1Cl)c1cnc2ccc(-n3cccn3)cn12 ; O=C(Nc1ccccc1Cl)c1cnc2ccc(-n3ccnc3)cn12 ; O=C(Nc1ccccc1Cl)c1cnc2ccc(-n3cnnc3)cn12] 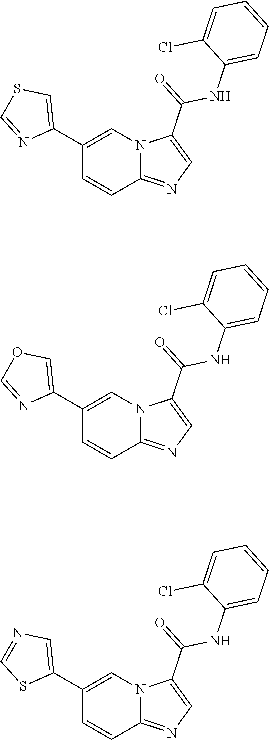 [SMILES: O=C(Nc1ccccc1Cl)c1cnc2ccc(-c3cscn3)cn12 ; O=C(Nc1ccccc1Cl)c1cnc2ccc(-c3cocn3)cn12 ; O=C(Nc1ccccc1Cl)c1cnc2ccc(-c3cncs3)cn12]
or a pharmaceutically acceptable salt thereof.

21. A compound of Formula (Ib): 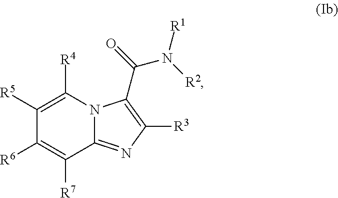
or a pharmaceutically acceptable salt thereof, wherein:
R¹ is selected from 5-14 membered heteroaryl and 4-10 membered heterocycloalkyl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

22. The compound of claim 21, wherein the compound of Formula (Ib) is selected from any one of the following compounds 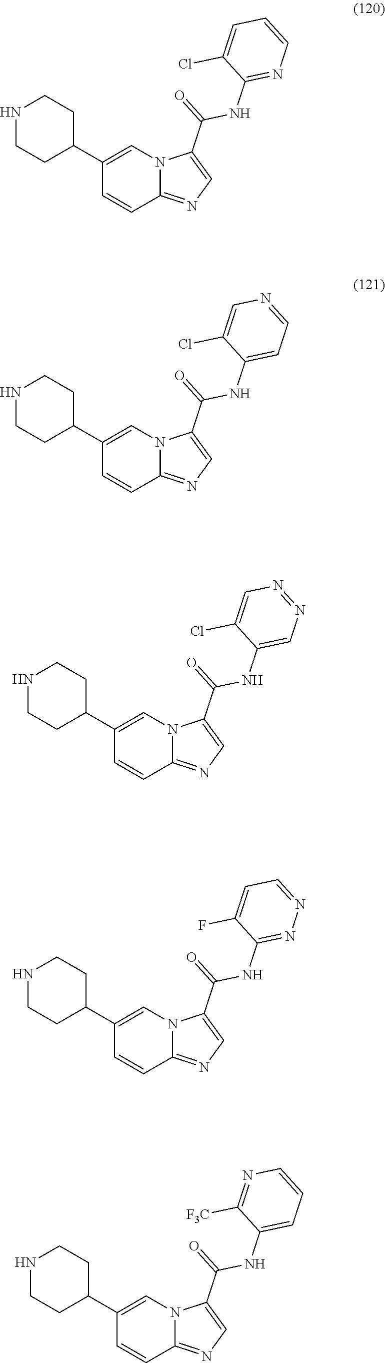 [SMILES: O=C(Nc1ncccc1Cl)c1cnc2ccc(C3CCNCC3)cn12 ; O=C(Nc1ccncc1Cl)c1cnc2ccc(C3CCNCC3)cn12 ; O=C(Nc1cnncc1Cl)c1cnc2ccc(C3CCNCC3)cn12 ; O=C(Nc1nnccc1F)c1cnc2ccc(C3CCNCC3)cn12 ; O=C(Nc1cccnc1C(F)(F)F)c1cnc2ccc(C3CCNCC3)cn12] 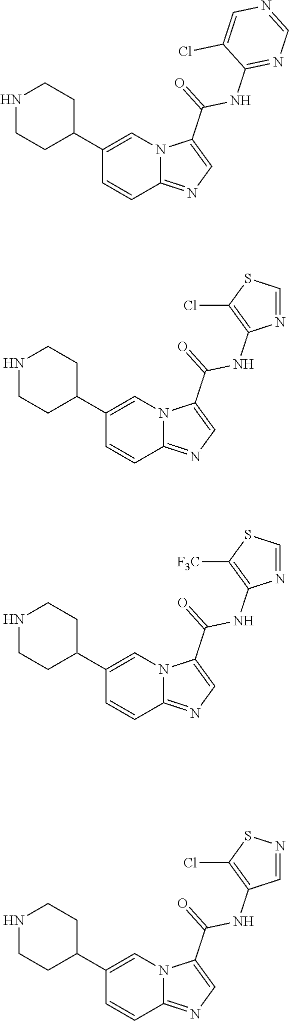 [SMILES: O=C(Nc1ncncc1Cl)c1cnc2ccc(C3CCNCC3)cn12 ; O=C(Nc1ncsc1Cl)c1cnc2ccc(C3CCNCC3)cn12 ; O=C(Nc1ncsc1C(F)(F)F)c1cnc2ccc(C3CCNCC3)cn12 ; O=C(Nc1cnsc1Cl)c1cnc2ccc(C3CCNCC3)cn12]
or a pharmaceutically acceptable salt thereof.

23. A compound of Formula (Ic): 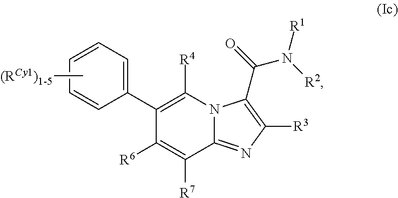
or a pharmaceutically acceptable salt thereof, wherein:
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino, provided that the compound is not:

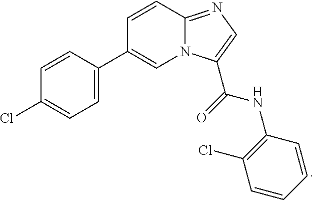 [SMILES: O=C(Nc1ccccc1Cl)c1cnc2ccc(-c3ccc(Cl)cc3)cn12]

24. The compound of claim 23, wherein the compound is selected from any one of the following compounds: 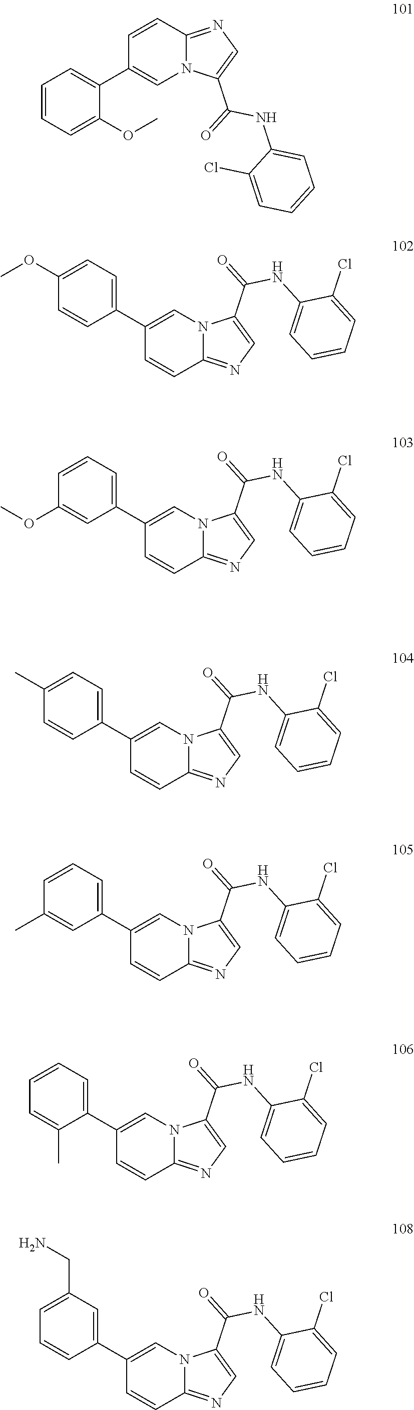 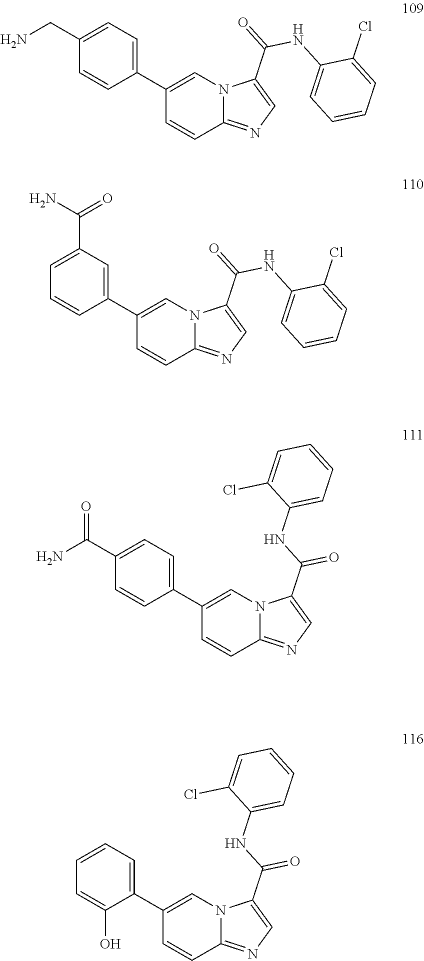 [SMILES: NCc1ccc(-c2ccc3ncc(C(=O)Nc4ccccc4Cl)n3c2)cc1 ; NC(=O)c1cccc(-c2ccc3ncc(C(=O)Nc4ccccc4Cl)n3c2)c1 ; NC(=O)c1ccc(-c2ccc3ncc(C(=O)Nc4ccccc4Cl)n3c2)cc1 ; O=C(Nc1ccccc1Cl)c1cnc2ccc(-c3ccccc3O)cn12]
or a pharmaceutically acceptable salt thereof.

25. The compound of claim 23, wherein the compound is selected from any one of the following compounds: 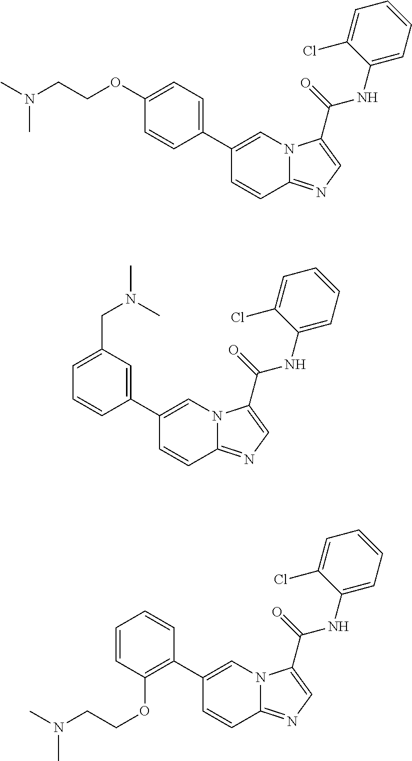 [SMILES: CN(C)CCOc1ccc(-c2ccc3ncc(C(=O)Nc4ccccc4Cl)n3c2)cc1 ; CN(C)Cc1cccc(-c2ccc3ncc(C(=O)Nc4ccccc4Cl)n3c2)c1 ; CN(C)CCOc1ccccc1-c1ccc2ncc(C(=O)Nc3ccccc3Cl)n2c1]
or a pharmaceutically acceptable salt thereof.

26. A compound of Formula (Id): 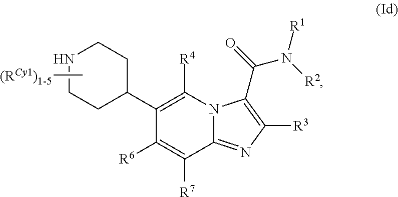
or a pharmaceutically acceptable salt thereof, wherein:
R¹ is selected from C₆₋₁₀ aryl, 4-10 membered heterocycloalkyl, and 5-14 membered heteroaryl, each of which is optionally substituted with 1, 2, or 3 substituents independently selected from halo, CN, OR^{a1}, C₁₋₃ alkyl, and C₁₋₃ haloalkyl, wherein said C₁₋₃ alkyl is optionally substituted with 1 or 2 independently selected R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino, provided that the compound is not:

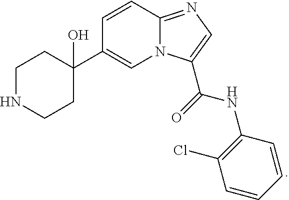 [SMILES: O=C(Nc1ccccc1Cl)c1cnc2ccc(C3(O)CCNCC3)cn12]

27. The compound of claim 26, wherein the compound of Formula (Id) is: 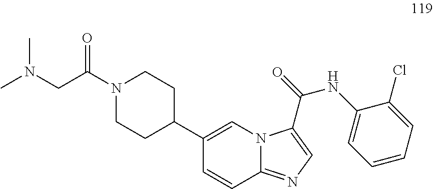 [SMILES: CN(C)CC(=O)N1CCC(c2ccc3ncc(C(=O)Nc4ccccc4Cl)n3c2)CC1]
or a pharmaceutically acceptable salt thereof.

28. The compound of claim 26, wherein the compound of Formula (Id) is selected from any one of the following compounds: 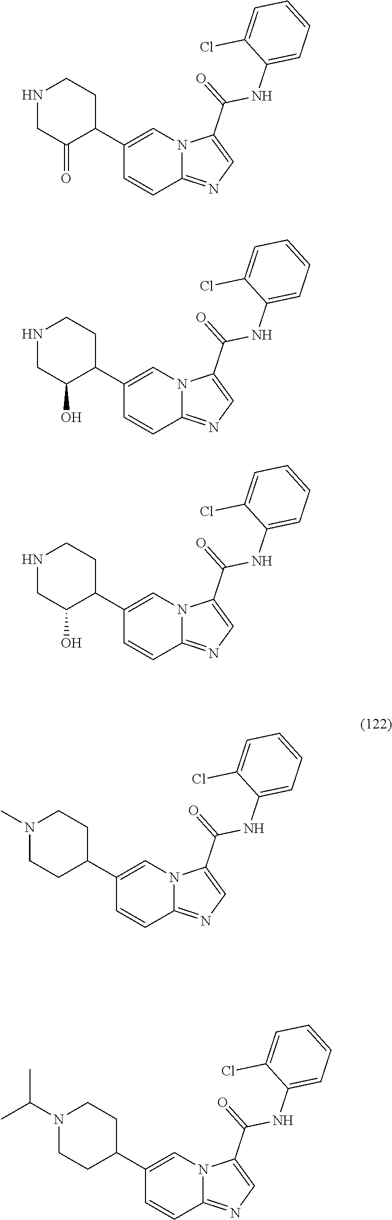 [SMILES: O=C(Nc1ccccc1Cl)c1cnc2ccc(C3CCNCC3=O)cn12 ; O=C(Nc1ccccc1Cl)c1cnc2ccc(C3CCNC[C@@H]3O)cn12 ; O=C(Nc1ccccc1Cl)c1cnc2ccc(C3CCNC[C@H]3O)cn12 ; CN1CCC(c2ccc3ncc(C(=O)Nc4ccccc4Cl)n3c2)CC1 ; CC(C)N1CCC(c2ccc3ncc(C(=O)Nc4ccccc4Cl)n3c2)CC1] 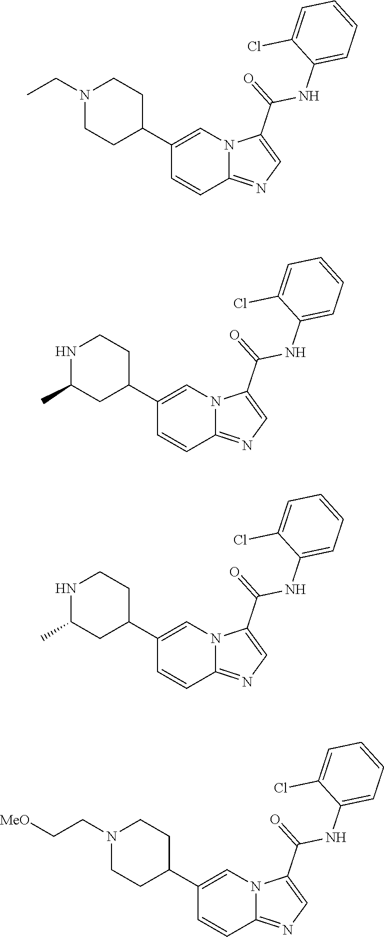 [SMILES: CCN1CCC(c2ccc3ncc(C(=O)Nc4ccccc4Cl)n3c2)CC1 ; C[C@@H]1CC(c2ccc3ncc(C(=O)Nc4ccccc4Cl)n3c2)CCN1 ; C[C@H]1CC(c2ccc3ncc(C(=O)Nc4ccccc4Cl)n3c2)CCN1 ; COCCN1CCC(c2ccc3ncc(C(=O)Nc4ccccc4Cl)n3c2)CC1]
or a pharmaceutically acceptable salt thereof.

29. A compound of Formula (If): 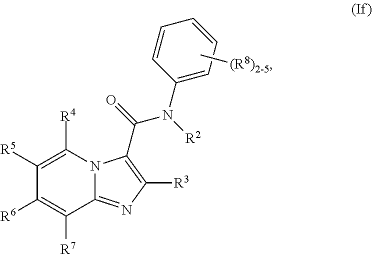
or a pharmaceutically acceptable salt thereof, wherein:
each R⁸ is independently selected from halo, CN, OR^{a1}, C₁₋₃ alkyl, and C₁₋₃ haloalkyl, wherein said C₁₋₃ alkyl is optionally substituted with 1 or 2 independently selected R^{g};
or any two adjacent R⁸ groups together with the carbon atoms to which they are attached from a ring selected from 5-6 membered heteroaryl and 4-6 membered heterocycloalkyl, each of which is optionally substituted with 1, 2, or 3 substituents independently selected from R^{g};
R² is selected from H and C₁₋₃ alkyl;
R³ is selected from H, C₁₋₃ alkyl, and C₁₋₃ haloalkyl, wherein said C₁₋₃ alkyl is optionally substituted with 1 or 2 independently selected R^{g};
R⁵ is selected from H, halo, CN, OR^{a1}, NR^{c1}R^{d1}, C₁₋₆ alkyl, C₁₋₆ haloalkyl, and Cy¹, wherein said C₁₋₆ alkyl is optionally substituted with 1, 2, or 3 substituents independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy¹ is independently selected from C₆₋₁₀ aryl, 5-14 membered heteroaryl, C₃₋₁₀ cycloalkyl, and 4-10 membered heterocycloalkyl, each of which is optionally substituted with 1, 2, 3, 4, or 5 substituents independently selected R^{Cy1};
each R^{Cy1} is independently selected from halo, CN, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, Cy², C₁₋₆ alkyl, C₁₋₆ haloalkyl, and oxo, wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1};
each Cy² is independently selected from C₆₋₁₀ aryl, 5-14 membered heteroaryl, C₃₋₁₀ cycloalkyl, and 4-10 membered heterocycloalkyl, each of which is independently selected from 1, 2, or 3 substituents independently selected from R^{Cy2};
R^{Cy2} is independently selected from halo, CN, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1} wherein said C₁₋₆ alkyl is optionally substituted with 1, 2, or 3 independently selected R^{g};
R⁴ and R⁷ are each independently selected from H, halo, CN, OR^{a1}, S(O)₂R^{b1}, S(O)R^{b1}, C(O)NR^{c1}R^{d1}, C(O)OR^{a1}, C₁₋₃ alkyl, C₁₋₃ haloalkyl, and Cy², wherein said C₁₋₃ alkyl is optionally substituted with 1 or 2 independently selected R^{g};
R⁶ is selected from H, halo, CN, OR^{a1}, S(O)₂R^{b1}, S(O)R^{b1}, C(O)NR^{c1}R^{d1}, C(O)OR^{a1}, C₁₋₆ alkyl, C₁₋₃ haloalkyl, Cy², and a reactive electrophilic warhead group, wherein said C₁₋₆ alkyl is optionally substituted with 1, 2, or 3 substituents independently selected from halo, CN, OR^{a1}, and NR^{c1}R^{d1};
R^{a1}, R^{b1}, R^{c1}, and R^{d1} are each independently selected from H, C₁₋₆ alkyl, and C₁₋₄ haloalkyl, wherein said C₁₋₆ alkyl is optionally substituted with 1, 2, or 3 substituents independently selected from R^{g}; and
each R^{g} is independently selected from halo, CN, OH, C₁₋₃ alkoxy, C₁₋₃ haloalkoxy, carboxy, C₁₋₆ alkoxycarbonyl, amino, C₁₋₆ alkylamino, and di(C₁₋₆ alkyl)amino.

30. The compound of claim 29, wherein the compound of Formula (If) is selected from any one of the following compounds: 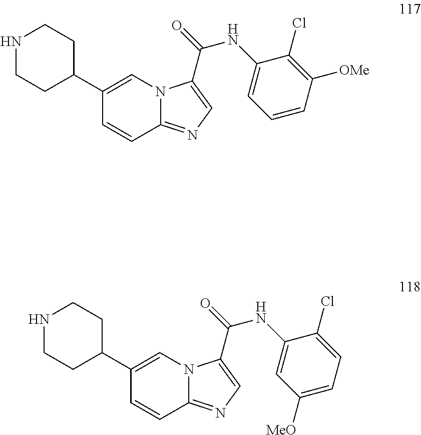 [SMILES: COc1cccc(NC(=O)c2cnc3ccc(C4CCNCC4)cn23)c1Cl ; COc1ccc(Cl)c(NC(=O)c2cnc3ccc(C4CCNCC4)cn23)c1]
or a pharmaceutically acceptable salt thereof.

31. The compound of the above claim, wherein the compound of Formula (If) is selected from any one of the following compounds: 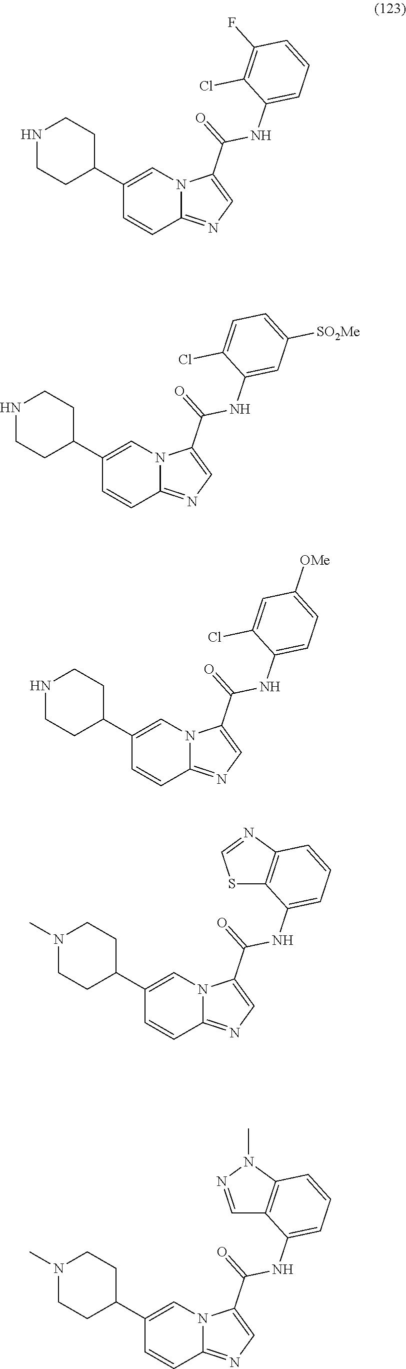 [SMILES: O=C(Nc1cccc(F)c1Cl)c1cnc2ccc(C3CCNCC3)cn12 ; CS(=O)(=O)c1ccc(Cl)c(NC(=O)c2cnc3ccc(C4CCNCC4)cn23)c1 ; COc1ccc(NC(=O)c2cnc3ccc(C4CCNCC4)cn23)c(Cl)c1 ; CN1CCC(c2ccc3ncc(C(=O)Nc4cccc5ncsc45)n3c2)CC1 ; CN1CCC(c2ccc3ncc(C(=O)Nc4cccc5c4cnn5C)n3c2)CC1] 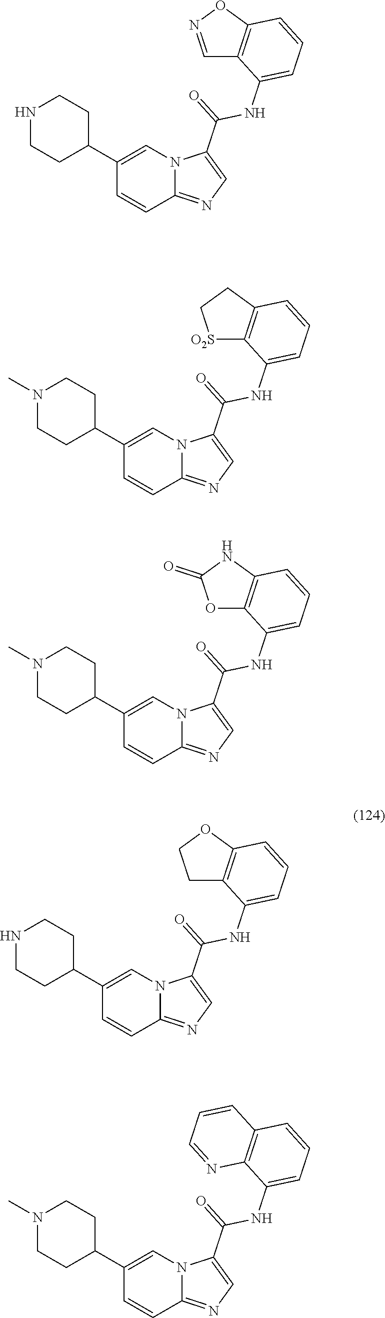 [SMILES: O=C(Nc1cccc2oncc12)c1cnc2ccc(C3CCNCC3)cn12 ; CN1CCC(c2ccc3ncc(C(=O)Nc4cccc5c4S(=O)(=O)CC5)n3c2)CC1 ; CN1CCC(c2ccc3ncc(C(=O)Nc4cccc5[nH]c(=O)oc45)n3c2)CC1 ; O=C(Nc1cccc2c1CCO2)c1cnc2ccc(C3CCNCC3)cn12 ; CN1CCC(c2ccc3ncc(C(=O)Nc4cccc5cccnc45)n3c2)CC1] 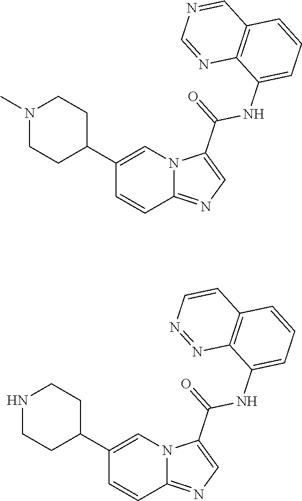 [SMILES: CN1CCC(c2ccc3ncc(C(=O)Nc4cccc5cncnc45)n3c2)CC1 ; O=C(Nc1cccc2ccnnc12)c1cnc2ccc(C3CCNCC3)cn12]
or a pharmaceutically acceptable salt thereof.

32. A pharmaceutical composition comprising a compounds of any one of claims 17-31, or a pharmaceutically acceptable salt thereof, and a pharmaceutically acceptable carrier.

33. A method of treating a neurodegenerative or a demyelinating disease or condition, the method comprising administering to a subject in need thereof a therapeutically effective amount of a compound of any one of claims 17-31, or a pharmaceutically acceptable salt thereof.

34. The method of claim 33, wherein the neurodegenerative or a demyelinating disease or condition is selected from autoimmune encephalomyelitis, chronic inflammatory demyelinating polyneuropathy, multiple sclerosis, amyotrophic lateral sclerosis, schizophrenia, Alzheimer's disease, Parkinson's disease, acute disseminated encephalomyelitis (“ADEM”), concentric sclerosis, Charcot-Marie-Tooth disease, Guillain-Barre syndrome, HTLV-I associated myelopathy (“HAM”), neuromyelitis optica, Schilder's disease, transverse myelitis, dementia, frontotemporal lobar dementia, Huntington's disease, accessory nerve disorder, autonomic dysreflexia, peripheral neuropathy, chemotherapy-induced peripheral neuropathies, mononeuropathy, polyneuropathy, radial neuropathy, ulnar neuropathy, Villaret's syndrome, diabetic neuropathy, nerve paralysis, progressive bulbar palsy, pseudobulbar palsy, spinal bulbar muscular atrophy, myotonic dystrophy, inclusion body myositis, prion disease, seizure disorders, lysosomal storage disorders, transmissible spongiform encephalopathy, Creutzfeldt-Jacob disease (CJD), spinocerebellar ataxia, spinal muscular atrophy, Horner's syndrome, adrenoleukodystrophy, macular degeneration, glaucoma, optic neuritis, and Lewy Body syndrome.

35. The method of claim 34, wherein the disease or condition is autoimmune encephalomyelitis or multiple sclerosis.
